(19) United States
(12) Patent Application Publication
(10) Pub. No.: US 2023/0271965 A1
(43) Pub. Date: Aug. 31, 2023

(54) BICYCLIC HETEROARENES AND METHODS OF THEIR USE

(71) Applicant: Kineta, Inc., Seattle, WA (US)

(72) Inventors: Kerem OZBOYA, Cambridge, MA (US); Iwona WRONA, Sharon, MA (US); Bertrand LE BOURDONNEC, Northborough, MA (US); Matthew LUCAS, Lexington, MA (US); Vanessa KURIA, Somerville, MA (US); Madeline MACDONNELL, Brighton, MA (US); Byron DELABARRE, Arlington, MA (US)

(21) Appl. No.: 18/007,797

(22) PCT Filed: Jun. 3, 2021
(86) PCT No.: PCT/US21/35701

§ 371 Date: Dec. 2, 2022

Related U.S. Application Data
(60) Provisional application No. 63/034,283, filed on Jun. 3, 2020.

(51) Int. Cl. C07D 487/04; C07D 498/04; C07D 519/00

(52) U.S. Cl. CPC C07D 487/04; C07D 498/04; C07D 519/00

(57) ABSTRACT

Disclosed are compounds useful in the treatment of neurological disorders. The compounds described herein, alone or in combination with other pharmaceutically active agents, can be used for treating or preventing neurological diseases.

### FIELD OF THE INVENTION

The invention relates to bicyclic heteroarenes and their use for therapeutic treatment of neurological disorders in patients, such as human patients.

### BACKGROUND

An incomplete understanding of the molecular perturbations that cause disease, as well as a limited arsenal of robust model systems, has contributed to a failure to generate successful disease-modifying therapies against common and progressive neurological disorders, such as ALS and FTD. Progress is being made on many fronts to find agents that can arrest the progress of these disorders. However, the present therapies for most, if not all, of these diseases provide very little relief. Accordingly, a need exists to develop therapies that can alter the course of neurodegenerative diseases. More generally, a need exists for better methods and compositions for the treatment of neurodegenerative diseases in order to improve the quality of the lives of those afflicted by such diseases.

### SUMMARY

TDP-43 is a nuclear DNA/RNA binding protein involved in RNA splicing. Under pathological cell stress, TDP-43 translocates to the cytoplasm and aggregates into stress granules and related protein inclusions. These phenotypes are hallmarks of degenerating motor neurons and are found in 97% of all ALS cases. The highly penetrant nature of this pathology indicates that TDP-43 is broadly involved in both familial and sporadic ALS. Additionally, TDP-43 mutations that promote aggregation are linked to higher risk of developing ALS, suggesting protein misfolding and aggregation act as drivers of toxicity. TDP-43 toxicity can be recapitulated in yeast models, where the protein induces a viability deficit and localizes to stress granules.

In an aspect, the invention provides a compound of formula (I)

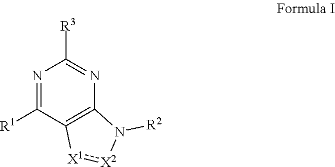

or a pharmaceutically acceptable salt thereof, [symbol]
wherein
   [symbol] is a single bond, X¹ is (C(R^{A})₂)ₘ or —OC(R^{A})₂—R^{X}, and X² is C(R^{A})₂ or CO; or [symbol] is a double bond, and each of X¹ and X² is independently CR^{A} or N, wherein R^{X} is a bond to X²;
   R¹ is -(L)ₙ-R^{B}; optionally substituted C₁₋₆ alkoxy; optionally substituted C₁₋₉ heterocyclyl comprising at least one endocyclic oxygen; unsubstituted pyrimidinyl; optionally substituted pyridazinyl; optionally substituted oxazolyl, or pyrid-2-on-1-yl;
   R² is optionally substituted C₆-10 aryl, optionally substituted C₁₋₉ heterocyclyl, or optionally substituted C₁₋₉ heteroaryl;
   R³ is a group of one of the following structures:

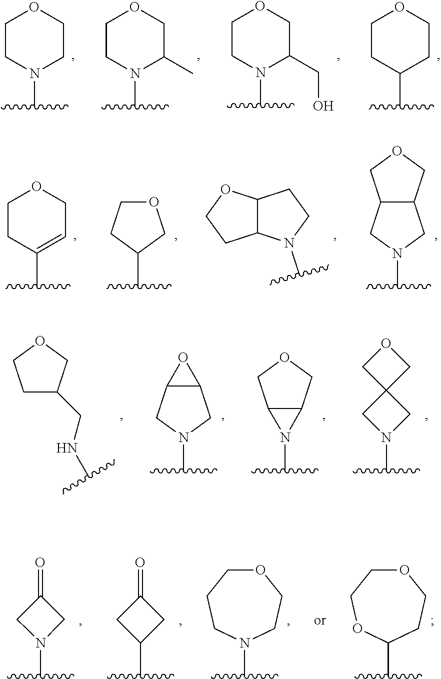

each R^{A} is independently H, optionally substituted C₁₋₅ alkyl, or optionally substituted C₆₋₁₀ aryl;
R^{B} is optionally substituted C₆₋₁₀ aryl, optionally substituted C₁₋₉ heteroaryl, optionally substituted C₃₋₈ cycloalkyl, or optionally substituted C₁₋₉ heterocyclyl;
R^{C} is H or optionally substituted C₁₋₆ alkyl;
each L is independently optionally substituted C₁₋₆ alkylene, O, or NR^{C}; and
n is 1, 2, or 3; and
m is 0, 1, or 2.

In some embodiments, [symbol] is a single bond. In some embodiments, X¹ is (C(R^{A})₂)ₘ. In some embodiments, m is 1. In some embodiments, X² is C(R^{A})₂. In some embodiments, each R^{A} is hydrogen.

In some embodiments, the compound is of formula (Ia)

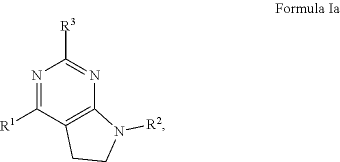

or a pharmaceutically acceptable salt thereof.

Preferably, the compound of formula Ia is of the following structure:

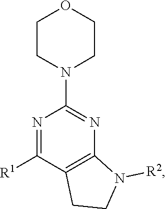

or a pharmaceutically acceptable salt thereof.

In some embodiments, the compound is of formula (Ia′):

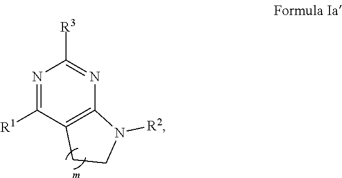

or a pharmaceutically acceptable salt thereof.

Preferably, the compound of formula Ia′ is of the following structure:

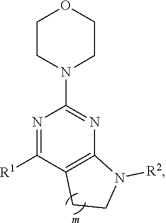

or a pharmaceutically acceptable salt thereof.

In some embodiments, the compound is of formula (Ib)

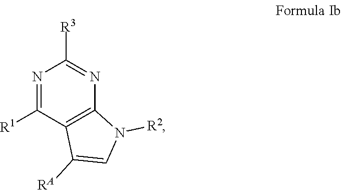

or a pharmaceutically acceptable salt thereof.

In some embodiments, the compound is of formula (Ic)

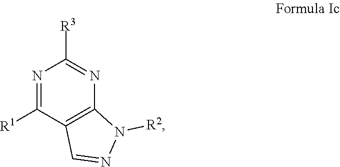

or a pharmaceutically acceptable salt thereof.

In some embodiments, the compound is of formula (Id)

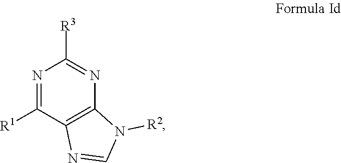

or a pharmaceutically acceptable salt thereof.

In some embodiments, the compound is of formula (Ie)

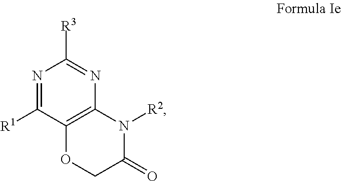

or a pharmaceutically acceptable salt thereof.

In some embodiments, R¹ is —O-(L)₍ₙ₋₁₎-R^{B}. In some embodiments, n is 2. In some embodiments, n is 1. In some embodiments, at least one L is optionally substituted C₁₋₆ alkylene. In some embodiments, the optionally substituted C₁₋₆ alkylene is methylene. In some embodiments, the optionally substituted C₁₋₆ alkylene is ethylene. In some embodiments, R^{B} is optionally substituted non-aromatic C₁₋₉ heterocyclyl. In some embodiments, R^{B} is optionally substituted C₁₋₉ heteroaryl. In some embodiments, R^{B} is optionally substituted C₁₋₆ alkyl.

In some embodiments, R¹ is:

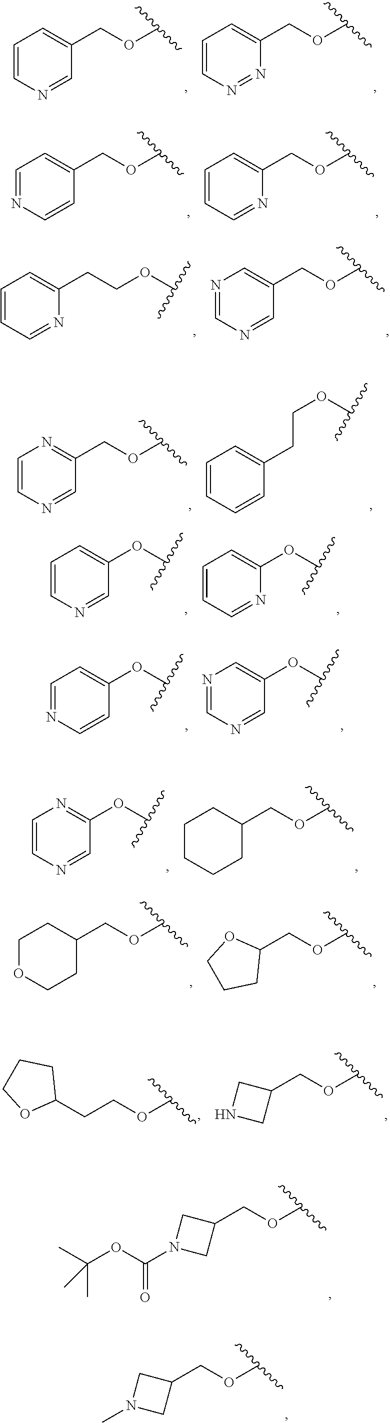

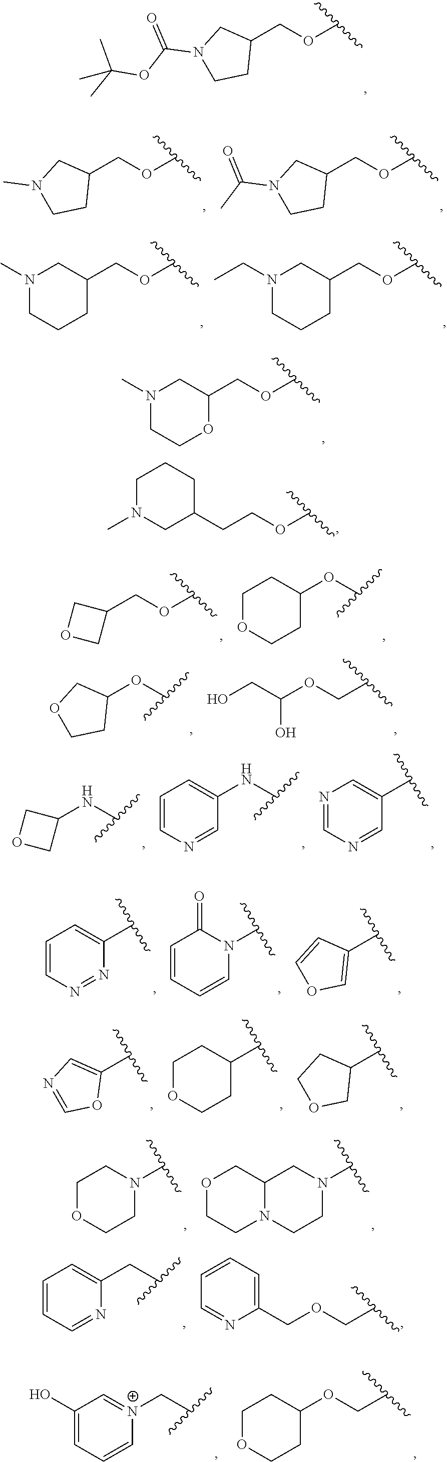

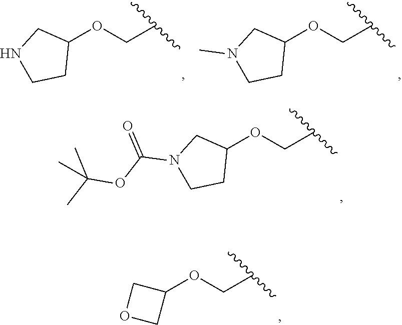

or methoxy.

In some embodiments, R¹ is

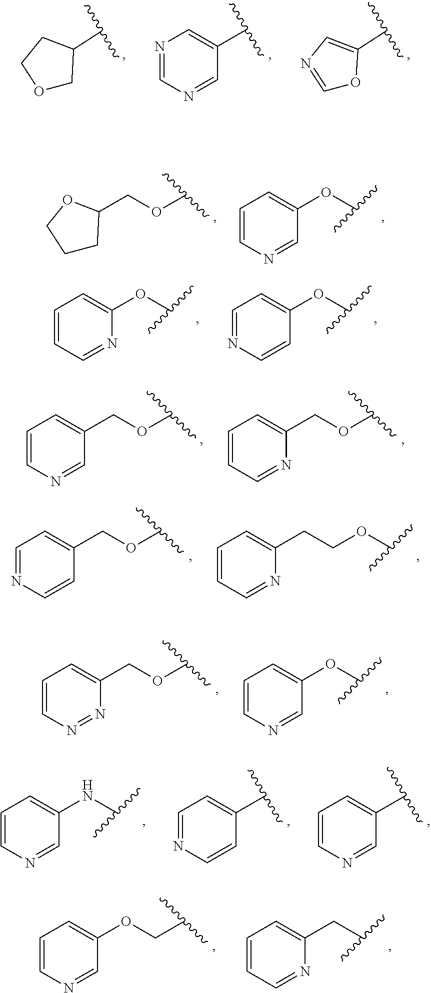

or methoxy.

In some embodiments, R² is:

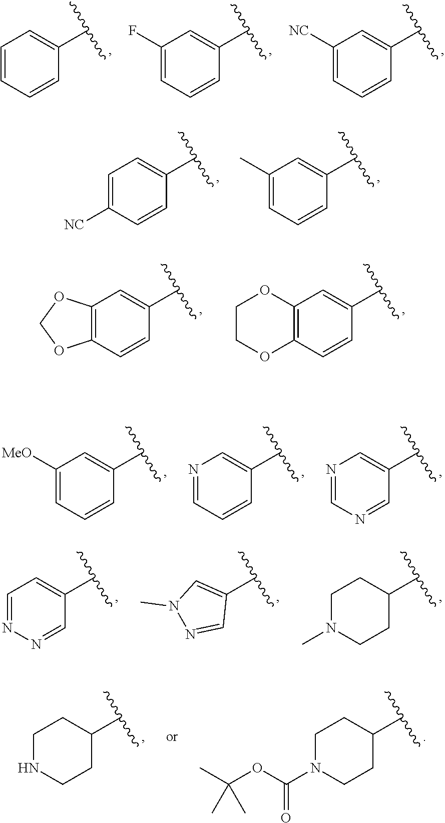

In some embodiments, R² is:

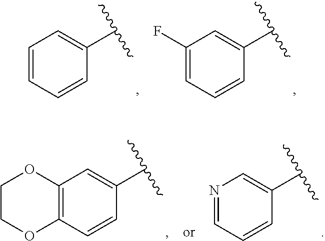

In some embodiments, R³ is:

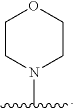 [SMILES: *N1CCOCC1]

Non-limiting examples of the compounds of the invention include:

| # | Compound |
|---|---|
| 1 | 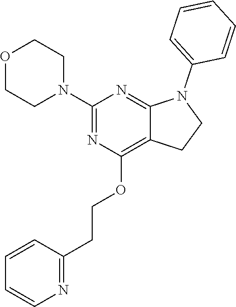 [SMILES: c1ccc(N2CCc3c(OCCc4ccccn4)nc(N4CCOCC4)nc32)cc1] |
| 2 | 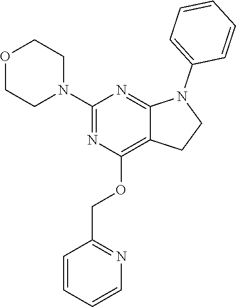 [SMILES: c1ccc(N2CCc3c(OCc4ccccn4)nc(N4CCOCC4)nc32)cc1] |
| 3 | 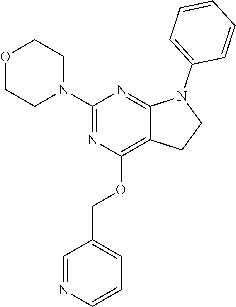 [SMILES: c1ccc(N2CCc3c(OCc4cccnc4)nc(N4CCOCC4)nc32)cc1] |
| 4 | 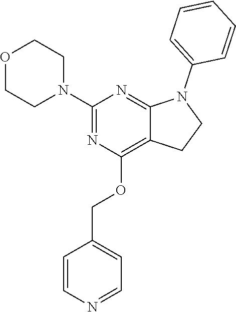 [SMILES: c1ccc(N2CCc3c(OCc4ccncc4)nc(N4CCOCC4)nc32)cc1] |
| 5 | 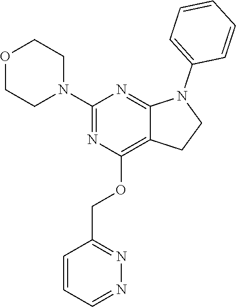 [SMILES: c1ccc(N2CCc3c(OCc4cccnn4)nc(N4CCOCC4)nc32)cc1] |
| 6 | 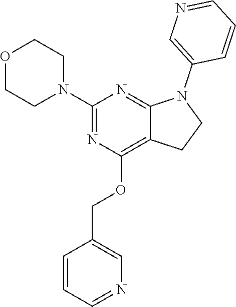 [SMILES: c1cncc(COc2nc(N3CCOCC3)nc3c2CCN3c2cccnc2)c1] |
| 7 | 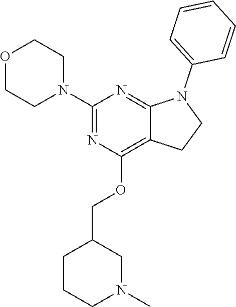 [SMILES: CN1CCCC(COc2nc(N3CCOCC3)nc3c2CCN3c2ccccc2)C1] |
| 8 | 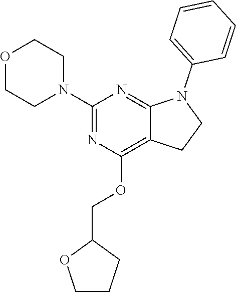 [SMILES: c1ccc(N2CCc3c(OCC4CCCO4)nc(N4CCOCC4)nc32)cc1] |
| 9 | 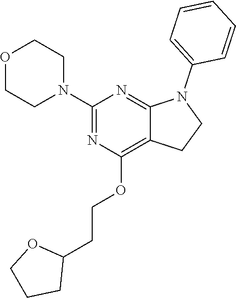 [SMILES: c1ccc(N2CCc3c(OCCC4CCCO4)nc(N4CCOCC4)nc32)cc1] |
| 10 | 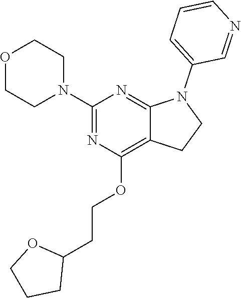 [SMILES: c1cncc(N2CCc3c(OCCC4CCCO4)nc(N4CCOCC4)nc32)c1] |
| 11 | 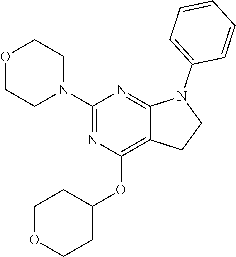 [SMILES: c1ccc(N2CCc3c(OC4CCOCC4)nc(N4CCOCC4)nc32)cc1] |
| 12 | 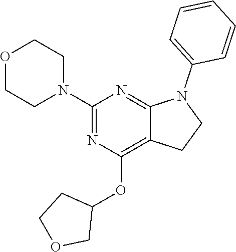 [SMILES: c1ccc(N2CCc3c(OC4CCOC4)nc(N4CCOCC4)nc32)cc1] |
| 13 | 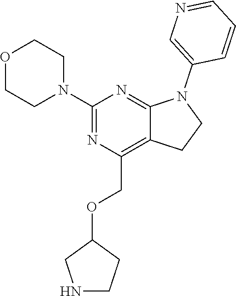 [SMILES: c1cncc(N2CCc3c(COC4CCNC4)nc(N4CCOCC4)nc32)c1] |
| 14 | 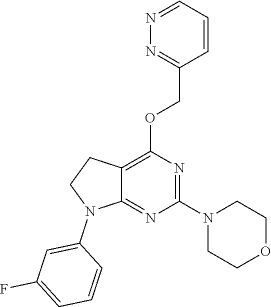 [SMILES: Fc1cccc(N2CCc3c(OCc4cccnn4)nc(N4CCOCC4)nc32)c1] |
| 15 | 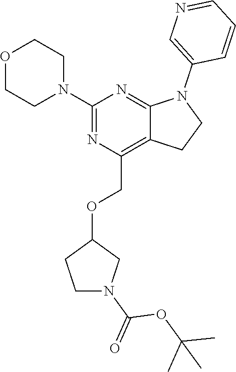 [SMILES: CC(C)(C)OC(=O)N1CCC(OCc2nc(N3CCOCC3)nc3c2CCN3c2cccnc2)C1] |
| 16 | 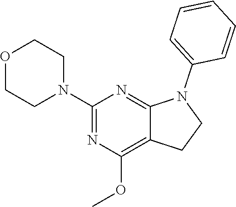 [SMILES: COc1nc(N2CCOCC2)nc2c1CCN2c1ccccc1] |
| 17 | 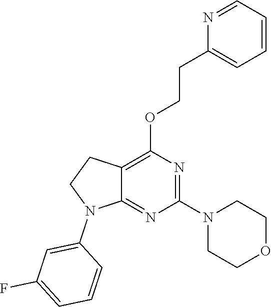 [SMILES: Fc1cccc(N2CCc3c(OCCc4ccccn4)nc(N4CCOCC4)nc32)c1] |
| 18 | 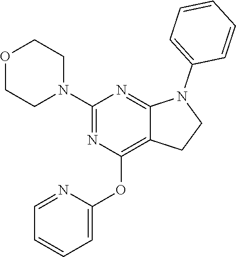 [SMILES: c1ccc(N2CCc3c(Oc4ccccn4)nc(N4CCOCC4)nc32)cc1] |
| 19 | 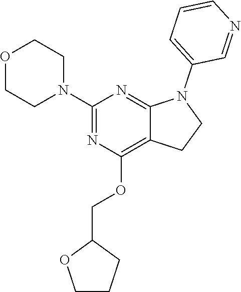 [SMILES: c1cncc(N2CCc3c(OCC4CCCO4)nc(N4CCOCC4)nc32)c1] |
| 20 | 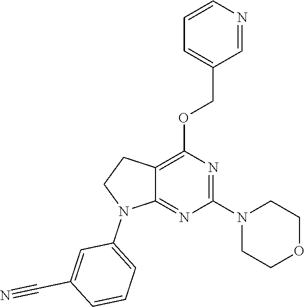 [SMILES: N#Cc1cccc(N2CCc3c(OCc4cccnc4)nc(N4CCOCC4)nc32)c1] |
| 21 | 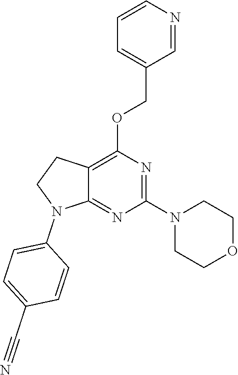 [SMILES: N#Cc1ccc(N2CCc3c(OCc4cccnc4)nc(N4CCOCC4)nc32)cc1] |
| 22 | 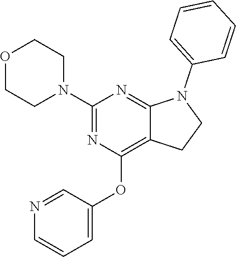 [SMILES: c1ccc(N2CCc3c(Oc4cccnc4)nc(N4CCOCC4)nc32)cc1] |
| 23 | 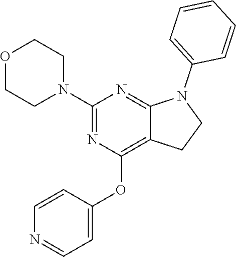 [SMILES: c1ccc(N2CCc3c(Oc4ccncc4)nc(N4CCOCC4)nc32)cc1] |
| 24 | 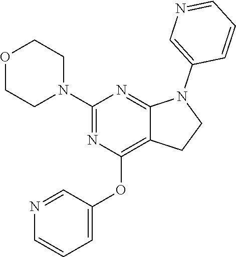 [SMILES: c1cncc(Oc2nc(N3CCOCC3)nc3c2CCN3c2cccnc2)c1] |
| 25 | 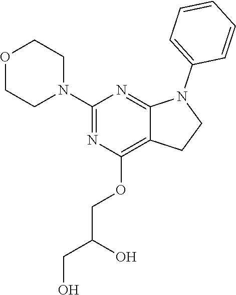 [SMILES: OCC(O)COc1nc(N2CCOCC2)nc2c1CCN2c1ccccc1] |
| 26 | 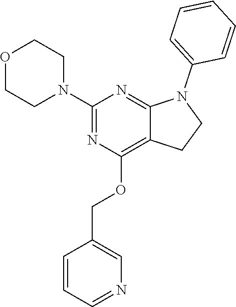 [SMILES: c1ccc(N2CCc3c(OCc4cccnc4)nc(N4CCOCC4)nc32)cc1] |
| 27 | 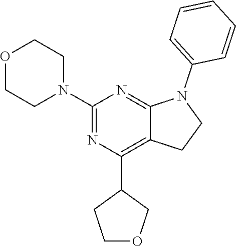 [SMILES: c1ccc(N2CCc3c(C4CCOC4)nc(N4CCOCC4)nc32)cc1] |
| 28 | 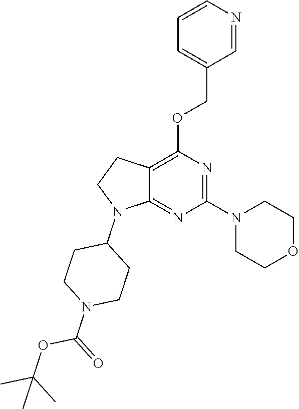 [SMILES: CC(C)(C)OC(=O)N1CCC(N2CCc3c(OCc4cccnc4)nc(N4CCOCC4)nc32)CC1] |
| 29 | 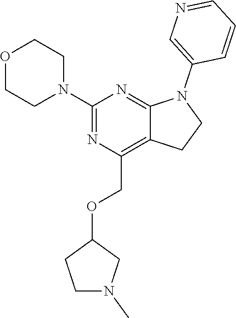 [SMILES: CN1CCC(OCc2nc(N3CCOCC3)nc3c2CCN3c2cccnc2)C1] |
| 30 | 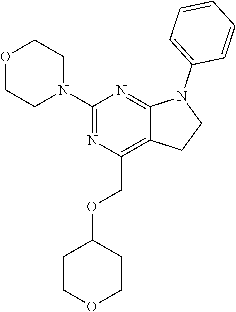 [SMILES: c1ccc(N2CCc3c(COC4CCOCC4)nc(N4CCOCC4)nc32)cc1] |
| 31 | 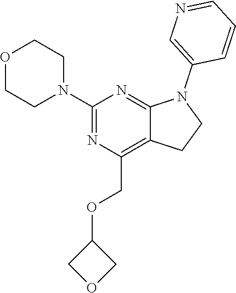 [SMILES: c1cncc(N2CCc3c(COC4COC4)nc(N4CCOCC4)nc32)c1] |

and pharmaceutically acceptable salts thereof.

Further non-limiting examples of the compounds of the invention include:

| # | Compound |
|---|---|
| 32 | 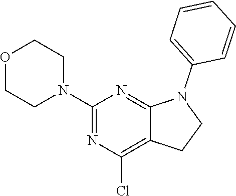 [SMILES: Clc1nc(N2CCOCC2)nc2c1CCN2c1ccccc1] |
| 33 | 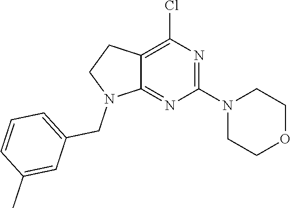 [SMILES: Cc1cccc(CN2CCc3c(Cl)nc(N4CCOCC4)nc32)c1] |
| 34 | 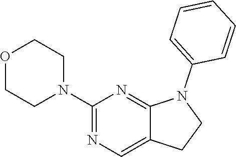 [SMILES: c1ccc(N2CCc3cnc(N4CCOCC4)nc32)cc1] |
| 35 | 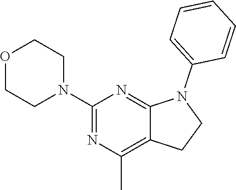 [SMILES: Cc1nc(N2CCOCC2)nc2c1CCN2c1ccccc1] |
| 36 | 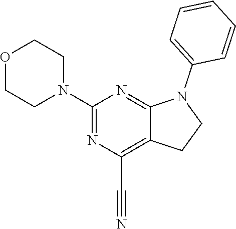 [SMILES: N#Cc1nc(N2CCOCC2)nc2c1CCN2c1ccccc1] |
| 37 | 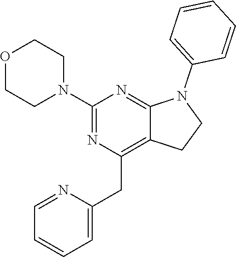 [SMILES: c1ccc(N2CCc3c(Cc4ccccn4)nc(N4CCOCC4)nc32)cc1] |
| 38 | 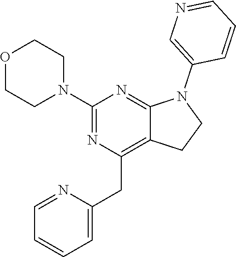 [SMILES: c1ccc(Cc2nc(N3CCOCC3)nc3c2CCN3c2cccnc2)nc1] |
| 39 | 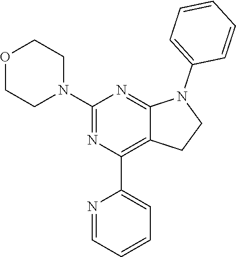 [SMILES: c1ccc(N2CCc3c(-c4ccccn4)nc(N4CCOCC4)nc32)cc1] |
| 40 | 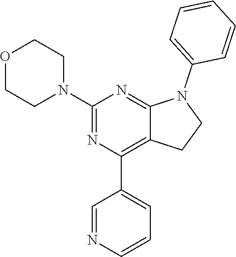 [SMILES: c1ccc(N2CCc3c(-c4cccnc4)nc(N4CCOCC4)nc32)cc1] |
| 41 | 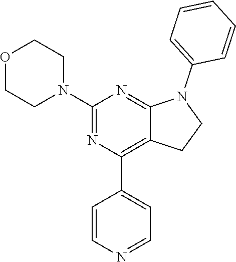 [SMILES: c1ccc(N2CCc3c(-c4ccncc4)nc(N4CCOCC4)nc32)cc1] |
| 42 | 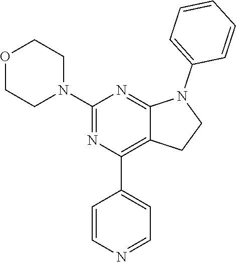 [SMILES: c1ccc(N2CCc3c(-c4ccncc4)nc(N4CCOCC4)nc32)cc1] |
| 43 | 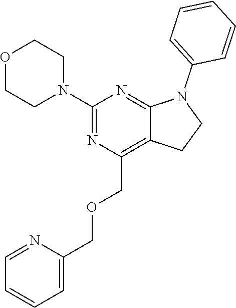 [SMILES: c1ccc(N2CCc3c(COCc4ccccn4)nc(N4CCOCC4)nc32)cc1] |
| 44 | 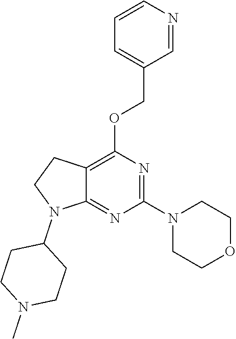 [SMILES: CN1CCC(N2CCc3c(OCc4cccnc4)nc(N4CCOCC4)nc32)CC1] |
| 45 | 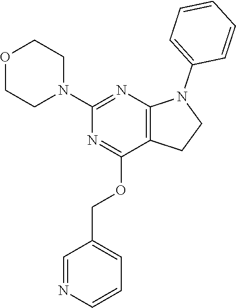 [SMILES: c1ccc(N2CCc3c(OCc4cccnc4)nc(N4CCOCC4)nc32)cc1] |
| 46 | 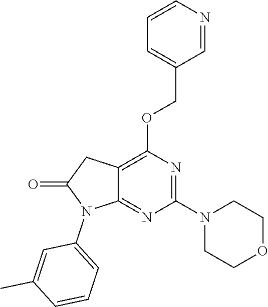 [SMILES: Cc1cccc(N2C(=O)Cc3c(OCc4cccnc4)nc(N4CCOCC4)nc32)c1] |
| 47 | 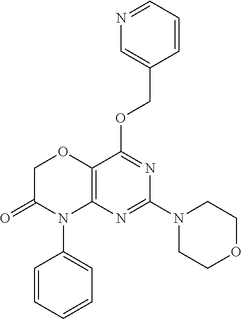 [SMILES: O=C1COc2c(OCc3cccnc3)nc(N3CCOCC3)nc2N1c1ccccc1] |
| 48 | 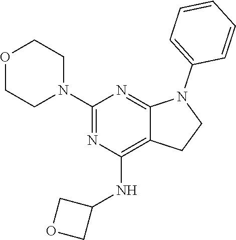 [SMILES: c1ccc(N2CCc3c(NC4COC4)nc(N4CCOCC4)nc32)cc1] |
| 49 | 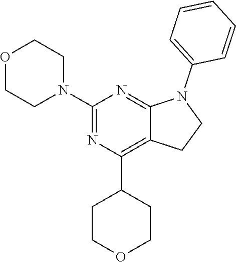 [SMILES: c1ccc(N2CCc3c(C4CCOCC4)nc(N4CCOCC4)nc32)cc1] |
| 50 | 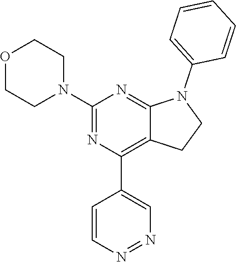 [SMILES: c1ccc(N2CCc3c(-c4ccnnc4)nc(N4CCOCC4)nc32)cc1] |
| 51 | 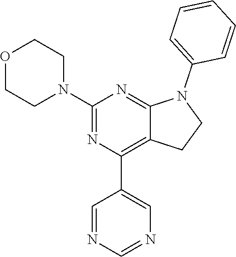 [SMILES: c1ccc(N2CCc3c(-c4cncnc4)nc(N4CCOCC4)nc32)cc1] |
| 52 | 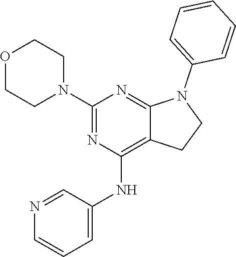 [SMILES: c1ccc(N2CCc3c(Nc4cccnc4)nc(N4CCOCC4)nc32)cc1] |
| 53 | 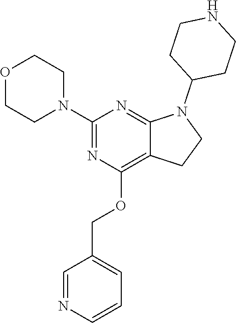 [SMILES: c1cncc(COc2nc(N3CCOCC3)nc3c2CCN3C2CCNCC2)c1] |
| 54 | 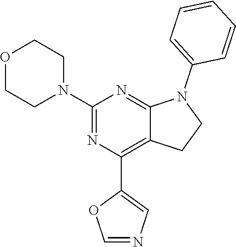 [SMILES: c1ccc(N2CCc3c(-c4cnco4)nc(N4CCOCC4)nc32)cc1] |
| 55 | 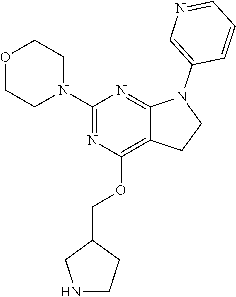 [SMILES: c1cncc(N2CCc3c(OCC4CCNC4)nc(N4CCOCC4)nc32)c1] |
| 56 | 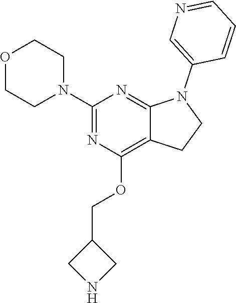 [SMILES: c1cncc(N2CCc3c(OCC4CNC4)nc(N4CCOCC4)nc32)c1] |
| 57 | 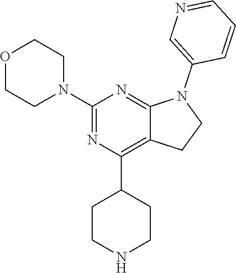 [SMILES: c1cncc(N2CCc3c(C4CCNCC4)nc(N4CCOCC4)nc32)c1] |
| 58 | 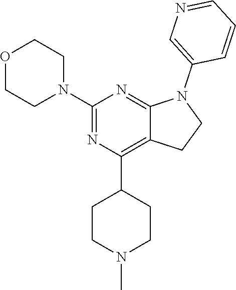 [SMILES: CN1CCC(c2nc(N3CCOCC3)nc3c2CCN3c2cccnc2)CC1] |
| 59 | 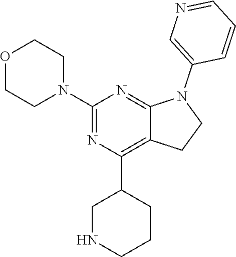 [SMILES: c1cncc(N2CCc3c(C4CCCNC4)nc(N4CCOCC4)nc32)c1] |
| 60 | 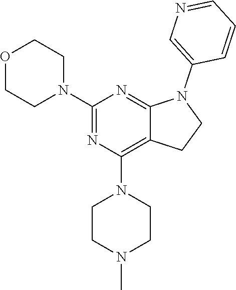 [SMILES: CN1CCN(c2nc(N3CCOCC3)nc3c2CCN3c2cccnc2)CC1] |
| 61 | 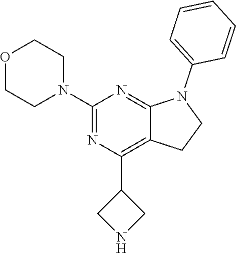 [SMILES: c1ccc(N2CCc3c(C4CNC4)nc(N4CCOCC4)nc32)cc1] |
| 62 | 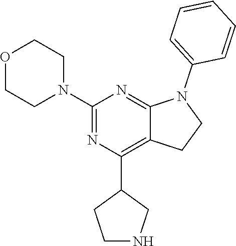 [SMILES: c1ccc(N2CCc3c(C4CCNC4)nc(N4CCOCC4)nc32)cc1] |
| 63 | 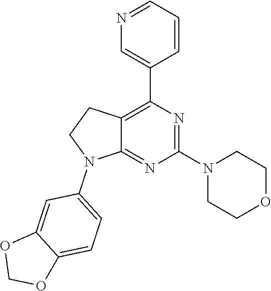 [SMILES: c1cncc(-c2nc(N3CCOCC3)nc3c2CCN3c2ccc3c(c2)OCO3)c1] |
| 64 | 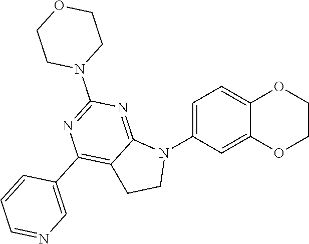 [SMILES: c1cncc(-c2nc(N3CCOCC3)nc3c2CCN3c2ccc3c(c2)OCCO3)c1] |
| 65 | 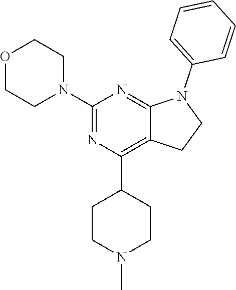 [SMILES: CN1CCC(c2nc(N3CCOCC3)nc3c2CCN3c2ccccc2)CC1] |
| 66 | 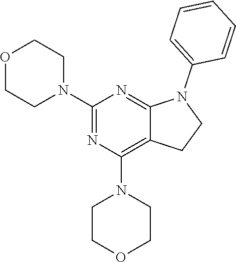 [SMILES: c1ccc(N2CCc3c(N4CCOCC4)nc(N4CCOCC4)nc32)cc1] |
| 67 | 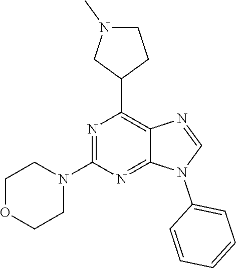 [SMILES: CN1CCC(c2nc(N3CCOCC3)nc3c2ncn3-c2ccccc2)C1] |
| 68 | 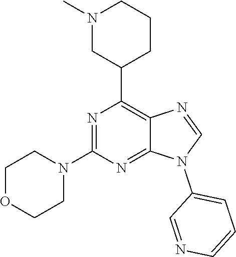 [SMILES: CN1CCCC(c2nc(N3CCOCC3)nc3c2ncn3-c2cccnc2)C1] |
| 69 | 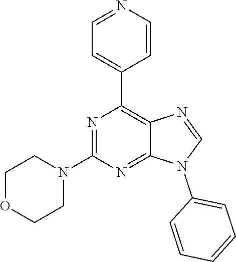 [SMILES: c1ccc(-n2cnc3c(-c4ccncc4)nc(N4CCOCC4)nc32)cc1] |
| 70 | 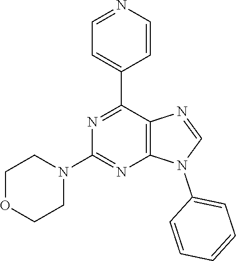 [SMILES: c1ccc(-n2cnc3c(-c4ccncc4)nc(N4CCOCC4)nc32)cc1] |
| 71 | 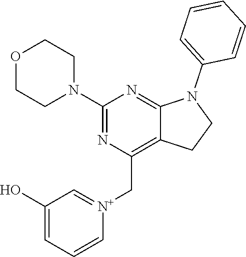 [SMILES: Oc1ccc[n+](Cc2nc(N3CCOCC3)nc3c2CCN3c2ccccc2)c1] |
| 72 | 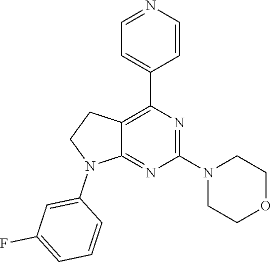 [SMILES: Fc1cccc(N2CCc3c(-c4ccncc4)nc(N4CCOCC4)nc32)c1] |
| 73 | 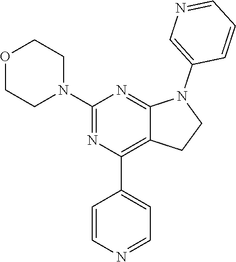 [SMILES: c1cncc(N2CCc3c(-c4ccncc4)nc(N4CCOCC4)nc32)c1] |
| 74 | 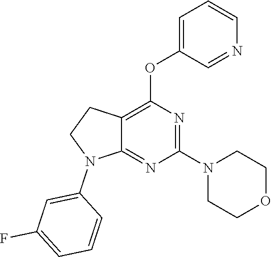 [SMILES: Fc1cccc(N2CCc3c(Oc4cccnc4)nc(N4CCOCC4)nc32)c1] |
| 75 | 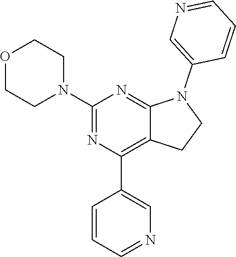 [SMILES: c1cncc(-c2nc(N3CCOCC3)nc3c2CCN3c2cccnc2)c1] |
| 76 | 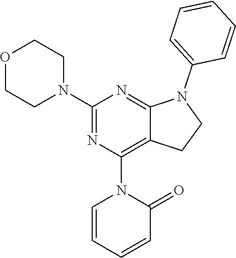 [SMILES: O=c1ccccn1-c1nc(N2CCOCC2)nc2c1CCN2c1ccccc1] |
| 77 | 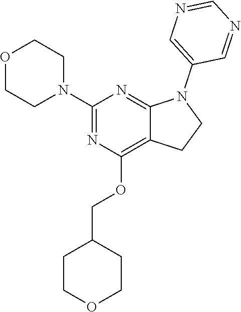 [SMILES: c1ncc(N2CCc3c(OCC4CCOCC4)nc(N4CCOCC4)nc32)cn1] |
| 78 | 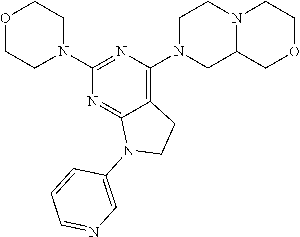 [SMILES: c1cncc(N2CCc3c(N4CCN5CCOCC5C4)nc(N4CCOCC4)nc32)c1] |
| 79 | 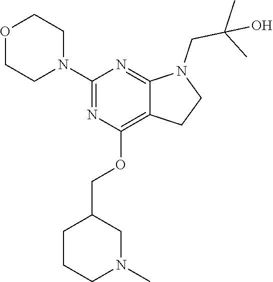 [SMILES: CN1CCCC(COc2nc(N3CCOCC3)nc3c2CCN3CC(C)(C)O)C1] |
| 80 | 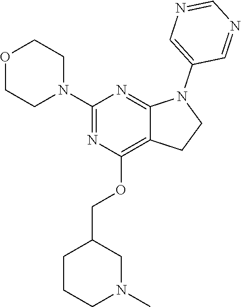 [SMILES: CN1CCCC(COc2nc(N3CCOCC3)nc3c2CCN3c2cncnc2)C1] |
| 81 | 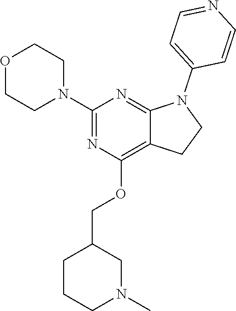 [SMILES: CN1CCCC(COc2nc(N3CCOCC3)nc3c2CCN3c2ccncc2)C1] |
| 82 | 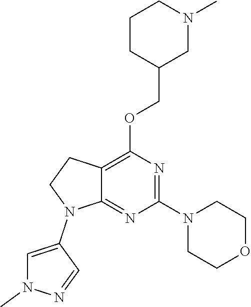 [SMILES: CN1CCCC(COc2nc(N3CCOCC3)nc3c2CCN3c2cnn(C)c2)C1] |
| 83 | 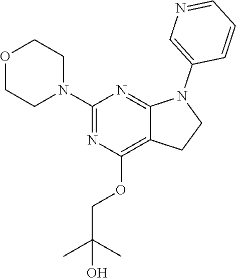 [SMILES: CC(C)(O)COc1nc(N2CCOCC2)nc2c1CCN2c1cccnc1] |
| 84 | 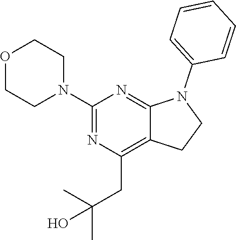 [SMILES: CC(C)(O)Cc1nc(N2CCOCC2)nc2c1CCN2c1ccccc1] |
| 85 | 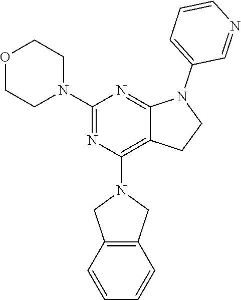 [SMILES: c1cncc(N2CCc3c(N4Cc5ccccc5C4)nc(N4CCOCC4)nc32)c1] |
| 86 | 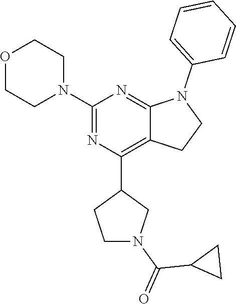 [SMILES: O=C(C1CC1)N1CCC(c2nc(N3CCOCC3)nc3c2CCN3c2ccccc2)C1] |
| 87 | 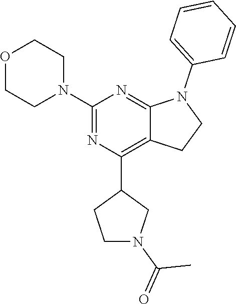 [SMILES: CC(=O)N1CCC(c2nc(N3CCOCC3)nc3c2CCN3c2ccccc2)C1] |
| 88 | 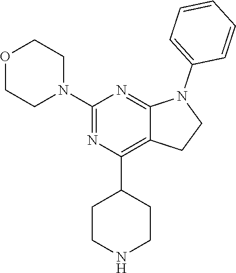 [SMILES: c1ccc(N2CCc3c(C4CCNCC4)nc(N4CCOCC4)nc32)cc1] |
| 89 | 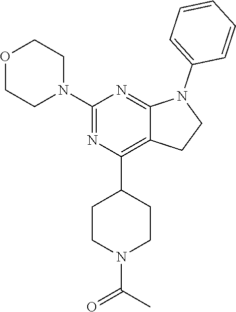 [SMILES: CC(=O)N1CCC(c2nc(N3CCOCC3)nc3c2CCN3c2ccccc2)CC1] |
| 90 | 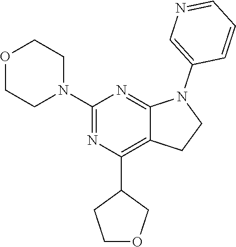 [SMILES: c1cncc(N2CCc3c(C4CCOC4)nc(N4CCOCC4)nc32)c1] |
| 91 | 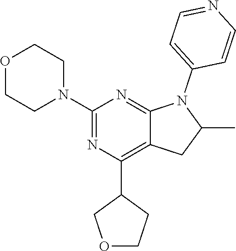 [SMILES: CC1Cc2c(C3CCOC3)nc(N3CCOCC3)nc2N1c1ccncc1] |
| 92 | 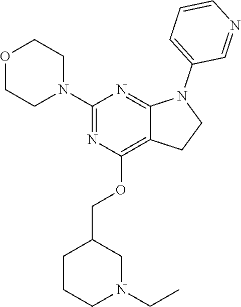 [SMILES: CCN1CCCC(COc2nc(N3CCOCC3)nc3c2CCN3c2cccnc2)C1] |
| 93 | 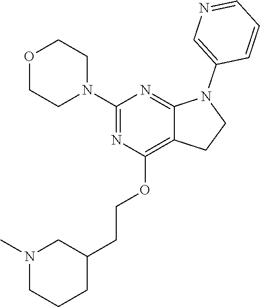 [SMILES: CN1CCCC(CCOc2nc(N3CCOCC3)nc3c2CCN3c2cccnc2)C1] |
| 94 | 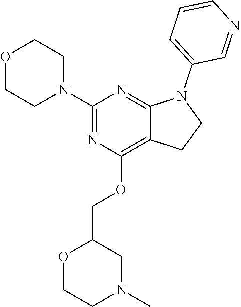 [SMILES: CN1CCOC(COc2nc(N3CCOCC3)nc3c2CCN3c2cccnc2)C1] |
| 95 | 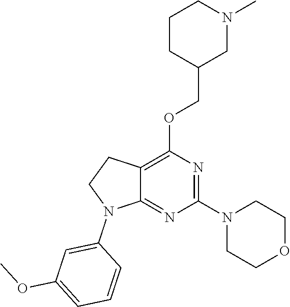 [SMILES: COc1cccc(N2CCc3c(OCC4CCCN(C)C4)nc(N4CCOCC4)nc32)c1] |
| 96 | 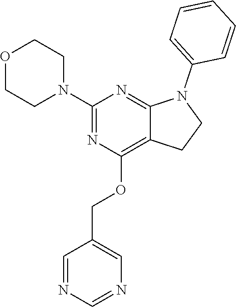 [SMILES: c1ccc(N2CCc3c(OCc4cncnc4)nc(N4CCOCC4)nc32)cc1] |
| 97 | 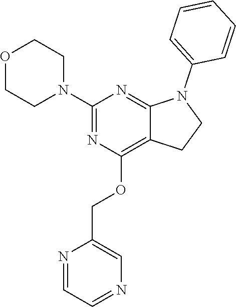 [SMILES: c1ccc(N2CCc3c(OCc4cnccn4)nc(N4CCOCC4)nc32)cc1] |
| 98 | 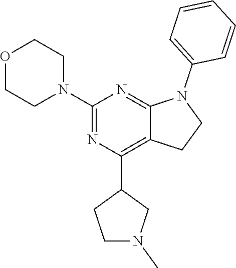 [SMILES: CN1CCC(c2nc(N3CCOCC3)nc3c2CCN3c2ccccc2)C1] |
| 99 | 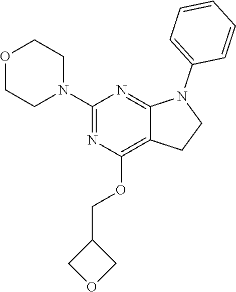 [SMILES: c1ccc(N2CCc3c(OCC4COC4)nc(N4CCOCC4)nc32)cc1] |
| 100 | 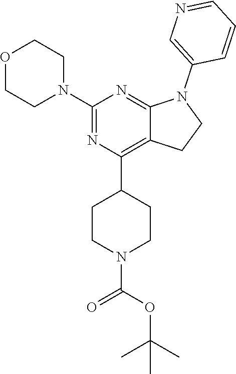 [SMILES: CC(C)(C)OC(=O)N1CCC(c2nc(N3CCOCC3)nc3c2CCN3c2cccnc2)CC1] |
| 101 | 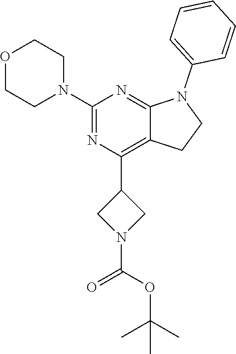 [SMILES: CC(C)(C)OC(=O)N1CC(c2nc(N3CCOCC3)nc3c2CCN3c2ccccc2)C1] |
| 102 | 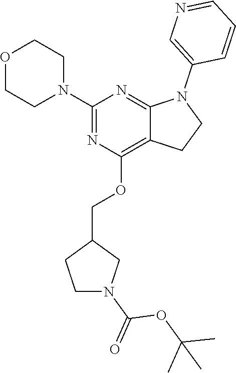 [SMILES: CC(C)(C)OC(=O)N1CCC(COc2nc(N3CCOCC3)nc3c2CCN3c2cccnc2)C1] |
| 103 | 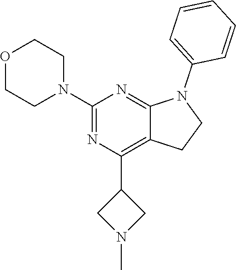 [SMILES: CN1CC(c2nc(N3CCOCC3)nc3c2CCN3c2ccccc2)C1] |
| 104 | 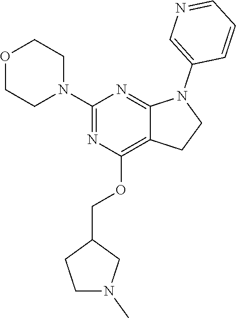 [SMILES: CN1CCC(COc2nc(N3CCOCC3)nc3c2CCN3c2cccnc2)C1] |
| 105 | 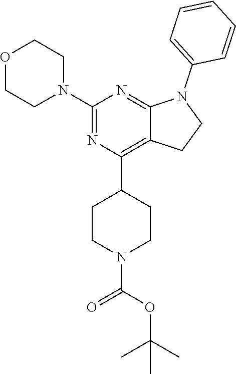 [SMILES: CC(C)(C)OC(=O)N1CCC(c2nc(N3CCOCC3)nc3c2CCN3c2ccccc2)CC1] |
| 106 | 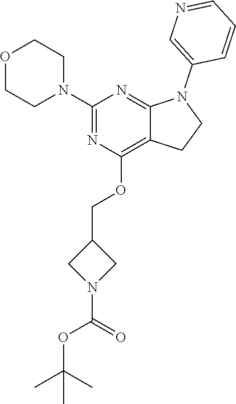 [SMILES: CC(C)(C)OC(=O)N1CC(COc2nc(N3CCOCC3)nc3c2CCN3c2cccnc2)C1] |
| 107 | 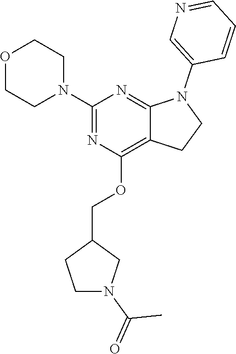 [SMILES: CC(=O)N1CCC(COc2nc(N3CCOCC3)nc3c2CCN3c2cccnc2)C1] |
| 108 | 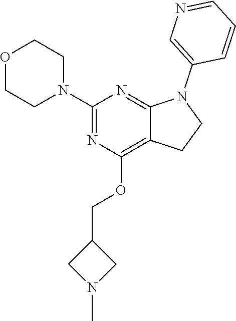 [SMILES: CN1CC(COc2nc(N3CCOCC3)nc3c2CCN3c2cccnc2)C1] |
| 109 | 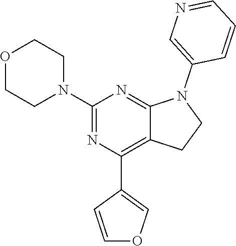 [SMILES: c1cncc(N2CCc3c(-c4ccoc4)nc(N4CCOCC4)nc32)c1] |
| 110 | 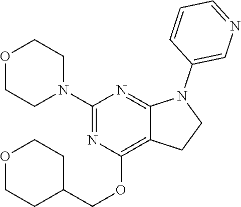 [SMILES: c1cncc(N2CCc3c(OCC4CCOCC4)nc(N4CCOCC4)nc32)c1] |
| 111 | 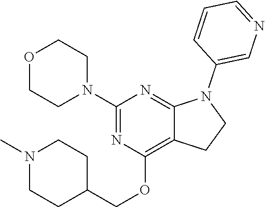 [SMILES: CN1CCC(COc2nc(N3CCOCC3)nc3c2CCN3c2cccnc2)CC1] |

and pharmaceutically acceptable salts thereof.

In an aspect, the invention features a pharmaceutical composition comprising any of the foregoing compounds and a pharmaceutically acceptable excipient.

In an aspect, the invention features a method of treating a neurological disorder (e.g., frontotemporal dementia (FTLD-TDP), chronic traumatic encephalopathy, ALS, Alzheimer's disease, limbic-predominant age-related TDP-43 encephalopathy (LATE), or frontotemporal lobar degeneration) in a subject in need thereof. This method includes administering an effective amount of any of the foregoing compounds or pharmaceutical compositions.

In an aspect, the invention features a method of inhibiting toxicity in a cell (e.g., mammalian neural cell) related to a protein (e.g., TDP-43 or C9orf72). This method includes administering an effective amount of any of the foregoing compounds or pharmaceutical compositions.

In an aspect, the invention features a method of treating a TDP-43-associated disorder or C9orf72-associated disorder (e.g., FTLD-TDP, chronic traumatic encephalopathy, ALS, Alzheimer's disease, LATE, or frontotemporal lobar degeneration) in a subject in need thereof. This method includes administering to the subject an effective amount of a compounds described herein or a pharmaceutical composition containing one or more compounds described herein. In some embodiments, the method includes administering to the subject in need thereof an effective amount of the compound of formula (I)

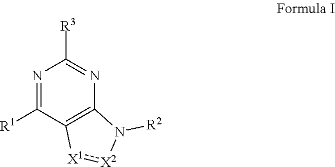

or a pharmaceutically acceptable salt thereof,
where
   [symbol] is a single bond, X¹ is (C(R^{A})₂)ₘ or —OC(R^{A})₂—R^{X}, and X² is C(R^{A})₂ or CO; or [symbol] is a double bond, and each of X¹ and X² is independently CR^{A} or N, wherein R^{X} is a bond to X²;
   R¹ is -(L)ₙ-R^{B}; hydrogen; halogen; cyano; optionally substituted C₁₋₆ alkyl; optionally substituted C₁₋₆ heteroalkyl; optionally substituted C₁₋₆ alkoxy; optionally substituted C₆-10 aryl, optionally substituted C₁₋₉ heterocyclyl, or optionally substituted C₁₋₉ heteroaryl;
   R² is hydrogen, optionally substituted C₁₋₆ alkyl, optionally substituted C₆-10 aryl, optionally substituted C₁₋₉ heterocyclyl, or optionally substituted C₁₋₉ heteroaryl;
   R³ is a group of the following structure:

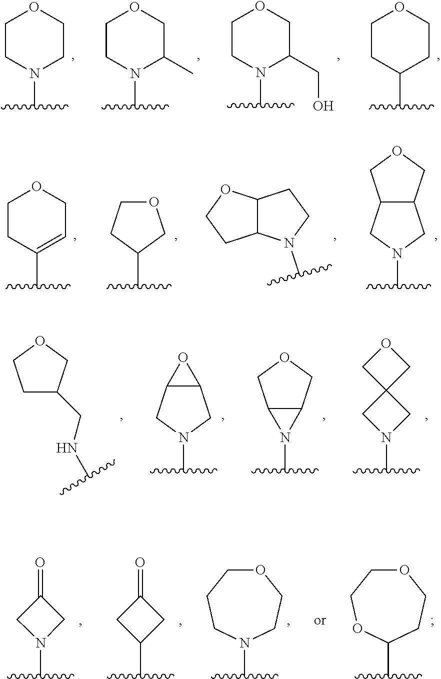

each R^{A} is independently H, optionally substituted C₁₋₆ alkyl, or optionally substituted C₆₋₁₀ aryl;
R^{B} is optionally substituted C₆₋₁₀ aryl, optionally substituted C₁₋₉ heteroaryl, optionally substituted C₃₋₈ cycloalkyl, or optionally substituted C₁₋₉ heterocyclyl;
R^{e} is H or optionally substituted C₁₋₆ alkyl;
each L is independently optionally substituted alkylene, O, or NR^{C}; and
n is 1, 2, or 3; and
m is 0, 1, or 2.

In some embodiments, [symbol] is a single bond. In some embodiments, X¹ is (C(R^{A})₂)ₘ. In some embodiments, m is 1. In some embodiments, X² is C(R^{A})₂. In some embodiments, each R^{A} is hydrogen.

In some embodiments, the compound is of formula (Ia)

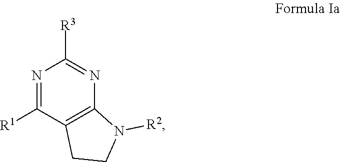

or a pharmaceutically acceptable salt thereof.

Preferably, the compound of formula Ia is of the following structure:

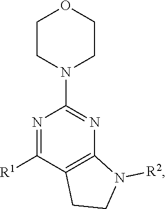

or a pharmaceutically acceptable salt thereof.

In some embodiments, the compound is of formula (Ia′):

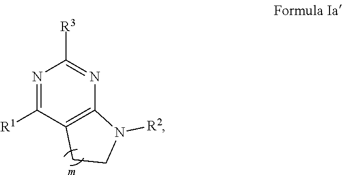

or a pharmaceutically acceptable salt thereof.

Preferably, the compound of formula Ia′ is of the following structure:

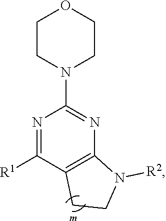

or a pharmaceutically acceptable salt thereof.

In some embodiments, the compound is of formula (Ib)

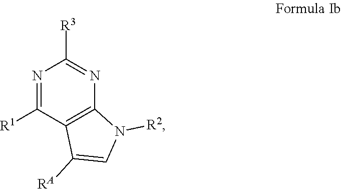

or a pharmaceutically acceptable salt thereof.

In some embodiments, the compound is of formula (Ic)

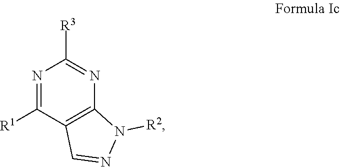

or a pharmaceutically acceptable salt thereof.

In some embodiments, the compound is of formula (Id)

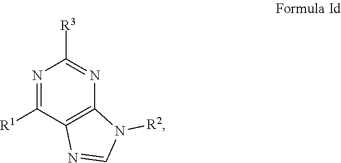

or a pharmaceutically acceptable salt thereof.

In some embodiments, the compound is of formula (Ie)

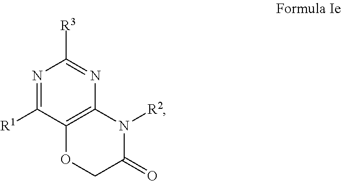

or a pharmaceutically acceptable salt thereof.

In some embodiments, R¹ is —O-(L)₍ₙ₋₁₎-R^{B}. In some embodiments, n is 2. In some embodiments, n is 1. In some embodiments, at least one L is optionally substituted C₁₋₆ alkylene. In some embodiments, the optionally substituted C₁₋₆ alkylene is methylene. In some embodiments, the optionally substituted C₁₋₆ alkylene is ethylene. In some embodiments, R^{B} is optionally substituted non-aromatic C₁₋₉ heterocyclyl. In some embodiments, R^{B} is optionally substituted C₁₋₉ heteroaryl. In some embodiments, R^{B} is optionally substituted C₁₋₆ alkyl.

In some embodiments, R¹ is:

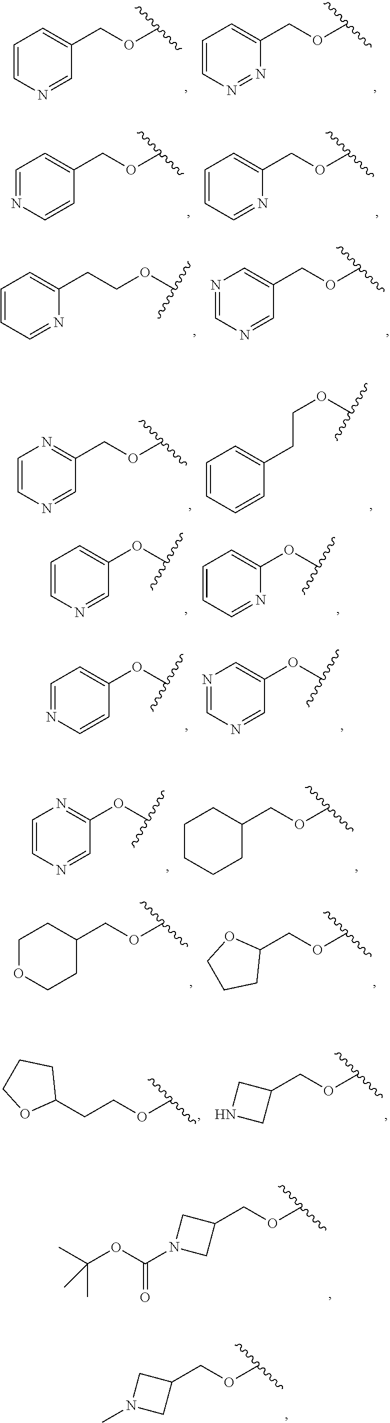

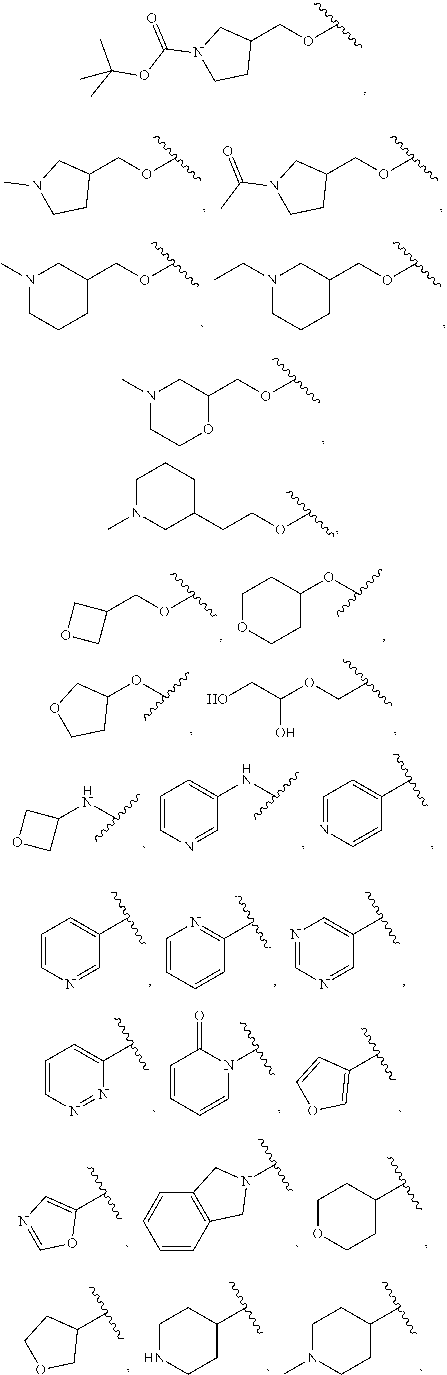

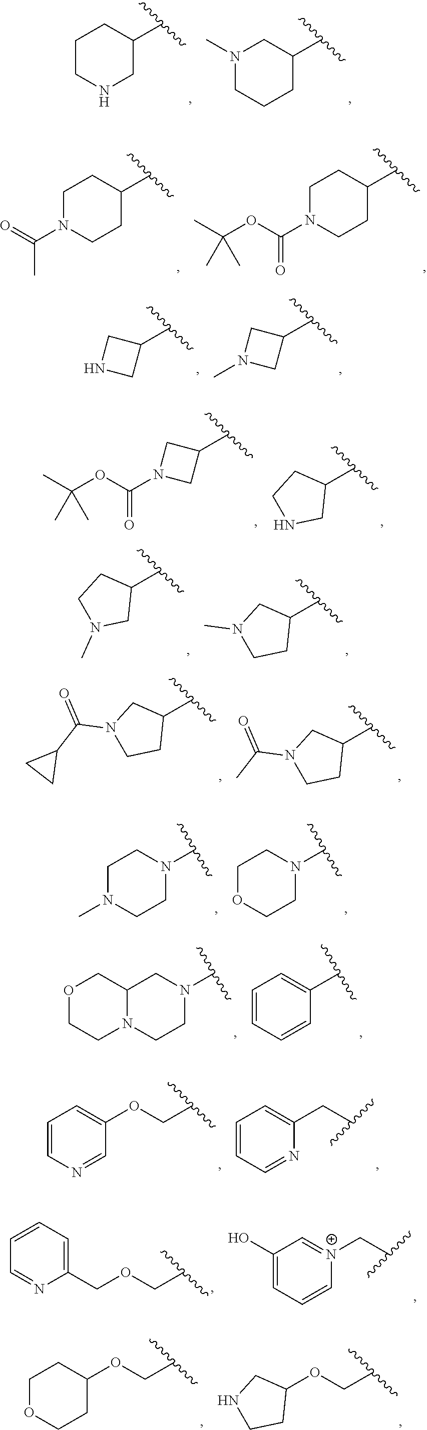

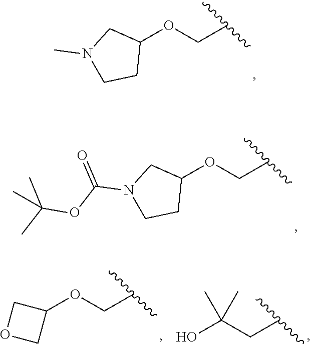

hydrogen, chloro, methyl, cyano, or methoxy.

In some embodiments, R¹ is:

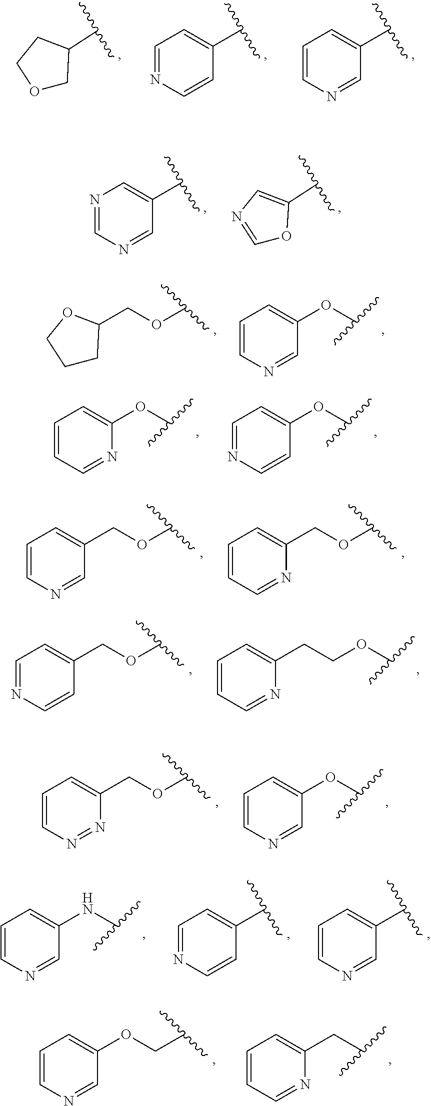

or methoxy.

In some embodiments, R² is:

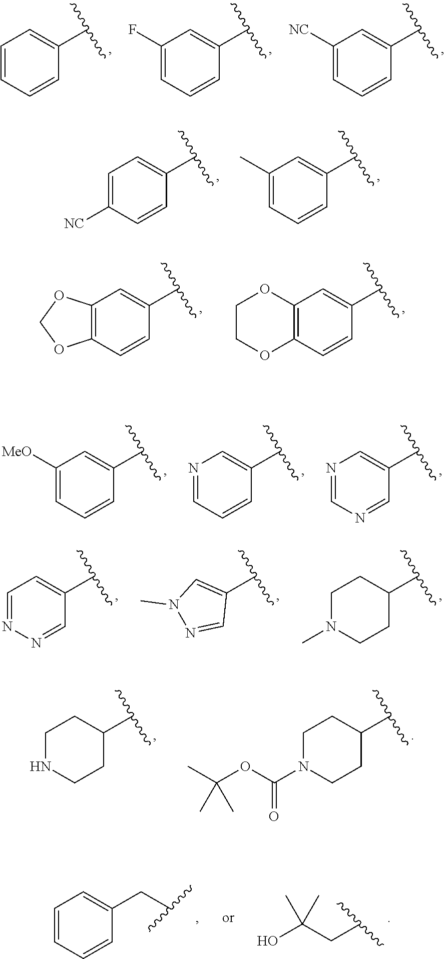

In some embodiments, R² is:

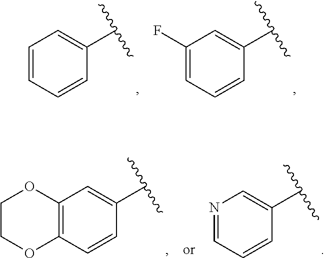

In some embodiments, R³ is:

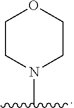 [SMILES: *N1CCOCC1]

In an aspect, the invention features a method of inhibiting PIKfyve. This method includes contacting a cell with an effective amount of any of the foregoing compounds or pharmaceutical compositions.

In another aspect, the invention features a method of treating a neurological disorder in a patient, such as a human patient, identified as likely to benefit from treatment with a compound of the invention on the basis of TDP-43 toxicity. In this aspect, the method may include (i) determining that the patient exhibits, or is prone to develop, TDP-43 toxicity, and (ii) providing to the patient a therapeutically effective amount of a compound of the invention. In some embodiments, the patient has previously been determined to exhibit, or to be prone to developing, TDP-43 toxicity, and the method includes providing to the patient a therapeutically effective amount of a compound of the invention. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331K, M337V, Q343R, N345K, R361S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In an additional aspect, the invention features a method of treating a neurological disorder in a patient, such as a human patient, identified as likely to benefit from treatment with a compound of the invention on the basis of TDP-43 expression. In this aspect, the method includes (i) determining that the patient expresses a mutant form of TDP-43 having a mutation associated with TDP-43 aggregation (e.g., a mutation selected from Q331K, M337V, Q343R, N345K, R361S, and N390D), and (ii) providing to the patient a therapeutically effective amount of a compound of the invention. In some embodiments, the patient has previously been determined to express a mutant form of TDP-43 having a mutation associated with TDP-43 aggregation, such as a Q331K, M337V, Q343R, N345K, R361S, or N390D mutation, and the method includes providing to the patient a therapeutically effective amount of a compound of the invention.

In another aspect, the invention features a method of determining whether a patient (e.g., a human patient) having a neurological disorder is likely to benefit from treatment with a compound of the invention by (i) determining whether the patient exhibits, or is prone to develop, TDP-43 aggregation and (ii) identifying the patient as likely to benefit from treatment with a compound of the invention if the patient exhibits, or is prone to develop, TDP-43 aggregation. In some embodiments, the method further includes the step of (iii) informing the patient whether he or she is likely to benefit from treatment with a compound of the invention. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331K, M337V, Q343R, N345K, R361S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In another aspect, the invention features a method of determining whether a patient (e.g., a human patient) having a neurological disorder is likely to benefit from treatment with a compound of the invention by (i) determining whether the patient expresses a TDP-43 mutant having a mutation associated with TDP-43 aggregation (e.g., a mutation selected from Q331K, M337V, Q343R, N345K, R361S, and N390D) and (ii) identifying the patient as likely to benefit from treatment with a compound of the invention if the patient expresses a TDP-43 mutant. In some embodiments, the method further includes the step of (iii) informing the patient whether he or she is likely to benefit from treatment with a compound of the invention. The TDP-43 isoform expressed by the patient may be assessed, for example, by isolated TDP-43 protein from a sample obtained from the patient and sequencing the protein using molecular biology techniques described herein or known in the art. In some embodiments, the TDP-43 isoform expressed by the patient is determined by analyzing the patient's genotype at the TDP-43 locus, for example, by sequencing the TDP-43 gene in a sample obtained from the patient. In some embodiments, the method includes the step of obtaining the sample from the patient.

In some embodiments of any of the above aspects, the compound of the invention is provided to the patient by administration of the compound of the invention to the patient. In some embodiments, the compound of the invention is provided to the patient by administration of a prodrug that is converted in vivo to the compound of the invention.

In some embodiments of any of the above aspects, the neurological disorder is a neuromuscular disorder, such as a neuromuscular disorder selected from amyotrophic lateral sclerosis, congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barre syndrome. In some embodiments, the neurological disorder is amyotrophic lateral sclerosis.

In some embodiments of any of the above aspects, the neurological disorder is selected from frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy.

In some embodiments, the neurological disorder is amyotrophic lateral sclerosis, and following administration of the compound of the invention to the patient, the patient exhibits one or more, or all, of the following responses:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the compound of the invention (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(ii) an increase in slow vital capacity, such as an increase in the patient's slow vital capacity within one or more days, weeks, or months following administration of the compound of the invention (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(iii) a reduction in decremental responses exhibited by the patient upon repetitive nerve stimulation, such as a reduction that is observed within one or more days, weeks, or months following administration of the compound of the invention (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the compound of the invention (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the compound of the invention (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(vi) a decrease in the frequency and/or severity of muscle cramps, such as a decrease in cramp frequency and/or severity within one or more days, weeks, or months following administration of the compound of the invention (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient); and/or
(vii) a decrease in TDP-43 aggregation, such as a decrease in TDP-43 aggregation within one or more days, weeks, or months following administration of the compound of the invention (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient.

### Chemical Terms

It is to be understood that the terminology employed herein is for the purpose of describing particular embodiments and is not intended to be limiting.

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion, e.g., the interconversion illustrated in the scheme below:

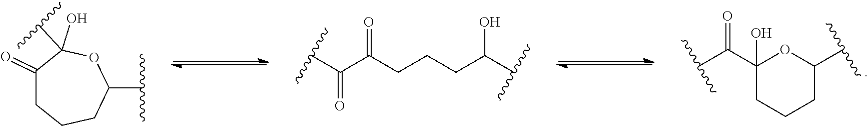 [SMILES: *C1CCCC(=O)C(*)(O)O1 ; *C(=O)C(=O)CCCC(*)O ; *C(=O)C1(O)CCCC(*)O1]

Those skilled in the art will appreciate that, in some embodiments, isotopes of compounds described herein may be prepared and/or utilized in accordance with the present invention. “Isotopes” refers to atoms having the same atomic number but different mass numbers resulting from a different number of neutrons in the nuclei. For example, isotopes of hydrogen include tritium and deuterium. In some embodiments, an isotopic substitution (e.g., substitution of hydrogen with deuterium) may alter the physiciochemical properties of the molecules, such as metabolism and/or the rate of racemization of a chiral center.

As is known in the art, many chemical entities (in particular many organic molecules and/or many small molecules) can adopt a variety of different solid forms such as, for example, amorphous forms and/or crystalline forms (e.g., polymorphs, hydrates, solvates, etc). In some embodiments, such entities may be utilized in any form, including in any solid form. In some embodiments, such entities are utilized in a particular form, for example in a particular solid form.

In some embodiments, compounds described and/or depicted herein may be provided and/or utilized in salt form.

In certain embodiments, compounds described and/or depicted herein may be provided and/or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substitutes are disclosed in groups or in ranges, unless otherwise indicated, the present disclosure is intended to cover individual compounds and groups of compounds (e.g., genera and subgenera) containing each and every individual subcombination of members at each position.

Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g. alkyl) per se is optional.

The term “acyl,” as used herein, represents a hydrogen or an alkyl group, as defined herein that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂RN², SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two RN¹ combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group.

Exemplary unsubstituted arylalkyl groups are from 7 to 30 carbons (e.g., from 7 to 16 or from 7 to 20 carbons, such as C₁-C₆ alkyl C₆-10 aryl, C₁-C₁₀ alkyl C₆₋₁₀ aryl, or C₁-C₂₀ alkyl C₆₋₁₀ aryl), such as, benzyl and phenethyl. In some embodiments, the akyl and the aryl each can be further substituted with 1, 2, 3, or 4 substituent groups as defined herein for the respective groups.

The term “azido,” as used herein, represents a —N₃ group.

The term “cyano,” as used herein, represents a CN group.

The term “carbocyclyl,” as used herein, refer to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, and adamantyl.

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic mono- or polycyclic radical of 5 to 12 atoms having at least one aromatic ring containing one, two, three, or four ring heteroatoms selected from N, O, and S, with the remaining ring atoms being C. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the akyl and the heteroaryl each can be further substituted with 1, 2, 3, or 4 substituent groups as defined herein for the respective groups.

The term “heterocyclyl,” as used herein, denotes a mono- or polycyclic radical having 3 to 12 atoms having at least one ring containing one, two, three, or four ring heteroatoms selected from N, O or S and no aromatic ring containing any N, O, or S atoms. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl. A heterocyclyl group may be aromatic or non-aromatic. An aromatic heterocyclyl is also referred to as heteroaryl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the akyl and the heterocyclyl each can be further substituted with 1, 2, 3, or 4 substituent groups as defined herein for the respective groups.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999). N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl.

Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an NO₂ group.

The term “oxyheteroaryl,” as used herein, represents a heteroaryl group having at least one endocyclic oxygen atom.

The term “oxyheterocyclyl,” as used herein, represents a heterocyclyl group having at least one endocyclic oxygen atom.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: aryl (e.g., substituted and unsubstituted phenyl), carbocyclyl (e.g., substituted and unsubstituted cycloalkyl), halo (e.g., fluoro), hydroxyl, oxo, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

As used herein, the term “administration” refers to the administration of a composition (e.g., a compound, a complex or a preparation that includes a compound or complex as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

As used herein, the term “animal” refers to any member of the animal kingdom. In some embodiments, “animal” refers to humans, at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically engineered animal, and/or a clone.

As used herein, the terms “approximately” and “about” are each intended to encompass normal statistical variation as would be understood by those of ordinary skill in the art as appropriate to the relevant context. In certain embodiments, the terms “approximately” or “about” each refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population).

As used herein, the terms “benefit” and “response” are used interchangeably in the context of a subject, such as a human subject undergoing therapy for the treatment of a neurological disorder, for example, amyotrophic lateral sclerosis, frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy. The terms “benefit” and “response” refer to any clinical improvement in the subject's condition. Exemplary benefits in the context of a subject undergoing treatment for a neurological disorder using the compositions and methods described herein (e.g., in the context of a human subject undergoing treatment for a neurological disorder described herein, such as amyotrophic lateral sclerosis, with a FYVE-type zinc finger containing phosphoinositide kinase (PIKfyve) inhibitor described herein, such as an inhibitory small molecule, antibody, antigen-binding fragment thereof, or interfering RNA molecule) include the slowing and halting of disease progression, as well as suppression of one or more symptoms associated with the disease. Particularly, in the context of a patient (e.g., a human patient) undergoing treatment for amyotrophic lateral sclerosis with a compound of the invention, examples of clinical “benefits” and “responses” are (i) an improvement in the subject's condition as assessed using the amyotrophic lateral sclerosis functional rating scale (ALSFRS) or the revised ALSFRS (ALSFRS-R) following administration of the compound of the invention, such as an improvement in the subject's ALSFRS or ALSFRS-R score within one or more days, weeks, or months following administration of the compound of the invention (e.g., an improvement in the subject's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (ii) an increase in the subject's slow vital capacity following administration of the compound of the invention, such as an increase in the subject's slow vital capacity within one or more days, weeks, or months following administration of the compound of the invention (e.g., an increase in the subject's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (iii) a reduction in decremental responses exhibited by the subject upon repetitive nerve stimulation, such as a reduction that is observed within one or more days, weeks, or months following administration of the compound of the invention (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (iv) an improvement in the subject's muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the compound of the invention (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (v) an improvement in the subject's quality of life, as assessed, for example, using the amyotrophic lateral sclerosis-specific quality of life (ALS-specific QOL) questionnaire, such as an improvement in the subject's quality of life that is observed within one or more days, weeks, or months following administration of the compound of the invention (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); and (vi) a decrease in the frequency and/or severity of muscle cramps exhibited by the subject, such as a decrease in cramp frequency and/or severity within one or more days, weeks, or months following administration of the compound of the invention (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject).

As used herein, the term “dosage form” refers to a physically discrete unit of an active compound (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

In the practice of the methods of the present invention, an “effective amount” of any one of the compounds of the invention or a combination of any of the compounds of the invention or a pharmaceutically acceptable salt thereof, is administered via any of the usual and acceptable methods known in the art, either singly or in combination.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of formula (I). For example pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

The terms “PIKfyve” and “FYVE-type zinc finger containing phosphoinositide kinase” are used interchangeably herein and refer to the enzyme that catalyzes phosphorylation of phosphatidylinositol 3-phosphate to produce phosphatidylinositol 3,5-bisphosphate, for example, in human subjects. The terms “PIKfyve” and “FYVE-type zinc finger containing phosphoinositide kinase” refer not only to wild-type forms of PIKfyve, but also to variants of wild-type PIKfyve proteins and nucleic acids encoding the same. The gene encoding PIKfyve can be accessed under NCBI Reference Sequence No. NG_021188.1. Exemplary transcript sequences of wild-type form of human PIKfyve can be accessed under NCBI Reference Sequence Nos. NM_015040.4, NM_152671.3, and NM_001178000.1. Exemplary protein sequences of wild-type form of human PIKfyve can be accessed under NCBI Reference Sequence Nos. NP_055855.2, NP_689884.1, and NP_001171471.1.

As used herein, the term “PIKfyve inhibitor” refers to substances, such as compounds of Formula I. Inhibitors of this type may, for example, competitively inhibit PIKfyve activity by specifically binding the PIKfyve enzyme (e.g., by virtue of the affinity of the inhibitor for the PIKfyve active site), thereby precluding, hindering, or halting the entry of one or more endogenous substrates of PIKfyve into the enzyme's active site. Additional examples of PIKfyve inhibitors that suppress the activity of the PIKfyve enzyme include substances that may bind PIKfyve at a site distal from the active site and attenuate the binding of endogenous substrates to the PIKfyve active site by way of a change in the enzyme's spatial conformation upon binding of the inhibitor. In addition to encompassing substances that modulate PIKfyve activity, the term “PIKfyve inhibitor” refers to substances that reduce the concentration and/or stability of PIKfyve mRNA transcripts in vivo, as well as those that suppress the translation of functional PIKfyve enzyme.

The term “pure” means substantially pure or free of unwanted components (e.g., other compounds and/or other components of a cell lysate), material defilement, admixture or imperfection.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

A variety of clinical indicators can be used to identify a patient as “at risk” of developing a particular neurological disease. Examples of patients (e.g., human patients) that are “at risk” of developing a neurological disease, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include (i) subjects exhibiting or prone to exhibit aggregation of TAR-DNA binding protein (TDP)-43, and (ii) subjects expressing a mutant form of TDP-43 containing a mutation associated with TDP-43 aggregation and toxicity, such as a mutation selected from Q331K, M337V, Q343R, N345K, R361S, and N390D. Subjects that are “at risk” of developing amyotrophic lateral sclerosis may exhibit one or both of these characteristics, for example, prior to the first administration of a PIKfyve inhibitor in accordance with the compositions and methods described herein.

As used herein, the terms “TAR-DNA binding protein-43” and “TDP-43” are used interchangeably and refer to the transcription repressor protein involved in modulating HIV-1 transcription and alternative splicing of the cystic fibrosis transmembrane conductance regulator (CFTR) pre-mRNA transcript, for example, in human subjects. The terms “TAR-DNA binding protein-43” and “TDP-43” refer not only to wild-type forms of TDP-43, but also to variants of wild-type TDP-43 proteins and nucleic acids encoding the same. The amino acid sequence and corresponding mRNA sequence of a wild-type form of human TDP-43 are provided under NCBI Reference Sequence Nos. NM_007375.3 and NP_031401.1, respectively.

The terms “TAR-DNA binding protein-43” and “TDP-43” as used herein include, for example, forms of the human TDP-43 protein that have an amino acid sequence that is at least 85% identical to the amino acid sequence of NCBI Reference Sequence No. NP_031401.1 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence No. NP_031401.1) and/or forms of the human TDP-43 protein that contain one or more substitutions, insertions, and/or deletions (e.g., one or more conservative and/or nonconservative amino acid substitutions, such as up to 5, 10, 15, 20, 25, or more, conservative or nonconservative amino acid substitutions) relative to a wild-type TDP-43 protein. For instance, patients that may be treated for a neurological disorder as described herein, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include human patients that express a form of TDP-43 having a mutation associated with elevated TDP-43 aggregation and toxicity, such as a mutation selected from Q331K, M337V, Q343R, N345K, R361S, and N390D. Similarly, the terms “TAR-DNA binding protein-43” and “TDP-43” as used herein include, for example, forms of the human TDP-43 gene that encode an mRNA transcript having a nucleic acid sequence that is at least 85% identical to the nucleic acid sequence of NCBI Reference Sequence No. NM_007375.3 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence No. NM_007375.3).

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

A “therapeutic regimen” refers to a dosing regimen whose administration across a relevant population is correlated with a desired or beneficial therapeutic outcome.

The term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

### BRIEF DESCRIPTION OF THE DRAWINGS

FIG. 1 is a scheme showing an approach to generation of a control TDP-43 yeast model (FAB1 TDP-43). A control yeast TDP-43 model was generated by integrating the human TDP-43 gene and the GAL1 promoter into the yeast genome. The yeast ortholog of human PIKFYVE is FAB1.

FIG. 2 is a scheme showing an approach to generation of a humanized PIKFYVE TDP-43 yeast model (PIKFYVE TDP-43). FAB1 gene through homologous recombination with a G418 resistance cassette (fab1::G418^{R}) (FIG. 2). PIKFYVE was cloned downstream of the GPD promoter harbored on a URA3-containing plasmid and introduced into the fab1::G418R ura3 strain. The pGAL1-TDP-43 construct was then introduced into the “humanized” yeast strain and assessed for cytotoxicity.

FIG. 3 is a histogram generated from the flow cytometry-based viability assay of FAB1 TDP-43.

FIG. 4 is a histogram generated from the flow cytometry-based viability assay of PIKFYVE TDP-43. Upon induction of TDP-43, there was a marked increase in inviable cells (rightmost population), with a more pronounced effect in PIKFYVE TDP-43 than in FAB1 TDP-43 strain (see FIG. 3).

FIG. 5 is an overlay of histograms generated from the flow cytometry-based viability assay of FAB1 TDP-43 in the presence of APY0201.

FIG. 6 is an overlay of histograms generated from the flow cytometry-based viability assay of PIKFYVE TDP-43 in the presence of APY0201.

FIG. 7 is a scatter plot comparing cytoprotection efficacy in PIKFYVE TDP-43 to PIKfyve inhibitory activity of test compounds.

### DETAILED DESCRIPTION

The present invention features compositions and methods for treating neurological disorders, such as amyotrophic lateral sclerosis and other neuromuscular disorders, as well as frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy among others. Particularly, the invention provides inhibitors of FYVE-type zinc finger containing phosphoinositide kinase (PIKfyve), that may be administered to a patient (e.g., a human patient) so as to treat or prevent a neurological disorder, such as one or more of the foregoing conditions. In the context of therapeutic treatment, the PIKfyve inhibitor may be administered to the patient to alleviate one or more symptoms of the disorder and/or to remedy an underlying molecular pathology associated with the disease, such as to suppress or prevent aggregation of TAR-DNA binding protein (TDP)-43.

The disclosure herein is based, in part, on the discovery that PIKfyve inhibition modulates TDP-43 aggregation in cells. Suppression of TDP-43 aggregation exerts beneficial effects in patients suffering from a neurological disorder. Many pathological conditions have been correlated with TDP-43-promoted aggregation and toxicity, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy. Without being limited by mechanism, by administering an inhibitor of PIKfyve, patients suffering from diseases associated with TDP-43 aggregation and toxicity may be treated, for example, due to the suppression of TDP-43 aggregation induced by the PIKfyve inhibitor.

Patients that are likely to respond to PIKfyve inhibition as described herein include those that have or are at risk of developing TDP-43 aggregation, such as those that express a mutant form of TDP-43 associated with TDP-43 aggregation and toxicity in vivo. Examples of such mutations in TDP-43 that have been correlated with elevated TDP-43 aggregation and toxicity include Q331K, M337V, Q343R, N345K, R361S, and N390D, among others. The compositions and methods described herein thus provide the additional clinical benefit of enabling the identification of patients that are likely to respond to PIKfyve inhibitor therapy, as well as processes for treating these patients accordingly.

The sections that follow provide a description of exemplary PIKfyve inhibitors that may be used in conjunction with the compositions and methods disclosed herein. The sections below additionally provide a description of various exemplary routes of administration and pharmaceutical compositions that may be used for delivery of these substances for the treatment of a neurological disorder.

### PIKfyve Inhibitors

Exemplary PIKfyve inhibitors described herein include compounds of formula (I)

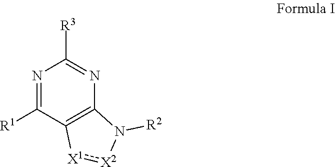

or a pharmaceutically acceptable salt thereof,
where
   [symbol] is a single bond, X¹ is C(R^{A})₂ or —OC(R^{A})₂—R^{X}, and X² is C(R^{A})₂ or CO; or [symbol] is a double bond, and each of X¹ and X² is independently CR^{A} or N, wherein R^{X} is a bond to X²;
   R¹ is -(L)ₙ-R^{B}; hydrogen; halogen; cyano; optionally substituted C₁₋₆ alkyl; optionally substituted C₁₋₆ heteroalkyl; optionally substituted C₁₋₆ alkoxy; optionally substituted C₆₋₁₀ aryl, optionally substituted C₁₋₉ heterocyclyl, or optionally substituted C₁₋₉ heteroaryl;
   R² is hydrogen, optionally substituted C₁₋₆ alkyl, optionally substituted C₆₋₁₀ aryl, optionally substituted C₁₋₉ heterocyclyl, or optionally substituted C₁₋₉ heteroaryl;
   R³ is a group of the following structure:

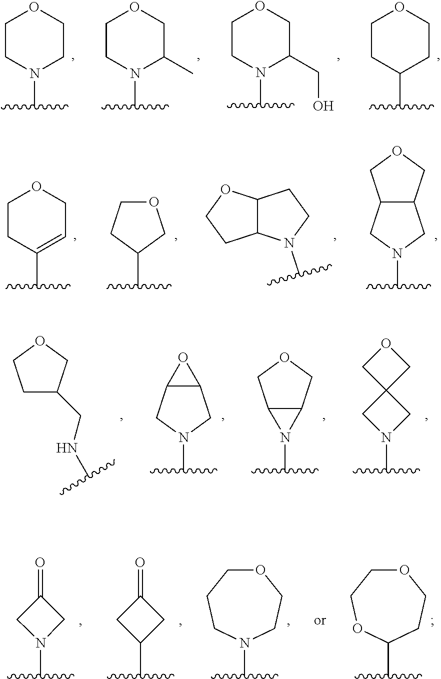

each R^{A} is independently H, optionally substituted C₁₋₆ alkyl, or optionally substituted C₆₋₁₀ aryl;
R^{B} is optionally substituted C₆₋₁₀ aryl, optionally substituted C₁₋₉ heteroaryl, optionally substituted C₃₋₈ cycloalkyl, or optionally substituted C₁₋₉ heterocyclyl;
R^{C} is H or optionally substituted C₁₋₆ alkyl;
each L is independently optionally substituted alkylene, O, or NR^{C}; and
n is 1, 2, or 3.

In some embodiments, R¹ is -(L)ₙ-R^{B}; optionally substituted C₁₋₆ alkoxy; optionally substituted C₁₋₉ heterocyclyl comprising at least one endocyclic oxygen; unsubstituted pyrimidinyl; optionally substituted pyridazinyl; optionally substituted oxazolyl, or pyrid-2-on-1-yl. In some embodiments, R² is optionally substituted C₆₋₁₀ aryl, optionally substituted C₁₋₉ heterocyclyl, or optionally substituted C₁₋₉ heteroaryl.

Methods of Treatment

Suppression of PIKfyve Activity and TDP-43 Aggregation to Treat Neurological Disorders

Using the compositions and methods described herein, a patient suffering from a neurological disorder may be administered a PIKfyve inhibitor, such as a small molecule described herein, so as to treat the disorder and/or to suppress one or more symptoms associated with the disorder. Exemplary neurological disorders that may be treated using the compositions and methods described herein are, without limitation, amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy, as well as neuromuscular diseases such as congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barré syndrome.

The present disclosure is based, in part, on the discovery that PIKfyve inhibitors, such as the agents described herein, are capable of attenuating TDP-43 toxicity. TDP-43-promoted toxicity has been associated with various neurological diseases. The discovery that PIKfyve inhibitors modulate TDP-43 aggregation provides an important therapeutic benefit. Using a PIKfyve inhibitor, such as a PIKfyve inhibitor described herein, a patient suffering from a neurological disorder or at risk of developing such a condition may be treated in a manner that remedies an underlying molecular etiology of the disease. Without being limited by mechanism, the compositions and methods described herein can be used to treat or prevent such neurological conditions, for example, by suppressing the TDP-43 aggregation that promotes pathology.

Additionally, the compositions and methods described herein provide the beneficial feature of enabling the identification and treatment of patients that are likely to respond to PIKfyve inhibitor therapy. For example, in some embodiments, a patient (e.g., a human patient suffering from or at risk of developing a neurological disease described herein, such as amyotrophic lateral sclerosis) is administered a PIKfyve inhibitor if the patient is identified as likely to respond to this form of treatment. Patients may be identified as such on the basis, for example, of susceptibility to TDP-43 aggregation. In some embodiments, the patient is identified is likely to respond to PIKfyve inhibitor treatment based on the isoform of TDP-43 expressed by the patient. For example, patients expressing TDP-43 isoforms having a mutation selected from Q331K, M337V, Q343R, N345K, R361S, and N390D, among others, are more likely to develop TDP-43-promoted aggregation and toxicity relative to patients that do not express such isoforms of TDP-43. Using the compositions and methods described herein, a patient may be identified as likely to respond to PIKfyve inhibitor therapy on the basis of expressing such an isoform of TDP-43, and may subsequently be administered a PIKfyve inhibitor so as to treat or prevent one or more neurological disorders, such as one or more of the neurological disorders described herein.

Assessing Patient Response

A variety of methods known in the art and described herein can be used to determine whether a patient having a neurological disorder (e.g., a patient at risk of developing TDP-43 aggregation, such as a patient expressing a mutant form of TDP-43 having a mutation associated with elevated TDP-43 aggregation and toxicity, for example, a mutation selected from Q331K, M337V, Q343R, N345K, R361S, and N390D) is responding favorably to PIKfyve inhibition. For example, successful treatment of a patient having a neurological disease, such as amyotrophic lateral sclerosis, with a PIKfyve inhibitor described herein may be signaled by:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the PIKfyve inhibitor (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient); (ii) an increase in slow vital capacity, such as an increase in the patient's slow vital capacity within one or more days, weeks, or months following administration of the PIKfyve inhibitor (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iii) a reduction in decremental responses exhibited by the patient upon repetitive nerve stimulation, such as a reduction that is observed within one or more days, weeks, or months following administration of the PIKfyve inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the PIKfyve inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the PIKfyve inhibitor (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(vi) a decrease in the frequency and/or severity of muscle cramps, such as a decrease in cramp frequency and/or severity within one or more days, weeks, or months following administration of the PIKfyve inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient); and/or
(vii) a decrease in TDP-43 aggregation, such as a decrease in TDP-43 aggregation within one or more days, weeks, or months following administration of the PIKfyve inhibitor (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient.

### Combination Formulations and Uses Thereof

The compounds of the invention can be combined with one or more therapeutic agents. In particular, the therapeutic agent can be one that treats or prophylactically treats any neurological disorder described herein.

Combination Therapies

A compound of the invention can be used alone or in combination with other agents that treat neurological disorders or symptoms associated therewith, or in combination with other types of treatment to treat, prevent, and/or reduce the risk of any neurological disorders. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

### Pharmaceutical Compositions

The compounds of the invention are preferably formulated into pharmaceutical compositions for administration to human subjects in a biologically compatible form suitable for administration in vivo. Accordingly, in another aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers.

A compound of the invention may also be administered parenterally. Solutions of a compound of the invention can be prepared in water suitably mixed with a surfactant. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20^{th} ed.) and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg.

Alternatively, the dosage amount can be calculated using the body weight of the patient. For example, the dose of a compound, or pharmaceutical composition thereof, administered to a patient may range from 0.1-50 mg/kg.

The following examples are meant to illustrate the invention. They are not meant to limit the invention in any way.

### EXAMPLES

### List of Abbreviations:

DIPEA=N,N-diisopropylethylamine
EtOH=ethanol
THF=tetrahydrofuran
nBuLi=n-butyl lithium
I₂=iodine
Pd(dppf)Cl₂=[1,1′-Bis(diphenylphosphino)ferrocene]dichloropalladium(II)
Cs₂CO₃=cesium carbonate
H₂O=water
Pd(PPh₃)Cl₂=Bis(triphenylphosphine)palladium(II) dichloride
Pd(PPh₃)₄=tetrakis(triphenylphosphine)palladium(0)
LiCl=lithium chloride
MecOH=methanol
NBS=N-bromosuccinimide
ACN=acetonitrile
K₂CO₃=potassium carbonate
DMA=N,N-dimethylacetamide
Zn(CN)₂=zinc cyanide
HATU=1-[Bis(dimethylamino)methylene]-1H-1,2,3-triazolo[4,5-b]pyridinium 3-oxid hexafluorophosphate
DMF=N,N-dimethylformamide
Pd(t-Bu₃P)₂=Bis(tri-tert-butylphosphine)palladium(0)
DMF-DMA=N,N-dimethylformamide dimethyl acetal
N₂H₄H₂O=hydrazine hydrate
Pd₂(dba)₃=tris(dibenzylideneacetone) dipalladium
X-Phos=2-dicyclohexylphosphino-2′,4′,6′-triisopropylbiphenyl
Pd(PPh₃)Cl₂ DCM=Bis(triphenylphosphine)palladium(II) dichloride dichloromethane complex
DMSO=dimethylsulfoxide
DPPA=diphenyl phosphorylazide
Et₃N=triethylamine
HCl=hydrochloric acid

### Example 1. Preparation of Compounds

### General Schemes

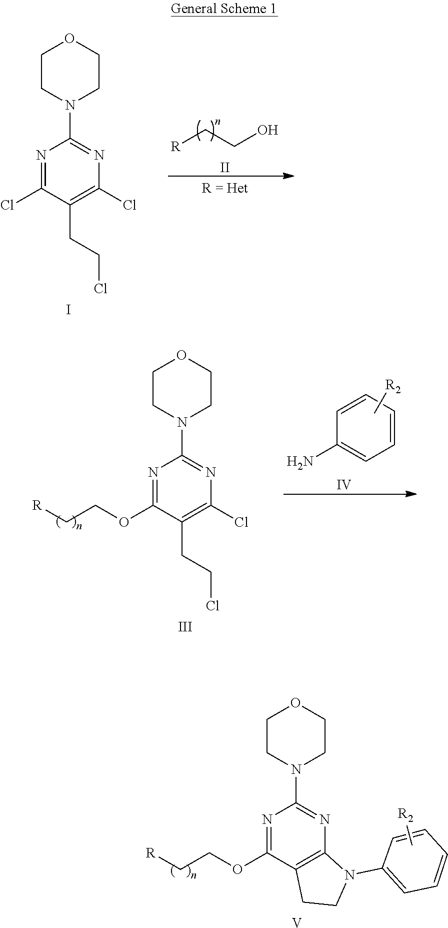

An appropriately substituted morpholine I can be reacted with an appropriated substituted aryl alcohol II under basic conditions to afford an appropriately substituted morpholine ether intermediate III. The morpholine ether intermediate III is then coupled with an appropriately substituted aryl amine IV under Buchwald-Hartwig conditions to yield an appropriately substituted aryl morpholine ether product V.

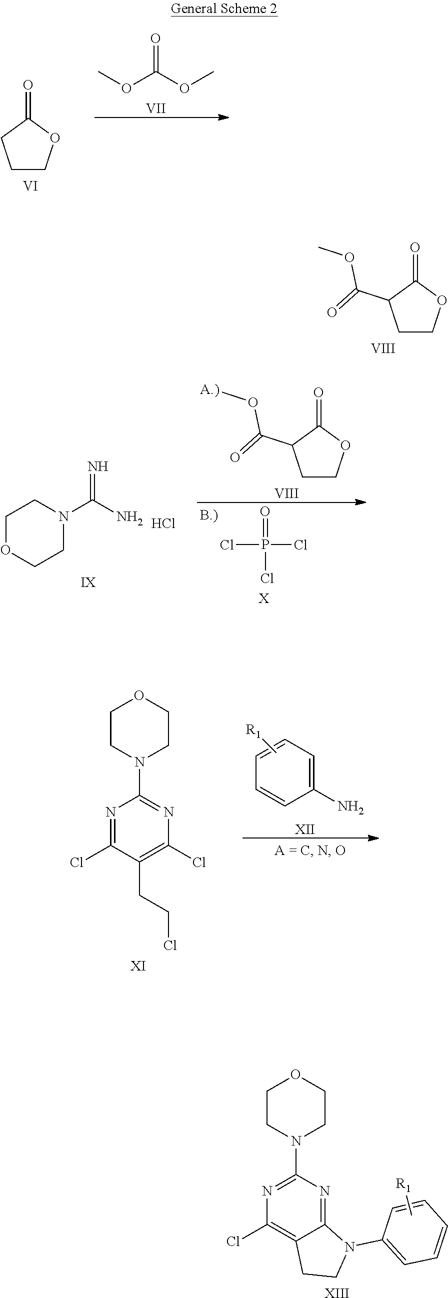

An appropriately substituted dihydrofuran VI is reacted with an appropriately substituted carbonate VII under basic conditions to afford the appropriately substituted tetrahydrofuran ester intermediate VIII. An appropriately substituted morpholine imine intermediate IX is either (A) reacted with the tetrahydrofuran ester intermediate VIII under Claisen condensation conditions or (B) reacted with phosphoryl chloride X under basic conditions to afford the appropriately substituted pyrimidine morpholine intermediate XI. This pyrimidine morpholine intermediate XI is reacted with an appropriately substituted aryl amine XII under basic conditions to afford the appropriately substituted pyrrolopyrimidine product XIII.

### Synthesis of 4-(7-phenyl-4-(2-(pyridin-2-yl)ethoxy)-6,7-dihydro-5H-pyrrolo[2,3-d]pyrimidin-2-yl)morpholine (Compound 1)

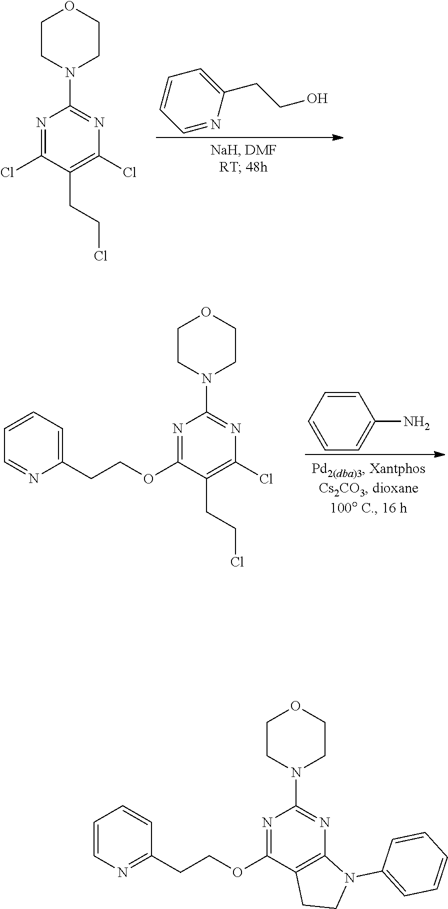 [SMILES: ClCCc1c(Cl)nc(N2CCOCC2)nc1Cl ; OCCc1ccccn1 ; ClCCc1c(Cl)nc(N2CCOCC2)nc1OCCc1ccccn1 ; Nc1ccccc1 ; c1ccc(N2CCc3c(OCCc4ccccn4)nc(N4CCOCC4)nc32)cc1]

### Step 1: Synthesis of 4-(4-chloro-5-(2-chloroethyl)-6-(2-(pyridin-2-yl)ethoxy)pyrimidin-2-yl)morpholine

A solution of 2-(pyridin-2-yl)ethanol (830 mg, 6.74 mmol) in DMF was added to a solution of sodium hydride (270 mg, 6.74 mmol) in DMF (60 mL) at 0° C. The resultant mixture was warmed up and stirred at room temperature for 10 min followed by the addition of 4-(4,6-dichloro-5-(2-chloroethyl)pyrimidin-2-yl)morpholine (2 g, 6.74 mmol). The reaction mixture was stirred further at room temperature for 48 h. It was quenched with water (200 mL) and extracted with ethyl acetate (300 mL×2). The combined organic layers were washed with water (200 mL×2), brine (200 mL), dried over sodium sulfate, filtered and concentrated. The crude product obtained was purified by silica gel column chromatography, eluting with petroleum ether/ethyl acetate=3/1 to obtain 4-(4-chloro-5-(2-chloroethyl)-6-(2-(pyridin-2-yl)ethoxy)pyrimidin-2-yl)morpholine (1.9 g, 74%) as off-white solid. LCMS (ESI) m/z: 398.1 [M+16]⁺.

### Step 2: Synthesis of 4-(7-phenyl-4-(2-(pyridin-2-yl)ethoxy)-6,7-dihydro-5H-pyrrolo[2,3-d]pyrimidin-2-yl)morpholine

A mixture of 4-(4-chloro-5-(2-chloroethyl)-6-(2-(pyridin-2-yl)ethoxy)pyrimidin-2-yl)morpholine (100 mg, 0.261 mmol), aniline (49 mg, 0.526 mmol), tris(dibenzylideneacetone)dipalladium (24 mg, 0.026 mmol), 4,5-bis(diphenylphosphino)-9,9-dimethylxanthene (30 mg, 0.052 mmol) and cesium carbonate (170 mg, 0.522 mmol) in dioxane (5 mL) was stirred at 100° C. for 16 h under nitrogen atmosphere. After cooling to room temperature, the reaction mixture was diluted with ethyl acetate (100 mL), washed with water (30 mL×2), brine (30 mL), dried over sodium sulfate, filtered and concentrated. The residue obtained was subjected to silica gel column chromatography (eluting with petroleum ether/ethyl acetate=2/1) and then to PREP-HPLC (SunFire C18, 4.6*50 mm, 3.5 um column Xbridge C18 3.5 μm 4.6×50 mm column. The elution system used was a gradient of 5%˜95% over 1.5 min at 2 ml/min and the solvent was acetonitrile/0.01% aqueous ammonium bicarbonate.) to obtain 4-(7-phenyl-4-(2-(pyridin-2-yl)ethoxy)-6,7-dihydro-5H-pyrrolo[2,3-d]pyrimidin-2-yl)morpholine (53.8 mg, 51%) as white solid. ¹H NMR (400 MHz, DMSO-de) b 8.50 (dd, J=4.8, 0.8 Hz, 1H), 7.75-7.70 (m, 2H), 7.35-7.31 (m, 3H), 7.25-7.22 (m, 1H), 6.96 (t, J=7.2 Hz, 1H), 4.63 (t, J=6.8 Hz, 2H), 3.99 (t, J=8.6 Hz, 2H), 3.66 (s, 8H), 3.16 (t, J=6.8 Hz, 2H), 2.79 (t, J=8.6 Hz, 2H). LCMS (ESI) m/z: 404.2 [M+H]⁺.

### Synthesis of 4-(7-phenyl-4-(pyridin-2-ylmethoxy)-6,7-dihydro-5H-pyrrolo[2,3-d]pyrimidin-2-yl)morpholine (Compound 2)

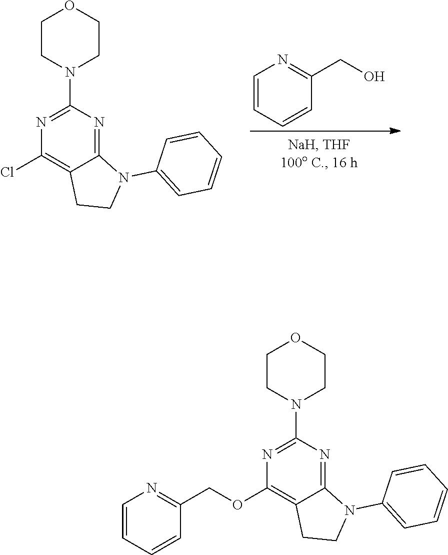 [SMILES: Clc1nc(N2CCOCC2)nc2c1CCN2c1ccccc1 ; OCc1ccccn1 ; c1ccc(N2CCc3c(OCc4ccccn4)nc(N4CCOCC4)nc32)cc1]

To a solution of pyridin-2-ylmethanol (52 mg, 0.47 mmol) in dry THE (10 mL) was added NaH (28 mg, 0.71 mmol) and the mixture was stirred at 0° C. 15 min. A solution of 4-(4-chloro-7-phenyl-6,7-dihydro-5H-pyrrolo[2,3-d]pyrimidin-2-yl)morpholine (150 mg, 0.47 mmol) in THE (5 mL) was then added and the resultant mixture stirred for another 16 h at 100° C. The reaction was quenched with ice water (20 mL) and extracted with EtOAc (20 mL*3). The organic layer was dried over sodium sulfate, filtered and concentrated in vacuo. The residue was purified by prep-HPLC to give 4-(7-phenyl-4-(pyridin-2-ylmethoxy)-6,7-dihydro-5H-pyrrolo[2,3-d]pyrimidin-2-yl)morpholine (28.4 mg, 16%) as white solid. ¹H NMR (400 MHz, DMSO-d₆) δ 8.54 (dd, J=4.8, 0.8 Hz, 1H), 7.82-7.78 (m, 1H), 7.76 (s, 1H), 7.74 (s, 1H), 7.42 (d, J=8.0 Hz, 1H), 7.36-7.29 (m, 3H), 6.97 (t, J=7.2 Hz, 1H), 5.44 (s, 2H), 4.04 (t, J=8.8 Hz, 2H), 3.59 (s, 8H), 2.94 (t, J=8.8 Hz, 2H); LCMS (ESI) m/z:390.3 [M+H]⁺.

The following compounds were synthesized according to the protocol described above:

| Compound | | | |
|---|---|---|---|
| # | Structure | LCMS | NMR |
| 3 | 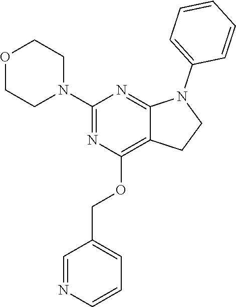 [SMILES: c1ccc(N2CCc3c(OCc4cccnc4)nc(N4CCOCC4)nc32)cc1] | LCMS (ESI) m/z: 390 [M + H]⁺. | 1H NMR (400 MHz, DMSO-d6) δ 8.67 (s, 1H), 8.53 (s, 1H), 7.85 (d, J = 7.6 Hz, 1H), 7.75 (d, J = 8.0 Hz, 1H), 7.30-7.50 (m, 3H), 6.98 (m, 1H), 5.44 (s, 2H), 4.03 (t, J = 8.0 Hz, 2H), 3.66 (s, 8H), 2.90 (t, J = 8.4 Hz, 2H) |
| 4 | 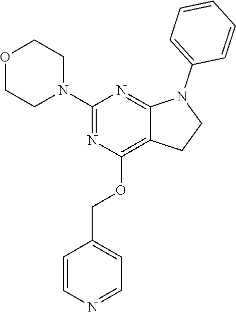 [SMILES: c1ccc(N2CCc3c(OCc4ccncc4)nc(N4CCOCC4)nc32)cc1] | LCMS (ESI) m/z: 390 [M + H]⁺ | 1H NMR (400 MHz, DMSO-d6) δ 8.56 (s, 2H), 7.75 (d, J = 6.4 Hz, 2H), 7.25-7.45 (m, 4H), 6.98 (t, J = 6.0 Hz, 1H), 5.44 (s, 2H), 4.06 (t, J = 2.8 Hz, 2H), 3.61 (s, 8H), 2.96 (t, J = 3.2 Hz, 2H) |
| 5 | 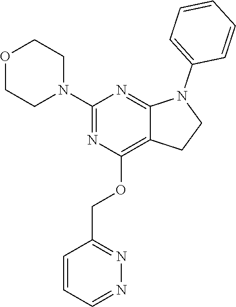 [SMILES: c1ccc(N2CCc3c(OCc4cccnn4)nc(N4CCOCC4)nc32)cc1] | LCMS (ESI) m/z: 391.2 [M + H]⁺. | 1H NMR (500 MHz, DMSO-d6) δ 2.95 (t, J = 9.0 Hz, 2H), 3.57 (s, 8H), 4.05 (t, J = 8.0 Hz, 2H), 5.65 (s, 2H), 6.97 (t, J = 7.5 Hz, 1H), 7.34 (q, J = 7.0 Hz, 2H), 7.70 (d, J = 3.5 Hz, 2H), 7.75 (d, J = 8.0 Hz, 2H), 9.17 (t, J = 3.0 Hz, 1H) |
| 6 | 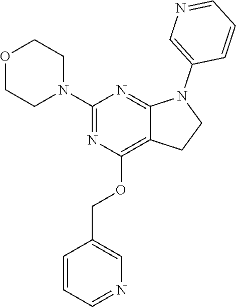 [SMILES: c1cncc(COc2nc(N3CCOCC3)nc3c2CCN3c2cccnc2)c1] | LCMS (ESI) m/z: 391 [M + H]⁺. | 1H NMR (400 MHz, DMSO-d6) δ 9.00 (s, 1H), 8.67 (s, 1H), 8.54 (s, 1H), 8.18 (m, 2H), 7.86 (m, 1H), 7.40 (m, 2H), 5.45 (s, 2H), 4.06 (t, J = 8.4 Hz, 2H) 3.67 (s, 8H), 2.93 (t, J = 8.4 Hz, 2H) |
| 7 | 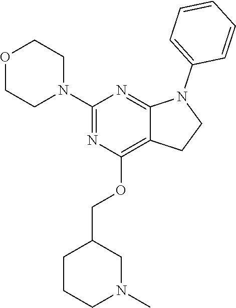 [SMILES: CN1CCCC(COc2nc(N3CCOCC3)nc3c2CCN3c2ccccc2)C1] | LCMS (ESI) m/z: 410.3 [M + H]⁺ | 1H NMR (500 MHz, DMSO-d6) δ 8.22 (s, 1H), 7.76 (d, J = 8.0 Hz, 2H), 7.34 (t, J = 8.0 Hz, 2H), 6.97 (t, J = 7.5 Hz, 1H), 4.22-4.19 (m, 1H), 4.15-4.12 (m, 1H), 4.02 (t, J = 8.5 Hz, 2H), 3.66 (s, 8H), 2.88 (t, J = 9.0 Hz, 2H), 2.83 (d, J = 10.5 Hz, 1H), 2.70 (d, J = 10.5 Hz, 1H), 2.21 (s, 3H), 1.98 (t, J = 10.0 Hz, 2H), 1.85 (t, J = 10.0 Hz, 1H), 1.70-1.63 (m, 2H), 1.53-1.49 (m, 1H), 1.08-1.01 (m, 1H) |
| 8 | 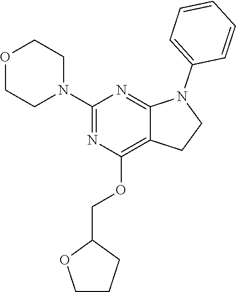 [SMILES: c1ccc(N2CCc3c(OCC4CCCO4)nc(N4CCOCC4)nc32)cc1] | LCMS (ESI) m/z: 383.1 [M + H]⁺ | 1H NMR (400 MHz, DMSO-d6) δ 7.76-7.74 (d, J = 8.0 Hz, 2H), 7.36-7.32 (m, 2H), 6.97 (m, 1H), 4.28-4.21 (m, 2H), 4.13 (m, 1H), 4.01 (t, J = 8.6 Hz, 2H), 3.80-3.75 (m, 1H), 3.69-3.64 (m, 9H), 2.86 (t, J = 8.6 Hz, 2H), 1.97-1.81 (m, 3H), 1.63 (m, 1H) |
| 9 | 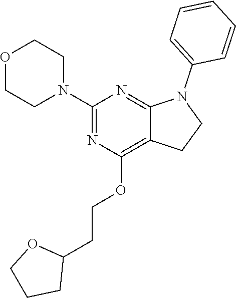 [SMILES: c1ccc(N2CCc3c(OCCC4CCCO4)nc(N4CCOCC4)nc32)cc1] | LCMS (ESI) m/z: 397.2 [M + H]⁺ | 1H NMR (500 MHz, DMSO-d6) δ 7.75 (d, J = 5.0 Hz, 2H), 7.34 (t, J = 8.0 Hz, 2H), 6.96 (t, J = 7.5 Hz, 1H), 4.42-4.29 (m, 2H), 4.01 (t, J = 8.5 Hz, 2H), 3.90-3.84 (m, 1H), 3.75 (dd, J = 14.0, 7.5 Hz, 1H), 3.66 (s, 8H), 3.62-3.58 (m, 1H), 2.86 (t, J = 8.0 Hz, 2H), 2.00-1.97 (m, 1H), 1.88-1.80 (m, 4 H), 1.49-1.45 (m, 1H) |
| 10 | 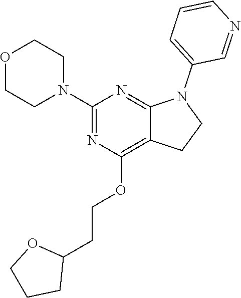 [SMILES: c1cncc(N2CCc3c(OCCC4CCCO4)nc(N4CCOCC4)nc32)c1] | LCMS (ESI) m/z: 398.1 [M + H]⁺. | 1H NMR (400 MHz, DMSO-d6) δ 8.99 (d, J = 2.4 Hz, 1H), 8.18-8.14 (m, 2H), 7.36 (m, 1H), 4.41-4.31 (m, 2H), 4.04 (t, J = 8.6 Hz, 2H), 3.87 (m, 1H), 3.78-3.57 (m, 10H), 2.89 (t, J = 8.6 Hz, 2H), 1.98 (m, 1H), 1.88-1.79 (m, 2H), 1.47 (m, 1H) |

### Synthesis of tert-butyl 4-(2-morpholino-4-(pyridin-3-ylmethoxy)-5H-pyrrolo[2,3-d]pyrimidin-7(6H)-yl)piperidine-1-carboxylate (Compound 28) and 4-(7-(piperidin-4-yl)-4-(pyridin-3-ylmethoxy)-6,7-dihydro-5H-pyrrolo[2,3-d]pyrimidin-2-yl)morpholine (Compound 53)

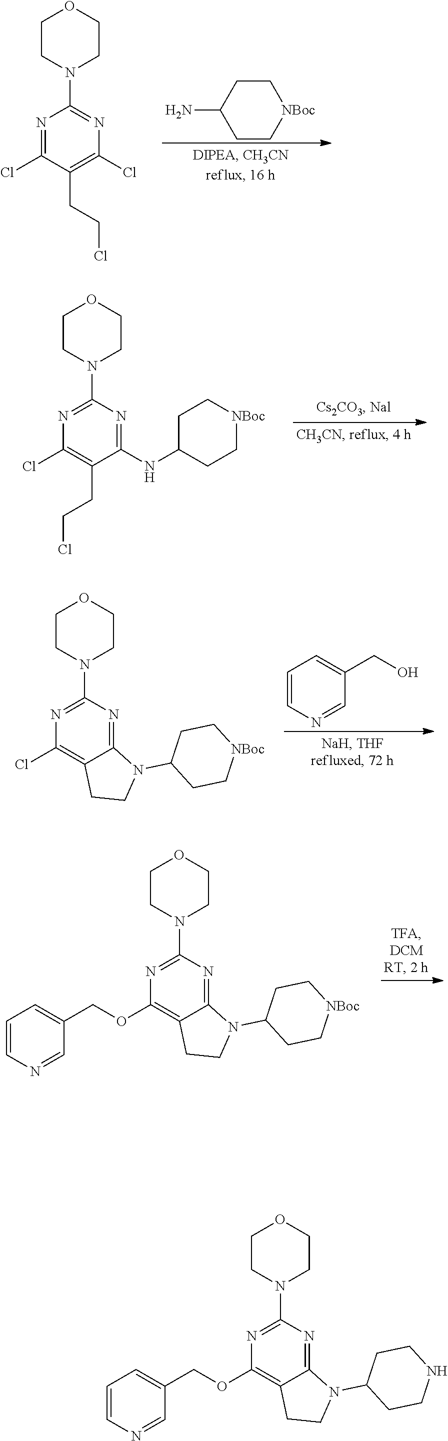

### Step 1: Synthesis of tert-butyl 4-(6-chloro-5-(2-chloroethyl)-2-morpholinopyrimidin-4-ylamino)piperidine-1-carboxylate

To a stirred solution of 4-(4,6-dichloro-5-(2-chloroethyl)pyrimidin-2-yl)morpholine (100 mg, 0.337 mmol) and tert-butyl 4-aminopiperidine-1-carboxylate (135 mg, 0.674 mmol) in acetonitrile (5 mL) at room temperature was added DIPEA (109 mg, 0.843 mmol) and the resultant mixture was refluxed for 16 h. After cooling to room temperature, the mixture was diluted with ethyl acetate (50 mL), washed with water (20 mL), brine (20 mL), dried over sodium sulfate, filtered and concentrated to obtain tert-butyl 4-(6-chloro-5-(2-chloroethyl)-2-morpholinopyrimidin-4-ylamino)piperidine-1-carboxylate (100 mg, 64%) as white solid. This material was used in the next step without further purification. LCMS (ESI) m/z: 460.1 [M+H]⁺.

### Step 2: Synthesis of tert-butyl 4-(4-chloro-2-morpholino-5H-pyrrolo[2,3-d]pyrimidin-7(6H)-yl)piperidine-1-carboxylate

Cesium carbonate (177 mg, 0.543 mmol) was added to a mixture of tert-butyl 4-(6-chloro-5-(2-chloroethyl)-2-morpholinopyrimidin-4-ylamino)piperidine-1-carboxylate (100 mg, 0.217 mmol) and sodium iodide (7 mg, 0.047 mmol) in acetonitrile (10 mL) at room temperature. The resultant mixture was refluxed for 4 h under nitrogen atmosphere. After cooling to room temperature, the mixture was diluted with ethyl acetate (80 mL), washed with water (30 mL) and brine (30 mL). The organics were dried over sodium sulfate, filtered and concentrated. The crude product obtained was purified by silica gel column chromatography, eluting with petroleum ether/ethyl acetate=9/1 then 3/1 to obtain tert-butyl 4-(4-chloro-2-morpholino-5H-pyrrolo[2,3-d]pyrimidin-7(6H)-yl)piperidine-1-carboxylate (20 mg, 22%) as white solid. LCMS (ESI) m/z: 424.3 [M+H]⁺.

### Step 3: Synthesis of tert-butyl 4-(2-morpholino-4-(pyridin-3-ylmethoxy)-5H-pyrrolo[2,3-d]pyrimidin-7(6H)-yl)piperidine-1-carboxylate

A suspension of pyridin-3-ylmethanol (10 mg, 0.092 mmol) and sodium hydride (5 mg, 0.125 mmol) in tetrahydrofuran (3 mL) was stirred at room temperature for 10 min followed by the addition of tert-butyl 4-(4-chloro-2-morpholino-5H-pyrrolo[2,3-d]pyrimidin-7(6H)-yl)piperidine-1-carboxylate (20 mg, 0.047 mmol). The reaction mixture was then refluxed for 72 h and cooled. It was then diluted with ethyl acetate (80 mL), washed with water (30 mL×2) and brine (20 mL), dried over sodium sulfate, filtered and concentrated. The crude product obtained was purified by prep-HPLC (SunFire C18, 4.6*50 mm, 3.5 um column Xbridge C18 3.5 μm 4.6×50 mm column. The elution system used was a gradient of 5%˜95% over 1.5 min at 2 ml/min and the solvent was acetonitrile/0.01% aqueous ammonium bicarbonate.) to obtain tert-butyl 4-(2-morpholino-4-(pyridin-3-ylmethoxy)-5H-pyrrolo[2,3-d]pyrimidin-7(6H)-yl)piperidine-1-carboxylate (14.6 mg, 62%) as white solid. ¹H NMR (400 MHz, MeOD) δ 8.60 (d, J=1.6 Hz, 1H), 8.47 (dd, J=5.2, 1.6 Hz, 1H), 7.90 (dt, J=8.0, 1.6 Hz, 1H), 7.44 (dd, J=8.0, 0.8 Hz, 1H), 5.42 (s, 2H), 4.18 (d, J=124 Hz, 2H), 4.03 (pent, J=7.6 Hz, 11H), 3.68 (s, 8H), 3.54 (t, J=8.4 Hz, 2H), 2.82-2.78 (m, 4H), 1.73-1.68 (m, 4H), 1.48 (s, 9H). LCMS (ESI) m/z: 497.1 [M+H]⁺.

### Step 4: Synthesis of 4-(7-(piperidin-4-yl)-4-(pyridin-3-ylmethoxy)-6,7-dihydro-5H-pyrrolo[2,3-d]pyrimidin-2-yl)morpholine

Trifluoroacetic acid (1 ml) was added to a solution of tert-butyl 4-(2-morpholino-4-(pyridin-3-ylmethoxy)-5H-pyrrolo[2,3-d]pyrimidin-7(6H)-yl)piperidine-1-carboxylate (60 mg, 0.121 mmol) in dichloromethane (2 mL) at room temperature. After stirring at room temperature for 2 h, the reaction mixture was concentrated. The residue was subjected to prep-HPLC (SunFire C18, 4.6*50 mm, 3.5 um column Xbridge C18 3.5 μm 4.6×50 mm column. The elution system used was a gradient of 5%˜95% over 1.5 min at 2 ml/min and the solvent was acetonitrile/0.0% aqueous ammonium bi(arbonaie) to obtain 4-(7-(piperidin-4-yl)-4-(pyridin-3-ylmethoxy)-6,7-dihydro-5H-pyrrolo[2,3-d]pyrimidin-2-yl)morpholine (9.5 mg, 20%) as white solid. ¹H NMR (400 MHz, CD3OD) δ 8.60 (s, 1H), 8.48 (d, J=4.4 Hz, 1H), 7.90 (d, J=8.0 Hz, 1H), 7.44 (dd, J=8.0, 4.8 Hz, 1H), 5.42 (s, 2H), 4.08-4.03 (m, 1H), 3.69 (s, 8H), 3.58 (t, J=8.0 Hz, 2H), 3.32-3.30 (m, 2H), 2.91-2.80 (m, 4H), 1.90-1.81 (m, 4H). LCMS (ESI)/z: 397.1 [M+H].

The following compounds were synthesized according to the protocol described above:

| Name | Structure | NMR, MS | # |
|---|---|---|---|
| 4-(2-morpholino-4- (pyridin-3-ylmethoxy)- 5H-pyrrolo[2,3- d]pyrimidin-7(6H)- yl)benzonitrile | 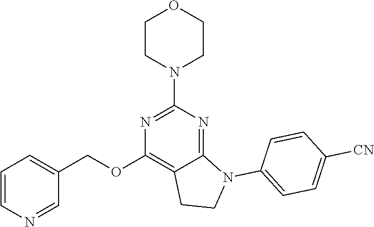 [SMILES: N#Cc1ccc(N2CCc3c(OCc4cccnc4)nc(N4CCOCC4)nc32)cc1] | ¹H NMR (400 MHz, DMSO-d₆) δ 8.66 (d, J = 1.6 Hz, 1H), 8.53 (dd, J = 4.8, 1.6 Hz, 1H), 7.93 (d, J = 9.2 Hz, 2H), 7.85 (d, J = 8.0 Hz, 1H), 7.75 (d, J = 8.8 Hz, 2H), 7.41 (dd, J = 7.6, 4.8 Hz, 1H), 5.45 (s, 2H), 4.06 (t, J = 8.4 Hz, 2H), 3.67 (s, 8H), 2.92 (t, J = 8.4 Hz, 2H). LCMS (ESI) m/z: 415.2 [M + H]⁺. | 21 |
| 4-(7-(3-fluorophenyl)-4- (pyridazin-3-ylmethoxy)- 6,7-dihydro-5H- pyrrolo[2,3-d]pyrimidin- 2-yl)morpholine | 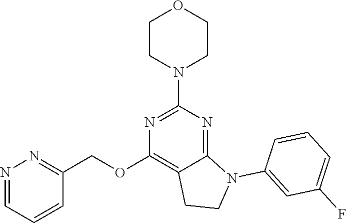 [SMILES: Fc1cccc(N2CCc3c(OCc4cccnn4)nc(N4CCOCC4)nc32)c1] | ¹H NMR (400 MHz, DMSO-d₆) δ 9.18 (t, J = 3.2 Hz, 1H), 7.76 (dt, J = 11.2, 2.4 Hz, 1H), 7.71 (d, J = 3.2 Hz, 2H), 7.50 (dd, J = 8.0, 1.2 Hz, 1H), 7.37 (q, J = 4.4 Hz, 1H), 6.79 (dt, J = 10.4, 2.0 Hz, 1H), 5.658 (s, 2H), 4.05 (t, J = 8.4 Hz, 2H), 3.58 (s, 8H), 2.95 (t, J = 8.8 Hz, 2H); LC-MS: m/z = 409.2 (M + H)⁺. | 14 |
| 4-(7-(pyrimidin-5-yl)-4- ((tetrahydro-2H-pyran-4- yl)methoxy)-6,7-dihydro- 5H-pyrrolo[2,3- d]pyrimidin-2- yl)morpholine | 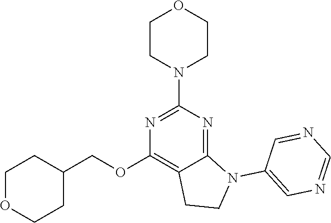 [SMILES: c1ncc(N2CCc3c(OCC4CCOCC4)nc(N4CCOCC4)nc32)cn1] | ¹H NMR (400 MHz, Chloroform-d) δ 9.19 (s, 2H), 8.83 (s, 1H), 4.21 (d, J = 6.5 Hz, 2H), 4.03 (t, J = 9.2 Hz, 4H), 3.80 (m, 8H), 3.45 (dt, J = 9.6, 2.0 Hz, 2H), 3.02 (t, J = 9.2, 7.8 Hz, 2H), 2.09-1.96 (m, 1H), 1.74-1.66 (m, 2H), 1.52-1.39 (m, 2H). LCMS (ESI) m/z: 399.3 [M + H]⁺. | 77 |
| 4-(4-((1-ethylpiperidin-3- yl)methoxy)-7-(pyridin- 3-yl)-6,7-dihydro-5H- pyrrolo[2,3-d]pyrimidin- 2-yl)morpholine | 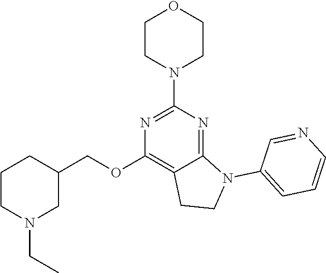 [SMILES: CCN1CCCC(COc2nc(N3CCOCC3)nc3c2CCN3c2cccnc2)C1] | ¹H NMR (400 MHz, Chloroform-d) δ 9.06 (d, J = 2.7 Hz, 1H), 8.24 (d, J = 4.8 Hz, 1H), 8.08 (dd, J = 9.8, 2.4 Hz, 1H), 7.28 (d, J = 2.4 Hz, 1H), 4.27-4.22 (m, 1H), 4.18-4.10 (m, 1H), 4.03 (t, J = 8.5 Hz, 2H), 3.78 (s, 8H), 3.06-2.91 (m, 4H), 2.49-2.35 (m, 2H), 2.17-2.03 (m, 1H), 1.92-1.84 (m, 1H), 1.83-1.72 (m, 3H), 1.67-1.60 (m, 1H), 1.13-1.00 (m, 4H). LCMS (ESI) m/z: 425.3 [M + H]⁺. | 92 |
| 4-(4-(2-(1- methylpiperidin-3- yl)ethoxy)-7-(pyridin-3- yl)-6,7-dihydro-5H- pyrrolo[2,3-d]pyrimidin- 2-yl)morpholine | 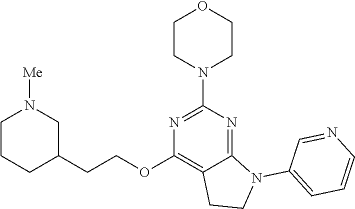 [SMILES: CN1CCCC(CCOc2nc(N3CCOCC3)nc3c2CCN3c2cccnc2)C1] | ¹H NMR (400 MHz, Chloroform-d) δ 9.05 (d, J = 2.7 Hz, 1H), 8.22 (dd, J = 4.4, 1.2 Hz, 1H), 8.06 (dd, J = 11.2, 2.4 Hz, 1H), 7.26-7.22 (m, 1H), 4.36 (t, J = 6.4 Hz, 2H), 4.01 (t, J = 8.6 Hz, 2H), 3.76 (s, 8H), 2.97 (t, J = 9.2 Hz, 2H), 2.90-2.76 (m, 2H), 2.27 (s, 3H), 1.92-1.76 (m, 4H), 1.70-1.57 (m, 4H), 0.97-0.88 (m, 1H). LCMS (ESI) m/z: 425.4 [M + H]⁺. | 93 |
| 4-methyl-2-((2- morpholino-7-(pyridin-3- yl)-6,7-dihydro-5H- pyrrolo[2,3-d]pyrimidin- 4- yloxy)methyl)morpholine | 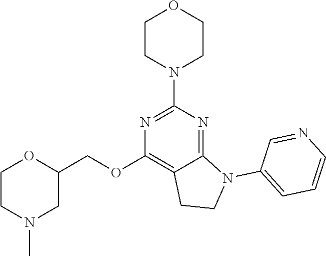 [SMILES: CN1CCOC(COc2nc(N3CCOCC3)nc3c2CCN3c2cccnc2)C1] | ¹H NMR (400 MHz, Chloroform-d) δ 9.05 (s, 1H), 8.23 (d, J = 4.6 Hz, 1H), 8.05 (dd, J = 9.6, 1.2 Hz, 1H), 7.28 (d, J = 4.8 Hz, 1H), 4.40 (dd, J = 17.2, 6.0 Hz, 1H), 4.32 (dd, J = 16.4, 5.2 Hz, 1H), 4.01 (t, J = 9.2, 8.0 Hz, 2H), 3.96-3.87 (m, 2H), 3.79-3.67 (m, 9H), 3.01 (t, J = 9.3, 7.9 Hz, 2H), 2.87-2.80 (m, 1H), 2.70-2.63 (m, 1H), 2.32 (s, 3H), 2.21-2.11 (m, 1H), 1.97 (t, J = 11.2, 10.3 Hz, 1H). LCMS (ESI) m/z: 413.3 [M + H]⁺. | 94 |
| 4-(7-(3-methoxyphenyl)- 4-((1-methylpiperidin-3- yl)methoxy)-6,7-dihydro- 5H-pyrrolo[2,3- d]pyrimidin-2- yl)morpholine | 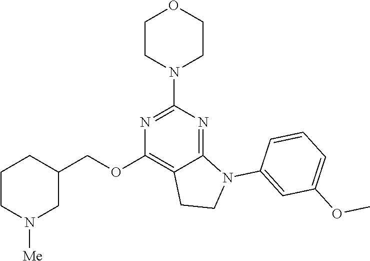 [SMILES: COc1cccc(N2CCc3c(OCC4CCCN(C)C4)nc(N4CCOCC4)nc32)c1] | ¹H NMR (400 MHz, DMSO-d₆) δ 7.67 (s, 1H), 7.21 (t, J = 8.0 Hz, 1H), 7.11 (d, J = 8.8 Hz, 1H), 6.56 (dd, J = 8.0, 2.4 Hz, 1H), 4.25 (dd, J = 10.8, 5.6 Hz, 1H), 4.13 (dd, J = 10.8, 7.2 Hz, 1H), 3.99 (t, J = 8.4 Hz, 2H), 3.79 (s, 3H), 3.75 (s, 8H), 3.01-2.99 (m, 2H), 2.91-2.86 (m, 3H), 2.32 (s, 3H), 2.11-2.04 (m, 1H), 2/04-1.99 (m, 1H), 1.78-1.77 (m, 3H), 1.67-1.65 (m, 1H), 1.19-1.06 (m, 1H); LC- MS : m/z = 440.3 (M + H)⁺. | 95 |
| 4-(7-phenyl-4- (pyrimidin-5- ylmethoxy)-6,7-dihydro- 5H-pyrrolo[2,3- d]pyrimidin-2- yl)morpholine | 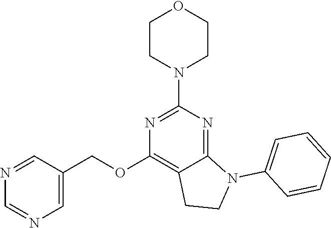 [SMILES: c1ccc(N2CCc3c(OCc4cncnc4)nc(N4CCOCC4)nc32)cc1] | ¹H NMR (400 MHz, DMSO-d₆) δ 9.15 (s, 1H), 8.90 (s, 2H), 7.74 (d, J = 8.4 Hz, 2H), 7.34 (t, J = 7.6 Hz, 2H), 7.00 (t, J = 7.2 Hz, 1H), 5.46 (s, 2H), 4.03 (t, J = 8.4 Hz, 2H), 3.65 (s, 8H), 2.91 (t, J = 8.8 Hz, 2H); LC-MS: m/z = 391.2 (M + H)⁺. | 96 |
| 4-(7-phenyl-4-(pyrazin- 2-ylmethoxy)-6,7- dihydro-5H-pyrrolo[2,3- d]pyrimidin-2- yl)morpholine | 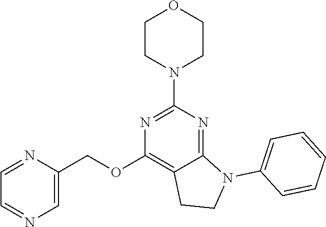 [SMILES: c1ccc(N2CCc3c(OCc4cnccn4)nc(N4CCOCC4)nc32)cc1] | ¹H NMR (400 MHz, DMSO-d₆) δ 8.75 (s, 1H), 8.62 (s, 1H), 8.56 (s, 1H), 7.77 (d, J = 8.4 Hz, 2H), 7.33 (t, J = 8 Hz, 2H), 7.01-6.99 (m, 1H), 5.56 (s, 2H), 4.09 (t, J = 8.0 Hz, 2H), 3.68 (s, 8H), 3.04 (t, J = 8.0 Hz, 2H); LC-MS: m/z = 391.2 (M + H)⁺. | 97 |
| 4-(4-((1-methylpiperidin- 3-yl)methoxy)-7- (pyrimidin-5-yl)-6,7- dihydro-5H-pyrrolo[2,3- d]pyrimidin-2- yl)morpholine | 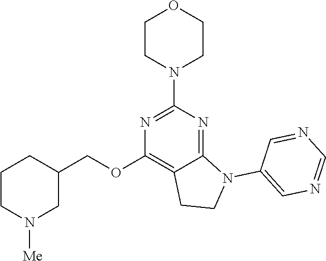 [SMILES: CN1CCCC(COc2nc(N3CCOCC3)nc3c2CCN3c2cncnc2)C1] | ¹H NMR (500 MHz, DMSO-d₆) δ 9.20 (s, 2H), 8.78 (s, 1H), 4.23-4.04 (m, 4H), 3.66 (s, 8H), 2.93 (t, J = 8.4 Hz, 2H), 2.78-2.62 (m, 2H), 2.16 (s, 3H), 1.98-1.89 (m, 2H), 1.76-1.61 (m, 3H), 1.49 (m, 1H), 1.03 (m, 1H). LCMS (ESI) m/z: 412.1 [M + H]⁺. | 80 |
| 4-(4-((1-methylpiperidin- 3-yl)methoxy)-7- (pyridazin-4-yl)-6,7- dihydro-5H-pyrrolo[2,3- d]pyrimidin-2- yl)morpholine | 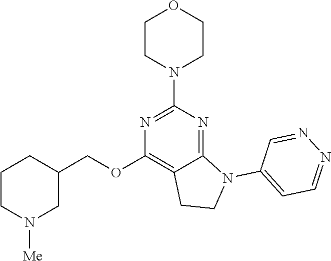 [SMILES: CN1CCCC(COc2nc(N3CCOCC3)nc3c2CCN3c2ccnnc2)C1] | ¹H NMR (500 MHz, DMSO-d₆) δ 9.77 (d, J = 2.4 Hz, 1H), 8.93 (d, J = 6.0 Hz, 1H), 7.80 (dd, J = 6.0, 2.4 Hz, 1H), 4.24-4.13 (m, 2H), 4.06 (t, J = 8.4 Hz, 2H), 3.34 (s, 8H), 2.93 (t, J = 8.4 Hz, 2H), 2.79-2.63 (m, 2H), 2.17 (s, 3H), 2.00-1.79 (m, 3H), 1.70-1.46 (m, 3H), 1.01 (m, 1H). LCMS (ESI) m/z: 412.3 [M + H]⁺. | 81 |
| 4-(7-(1-methyl-1H- pyrazol-4-yl)-4-((1- methylpiperidin-3- yl)methoxy)-6,7-dihydro- 5H-pyrrolo[2,3- d]pyrimidin-2- yl)morpholine | 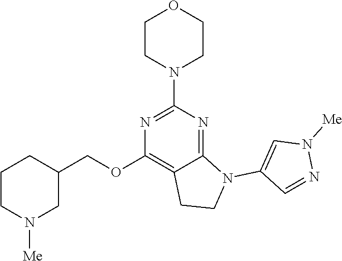 [SMILES: CN1CCCC(COc2nc(N3CCOCC3)nc3c2CCN3c2cnn(C)c2)C1] | ¹H NMR (400 MHz, DMSO-d₆) δ 7.80 (s, 1H), 7.63 (s, 1H), 4.18-4.07 (m, 2H), 3.84-3.79 (m, 5H), 3.65 (s, 8H), 2.88 (t, J = 8.6 Hz, 2H), 2.77-2.62 (m, 2H), 2.15 (s, 3H), 1.97-1.92 (m, 2H), 1.77-1.59 (m, 3H), 1.47 (m, 1H), 0.98 (m, 1H). LCMS (ESI) m/z: 414.2 [M + H]⁺. | 82 |
| 2-methyl-1-(2- morpholino-7-(pyridin-3- yl)-6,7-dihydro-5H- pyrrolo[2,3-d]pyrimidin- 4-yloxy)propan-2-ol | 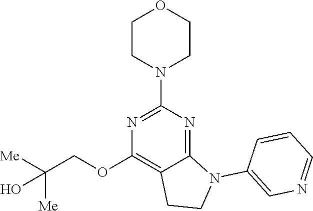 [SMILES: CC(C)(O)COc1nc(N2CCOCC2)nc2c1CCN2c1cccnc1] | ¹H NMR (400 MHz, Chloroform-d) δ 9.05 (s, 1H), 8.25 (s, 1H), 8.08-8.06 (m , 1H), 7.32-7.27 (m, 1H), 4.25 (s, 2H), 4.05 (t, J = 8.5 Hz, 2H), 3.81-3.68 (m, 8H), 3.61 (s, 1H), 3.02 (t, J = 8.5 Hz, 2H), 1.30 (s, 6H). LCMS (ESI) m/z: 372.2 [M + H]⁺. | 83 |
| 4-(4-(oxetan-3- ylmethoxy)-7-phenyl- 6,7-dihydro-5H- pyrrolo[2,3-d]pyrimidin- 2-yl)morpholine | 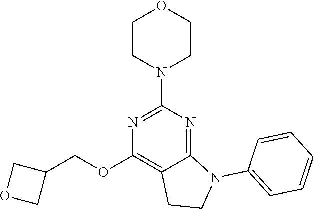 [SMILES: c1ccc(N2CCc3c(OCC4COC4)nc(N4CCOCC4)nc32)cc1] | ¹H NMR (400 MHz, DMSO-d₆) δ 7.75 (d, J = 8.0 Hz, 2H), 7.34 (t, J = 7.6 Hz, 2H), 6.97 (t, J = 7.6 Hz, 1H), 4.68 (dd, J = 7.6, 6.0 Hz, 2H), 4.52 (d, J = 6.8 Hz, 2H), 4.40 (t, J = 6.0 Hz, 2H), 4.00 (t, J = 8.4 Hz, 2H), 3.66 (s, 8H), 3.36-3.350 (m, 1H), 2.85 (t, J = 7.2 Hz, 2H); LC-MS: m/z = 369.3 (M + H)⁺. | 99 |
| 4-(7-phenyl-4- ((tetrahydro-2H-pyran-4- yl)oxy)-6,7-dihydro-5H- pyrrolo[2,3-d]pyrimidin- 2-yl)morpholine | 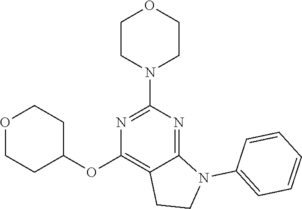 [SMILES: c1ccc(N2CCc3c(OC4CCOCC4)nc(N4CCOCC4)nc32)cc1] | ¹H NMR (400 MHz, DMSO-d₆) δ 7.75 (d, J = 8.0 Hz, 1H), 7.34 (t, J = 8 Hz, 2H), 6.97 (t, J = 7.2 Hz, 1H), 5.20 (sept, J = 4.4 Hz, 1H), 4.02 (t, J = 8.4 Hz, 2H), 3.88-3.82 (m, 2H), 3.65 (d, J = 4.4 Hz, 8H), 3.53-3.47 (m, 2H), 2.88 (t, J = 8.4 Hz, 2H), 2.01-1.97 (m, 2H), 1.66-1.59 (m, 2H); LCMS (ESI) m/z: 383.1 [M + H]⁺. | 11 |
| 4-(7-phenyl-4- ((tetrahydrofuran-3- yl)oxy)-6,7-dihydro-5H- pyrrolo[2,3-d]pyrimidin- 2-yl)morpholine | 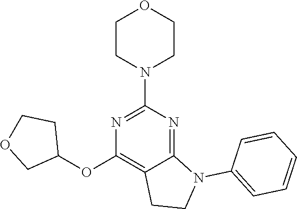 [SMILES: c1ccc(N2CCc3c(OC4CCOC4)nc(N4CCOCC4)nc32)cc1] | ¹H NMR (400 MHz, DMSO-d₆) δ 7.75 (d, J = 8.0 Hz, 2H), 7.34 (t, J = 8 Hz, 2H), 6.98 (t, J = 7.2 Hz, 1H), 5.52 (sept, J = 2.0 Hz, 1H), 4.02 (t, J = 8.4 Hz, 2H), 3.92 (dd, J = 10.0, 4.8 Hz, 1H), 3.84 (dd, J = 15.6, 8.0 Hz, 1H), 3.77-3.70 (m, 2H), 3.66 (s, 8H), 2.89-2.85 (m, 2H), 2.22 (dd, J = 13.6, 6.8 Hz, 1H), 2.00 (d, J = 6.8 Hz, 1H); LCMS (ESI) m/z: 369.1 [M + H]⁺. | 12 |

### Synthesis of 4-(7-phenyl-4-((pyridin-2-ylmethoxy)methyl)-6,7-dihydro-5H-pyrrolo[2,3-d]pyrimidin-2-yl)morpholine (Compound 43)

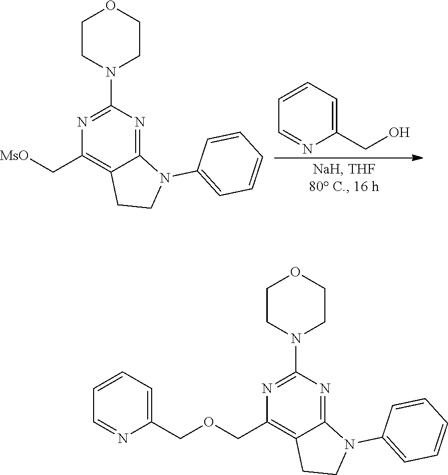

To a solution of pyridin-2-ylmethanol (29 mg, 0.27 mmol) in THE (8 mL) was added sodium hydride (11 mg, 0.27 mmol) at 0° C. portion wise. The mixture was stirred at 0° C. for 30 min followed by the addition of (2-morpholino-7-phenyl-6,7-dihydro-5H-pyrrolo[2,3-d]pyrimidin-4-yl)methyl methanesulfonate (70 mg, 0.18 mmol). The resultant mixture was stirred at 80° C. for 16 h, then quenched with water (10 mL) and extracted with dichloromethane (20 mL*3). The organic layer was washed with brine (20 mL), dried over sodium sulfate, filtered and concentrated. The residue was purified by prep-HPLC (0.05% NH4HCO3/H2O:CH3CN=5%˜95%) to obtain 4-(7-phenyl-4-((pyridin-2-ylmethoxy)methyl)-6,7-dihydro-5H-pyrrolo[2,3-d]pyrimidin-2-yl)morpholine (1.8 mg, 15%,) as white solid. ¹H NMR (400 MHz, CDCl3) δ 8.59 (d, J=4.4 Hz, 1H), 7.79 (d, J=8.0 Hz, 2H), 7.74 (dt, J=8.0, 2.0 Hz, 1H), 7.53 (d, J=8.0 Hz, 1H), 7.40 (t, J=7.6 Hz, 2H), 7.25-7.22 (m, 1H), 7.06 (t, J=7.6 Hz, 1H), 4.77 (s, 2H), 4.56 (s, 2H), 4.06 (t, J=8.4 Hz, 2H), 3.79 (s, 8H), 3.16 (t, J=8.4 Hz, 2H); LCMS (ESI) m/z: 404.1 [M+H]⁺.

The following compounds were synthesized according to the protocol described above:

| Name | Structure | NMR, MS | # |
|---|---|---|---|
| tert-butyl 3-((2- morpholino-7- (pyridin- 3-yl)-6,7- dihydro-5H- pyrrolo[2,3-d] pyrimidin- 4- yl)methoxy) pyrrolidine- 1-carboxylate | 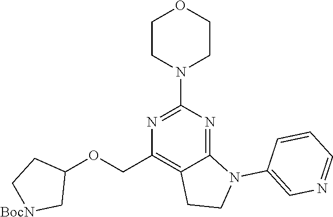 [SMILES: CC(C)(C)OC(=O)N1CCC(OCc2nc(N3CCOCC3)nc3c2CCN3c2cccnc2)C1] | ¹H NMR (400 MHz, CDCl₃) δ 9.10 (bs, 1H), 8.29 (bs, 1H), 8.10 (d, J = 8 Hz, 1H), 7.29 (d, J = 3.6 Hz, 1H), 4.41 (s, 2H), 4.16 (bs, 1H), 4.03 (t, J = 8.4 Hz, 2H), 3.76 (s, 8H), 3.57-3.43 (m, 4H), 3.15 (t, J = 8.4 Hz, 2H), 2.08-1.96 (m, 2H), 1.46 (s, 9H). LCMS (ESI) m/z: 483.1 [M + H]⁺. | 15 |
| 4-(7-(pyridin- 3-yl)-4- ((pyrrolidin-3- yloxy)methyl)- 5,6- dihydro-5H- pyrrolo[2,3- d]pyrimidin-2- yl)morpholine | 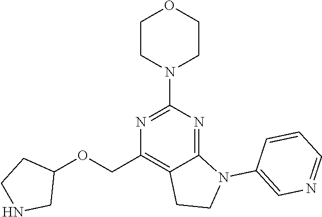 [SMILES: c1cncc(N2CCc3c(COC4CCNC4)nc(N4CCOCC4)nc32)c1] | ¹H NMR (400 MHz, DMSO-d₆) δ 9.05 (d, J = 2.4 Hz, 1H), 8.24-8.21 (m, 2H), 7.42-7.38 (m, 1H), 4.35 (s, 2H), 4.21 (bs, 1H), 4.07 (t, J = 8.4 Hz, 2H), 3.65 (s, 8H), 3.41-3.29 (m, 2H), 3.12-3.04 (m, 5H), 1.99-1.87 (m, 2H). LCMS (ESI) m/z: 383.1 [M + H]⁺. | 13 |
| 4-(4-((oxetan-3- yloxy)methyl)-7- (pyridin-3-yl)- 6,7- dihydro-5H- pyrrolo[2,3- d]pyrimidin-2- yl)morpholine | 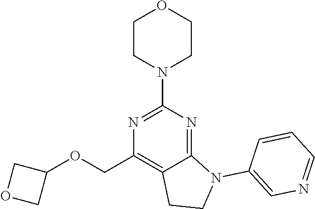 [SMILES: c1cncc(N2CCc3c(COC4COC4)nc(N4CCOCC4)nc32)c1] | ¹H NMR (400 MHz, DMSO-d₆) δ 9.05 (d, J = 2.8 Hz, 1H), 8.24-8.20 (m, 2H), 7.42-7.29 (m, 1H), 4.70-4.66 (m, 3H), 4.46-4.44 (m, 2H), 4.30 (s, 2H), 4.08 (t, J = 8.4 Hz, 2H), 3.65 (s, 8H), 3.10 (t, J = 8.4 Hz, 2H). LCMS (ESI) m/z: 370.1 [M + H]⁺. | 31 |
| 4-(4-((1- methylpyrrolidin- 3- yloxy)methyl)-7- (pyridin-3-yl)-6,7- dihydro-5H- pyrrolo[2,3- d]pyrimidin-2- yl)morpholine | 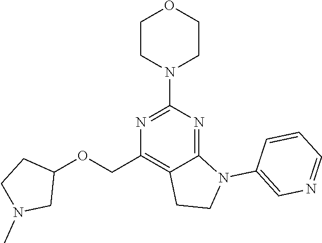 [SMILES: CN1CCC(OCc2nc(N3CCOCC3)nc3c2CCN3c2cccnc2)C1] | ¹H NMR (400 MHz, DMSO-d₆) δ 9.05 (d, J = 2.0 Hz, 1H), 8.24-8.20 (m, 2H), 7.42-7.39 (m, 1H), 4.31 (s, 2H), 4.16 (bs, 1H), 4.07 (t, J = 8.4 Hz, 2H), 3.65 (s, 8H), 3.10 (t, J = 8.4 Hz, 2H), 2.86-2.73 (m, 3H), 2.60-2.55 (m, 1H), 2.40 (s, 3H), 2.12-2.02 (m, 1H), 1.85-1.75 (m, 1H). LCMS (ESI) m/z: 397.1 [M + H]⁺. | 29 |

### Synthesis of 4-(4-methoxy-7-phenyl-6,7-dihydro-5H-pyrrolo[2,3-d]pyrimidin-2-yl)morpholine (Compound 16)

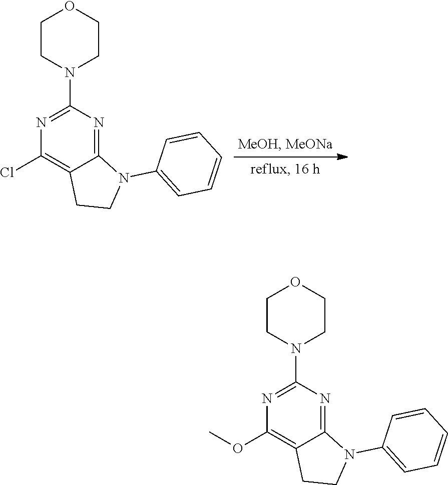 [SMILES: Clc1nc(N2CCOCC2)nc2c1CCN2c1ccccc1 ; COc1nc(N2CCOCC2)nc2c1CCN2c1ccccc1]

To a solution of 4-(4-chloro-7-phenyl-6,7-dihydro-5H-pyrrolo[2,3-d]pyrimidin-2-yl)morpholine (100 mg, 0.34 mmol) in methanol (80 mL) was added sodium methoxide (8 mL). The mixture was refluxed overnight and concentrated. The crude product obtained was purified by prep-HPLC to give 4-(4-methoxy-7-phenyl-6,7-dihydro-5H-pyrrolo[2,3-d]pyrimidin-2-yl)morpholine (18.3) mg as white solid. ¹H NMR (400 MHz, DMSO-d₆) δ 7.75 (d, J=6.8 Hz, 2H), 7.34 (t, J=6.0 Hz, 2H), 6.97 (t, J=5.6 Hz, 1H), 4.01 (t, J=6.8 Hz, 2H), 3.85 (s, 3H), 3.67 (s, 8H), 2.86 (t, J=6.8 Hz, 2H); LCMS (ESI) m/z: 313 [M+H]⁺.

### Synthesis of 4-(7-(3-fluorophenyl)-4-(pyridin-3-ylmethoxy)-6,7-dihydro-5H-pyrrolo[2,3-d]pyrimidin-2-yl)morpholine (Compound 17)

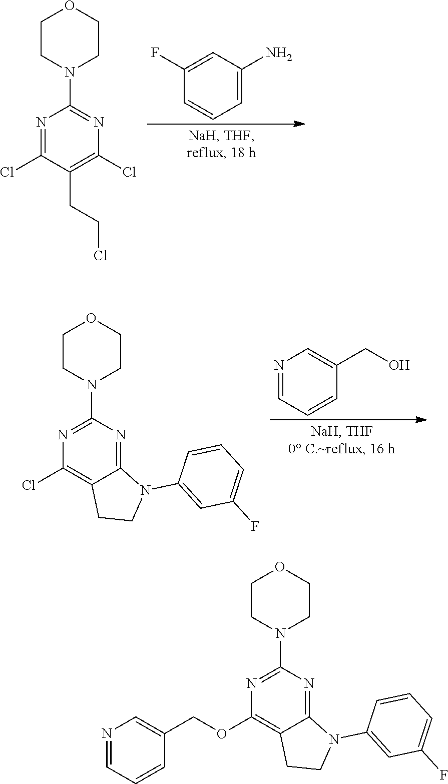 [SMILES: ClCCc1c(Cl)nc(N2CCOCC2)nc1Cl ; Nc1cccc(F)c1 ; Fc1cccc(N2CCc3c(Cl)nc(N4CCOCC4)nc32)c1 ; OCc1cccnc1 ; Fc1cccc(N2CCc3c(OCc4cccnc4)nc(N4CCOCC4)nc32)c1]

### Step 1: Synthesis of 4-(4-chloro-7-(3-fluorophenyl)-6,7-dihydro-5H-pyrrolo[2,3-d]pyrimidin-2-yl)morpholine

To a solution of 3-fluoroaniline (181 mg, 1.63 mmol) in THE (20 mL) was added NaH (130 mg, 3.25 mmol) at 0° C. slowly. The mixture was stirred at 60° C. for 2 h and then 4-(4,6-dichloro-5-(2-chloroethyl)pyrimidin-2-yl)morpholine (400 mg, 1.35 mmol) was added. The resultant mixture was stirred at 110° C. for 16 h and then quenched with saturated aqueous NH₄Cl solution (20 mL). The mixture was extracted with EtOAc (50*3 mL), the combined organics were washed with brine (100 mL), dried over Na2SO4, filtered and concentrated. The residue was purified by SGC (PE/EA=1:1) to obtain 4-(4-chloro-7-(3-fluorophenyl)-6,7-dihydro-5H-pyrrolo[2,3-d]pyrimidin-2-yl)morpholine (270 mg, 59%) as yellow solid. LCMS (ESI) m/z: 335.0 [M+H]⁺.

### Step 2: Synthesis of 4-(7-(3-fluorophenyl)-4-(pyridin-3-ylmethoxy)-6,7-dihydro-5H-pyrrolo[2,3-d]pyrimidin-2-yl)morpholine

To a solution of pyridin-3-ylmethanol (86 mg, 0.79 mmol) in THE (20 mL) was added NaH (32 mg, 0.79 mmol) at 0° C. slowly. The mixture was stirred at 0° C. for 2 h and then 4-(4-chloro-7-(3-fluorophenyl)-6,7-dihydro-5H-pyrrolo[2,3-d]pyrimidin-2-yl)morpholine (220 mg, 0.66 mmol) was added. The resultant mixture was stirred at 110° C. for 16 h and concentrated. The crude product obtained was purified by prep-HPLC (0.05% FA/H2O:CH3CN=5%˜95%) to obtain 4-(7-(3-fluorophenyl)-4-(pyridin-3-ylmethoxy)-6,7-dihydro-5H-pyrrolo[2,3-d]pyrimidin-2-yl)morpholine (47.2 mg, 18%,) as white solid. ¹H NMR (400 MHz, DMSO-d₆) δ 8.67 (s, 1H), 8.54 (d, J=4.0 Hz, 1H), 7.86 (d, J=8.0 Hz, 1H), 7.77 (d, J=12.8 Hz, 1H), 7.49 (d, J=8.4 Hz, 1H), 7.43-7.33 (m, 2H), 6.81-6.76 (m, 1H), 5.45 (s, 2H), 4.03 (t, J=8.8 Hz, 2H), 3.67 (s, 8H), 2.90 (t, J=8.8 Hz, 2H); LCMS (ESI) m/z: 408.1 [M+H]⁺.

### Synthesis of 4-(7-phenyl-4-(pyridin-2-yloxy)-6,7-dihydro-5H-pyrrolo[2,3-d]pyrimidin-2-yl)morpholine (Compound 18) and 1-(2-morpholino-7-phenyl-6,7-dihydro-5H-pyrrolo[2,3-d]pyrimidin-4-yl)pyridin-2(1H)-one (Compound 76)

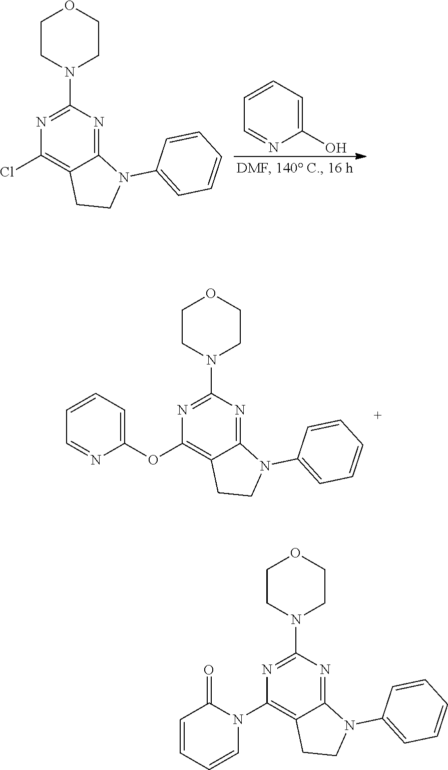

To a solution of 4-(4-chloro-7-phenyl-6,7-dihydro-5H-pyrrolo[2,3-d]pyrimidin-2-yl)morpholine (100 mg, 0.32 mmol) in DMF (10 mL) were added pyridin-2-ol (33 mg, 0.35 mmol) and K₂CO₃ (88 mg, 0.64 mmol) and the resultant mixture was stirred at 140° C. for 16 h. Then the reaction was quenched with water (5 mL) and was extracted with EtOAc (20*3 mL). The organic layer was combined, washed with brine (30 mL), dried over Na₂SO₄, filtered and concentrated. The residue was purified by prep-HPLC (0.05% NH₄HCO₃/H₂O:CH₃CN=5%˜95%) to offer 4-(7-phenyl-4-(pyridin-2-yloxy)-6,7-dihydro-5H-pyrrolo[2,3-d]pyrimidin-2-yl)morpholine (9.3 mg, 8%) and 1-(2-morpholino-7-phenyl-6,7-dihydro-5H-pyrrolo[2,3-d]pyrimidin-4-yl)pyridin-2(1H)-one (9.0 mg, 8%) as yellow solids.

Compound 18: ¹H NMR (400 MHz, CDCl₃) δ 8.31 (dd, J=4.8, 1.2 Hz, 1H), 7.79-7.75 (m, 3H), 7.41-7.36 (m, 2H), 7.15-7.04 (m, 3H), 4.07 (t, J=8.4 Hz, 2H), 3.74-3.69 (m, 8H), 2.92 (t, J=8.4 Hz, 2H); LCMS (ESI) m/z: 376.1 [M+H]⁺.

Compound 76: ¹H NMR (400 MHz, CDCl₃) δ 7.77 (d, J=7.6 Hz, 2H), 7.67 (dd, J=1.6, 6.4 Hz, 1H), 7.43-7.39 (m, 3H), 7.10 (t, J=7.6 Hz, 1H), 7.63 (d, J=9.2 Hz, 1H), 6.28 (t, J=6.4 Hz, 1H), 4.11 (t, J=8.4 Hz, 2H), 3.81-3.77 (m, 8H), 3.05 (t, J=8.4 Hz, 2H); LCMS (ESI) m/z: 376.1 [M+H]⁺.

The following compounds were synthesized according to the protocol described above:

| Name | Structure | NMR, MS | # |
|---|---|---|---|
| 4-(7-phenyl-4-(pyridin-3- yloxy)-6,7-dihydro-5H- pyrrolo[2,3-d]pyrimidin-2- yl)morpholine | 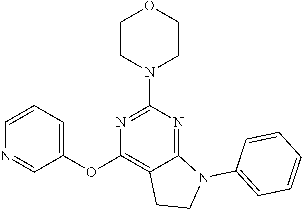 [SMILES: c1ccc(N2CCc3c(Oc4cccnc4)nc(N4CCOCC4)nc32)cc1] | ¹H NMR (400 MHz, DMSO-d₆) δ 8.48 (d, J = 2.6 Hz, 1H), 8.42 (dd, J = 4.7, 1.3 Hz, 1H), 7.79 (d, J = 7.9 Hz, 2H), 7.67 (ddd, J = 8.3, 2.8, 1.4 Hz, 1H), 7.46 (dd, J = 8.3, 4.7 Hz, 1H), 7.43-7.32 (m, 2H), 7.03 (t, J = 7.3 Hz, 1H), 4.11 (t, J = 8.5 Hz, 2H), 3.67-3.54 (m, 4H), 3.47 (s, 4H), 3.00 (t, J = 8.5 Hz, 2H); LCMS (ESI) m/z: 376.2 [M + H]⁺. | 22 |
| 4-(7-phenyl-4-(pyridin-4- yloxy)-6,7-dihydro-5H- pyrrolo[2,3-d]pyrimidin-2- yl)morpholine | 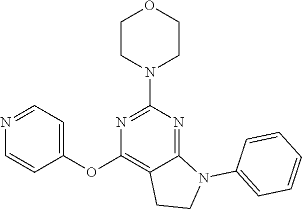 [SMILES: c1ccc(N2CCc3c(Oc4ccncc4)nc(N4CCOCC4)nc32)cc1] | ¹H NMR (400 MHz, DMSO-d₆) δ 8.38 (d, J = 7.8 Hz, 2H), 7.79 (d, J = 8.0 Hz, 2H), 7.47-7.31 (m, 2H), 7.09 (t, J = 7.2 Hz, 1H), 6.54 (d, J = 7.9 Hz, 2H), 4.22- 4.10 (m, 2H), 3.77 (dd, J = 19.2, 5.2 Hz, 8H), 3.26 (d, J = 8.4 Hz, 2H); LCMS (ESI) m/z: 376.0 [M + H]⁺. | 23 |
| 4-(7-(pyridin-3-yl)-4-(pyridin- 3-yloxy)-6,7-dihydro-5H- pyrrolo[2,3-d]pyrimidin-2- yl)morpholine | 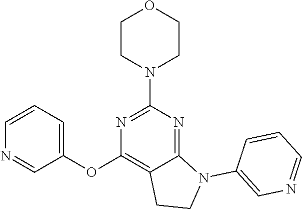 [SMILES: c1cncc(Oc2nc(N3CCOCC3)nc3c2CCN3c2cccnc2)c1] | ¹H NMR (400 MHz, DMSO-d₆) δ 9.03 (d, J = 2.0 Hz, 1H), 8.49 (d, J = 2.0 Hz, 1H), 8.43 (dd, J = 3.6, 0.9 Hz, 1H), 8.23 (dd, J = 3.6, 0.8 Hz, 1H), 8.20 (dq, J = 6.8, 1.2 Hz, 1H), 7.67 (dq, J = 6.4, 1.2 Hz, 1H), 7.48 (dd, J = 3.6, 0.8 Hz, 1H), 7.41 (dd, J = 6.8, 1.8 Hz, 1H), 4.15 (t, J = 6.8 Hz, 2H), 3.57 (s, 4H), 3.47 (s, 4H), 3.03 (t, J = 7.2 Hz, 2H); LCMS (ESI) m/z: 377.3 [M + H]+. | 24 |
| 4-(7-(3-fluorophenyl)-4- (pyridin-3-yloxy)-6,7-dihydro- 5H-pyrrolo[2,3-d]pyrimidin-2- yl)morpholine | 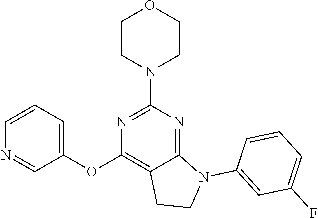 [SMILES: Fc1cccc(N2CCc3c(Oc4cccnc4)nc(N4CCOCC4)nc32)c1] | ¹H NMR (400 MHz, CDCL3) δ 8.53 (d, J = 2.0 Hz, 1H), 8.43 (dd, J = 3.8, 0.8 Hz, 1H), 7.75 (dt, J = 6.0, 2.0 Hz, 1H), 7.51 (dq, J = 6.4, 1.2 Hz, 1H), 7.36-7.26 (m, 3H), 6.75-6.71 (m, 1H), 4.08 (t, J = 6.8 Hz, 2H), 3.70- 3.60 (m, 8H), 3.06 (t, J = 7.0 Hz, 2H). LCMS (ESI) m/z: 394.2 [M + H]⁺. | 74 |

### Synthesis of 4-(7-(pyridin-3-yl)-4-((tetrahydrofuran-2-yl)methoxy)-6,7-dihydro-5H-pyrrolo[2,3-d]pyrimidin-2-yl)morpholine (Compound 19)

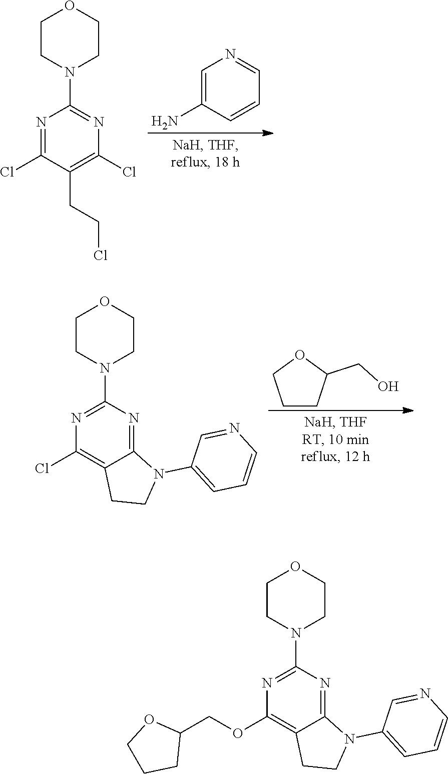

### Step 1: Synthesis of 4-(4-chloro-7-(pyridin-3-yl)-6,7-dihydro-5H-pyrrolo[2,3-d]pyrimidin-2-yl)morpholine

A solution of pyridin-3-amine (238 mg, 2.53 mmol) in tetrahydrofuran (15 mL) was added to a suspension of sodium hydride (202 mg, 5.06 mmol) in tetrahydrofuran (10 mL) at 0° C. The reaction mixture was then refluxed for 1 h. After cooling to room temperature, 4-(4,6-dichloro-5-(2-chloroethyl)pyrimidin-2-yl)morpholine (500 mg, 1.69 mmol) was added and the mixture was refluxed further for 16 h. The reaction mixture was poured then into ice water (50 mL) and extracted with ethyl acetate (50 mL×2). The organic layer was washed with brine (40 mL), dried over sodium sulfate, filtered and concentrated. The resulting residue was purified by silica gel column chromatography (petroleum ether/ethyl acetate=1/3 to 0/100) to obtain 4-(4-chloro-7-(pyridin-3-yl)-6,7-dihydro-5H-pyrrolo[2,3-d]pyrimidin-2-yl)morpholine (400 mg, 74%). LCMS (ESI) m/z: 318.1 [M+H]⁺.

### Step 2: Synthesis 4-(7-(pyridin-3-yl)-4-((tetrahydrofuran-2-yl)methoxy)-6,7-dihydro-5H-pyrrolo[2,3-d]pyrimidin-2-yl)morpholine

A solution of (tetrahydrofuran-2-yl)methanol (80 mg, 0.78 mmol) in THE (3 mL) was added to a solution of sodium hydride (38 mg, 0.95 mmol) in tetrahydrofuran (5 mL) at 0° C. After stirring at room temperature for 10 min, 4-(4-chloro-7-(pyridin-3-yl)-6,7-dihydro-5H-pyrrolo[2,3-d]pyrimidin-2-yl)morpholine (100 mg, 0.31 mmol) was added. The resultant reaction mixture was refluxed for 12 h. After cooling, the reaction mixture was diluted with ethyl acetate (80 mL), washed with water (30 mL×2) and brine, dried over sodium sulphate, filtered and concentrated. The residue was purified by prep-HPLC (SunFire C18, 4.6*50 mm, 3.5 um column Xbridge C18 3.5 μm 4.6×50 mm column. The elution system used was a gradient of 5%˜95% over 1.5 min at 2 ml/min and the solvent was acetonitrile/0.01% aqueous ammonium bicarbonate.) to give 4-(7-(pyridin-3-yl)-4-((tetrahydrofuran-2-yl)methoxy)-6,7-dihydro-5H-pyrrolo[2,3-d]pyrimidin-2-yl)morpholine_(28.8 mg, 24%) as white solid. ¹H NMR (400 MHz, DMSO-d₆) δ 8.99 (d, J=2.4 hz, 1H), 8.18-8.15 (m, 2H), 7.36 (dd, J=8.4, 4.4 Hz, 1H), 4.30-4.22 (m, 2H), 4.20-4.13 (m, 1H), 4.04 (t, J=8.6 hz, 2H), 3.80-3.75 (m, 1H), 2.90 (t, J=8.6 hz, 2H), 1.99-1.81 (m, 3H), 1.70-1.64 (m, 1H). LCMS (ESI) m/z: 384.1 [M+H]⁺.

### Synthesis of 3-(2-morpholino-4-(pyridin-3-ylmethoxy)-5H-pyrrolo[2,3-d]pyrimidin-7(6H)-yl)benzonitrile (Compound 20)

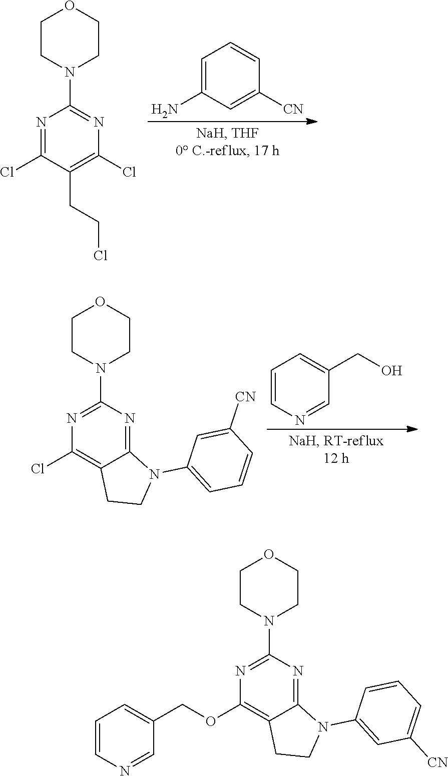

### Step 1: Synthesis of 3-(4-chloro-2-morpholino-5H-pyrrolo[2,3-d]pyrimidin-7(6H)-yl)benzonitrile

To a suspension of sodium hydride (40 mg, 1.0 mmol) in tetrahydrofuran (10 mL) was added 3-aminobenzonitrile (48 mg, 0.405 mmol) at 0° C. The reaction mixture was then refluxed for 1 h and cooled to room temperature. A solution of 4-(4,6-dichloro-5-(2-chloroethyl)pyrimidin-2-yl)morpholine (100 mg, 0.337 mmol) in THE was added to the mixture and then it was refluxed for another 16 h. After cooling to room temperature, the reaction mixture was quenched with water (50 mL) and the precipitate formed was collected by filtration, washed with methanol and dried to give 3-(4-chloro-2-morpholino-5H-pyrrolo[2,3-d]pyrimidin-7(6H)-yl)benzonitrile (60 mg, 52%). The crude product was used in next step without further purification. LCMS (ESI) m/z: 342.0 [M+H]⁺.

### Step 2: Synthesis of 3-(2-morpholino-4-(pyridin-3-ylmethoxy)-5H-pyrrolo[2,3-d]pyrimidin-7(6H)-yl)benzonitrile

Pyridin-3-ylmethanol (48 mg, 0.440 mmol) was added to a suspension of sodium hydride (21 mg, 0.525 mmol) in tetrahydrofuran (10 mL) at room temperature and stirred for 10 min. Then 3-(4-chloro-2-morpholino-5H-pyrrolo[2,3-d]pyrimidin-7(6H)-yl)benzonitrile (60 mg, 0.176 mmol) was added. The resultant mixture was refluxed for 12 h and cooled. Water (30 mL) was added to the mixture and the solids formed was collected by filtration to afford the crude product. It was then purified by prep-HPLC (SunFire C18, 4.6*50 mm, 3.5 um column Xbridge C18 3.5 μm 4.6×50 mm column. The elution system used was a gradient of 5%˜95% over 1.5 min at 2 ml/min and the solvent was acetonitrile/0.01% aqueous ammonium bicarbonate.) to obtain 3-(2-morpholino-4-(pyridin-3-ylmethoxy)-5H-pyrrolo[2,3-d]pyrimidin-7(6H)-yl)benzonitrile (11.3 mg, 16%) as white solid. ¹H NMR (400 MHz, DMSO-d₆) δ 8.67 (s, 1H), 8.53 (d, J=4.8 Hz, 1H), 8.16 (d, J=8 Hz, 1H), 8.12 (s, 1H), 7.85 (d, J=8 Hz, 1H), 7.55 (s, 1H), 7.43-7.39 (m, 2H), 5.45 (s, 2H), 4.06 (t, J=8.6 Hz, 2H), 3.67 (s, 8H), 2.92 (t, J=8.6 Hz, 2H). LCMS (ESI) m/z: 415.0 [M+H]⁺.

### Synthesis of 3-((2-morpholino-7-phenyl-6,7-dihydro-5H-pyrrolo[2,3-d]pyrimidin-4-yl)oxy)propane-1,2-diol (Compound 25)

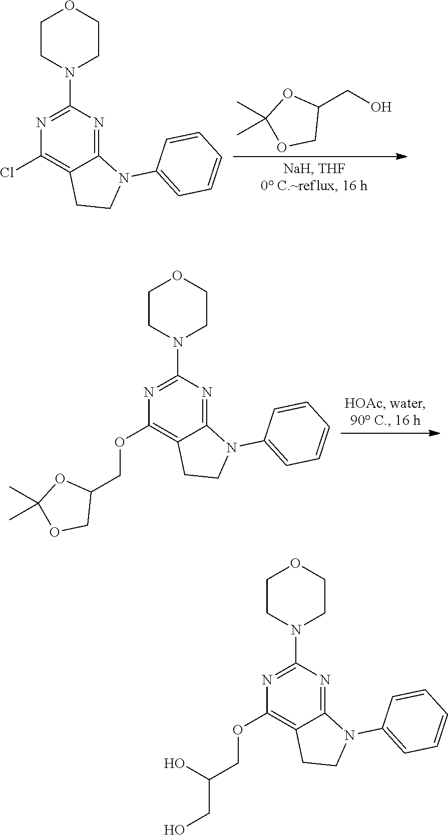

### Step 1: Synthesis of 4-(4-((2,2-dimethyl-1,3-dioxolan-4-yl)methoxy)-7-phenyl-6,7-dihydro-5H-pyrrolo[2,3-d]pyrimidin-2-yl)morpholine

A solution of (2,2-dimethyl-1,3-dioxolan-4-yl)methanol (90 mg, 0.68 mmol) in THF (5 mL) was added to a suspension of sodium hydride (27 mg, 0.68 mmol) in THF (5 mL) at 0° C. The reaction mixture was refluxed for 2 h and cooled. Then a solution of 4-(4-chloro-7-phenyl-6,7-dihydro-5H-pyrrolo[2,3-d]pyrimidin-2-yl)morpholine (100 mg, 0.34 mmol) in 3 mL of THE was added and the resultant mixture was stirred at reflux for 16 h. The reaction mixture was then diluted with ethyl acetate (30 mL), the resultant organic medium was washed with brine (10 mL), dried over sodium sulfate and concentrated to obtain 100 mg of the target compound. This was used in the next step without further purification.

### Step 2: Synthesis of 3-((2-morpholino-7-phenyl-6,7-dihydro-5H-pyrrolo[2,3-d]pyrimidin-4-yl)oxy)propane-1,2-diol

A solution of 4-(4-((2,2-dimethyl-1,3-dioxolan-4-yl)methoxy)-7-phenyl-6,7-dihydro-5H-pyrrolo[2,3-d]pyrimidin-2-yl)morpholine (100 mg, 0.24 mmol) in water (1 mL) and acetic acid (5 mL) was stirred at 80° C. overnight. The resultant mixture concentrated, and the crude product obtained was purified by prep-HPLC to give title compound 5.2 mg as white solid. ¹H NMR (400 MHz, DMSO-d₆) δ 7.76 (d, J=8.0 Hz, 2H), 7.34 (t, J=7.6 Hz, 2H), 6.97 (t, J=7.6 Hz, 1H), 4.87 (d, J=5.2 Hz 1H), 4.64 (t, J=8.8 Hz, 1H), 4.25-4.30 (m, 1H), 4.00-4.15 (m, 1H), 4.02 (t, J=8.4 Hz, 2H), 3.75-3.80 (m, 1H), 3.67 (s, 8H), 3.40 (t, J=5.6 Hz, 2H), 2.88 (t, J=9.2 Hz, 2H); LCMS (ESI) m/z: 373.0 [M+H]⁺.

### Synthesis of 4-(7-phenyl-4-((pyridin-3-yloxy)methyl)-6,7-dihydro-5H-pyrrolo[2,3-d]pyrimidin-2-yl)morpholine (Compound 26) and 5-hydroxy-1-((2-morpholino-7-phenyl-6,7-dihydro-5H-pyrrolo[2,3-d]pyrimidin-4-yl)methyl)pyridin-1-ium-3-ylium (Compound 71)

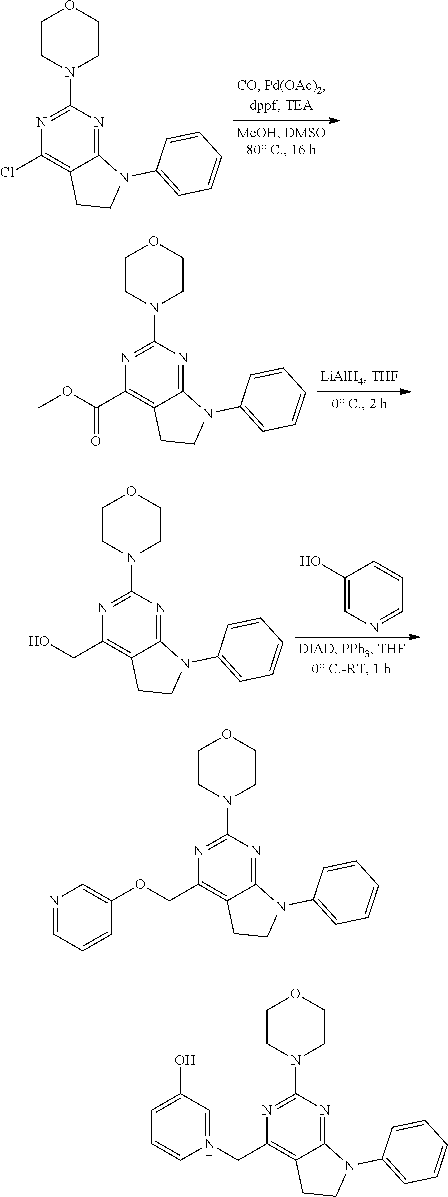

### Step 1: Synthesis of methyl 2-morpholino-7-phenyl-6,7-dihydro-5H-pyrrolo[2,3-d]pyrimidine-4-carboxylate

A solution of 4-(4-chloro-7-phenyl-6,7-dihydro-5H-pyrrolo[2,3-d]pyrimidin-2-yl)morpholine (500 mg, 1.578 mmol), triethylamine (479 mg, 4.734 mmol), palladium(II) acetate (36 mg, 0.160 mmol) and 1.1′-bis(diphenylphosphino)ferrocene (131 mg, 0.236 mmol) in methanol (12 mL) and dimethyl sulfoxide (15 mL) was stirred at 80° C. for 16 h under carbon monoxide atmosphere. After cooling to room temperature, the reaction mixture was filtered through celite, the filtrate was diluted with ethyl acetate (150 mL) and washed with water (40 mL×3) and brine (30 mL). The organics were dried over sodium sulfate, filtered, concentrated and the crude product obtained was purified by silica gel column chromatography, eluting with PE/EA=3/1 to obtain methyl 2-morpholino-7-phenyl-6,7-dihydro-5H-pyrrolo[2,3-d]pyrimidine-4-carboxylate (400 mg, 74%) as yellow solid. LCMS (ESI) m/z: 341.1 [M+H]⁺.

### Step 2: Synthesis of (2-morpholino-7-phenyl-6,7-dihydro-5H-pyrrolo[2,3-d]pyrimidin-4-yl)methanol

Lithium aluminum hydride (1.76 mL, 1.76 mmol) was added in portions to a solution of methyl 2-morpholino-7-phenyl-6,7-dihydro-5H-pyrrolo[2,3-d]pyrimidine-4-carboxylate (400 mg, 1.175 mmol) in tetrahydrofuran (20 mL) at 0° C. The solution was stirred at 0° C. for 1 h, then quenched with sodium sulfate decahydrate (2 g) and filtered through celite and washed with dichloromethane. The filtrate was concentrated to give (2-morpholino-7-phenyl-6,7-dihydro-5H-pyrrolo[2,3-d]pyrimidin-4-yl)methanol (170 mg, 46%) as white solid. LCMS (ESI) m/z: 313.1 [M+H]⁺. This crude product was used in the next step without further purification.

### Step 3: Synthesis of Compound 26 and Compound 71

To a solution of triphenylphosphine (118 mg, 0.450 mmol), pyridin-3-ol (43 mg, 0.452 mmol) and (2-morpholino-7-phenyl-6,7-dihydro-5H-pyrrolo[2,3-d]pyrimidin-4-yl)methanol (70 mg, 0.224 mmol) in tetrahydrofuran (15 mL) was added DIAD (91 mg, 0.450 mmol) at room temperature. The resultant mixture was stirred at room temperature for 1 h and concentrated. The residue was subjected to prep-HPLC (SunFire C18, 4.6*50 mm, 3.5 um column Xbridge C18 3.5 μm 4.6×50 mm column. The elution system used was a gradient of 5%˜95% over 1.5 min at 2 ml/min and the solvent was acetonitrile/0.01% aqueous ammonium bicarbonate.) to obtain compound 26 (17.7 mg, 20%) and compound 71 (12 mg, 14%) as white solids.

Compound 26: ¹H NMR (400 MHz, DMSO-d₆) δ 8.36 (d, J=2.8 Hz, 1H), 8.20-8.18 (m, 1H), 7.81 (d, J=8 Hz, 2H), 7.45-7.32 (m, 4H), 7.03 (t, J=7.6 Hz, 1H), 5.03 (s, 2H), 4.05 (t, J=8.4 Hz, 2H), 3.65 (s, 8H), 3.05 (t, J=8.4 Hz, 2H). LCMS (ESI) m/z: 390.1 [M+H]⁺.

Compound 71: ¹H NMR (400 MHz, DMSO-d₆) δ7.79 (d, J=6.0 Hz, 2H), 7.46-7.43 (m, 2H), 7.38 (t, J=6.0 Hz, 2H), 7.29 (dd, J=7.2, 4.4 Hz, 1H), 7.05 (t, J=6.0 Hz, 1H), 6.97-6.94 (m, 1H), 5.23 (s, 2H), 4.09 (t, J=6.6 Hz, 2H), 3.59 (dd, J=12.0, 3.6 Hz, 8H), 3.00 (t, J=6.6 Hz, 2H). LCMS (ESI) m/z: 390.1 [M].

### Synthesis of 4-(1-phenyl-6-(pyridin-3-ylmethoxy)-1H-pyrazolo[3,4-d]pyrimidin-4-yl)morpholine (Compound 27)

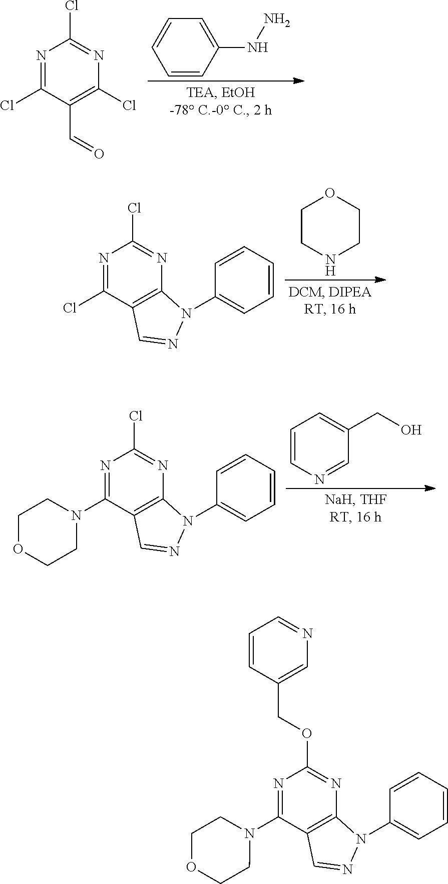

### Step 1: Synthesis of 4,6-dichloro-1-phenyl-1H-pyrazolo[3,4-d]pyrimidine

To a stirred solution of 2,4,6-trichloropyrimidine-5-carbaldehyde (630 mg, 3 mmol) in ethanol (20 mL) were added phenyl hydrazine (324 mg, 3 mmol) and triethylamine (910 mg, 9 mmol) dropwise at −78° C. in that order. The resultant mixture was stirred at −78° C. for 0.5 h and at 0° C. for 2 h. The mixture was then quenched with water (20 mL), the resultant precipitate was collected by filtration and dried to give 4,6-dichloro-1-phenyl-1H-pyrazolo[3,4-d]pyrimidine as white solid. (790 mg, 99%). LCMS (ESI) m/z: 265.0 [M+H]⁺.

### Step 2: Synthesis of 4-(6-chloro-1-phenyl-1H-pyrazolo[3,4-d]pyrimidin-4-yl)morpholine

To a mixture of 4,6-dichloro-1-phenyl-1H-pyrazolo[3,4-d]pyrimidine (792 mg, 3 mmol) in dichloromethane (10 mL) were added morpholine (520 mg, 6 mmol) and DIPEA (774 mg, 6 mmol) at 0° C. and the resultant mixture was stirred at room temperature for 16 h. The mixture was concentrated and purified by column chromatography eluting with 0-30% ethyl acetate in petroleum ether to give 4-(6-chloro-1-phenyl-1H-pyrazolo[3,4-d]pyrimidin-4-yl)morpholine as a yellow solid. (800 mg, 85%). LCMS (ESI) m/z: 316.0 [M+H]⁺.

### Step 3: Synthesis 4-(1-phenyl-6-(pyridin-3-ylmethoxy)-1H-pyrazolo[3,4-d]pyrimidin-4-yl)morpholine

To a mixture of pyridin-3-ylmethanol (218 mg, 2 mmol) in tetrahydrofuran (10 mL) was added sodium hydride (120 mg, 3 mmol) at 0° C. followed by 4-(6-chloro-1-phenyl-1H-pyrazolo[3,4-d]pyrimidin-4-yl)morpholine (315 mg, 1 mmol) and the resultant mixture was stirred at room temperature for 16 h. The reaction was quenched with water (10 mL) and the formed precipitate was collected by filtration and dried.

The crude product thus obtained was purified with Prep-HPLC (BOSTON pHlex ODS 10 um 21.2×250 mm120 A. The mobile phase was acetonitrile/0.1% Formic acid) to obtain 4-(1-phenyl-6-(pyridin-3-ylmethoxy)-1H-pyrazolo[3,4-d]pyrimidin-4-yl)morpholine as yellow solid. (75 mg, 19%). ¹H NMR (400 MHz, DMSO-d6) δ 8.70 (d, J=1.6 Hz, 1H), 8.54 (dd, J=4.8, 1.6 Hz, 1H), 8.47 (s, 1H), 8.11 (dd, J=4.8, 0.8 Hz, 2H), 7.90-7.87 (m, 1H), 7.56-7.52 (m, 2H), 7.43-7.32 (m, 2H), 5.44 (s, 2H), 3.92 (t, J=4.8 Hz, 4H), 3.75 (t, J=4.8 Hz, 4H); LCMS (ESI) m/z: 389.2 [M+H]⁺.

### Synthesis of 4-(7-phenyl-4-(((tetrahydro-2H-pyran-4-yl)oxy)methyl)-6,7-dihydro-5H-pyrrolo[2,3-d]pyrimidin-2-yl)morpholine (Compound 30)

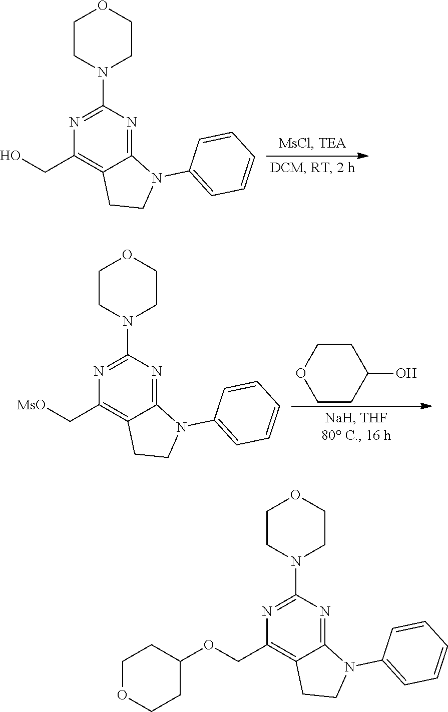

### Step 1: Synthesis of (2-morpholino-7-phenyl-6,7-dihydro-5H-pyrrolo[2,3-d]pyrimidin-4-yl)methyl methanesulfonate

Methanesulfonyl chloride (37 mg, 0.33 mmol) was added to a solution of (2-morpholino-7-phenyl-6,7-dihydro-5H-pyrrolo[2,3-d]pyrimidin-4-yl)methanol (70 mg, 0.22 mmol) and triethylamine (44 mg, 0.44 mmol) in dichloromethane (8 mL) at 0° C. The reaction mixture was stirred at 0° C. for 1 h under nitrogen atmosphere and then quenched with saturated aqueous sodium bicarbonate solution (10 mL) and extracted with dichloromethane (20 mL*3). The organic layer was washed with brine (20 mL), dried over sodium sulfate, filtered and concentrated to give (2-morpholino-7-phenyl-6,7-dihydro-5H-pyrrolo[2,3-d]pyrimidin-4-yl)methyl methanesulfonate (70 mg, 72%) as brown solid. The crude product was used in the next step without further purification. LCMS (ESI) m/z: 391.0 [M+H]⁺.

### Step 2: Synthesis of 4-(7-phenyl-4-(((tetrahydro-2H-pyran-4-yl)oxy)methyl)-6,7-dihydro-5H-pyrrolo[2,3-d]pyrimidin-2-yl)morpholine

A suspension of tetrahydro-2H-pyran-4-ol (27 mg, 0.27 mmol) and sodium hydride (11 mg, 0.27 mmol) in tetrahydrofuran (10 mL) was stirred at room temperature for 30 min, followed by the addition of (2-morpholino-7-phenyl-6,7-dihydro-5H-pyrrolo[2,3-d]pyrimidin-4-yl)methylmethane sulfonate (70 mg, 0.18 mmol) to the mixture. Then resultant mixture was stirred at 80° C. for 16 h, then quenched with water (10 mL) and extracted with dichloromethane (20*3 mL). The organic layer was washed with brine (20 mL), dried over sodium sulfate, filtered and concentrated. The residue was subjected to prep-HPLC (0.05% NH₄HCO₃/H₂O:CH₃CN=5%˜95%) to offer 4-(7-phenyl-4-(((tetrahydro-2H-pyran-4-yl)oxy)methyl)-6,7-dihydro-5H-pyrrolo[2,3-d]pyrimidin-2-yl)morpholine (4.3 mg, 6%,) as yellow solid. ¹H NMR (400 MHz, DMSO-d6) δ 7.79 (d, J=7.6 Hz, 2H), 7.41-7.37 (m, 2H), 7.07 (t, J=7.6 Hz, 1H), 4.46 (s, 2H), 4.06 (t, J=8.4 Hz, 2H), 4.01-3.96 (m, 2H), 3.78 (s, 8H), 3.69-3.62 (m, 1H), 3.53-3.47 (m, 2H), 3.17 (t, J=8.4 hz, 2H), 2.01-1.97 (m, 2H), 1.71-1.67 (m, 2H); LCMS (ESI) m/z: 397.2 [M+H]⁺.

### Synthesis of 4-(4-chloro-7-phenyl-6,7-dihydro-5H-pyrrolo[2,3-d]pyrimidin-2-yl)morpholine (Compound 32)

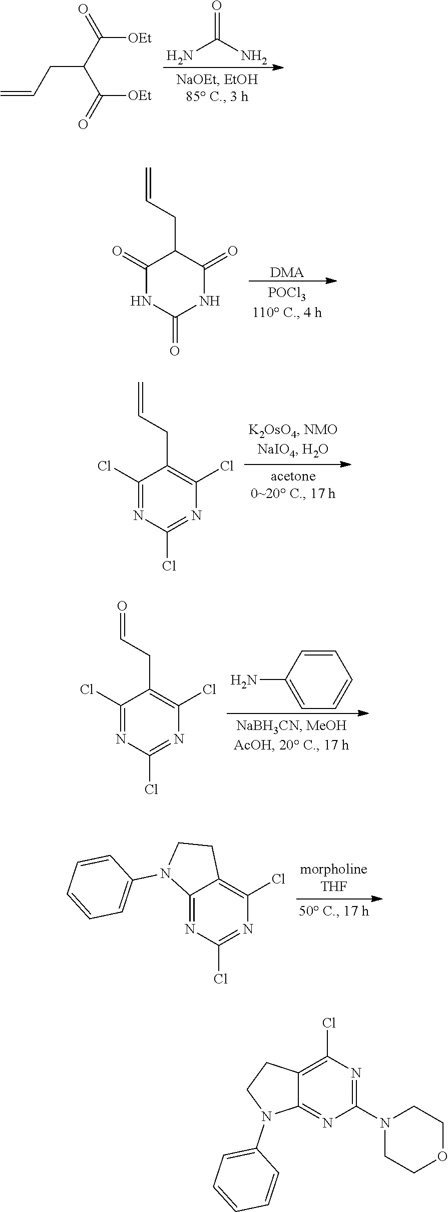

Step 1: Synthesis of 5-allylpyrimidine-2,4,6(1H,3H,5H)-trione.

To a solution of diethyl 2-allylmalonate (40.0 g, 200.0 mmol) and urea (12.0 g, 200.0 mmol) in ethanol (150 mL) was added sodium ethoxide (20% in ethanol) (80 mL) and the mixture was heated to 85° C. for 3 h. The resultant mixture was cooled to 20° C. and acetone (150 mL) was added. After stirring for 10 min and resultant precipitate was collected by filtration, washed with petroleum ether (150 mL) and then dissolved into water (150 mL). The pH of the resultant solution was adjusted between 3-4 with conc. HCl to obtain a precipitate which was stirred for 10 min. The solids were collected by filtration and dried under high vacuum to obtain 5-allylpyrimidine-2,4,6(1H,3H,5H)-trione as a brown solid (17.0 g, 51%). ¹H NMR (400 MHz, DMSO-d6) δ 11.25 (s, 2H), 5.63-5.73 (m, 1H), 5.03 (dd, J=12.0 Hz, J=3.6 Hz, 2H), 3.68 (t, J=5.2 Hz, 1H), 2.66 (t, J=5.62 Hz, 2H); LCMS (ESI) m/z: 169.1 [M+H]⁺.

### Step 2: Synthesis of 5-allyl-2,4,6-trichloropyrimidine

To a solution of 5-allylpyrimidine-2,4,6(1H,3H,5H)-trione (17.0 g, 101.2 mmol) in phosphorus oxychloride (60 mL) was added N,N-dimethylaniline (8.5 mL) and the solution was heated to 110° C. for 4 h. The dark solution was then cooled to 20° C. and concentrated. Ethyl acetate (300 mL) and ice water (200 mL) were added to the residue, the organic phase was separated and washed with brine (200 mL), dried, concentrated to obtain the crude product. It was purified by column chromatography (petroleum ether:ethyl acetate from 20:1 to 10:1) to obtain 5-allyl-2,4,6-trichloropyrimidine as off-white solid (16.0 g, 71%). ¹H NMR (400 MHz, CDCl₃) δ 5.79-5.89 (m, 1H), 5.11-5.21 (m, 2H), 3.63 (dt, J=6.0 Hz, J=1.6 Hz, 2H); LCMS (ESI) m/z: 223.1 [M+H]⁺.

### Step 3: Synthesis of 2-(2,4,6-trichloropyrimidin-5-yl)acetaldehyde

To a solution of 5-allyl-2,4,6-trichloropyrimidine (10.0 g, 44.7 mmol), potassium osmate(VI) dihydrate (330 mg, 0.89 mmol) and 4-methylmorpholine N-oxide (20.96 g, 89.4 mmol) in acetone (150 mL) and water (150 mL) was added sodium periodate (38.3 g, 178.8 mmol) at 0° C. and the mixture was stirred at 0-20° C. for 17 h. The resultant mixture was filtered and the filtrate was concentrated to remove the acetone and the aqueous phase was extracted with ethyl acetate (150 mL×2). The combined organic layer was washed with brine (150 mL), dried, concentrated to obtain the crude product. It was then purified by silica gel chromatography (petroleum ether:acetic ester from 10:1 to 3:1) to obtain 2-(2,4,6-trichloropyrimidin-5-yl)acetaldehyde as grey solid (5.9 g, 59%). ¹H NMR (400 MHz, CDCl₃) δ 9.80 (s, 1H), 4.14 (s, 2H).

### Step 4: Synthesis of 2,4-dichloro-7-phenyl-6,7-dihydro-5H-pyrrolo[2,3-d]pyrimidine

To a solution of 2-(2,4,6-trichloropyrimidin-5-yl)acetaldehyde (2.2 g, 9.76 mmol) and aniline (1.09 g, 11.71 mmol) in methanol (60 mL) were added acetic acid (1.0 mL) and sodium cyanoborohydride (1.23 g, 19.52 mmol) at 0° C. The resultant mixture was stirred between 0-20° C. for 17 h. Water (60 mL) was added to the mixture and after 10 mins the resultant precipitate was collected by filtration and dried under vacuum to obtain 2,4-dichloro-7-phenyl-6,7-dihydro-5H-pyrrolo[2,3-d]pyrimidine as white solid (2.0 g, 77%). ¹H NMR (400 MHz, CDCl₃) δ 7.69 (dd, J=8.8 Hz, J=1.2 Hz, 2H), 7.39-7.44 (m, 2H), 7.16 (t, J=7.2 Hz, 1H), 4.21 (t, J=8.8 Hz, 2H), 3.17 (t, J=8.8 Hz, 2H); LCMS (ESI) m/z: 266.1 [M+H]⁺.

### Step 5: Synthesis of 4-(4-chloro-7-phenyl-6,7-dihydro-5H-pyrrolo[2,3-d]pyrimidin-2-yl)morpholine

A solution of 2,4-dichloro-7-phenyl-6,7-dihydro-5H-pyrrolo[2,3-d]pyrimidine (100 mg, 0.376 mmol) and morpholine (164 mg, 1.88 mmol) in tetrahydrofuran (10 mL) was heated to 50° C. for 17 h. The mixture was concentrated to dryness followed by the addition of acetonitrile (5 mL) and water (20 mL) to the residue. The resultant precipitate was collected by filtration and dried under vacuum to obtain 4-(4-chloro-7-phenyl-6,7-dihydro-5H-pyrrolo[2,3-d]pyrimidin-2-yl)morpholine as white solid (67 mg, 56%). ¹H NMR (400 MHz, DMSO-d₆) δ 7.78 (d, J=7.6 Hz, 2H), 7.39 (t, J=6.8 Hz, J=2.0 Hz, 2H), 7.06 (t, J=7.2 Hz, 1H), 4.10 (t, J=8.8 Hz, 2H), 3.65 (s, 8H), 2.99 (t, J=8.8 Hz, 2H); LCMS (ESI) m/z: 317.1 [M+H]⁺.

### Synthesis of 4-(4-chloro-7-(3-methylbenzyl)-6,7-dihydro-5H-pyrrolo[2,3-d]pyrimidin-2-yl)morpholine (Compound 33)

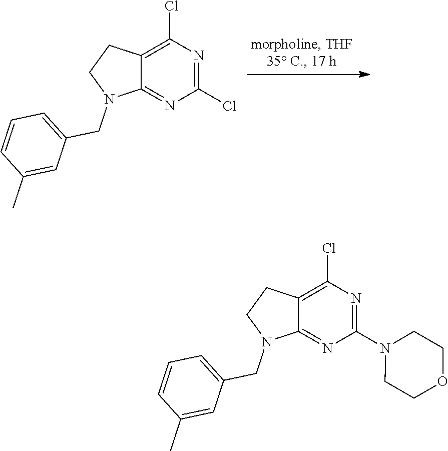 [SMILES: Cc1cccc(CN2CCc3c(Cl)nc(Cl)nc32)c1 ; Cc1cccc(CN2CCc3c(Cl)nc(N4CCOCC4)nc32)c1]

A mixture of 2,4-dichloro-7-(3-methylbenzyl)-6,7-dihydro-5H-pyrrolo[2,3-d]pyrimidine (2.0 g, 6.80 mmol) and morpholine (2.96 g, 34.0 mmol) in tetrahydrofuran (40 mL) was heated to 35° C. for 17 h and concentrated to dryness. MeOH (40 mL) and water (40 mL) were added to the residue and stirred. The resultant precipitate was collected by filtration and dried in vacuum to obtain 4-(4-chloro-7-(3-methylbenzyl)-6,7-dihydro-5H-pyrrolo[2,3-d]pyrimidin-2-yl)morpholine (2.0 g, 85%). ¹H NMR (400 MHz, DMSO-d₆) δ 7.23 (t, J=8.0 Hz, 2H), 7.05-7.10 (m, 3H), 4.48 (s, 2H), 3.61 (s, 8H), 3.48 (t, J=8.4 Hz, 2H), 2.85 (t, J=8.4 Hz, 2H), 2.29 (s, 3H); LCMS (ESI) m/z: 345.1 [M+H]⁺.

### Synthesis of 4-(7-phenyl-6,7-dihydro-5H-pyrrolo[2,3-d]pyrimidin-2-yl)morpholine (Compound 34)

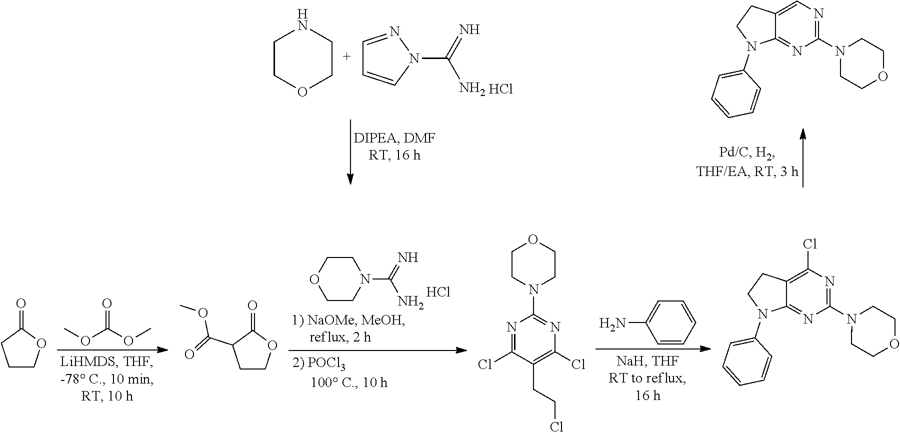

### Step 1a: Synthesis of morpholine-4-carboximidamide hydrochloride

N,N-Diisopropylethylamine (2.58 g, 20.00 mmol) was added to a solution of morpholine (1.74 g, 20.00 mmol) and 1H-pyrazole-1-carboximidamide hydrochloride (2.92 g, 20.00 mmol) in N,N-dimethylformamide (5 mL) at room temperature. The reaction mixture was stirred at room temperature for 16 h and ethyl ether (50 mL) was added to the mixture. The oily product at the bottom of the flask was solidified by repeated sonication and fresh ethyl ether. The solid was then collected by filtration and dried to obtain morpholine-4-carboximidamide hydrochloride (3 g, 91%) as white solid. LCMS (ESI) m/z: 130.1 [M+H]⁺.

### Step 1: Synthesis of methyl 2-oxotetrahydrofuran-3-carboxylate

A solution of dihydrofuran-2(3H)-one (3.36 g, 39.02 mmol) in tetrahydrofuran (5 mL) was added dropwise to lithium hexamethyldisilazide (1.0 M in tetrahydrofuran, 80.0 mL, 80.0 mmol) at −78° C. After stirring at −78° C. for 10 min, dimethyl carbonate (3.69 g, 40.98 mmol) was added at the same temperature. The reaction mixture was warmed up and stirred at room temperature for 16 h. Then it was poured onto a mixture of concentrated hydrochloric acid (15 mL) and ice (150 mL), followed by extraction with ethyl acetate (200 mL×2). The organic layer was washed by brine, dried over sodium sulfate and concentrated to obtain methyl 2-oxotetrahydrofuran-3-carboxylate (4.9 g, 87%). LCMS (ESI) m/z: 144.9 [M+H]⁺.

### Step 2: Synthesis of 4-(4,6-dichloro-5-(2-chloroethyl)pyrimidin-2-yl)morpholine

Morpholine-4-carboximidamide hydrochloride (575 mg, 3.47 mmol) was added to a solution of methyl 2-oxotetrahydrofuran-3-carboxylate (500 mg, 3.47 mmol) and sodium methoxide (287 mg, 5.31 mmol) in methanol (5 mL) at room temperature. The reaction mixture was refluxed for 2 h and concentrated. The resulting residue was dissolved in phosphorus oxychloride (5 mL) and heated with stirring at 100° C. for 16 h. Then the reaction mixture was added dropwise to water (100 mL), and then neutralized with 5 M aqueous sodium hydroxide solution. It was extracted with ethyl acetate (50 mL×2), the combined organic layer was washed with brine (30 mL), dried over sodium sulfate, filtered and concentrated. The crude product obtained was purified by silica gel column chromatography (n-hexane/ethyl acetate=10/1) to obtain 4-(4,6-dichloro-5-(2-chloroethyl)pyrimidin-2-yl)morpholine (236 mg, 23%) as white solid. LCMS (ESI) m/z: 298.0 [M+H]⁺.

### Step 3: Synthesis of 4-(4-chloro-7-phenyl-6,7-dihydro-5H-pyrrolo[2,3-d]pyrimidin-2-yl)morpholine

A solution of aniline (157 mg, 1.69 mmol) in tetrahydrofuran (3 mL) was added to a solution of sodium hydride (68 mg, 1.70 mmol) in tetrahydrofuran (2 mL) at 0° C. The reaction mixture was then refluxed for 2 h and cooled. Then 4-(4,6-dichloro-5-(2-chloroethyl)pyrimidin-2-yl)morpholine (100 mg, 0.34 mmol) was added at room temperature and the resultant mixture was refluxed for 16 h. It was cooled, then poured into ice water (30 mL) and extracted with ethyl acetate (20 mL×2). The organic layer was washed with brine (20 mL), dried over sodium sulfate and concentrated. The crude product obtained was purified by silica gel column chromatography (petroleum ether/ethyl acetate=9/1) to obtain 4-(4-chloro-7-phenyl-6,7-dihydro-5H-pyrrolo[2,3-d]pyrimidin-2-yl)morpholine (90 mg, 82%). LCMS (ESI) m/z: 317.0 [M+H]⁺.

### Step 4: Synthesis 4-(7-phenyl-6,7-dihydro-5H-pyrrolo[2,3-d]pyrimidin-2-yl)morpholine

A suspension of 4-(4-chloro-7-phenyl-6,7-dihydro-5H-pyrrolo[2,3-d]pyrimidin-2-yl)morpholine (80 mg, 0.25 mmol) and Pd/C (30 mg) in methanol (10 mL) and ethyl acetate (2 mL) was stirred at room temperature for 30 min under hydrogen atmosphere. The reaction solution was filtered through celite and the filtrated was concentrated. The crude product obtained was purified by PREP-HPLC (SunFire C18, 4.6*50 mm, 3.5 um column Xbridge C18 3.5 μm 4.6×50 mm column. The elution system used was a gradient of 5%˜95% over 1.5 min at 2 ml/min and the solvent was acetonitrile/0.01% aqueous ammonium bicarbonate) to afford 4-(7-phenyl-6,7-dihydro-5H-pyrrolo[2,3-d]pyrimidin-2-yl)morpholine (49.2 mg, 70%) as light-yellow solid. ¹H NMR (400 MHz, DMSO-d₆) δ 7.85 (s, 1H), 7.80 (d, J=8 Hz, 2H), 7.37 (t, J=8 Hz, 2H), 7.02 (t, J=7.6 Hz, 1H), 4.04 (t, J=8.4 Hz, 2H), 3.64 (s, 8H), 2.99 (t, J=8.4 Hz, 2H). LCMS (ESI) m/z: 283.1 [M+H]⁺.

### Synthesis of 4-(4-methyl-7-phenyl-6,7-dihydro-5H-pyrrolo[2,3-d]pyrimidin-2-yl)morpholine (Compound 35) and 2-methyl-1-(2-morpholino-7-phenyl-6,7-dihydro-5H-pyrrolo[2,3-d]pyrimidin-4-yl)propan-2-ol (Compound 84)

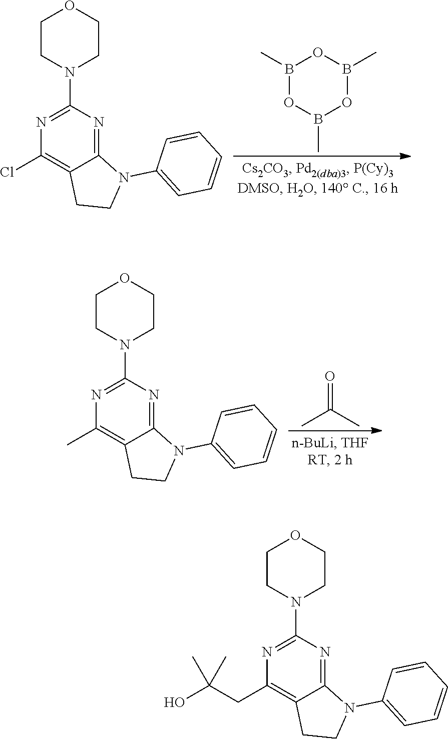

### Step 1: Synthesis of 4-(4-Methyl-7-phenyl-6,7-dihydro-5H-pyrrolo[2,3-d]pyrimidin-2-yl)morpholine

A mixture of 4-(4-chloro-7-phenyl-6,7-dihydro-5H-pyrrolo[2,3-d]pyrimidin-2-yl)morpholine (50 mg, 0.158 mmol), 2,4,6-trimethyl-1,3,5,2,4,6-trioxatriborinane (40 mg, 0.316 mmol), tris(dibenzylideneacetone)dipalladium (15 mg, 0.016 mmol), tris(dibenzylideneacetone)dipalladium (9 mg, 0.032 mmol) and cesium carbonate (103 mg, 0.316 mmol) in dimethyl sulfoxide (2 mL) and water (0.5 mL) was stirred at 140° C. for 16 h under nitrogen atmosphere. After cooling to room temperature, the reaction mixture was diluted with ethyl acetate (80 mL), washed with water (40 mL×3), brine (30 mL), dried over sodium sulfate, filtered and concentrated. The crude product obtained was purified by silica gel column chromatography, eluting with petroleum ether/ethyl acetate=6/1 to obtain 4-(4-methyl-7-phenyl-6,7-dihydro-5H-pyrrolo[2,3-d]pyrimidin-2-yl)morpholine (31.6 mg, 68%). ¹H NMR (500 MHz, DMSO-d₆) δ 7.80 (d, J=7.5 Hz, 2H), 7.36 (dd, J=8.5, 7.5 Hz, 2H), 7.00 (t, J=7.5 Hz, 1H), 4.03 (t, J=8.5 Hz, 2H), 3.64 (s, 8H), 2.95 (t, J=8.5 Hz, 2H), 2.13 (s, 3H). LCMS (ESI) m/z: 297.2 [M+H]⁺.

### Step 2: Synthesis of 2-methyl-1-(2-morpholino-7-phenyl-6,7-dihydro-5H-pyrrolo[2,3-d]pyrimidin-4-yl)propan-2-ol

To a stirred solution of 4-(4-methyl-7-phenyl-6,7-dihydro-5H-pyrrolo[2,3-d]pyrimidin-2-yl)morpholine (100 mg, 0.338 mmol) in tetrahydrofuran (5 mL) at 0° C. was added n-butyl lithium (0.25 mL, 0.506 mmol) and the resultant mixture was stirred at 0° C. for 0.5 h. Then propan-2-one (29 mg, 0.101 mmol) was added and the mixture was stirred further at room temperature for 2 h. Then water (20 mL) was added and the mixture was extracted with ethyl acetate (30 mL×3). The organic layer was dried over sodium sulfate, filtered and concentrated. The crude product obtained was purified by prep-HPLC (Column Xbridge 21.2*250 mm C18, 10 um, mobile phase A: water (10 mmol/L ammonium bicarbonate) B: acetonitrile) to obtain 2-methyl-1-(2-morpholino-7-phenyl-6,7-dihydro-5H-pyrrolo[2,3-d]pyrimidin-4-yl)propan-2-ol (35.7 mg, 30%). ¹H NMR (400 MHz, DMSO-d₆) δ 7.81 (d, J=8.4 Hz, 2H), 7.37 (t, J=7.6 Hz, 2H), 7.01 (t, J=6.4 Hz, 1H), 5.01 (s, 1H), 4.03 (t, J=8 Hz, 2H), 3.66-3.60 (m, 8H), 3.00 (t, J=8.4 Hz, 2H), 2.53 (s, 2H), 1.17 (s, 6H); LC-MS: m/z=355.2 (M+H)⁺.

### Synthesis of 2-morpholino-7-phenyl-6,7-dihydro-5H-pyrrolo[2,3-d]pyrimidine-4-carbonitrile (Compound 36)

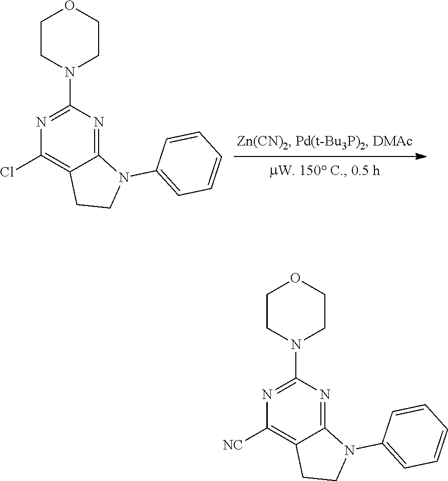 [SMILES: Clc1nc(N2CCOCC2)nc2c1CCN2c1ccccc1 ; N#Cc1nc(N2CCOCC2)nc2c1CCN2c1ccccc1]

A mixture of 4-(4-chloro-7-phenyl-6,7-dihydro-5H-pyrrolo[2,3-d]pyrimidin-2-yl)morpholine (100 mg, 0.316 mmol), zinc cyanide (74 mg, 0.631 mmol) and bis(tri-tert-butylphosphine)palladium(0) (32 mg, 0.063 mmol) in N,N-dimethylacetamide (4 mL) in a sealed vial was heated with microwave irradiation at 150° C. for 0.5 h under nitrogen atmosphere. After cooling to room temperature, the reaction mixture was diluted with ethyl acetate (80 mL), washed with water (40 mL×3) and brine (30 mL). The organic layer was dried over sodium sulfate, filtered and concentrated. The resultant crude product was purified by silica gel column chromatography, eluting with petroleum ether/ethyl acetate=6/1 to give 2-morpholino-7-phenyl-6,7-dihydro-5H-pyrrolo[2,3-d]pyrimidine-4-carbonitrile (33.0 mg, 34%) as yellow solid. 1H NMR (400 MHz, DMSO-d₆) δ 7.81 (d, J=8.0 Hz, 2H), 7.42 (t, J=8 Hz, 2H), 7.12 (s, 1H), 4.16 (t, J=8.0 Hz, 2H), 3.65 (s, 8H), 3.16 (t, J=8.0 Hz, 2H). LCMS (ESI) m/z: 308.1 [M+H]⁺.

### Synthesis of 4-(7-phenyl-4-(pyridin-2-ylmethyl)-6,7-dihydro-5H-pyrrolo[2,3-d]pyrimidin-2-yl)morpholine (Compound 37)

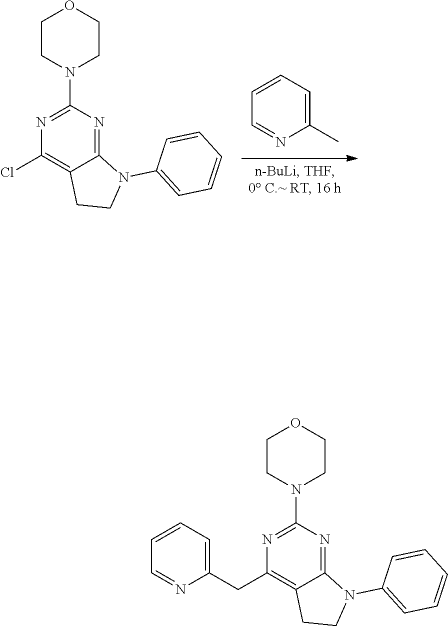

A solution of 2-methylpyridine (64 mg, 0.7 mmol) in tetrahydrofuran (15 mL) was added to n-BuLi (1 mL, 2.5 mmol, 2.5 M solution in hexanes) at 0° C. and stirred for 1 h. Then a solution of 4-(4-chloro-7-phenyl-6,7-dihydro-5H-pyrrolo[2,3-d]pyrimidin-2-yl)morpholine (200 mg, 0.64 mmol) in THE was added and the resultant mixture was warmed up to room temperature and stirred for 16 h. Then the reaction was quenched with saturated aqueous NH₄Cl solution (10 mL) and extracted with EtOAc (15*3 mL). The organic layer was combined, washed with brine (30 mL), dried over Na2SO4, filtered and concentrated. The residue was purified by prep-HPLC (0.05% FA/H₂O:CH₃CN=5%˜95%) to afford 4-(7-phenyl-4-(pyridin-2-ylmethyl)-6,7-dihydro-5H-pyrrolo[2,3-d]pyrimidin-2-yl)morpholine (39.3 mg, 17%,) as white solid. ¹H NMR (400 MHz, DMSO-d6) δ 8.47 (d, J=4.0 Hz, 1H), 7.80 (d, J=8.0 Hz, 2H), 7.74-7.00 (m, 1H), 7.39-7.33 (m, 3H), 7.25-7.22 (m, 1H), 7.01 (t, J=7.2 Hz, 1H), 4.02 (t, J=8.4 Hz, 2H), 3.95 (s, 2H), 3.63 (s, 8H), 2.92 (t, J=8.4 Hz, 2H); LCMS (ESI) m/z: 374.3 [M+H]⁺.

The following compound was synthesized according to the protocol described above:

| Name | Structure | NMR, MS | # |
|---|---|---|---|
| 4-(4-(pyridin- 2-ylmethyl)- 7-(pyridin-3- yl)-6,7- dihydro-5H- pyrrolo[2,3- d]pyrimidin- 2-yl) morpholine | 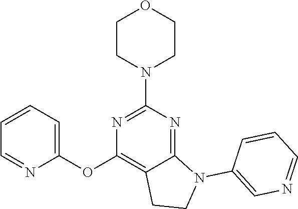 [SMILES: c1ccc(Oc2nc(N3CCOCC3)nc3c2CCN3c2cccnc2)nc1] | ¹H NMR (400 MHz, DMSO-d₆) δ 9.08 (d, J = 3.0 Hz, 1H), 8.54 (d, J = 5.6 Hz, 1H), 8.28 (dd, J = 4.8, 1.2 Hz, 1H), 8.13 (ddd, J = 8.4, 2.8, 1.2 Hz, 1H), 7.63 (t, J = 7.6 Hz, 1H), 7.36-7.28 (m, 2H), 7.18-7.15 (m, 1H), 4.06 (s, 2H), 4.02 (t, J = 8.8 Hz, 2H), 3.00 (t, J = 8.0 Hz, 2H); LCMS (ESI) m/z: 375.3 [M + H]+. | 38 |

### Synthesis of 4-(7-phenyl-4-(pyridin-2-yl)-6,7-dihydro-5H-pyrrolo[2,3-d]pyrimidin-2-yl)morpholine (Compound 39)

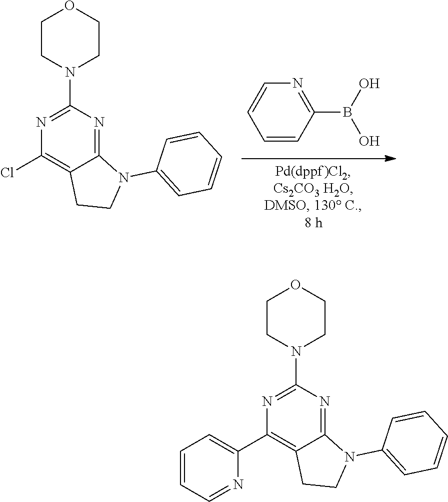

A solution of 4-(4-chloro-7-phenyl-6,7-dihydro-5H-pyrrolo[2,3-d]pyrimidin-2-yl)morpholine (250 mg, 0.79 mmol), pyridin-2-ylboronic acid (486 mg, 3.95 mmol), 1,1′-bis(diphenylphosphino)ferrocene-palladium(II)dichloride dichloromethane complex (131 mg, 0.16 mmol) and cesium carbonate (772 mg, 2.37 mmol) in water (5.0 mL) and DMSO (20 mL) was stirred at 130° C. for 8 h under argon atmosphere.

The mixture was diluted with ethyl acetate (150 mL), washed with water (150 mL) and the organic layer was concentrated. The crude product obtained was purified by prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous ammonium bicarbonate.) to obtain 4-(7-phenyl-4-(pyridin-2-yl)-6,7-dihydro-5H-pyrrolo[2,3-d]pyrimidin-2-yl)morpholine as yellow solid (44.3 mg, 15%). ¹H NMR (400 MHz, Chloroform-d) δ 8.67 (d, J=4.9, 1.7 Hz, 1H), 8.37 (d, J=8.0 Hz, 1H), 7.90-7.72 (m, 3H), 7.42 (t, J=8.0 Hz, 2H), 7.30-7.27 (m, 1H), 7.06 (t, J=7.4 Hz, 1H), 4.12 (t, J=8.3 Hz, 2H), 3.94-3.75 (m, 8H), 3.60 (t, J=8.3 Hz, 2H). LCMS (ESI) m/z: 360.2 [M+H]⁺.

The following compounds were synthesized according to the above protocol.

| Name | Structure | NMR, MS | # |
|---|---|---|---|
| 4-(7-phenyl-4- (pyridin-3-yl)- 6,7-dihydro- 5H-pyrrolo [2,3-d] pyrimidin-2- yl) morpholine | 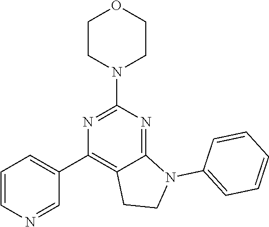 [SMILES: c1ccc(N2CCc3c(-c4cccnc4)nc(N4CCOCC4)nc32)cc1] | ¹H NMR (500 MHz, Chloroform-d) δ 9.12 (d, J = 2.2, 0.9 Hz, 1H), 8.64 (dd, J = 4.8, 1.7 Hz, 1H), 8.28 (dt, J = 8.0, 2.0 Hz, 1H), 7.81 (d, J = 8 Hz, 2H), 7.44-7.35 (m, 3H), 7.10 (t, J = 7.2 Hz, 1H), 4.12 (t, J = 8.2 Hz, 2H), 3.91-3.75 (m, 8H), 3.35 (t, J = 8.2 Hz, 2H). LCMS (ESI) m/z: 360.1 [M + H]⁺. | 40 |
| 4-(7-phenyl-4- (pyridin-4-yl)- 6,7-dihydro- 5H-pyrrolo [2,3-d] pyrimidin-2- yl) morpholine | 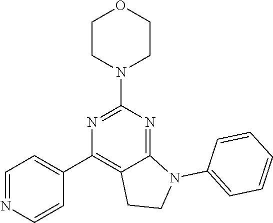 [SMILES: c1ccc(N2CCc3c(-c4ccncc4)nc(N4CCOCC4)nc32)cc1] | ¹H NMR (400 MHz, Chloroform-d) δ 8.74 (dd, J = 4.0, 1.2 Hz, 1H), 7.82-7.77 (m, 4H), 7.43 (t, J = 6.4 Hz, 2H), 7.09 (t, J = 7.4 Hz, 1H), 4.13 (t, J = 8.2 Hz, 2H), 3.90- 3.84 (m, 4H), 3.83-3.77 (m, 4H), 3.34 (t, J = 8.2 Hz, 2H). LCMS (ESI) m/z: 360.1 [M + H]⁺. | 41 |

### Synthesis of 4-(7-(1-methylpiperidin-4-yl)-4-(pyridin-3-ylmethoxy)-6,7-dihydro-5H-pyrrolo[2,3-d]pyrimidin-2-yl)morpholine (Compound 44)

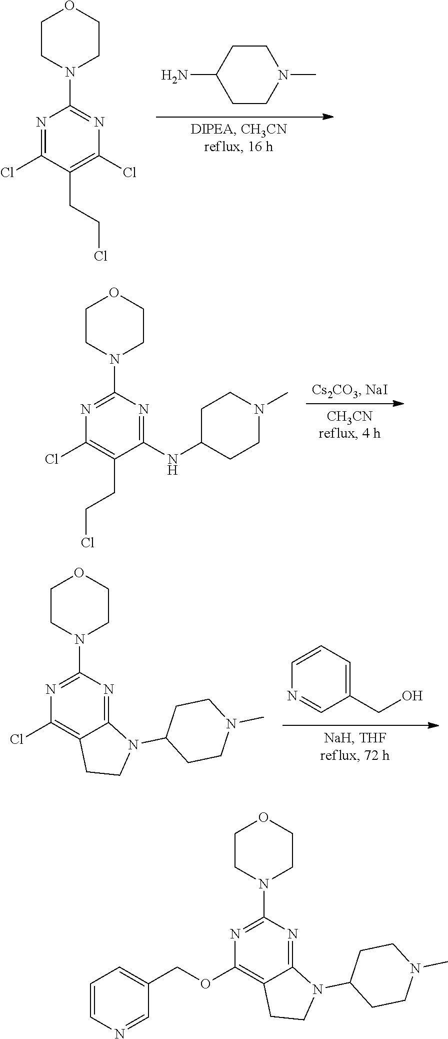

### Step 1: Synthesis of 6-chloro-5-(2-chloroethyl)-N-(1-methylpiperidin-4-yl)-2-morpholinopyrimidin-4-amine

To a stirred solution of 4-(4,6-dichloro-5-(2-chloroethyl)pyrimidin-2-yl)morpholine (100 mg, 0.337 mmol) and 1-methylpiperidin-4-amine (38 mg, 0.333 mmol) in acetonitrile (10 mL) at room temperature was added N-ethyl-N-isopropylpropan-2-amine (109 mg, 0.843 mmol). The reaction mixture was then refluxed for 16 h and cooled. It was diluted with ethyl acetate (80 mL), washed with water (20 mL), brine (20 mL), dried over sodium sulfate, filtered and concentrated to obtain 6-chloro-5-(2-chloroethyl)-N-(1-methylpiperidin-4-yl)-2-morpholinopyrimidin-4-amine (100 mg, 79%) as white solid. LCMS (ESI) m/z: 374.0 [M+H]⁺.

### Step 2: Synthesis of 4-(4-chloro-7-(1-methylpiperidin-4-yl)-6,7-dihydro-5H-pyrrolo[2,3-d]pyrimidin-2-yl)morpholine

Cesium carbonate (218 mg, 0.669 mmol) was added to a solution of 6-chloro-5-(2-chloroethyl)-N-(1-methylpiperidin-4-yl)-2-morpholinopyrimidin-4-amine (100 mg, 0.267 mmol) and sodium iodide (8 mg, 0.053 mmol) in acetonitrile (20 mL) at room temperature. The resultant mixture was refluxed for 4 h under nitrogen and cooled. It was diluted with ethyl acetate (150 mL), washed with water (50 mL) and brine (30 mL), dried over sodium sulfate, filtered and concentrated. The crude product obtained was purified by silica gel column chromatography, eluting with dichloromethane/methanol=9/1 to give 4-(4-chloro-7-(1-methylpiperidin-4-yl)-6,7-dihydro-5H-pyrrolo[2,3-d]pyrimidin-2-yl)morpholine (30 mg, 0.089 mmol, 33%) as white solid. LCMS (ESI) m/z: 338.1 [M+H]⁺.

### Step 3: Synthesis of 4-(7-(1-methylpiperidin-4-yl)-4-(pyridin-3-ylmethoxy)-6,7-dihydro-5H-pyrrolo[2,3-d]pyrimidin-2-yl)morpholine

To a suspension of sodium hydride (9 mg, 0.225 mmol) in tetrahydrofuran (5 mL) was added pyridin-3-ylmethanol (20 mg, 0.183 mmol) at room temperature and stirred for 10 min. Then a solution of 4-(4-chloro-7-(1-methylpiperidin-4-yl)-6,7-dihydro-5H-pyrrolo[2,3-d]pyrimidin-2-yl)morpholine (30 mg, 0.089 mmol) in THF was added to the mixture and the resultant mixture was refluxed for 48 h. It was cooled, diluted with ethyl acetate (80 mL), washed with water (30 mL) and brine (30 mL), dried over sodium sulfate, filtered and concentrated. The crude product obtained was purified by prep-HPLC (SunFire C18, 4.6*50 mm, 3.5 um column Xbridge C18 3.5 μm 4.6×50 mm column. The elution system used was a gradient of 5%˜95% over 1.5 min at 2 ml/min and the solvent was acetonitrile/0.01% aqueous ammonium bicarbonate.) to obtain 4-(7-(1-methylpiperidin-4-yl)-4-(pyridin-3-ylmethoxy)-6,7-dihydro-5H-pyrrolo[2,3-d]pyrimidin-2-yl)morpholine (14.5 mg, 40%) as white solid. ¹H NMR (400 MHz, DMSO-d₆) δ 8.61 (d, J=1.6 Hz, 1H), 8.50 (dd, J=4.8, 1.2 Hz, 1H), 7.80 (d, J=8.0 Hz, 1H), 7.39 (dd, J=7.6, 4.8 Hz, 1H), 5.35 (s, 2H), 3.74-3.70 (m, 1H), 3.59 (s, 8H), 3.48 (t, J=8.4 Hz, 2H), 2.82-2.79 (m, 2H), 2.71 (t, J=8.4 Hz, 2H), 2.15 (s, 3H), 1.94-1.89 (m, 2H), 1.74-1.68 (m, 2H), 1.56-1.53 (m, 2H). LCMS (ESI) m/z: 411.3 [M+H]⁺.

### Synthesis of 4-(1-phenyl-4-(pyridin-3-ylmethoxy)-1H-pyrazolo[3,4-d]pyrimidin-6-yl)morpholine (Compound 45)

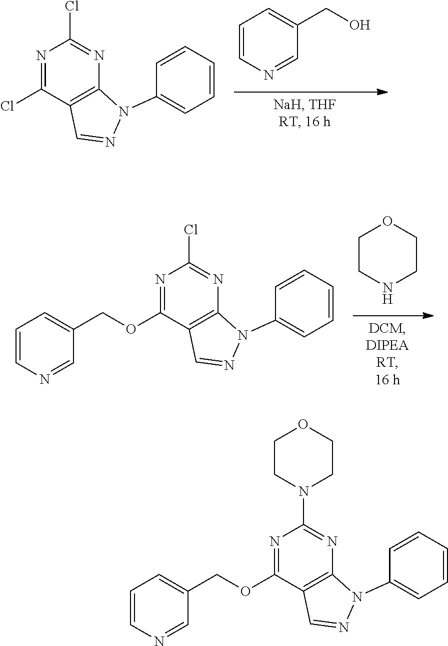

### Step 1: Synthesis of 6-chloro-1-phenyl-4-(pyridin-3-ylmethoxy)-1H-pyrazolo[3,4-d]pyrimidine

To a solution of pyridin-3-ylmethanol (109 mg, 1 mmol) in tetrahydrofuran (10 mL) was added sodium hydride (60 mg, 1.5 mmol, 60%) at 0° C. followed by 4,6-dichloro-1-phenyl-1H-pyrazolo[3,4-d]pyrimidine (264 mg, 1 mmol) and the resultant mixture was stirred at room temperature for 16 h. The reaction was quenched with water (10 mL) and the mixture was extracted with ethyl acetate (20 mL*2). The organic layer was dried and concentrated to give 6-chloro-1-phenyl-4-(pyridin-3-ylmethoxy)-1H-pyrazolo[3,4-d]pyrimidine as yellow solid. (250 mg, 74%). LCMS (ESI) m/z: 338.0 [M+H]⁺.

### Step 2: Synthesis 4-(1-phenyl-4-(pyridin-3-ylmethoxy)-1H-pyrazolo[3,4-d]pyrimidin-6-yl)morpholine

To a mixture of 6-chloro-1-phenyl-4-(pyridin-3-ylmethoxy)-1H-pyrazolo[3,4-d]pyrimidine (170 mg, 0.5 mmol) in dichloromethane (10 mL) was added morpholine (82 mg, 1 mmol) at 0° C. followed by DIPEA (129 mg, 1 mmol) and the resultant mixture was stirred at room temperature for 16 h. It was then concentrated and the obtained crude product was purified by prep-HPLC (BOSTON pHlex ODS 10 um 21.2×250 mm120 A. The mobile phase was acetonitrile/0.1% Ammonium bicarbonate) to obtain 4-(1-phenyl-4-(pyridin-3-ylmethoxy)-1H-pyrazolo[3,4-d]pyrimidin-6-yl)morpholine as yellow solid. (40 mg, 21%). ¹H NMR (400 MHz, DMSO-d6) δ 8.76 (d, J=4.6 Hz, 1H), 8.58 (dd, J=4.8 Hz, 1H), 8.20-8.18 (m, 3H), 7.95 (d, J=8 Hz, 1H), 7.53 (t, J=8 Hz, 2H), 7.45 (dd, J=7.6, 4.8 hz, 1H), 7.30 (t, J=6.8 Hz, 1H), 5.63 (s, 2H), 3.83 (t, J=4.4 Hz, 4H), 3.70 (t, J=4.8 Hz, 4H); LCMS (ESI) m/z: 389.1 [M+H]+.

### Synthesis of 2-morpholino-8-phenyl-4-(pyridin-3-ylmethoxy)-6H-pyrimido[5,4-b][1,4]oxazin-7(8H)-one (Compound 46)

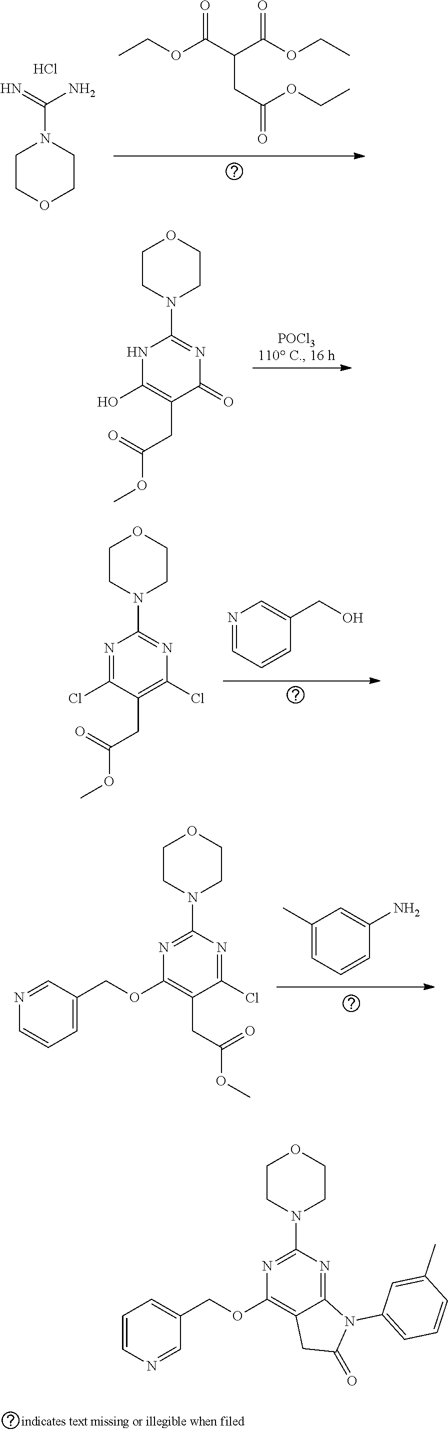

Step 1: Synthesis of methyl 2-(6-hydroxy-2-morpholino-4-oxo-1,4-dihydropyrimidin-5-yl)acetate.

To a solution of triethyl ethane-1,1,2-tricarboxylate (3 g, 12.18 mmol) and morpholine-4-carboximidamidehydrochloride (2 g, 12.18 mmol) in methanol (40 mL) was added sodium methanolate (30% solution in methanol, 6.7 mL, 34.35 mmol). After the addition, the mixture was stirred at 80° C. for 17 h and concentrated. The crude product methyl 2-(6-hydroxy-2-morpholino-4-oxo-1,4-dihydropyrimidin-5-yl)acetate (3 g, 91.56%) obtained as brown solid was used in the next step without further purification. LCMS (ESI) m/z: 270.0 [M+H]⁺.

### Step 2: Synthesis of methyl 2-(4,6-dichloro-2-morpholinopyrimidin-5-yl)acetate

A mixture of methyl 2-(6-hydroxy-2-morpholino-4-oxo-1,4-dihydropyrimidin-5-yl)acetate (3 g, 11.15 mmol) and phosphorus oxychloride (20 mL) was stirred at 110° C. for 16 h and then concentrated. The residue was diluted with ethyl acetate/water (20 mL/20 mL), organic layer separated and the aqueous layer was extracted with ethyl acetate (20 mL) twice. The combined organic phase was washed with brine (30 mL), dried over sodium sulfate, filtered and concentrated. The residue was purified by Combi-Flash (Biotage, 40 g silicagel, eluted with ethyl acetate in petro ether from 10% to 30%) to afford methyl 2-(4,6-dichloro-2-morpholinopyrimidin-5-yl)acetate (1.3 g, 38.2%) as white solid. LCMS (ESI) m/z: 306.1 [M+H]⁺.

### Step 3: Synthesis of methyl 2-(4-chloro-2-morpholino-6-(pyridin-3-ylmethoxy)pyrimidin-5-yl)acetate

To a solution of pyridin-3-ylmethanol (0.18 g, 1.65 mmol) in tetrahydrofuran (10 mL) was added sodium hydride (100 mg, 2.5 mmol) in portions and the mixture was stirred at 20° C. for 10 min. Then a solution of methyl 2-(4,6-dichloro-2-morpholinopyrimidin-5-yl)acetate (0.5 g, 1.64 mmol) in tetrahydrofuran (2 mL) was added slowly. After the addition, the mixture was stirred at 20° C. for 2 h, then quenched with water (15 mL) and extracted with ethyl acetate (20 mL). The organic phase was dried over sodium sulfate, filtered and concentrated. The residue was purified by Combi-Flash (Biotage, 40 g silica gel, eluted with ethyl acetate in petroleum ether from 30% to 40%) to afford methyl 2-(4-chloro-2-morpholino-6-(pyridin-3-ylmethoxy)pyrimidin-5-yl)acetate (0.3 g, 48.4%) as white solid. LCMS (ESI) m/z: 379.2 [M+H]⁺.

### Step 4: Synthesis of 2-morpholino-4-(pyridin-3-ylmethoxy)-7-m-tolyl-5H-pyrrolo[2,3-d]pyrimidin-6(7H)-one

A mixture of methyl 2-(4-chloro-2-morpholino-6-(pyridin-3-ylmethoxy)pyrimidin-5-yl)acetate (0.16 g, 0.42 mmol), tris(dibenzylideneacetone)dipalladium (39 mg, 0.042 mmol), 2-(Dicyclohexylphosphino)-2′,4′,6′-triisopropylbiphenyl (40 mg, 0.084 mmol) and cesium carbonate (0.34 g, 1.06 mmol) in toluene (15 mL) was stirred at 90° C. for 3 h under nitrogen atmosphere. The reaction mixture was filtered and concentrated. The residue was purified by prep-HPLC to afford 2-morpholino-4-(pyridin-3-ylmethoxy)-7-m-tolyl-5H-pyrrolo[2,3-d]pyrimidin-6(7H)-one (48 mg, 27.4%) as white solid. ¹H NMR (400 MHz, DMSO-d₆) δ 8.69 (d, J=1.6 Hz, 1H), 8.55 (dd, J=4.8, 1.2 hz, 1H), 7.88 (d, J=8 Hz, 1H), 7.46-7.34 (m, 2H), 7.27-7.16 (m, 3H), 5.49 (s, 2H), 3.64-3.51 (m, 10H), 2.35 (s, 3H); LCMS (ESI) m/z: 417.9 [M+H]⁺.

### Synthesis of 2-morpholino-8-phenyl-4-(pyridin-3-ylmethoxy)-6H-pyrimido[5,4-b][1,4]oxazin-7(8H)-one (Compound 47)

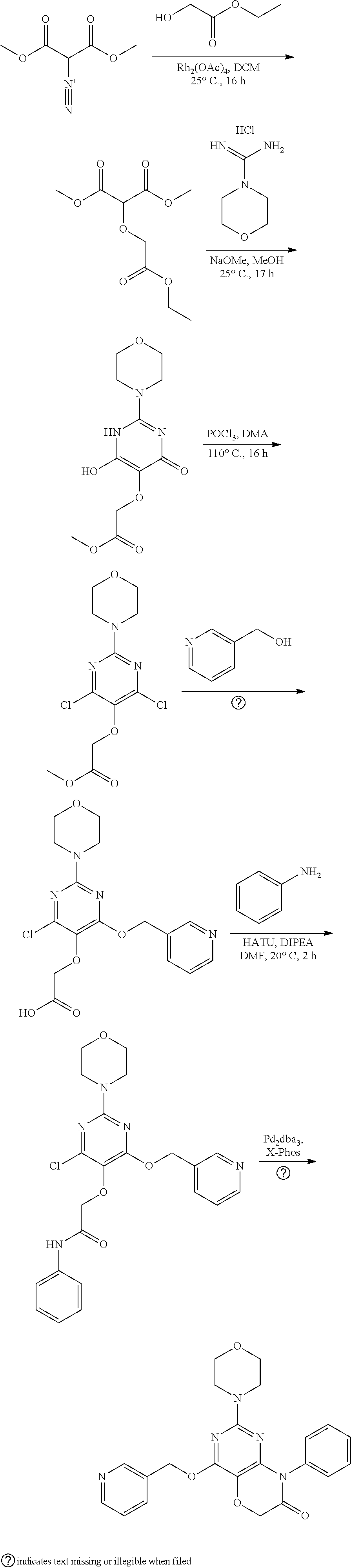

### Step 1: Synthesis of dimethyl 2-(2-ethoxy-2-oxoethoxy)malonate

A mixture of 1,3-dimethoxy-1,3-dioxopropane-2-diazonium (4 g, 25 mmol), ethyl 2-hydroxyacetate (1.2 mL, 12.5 mmol), and rhodium (II) acetate diner (2 g, 4.5 mmol) in dichloromethane (40 mL) was stirred at 25° C. for 16 h. The reaction mixture was diluted with dichloromethane (20 mL) and filtered. The filtrate was concentrated and the residue was purified by flash chromatography (Biotage, 40 g silica gel, eluted with ethyl acetate in petro ether from 30% to 60%) to afford dimethyl 2-(2-ethoxy-2-oxoethoxy)malonate (3.3 g, 56%) as colorless oil. LCMS (ESI) m/z: 235.1 [M+H]⁺.

### Step 2: Synthesis of methyl 2-(6-hydroxy-2-morpholino-4-oxo-1,4-dihydropyrimidin-5-yloxy)acetate

To a solution of dimethyl 2-(2-ethoxy-2-oxoethoxy)malonate (3 g, 12.82 mmol) and morpholine-4-carboximidamide hydrochloride (2.1 g, 12.82 mmol) in methanol (70 mL) was added sodium methanolate (30% solution in methanol, 7.5 mL, 38.46 mmol). After the addition, the mixture was stirred at 80° C. for 17 h and concentrated to afford methyl 2-(6-hydroxy-2-morpholino-4-oxo-1,4-dihydropyrimidin-5-yloxy)acetate (2.1 g, 57.5%) as brown solid, which was used directly in next step without further purification. LCMS (ESI) m/z: 286.1 [M+H]⁺.

### Step 3: Synthesis of methyl 2-(4,6-dichloro-2-morpholinopyrimidin-5-yloxy)acetate

A mixture of methyl 2-(6-hydroxy-2-morpholino-4-oxo-1,4-dihydropyrimidin-5-yloxy)acetate (2 g, 7 mmol), N,N-dimethylaniline (0.85 g, 7 mmol) and phosphorus oxychloride (15 mL) was stirred at 110° C. for 16 h. It was concentrated and the residue was diluted with ethyl acetate/water (20 mL/20 mL), the organic layer separated and the aqueous phase was extracted with ethyl acetate (20 mL) twice. The combined organic phase was washed with brine (30 mL), dried over sodium sulfate, filtered and concentrated. The residue was subjected to flask chromatography (Biotage, 40 g silicagel, eluted with ethyl acetate in petro ether from 10% to 30%) to obtain methyl 2-(4,6-dichloro-2-morpholinopyrimidin-5-yloxy)acetate (0.85 g, 37.8%) as yellow solid. LCMS (ESI) m/z: 322.1 [M+H]⁺.

### Step 4: Synthesis of 2-(4-chloro-2-morpholino-6-(pyridin-3-ylmethoxy)pyrimidin-5-yloxy)acetic acid

To a solution of pyridin-3-ylmethanol (68 mg, 0.62 mmol) in tetrahydrofuran (10 mL) was added sodium hydride (38 mg, 0.93 mmol) in portions and the mixture was stirred at 20° C. for 10 min. Then a solution of methyl 2-(4,6-dichloro-2-morpholinopyrimidin-5-yloxy)acetate (0.2 g, 0.62 mmol) in tetrahydrofuran (2 mL) was added slowly and the resultant mixture was stirred at 20° C. for 2 h. It was then quenched with water (15 mL) and extracted with ethyl acetate (20 mL). The aqueous phase was lyophilized to afford crude 2-(4-chloro-2-morpholino-6-(pyridin-3-ylmethoxy)pyrimidin-5-yloxy)acetic acid (0.2 g, 87%) as white solid, which was used directly in next step without further purification. LCMS (ESI) m/z: 381.1 [M+H]⁺.

### Step 5: Synthesis of 2-(4-chloro-2-morpholino-6-(pyridin-3-ylmethoxy)pyrimidin-5-yloxy)-N-phenylacetamide

To a solution of 2-(4-chloro-2-morpholino-6-(pyridin-3-ylmethoxy)pyrimidin-5-yloxy)acetic acid (0.18 g, 0.47 mmol) and aniline (66 mg, 0.71 mmol) in N,N-dimethylformamide (15 mL) was added 2-(7-Aza-1H-benzotriazole-1-yl)-1,1,3,3-tetramethyluronium hexafluorophosphate (0.32 g, 0.85 mmol) in portions, followed by N,N-diisopropylethylamine (0.18 g, 1.42 mmol). The resultant mixture was stirred at 20° C. for 2 h and then diluted with ethyl acetate/water (30 mL, 1:1). The layers were separated and the aqueous phase was extracted with ethyl acetate (20 mL) twice. The combined organic phase was washed with brine (20 mL), dried over sodium sulfate, filtered and concentrated. The residue was purified by flash chromatography (Biotage, 20 g silicagel, eluted with 7N ammonia in methanol:dichloromethane=1:10 in dichloromethane from 15% to 20%) to afford 2-(4-chloro-2-morpholino-6-(pyridin-3-ylmethoxy)pyrimidin-5-yloxy)-N-phenylacetamide (0.11 g, 51%) as yellow oil. LCMS (ESI) m/z: 456.1 [M+H]⁺.

### Step 6: Synthesis of 2-morpholino-8-phenyl-4-(pyridin-3-ylmethoxy)-6H-pyrimido[5,4-b][1,4]oxazin-7(8H)-one

A mixture of 2-(4-chloro-2-morpholino-6-(pyridin-3-ylmethoxy)pyrimidin-5-yloxy)-N-phenylacetamide (0.1 g, 0.22 mmol), tris(dibenzylideneacetone)dipalladium (20 mg, 0.022 mmol), 2-(dicyclohexylphosphino)-2′,4′,6′-triisopropylbiphenyl (21 mg, 0.044 mmol) and cesium carbonate (0.18 g, 0.55 mmol) in toluene (10 mL) was stirred at 100° C. for 16 h under nitrogen atmosphere. It was filtered and concentrated and the residue was subjected to prep-HPLC to afford 2-morpholino-8-phenyl-4-(pyridin-3-ylmethoxy)-6H-pyrimido[5,4-b][1,4]oxazin-7(8H)-one (5 mg, 5.4%) as white solid. ¹H NMR (400 MHz, DMSO-d₆) δ 8.69 (s, 1H), 8.56 (d, J=4.8 Hz, 1H), 7.89 (d, J=8 Hz, 1H), 7.52-7.38 (m, 4H), 7.28 (d, J=7.2 Hz, 2H), 5.46 (s, 2H), 4.77 (s, 2H), 3.54-3.57 (m, 4H), 3.34-3.27 (m, 4H); LCMS (ESI) m/z: 420.0 [M+H]⁺.

### Synthesis of 2-morpholino-N-(oxetan-3-yl)-7-phenyl-6,7-dihydro-5H-pyrrolo[2,3-d]pyrimidin-4-amine (Compound 48)

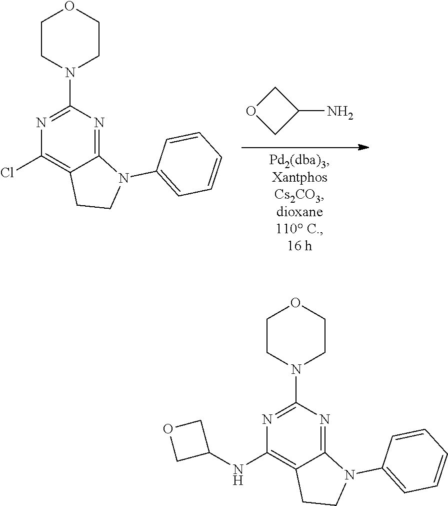

To a solution of 4-(4-chloro-7-phenyl-6,7-dihydro-5H-pyrrolo[2,3-d]pyrimidin-2-yl)morpholine (120 mg, 0.38 mmol) in dioxane (10 mL) were added oxetan-3-amine (55 mg, 0.76 mmol), Pd₂(dba)₃ (52 mg, 0.057 mmol), Xantphos (66 mg, 0.11 mmol) and Cs₂CO₃ (371 mg, 1.14 mmol). The resultant mixture was stirred at 110° C. for 16 h and concentrated. The crude product obtained was purified by prep-HPLC (0.05% FA/H2O:CH3CN=5%˜95%) to obtain 2-morpholino-N-(oxetan-3-yl)-7-phenyl-6,7-dihydro-5H-pyrrolo[2,3-d]pyrimidin-4-amine (14.3 mg, 11%,) as yellow solid. ¹H NMR (400 MHz, DMSO-d₆) δ 8.47 (s, 1H), 7.67 (d, J=8.0 Hz, 2H), 7.40 (t, J=7.6 Hz, 2H), 7.11 (t, J=7.6 Hz, 1H), 4.44 (t, J=9.6 Hz, 1H), 4.21 (t, J=8.4 Hz, 3H), 4.08 (dd, J=10.4, 5.6 Hz, 1H), 3.73 (t, J=4.4 Hz, 4H), 3.57-3.45 (m, 6H), 2.95-2.91 (m, 2H); LCMS (ESI) m/z: 354.1 [M+H]⁺.

The following compound were synthesized according to the protocol described above:

| Name | Structure | NMR, MS | # |
|---|---|---|---|
| 2- morpholino- 7- phenyl-N- (pyridin-3- yl)-6,7- dihydro-5H- pyrrolo [2,3-d] pyrimidin- 4-amine | 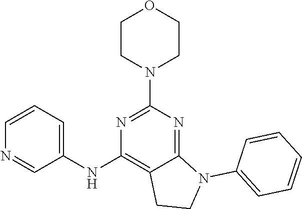 [SMILES: c1ccc(N2CCc3c(Nc4cccnc4)nc(N4CCOCC4)nc32)cc1] | ¹H NMR (400 MHz, CDCl₃) δ 8.74 (d, J = 2.4 Hz, 1H), 8.28 (dd, J = 4.8, 1.2 Hz, 1H), 7.98-7.95 (m, 1H), 7.74 (dd, J = 8.8, 1.2 Hz, 2H), 7.40-7.36 (m, 2H), 7.25-7.23 (m, 1H), 7.03 (t, J = 7.2 Hz, 1H), 5.94 (s, 1H), 4.11 (t, J = 8.4 Hz, 2H), 3.79 (s, 8H), 2.94 (t, J = 8.4 Hz, 2H); LCMS (ESI) m/z: 375.2 [M + H]+. | 52 |

### Synthesis of 4-(7-phenyl-4-(tetrahydro-2H-pyran-4-yl)-6,7-dihydro-5H-pyrrolo[2,3-d]pyrimidin-2-yl)morpholine (Compound 49)

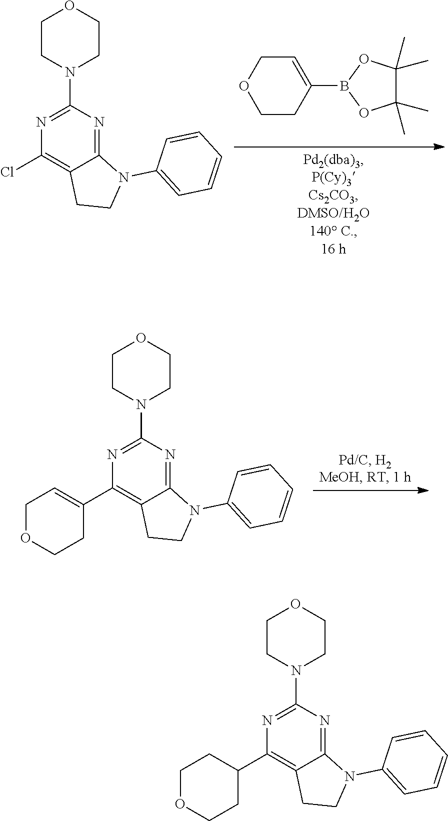

### Step 1: Synthesis of 4-(4-(3,6-dihydro-2H-pyran-4-yl)-7-phenyl-6,7-dihydro-5H-pyrrolo[2,3-d]pyrimidin-2-yl)morpholine

A mixture of 4-(4-chloro-7-phenyl-6,7-dihydro-5H-pyrrolo[2,3-d]pyrimidin-2-yl)morpholine (100 mg, 0.32 mmol), 2-(3,6-dihydro-2H-pyran-4-yl)-4,4,5,5-tetramethyl-1,3,2-dioxaborolane (199 mg, 0.95 mmol), Cs₂CO₃ (309 mg, 0.95 mmol), Pd₂(dba)₃ (29 mg, 0.03 mmol) and P(Cy)₃ (17 mg, 0.06 mmol) in DMSO (10 mL)/H2O (2 mL) was stirred at 140° C. for 16 h under nitrogen atmosphere. Then the reaction was quenched with water (10 mL) and the mixture was extracted with EtOAc (20*3 mL). The organic layers were combined, washed with brine (30 mL), dried over Na2SO4, filtered and concentrated. The residue was subjected to prep-TLC (PE/EA=4:1) to obtain 4-(4-(3,6-dihydro-2H-pyran-4-yl)-7-phenyl-6,7-dihydro-5H-pyrrolo[2,3-d]pyrimidin-2-yl)morpholine (70 mg, 60%) as yellow solid. LCMS (ESI) m/z: 365.2 [M+H]⁺.

### Step 2: Synthesis of 4-(7-phenyl-4-(tetrahydro-2H-pyran-4-yl)-6,7-dihydro-5H-pyrrolo[2,3-d]pyrimidin-2-yl)morpholine

A suspension of 4-(4-(3,6-dihydro-2H-pyran-4-yl)-7-phenyl-6,7-dihydro-5H-pyrrolo[2,3-d]pyrimidin-2-yl)morpholine (50 mg, 0.14 mmol) and 10% Pd/C (5 mg) in MeOH (10 mL) was stirred at room temperature for 1 h under hydrogen atmosphere. The mixture was then filtered, concentrated and subjected to prep-HPLC (0.05% FA/H2O:CH3CN=5%˜95%) to afford 4-(7-phenyl-4-(tetrahydro-2H-pyran-4-yl)-6,7-dihydro-5H-pyrrolo[2,3-d]pyrimidin-2-yl)morpholine (26.6 mg, 52%) as yellow solid. ¹H NMR (400 MHz, DMSO-d₆) δ 7.80 (d, J=8.0 Hz, 2H), 7.37 (t, J=8 Hz, 2H), 7.01 (t, J=7.2 Hz, 1H), 4.04 (t, J=8.4 Hz, 2H), 3.93 (dd, J=11.6, 3.2 Hz, 2H), 3.66 (s, 8H), 3.43 (t, J=10.8 Hz, 2H), 3.02 (t, J=8.4 Hz, 2H), 2.75 (t, J=11.6 Hz, 1H), 1.87-1.83 (m, 2H), 1.57 (d, J=11.2 Hz, 2H); LCMS (ESI) m/z: 367.2 [M+H]⁺.

### Synthesis of 4-(7-phenyl-4-(pyridazin-4-yl)-6,7-dihydro-5H-pyrrolo[2,3-d]pyrimidin-2-yl)morpholine (Compound 50)

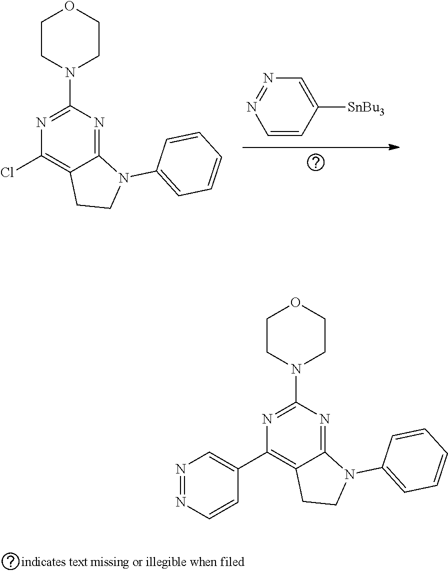

A solution of 4-(4-chloro-7-phenyl-6,7-dihydro-5H-pyrrolo[2,3-d]pyrimidin-2-yl)morpholine (60 mg, 0.19 mmol), 4-(tributylstannyl)pyridazine (70 mg, 0.19 mmol), LiCl (8 mg, 0.19 mmol) and Pd(PPh₃)₄ (22 mg, 0.019 mmol) in dioxane (5 mL) was stirred at 100° C. for 16 h under nitrogen atmosphere. It was concentrated and the residue was subjected to prep-HPLC (0.05% NH₄HCO3/H₂O:CH₃CN=5%˜95%) to obtain 4-(7-phenyl-4-(pyridazin-4-yl)-6,7-dihydro-5H-pyrrolo[2,3-d]pyrimidin-2-yl)morpholine (1.8 mg, 3%,) as yellow solid. ¹H NMR (400 MHz, CDCl₃) δ 9.74 (d, J=0.8 Hz, 1H), 9.33 (dd, J=5.2, 1.2 Hz, 1H), 8.00 (dd, J=5.2, 2.4 Hz, 1H), 7.81 (d, J=8.0 Hz, 2H), 7.44 (t, J=8.0 Hz, 2H), 7.14 (t, J=7.6 Hz, 1H), 4.20 (t, J=8.0 Hz, 2H), 3.90-3.82 (m, 8H), 3.42 (t, J=8.0 Hz, 2H); LCMS (ESI) m/z: 361.2 [M+H]⁺.

| Name | Structure | NMR, MS | # |
|---|---|---|---|
| 4-(7-phenyl-4-(pyrimidin- 5-yl)-6,7-dihydro-5H-pyrrolo [2,3-d]pyrimidin-2-yl) morpholine | 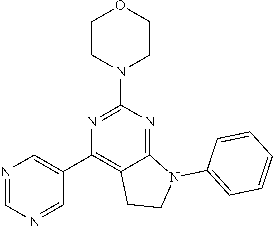 [SMILES: c1ccc(N2CCc3c(-c4cncnc4)nc(N4CCOCC4)nc32)cc1] | ¹H NMR (400 MHz, DMSO-d₆) δ 9.32 (s, 2H), 9.28 (s, 1H), 7.86 (d, J = 7.6 Hz, 2H), 7.42 (t, J = 8.4 Hz, 2H), 7.08 (t, J = 7.6 Hz, 1H), 4.15 (t, J = 7.6 Hz, 2H), 3.76-3.69 (m, 8H), 3.41-3.37 (m, 2H); LCMS (ESI) m/z: 361.1 [M + H]+. | 51 |
| 4-(4-(oxazol-5-yl)-7- phenyl-6,7-dihydro-5H- pyrrolo[2,3-d]pyrimidin- 2-yl)morpholine | 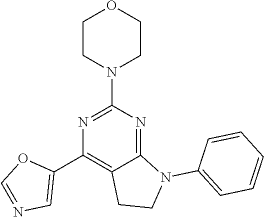 [SMILES: c1ccc(N2CCc3c(-c4cnco4)nc(N4CCOCC4)nc32)cc1] | ¹H NMR (400 MHz, CDCl₃) δ 8.03 (s, 1H), 7.82 (dd, J = 8.8, 0.8 Hz, 2H), 7.67 (s, 1H), 7.42 (dt, J = 7.6, 2.0 Hz, 2H), 7.10 (t, J = 7.2 Hz, 1H), 4.17 (t, J = 8.0 Hz, 2H), 3.87-3.81 (m, 8H), 3.34 (t, J = 8.0 Hz, 2H); LCMS (ESI) m/z: 350.2 [M + H]+. | 51 |
| 4-(7-(benzo[d][1,3]dioxol- 5-yl)-4-(pyridin-3-yl)-6,7- dihydro-5H-pyrrolo[2,3- d]pyrimidin-2-yl)morpholine | 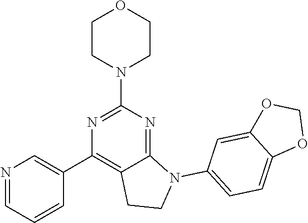 [SMILES: c1cncc(-c2nc(N3CCOCC3)nc3c2CCN3c2ccc3c(c2)OCO3)c1] | ¹H NMR (400 MHz, DMSO-d₆) δ 9.10 (d, J = 2.0 Hz, 1H), 8.64 (dd, J = 4.8, 1.2 Hz, 1H), 8.28 (d, J = 8 Hz, 1H), 7.70 (d, J = 2.4 Hz, 1H), 7.53 (dd, J = 8.4, 4.8 Hz, 1H), 7.11 (dd, J = 8.4, 2.4 Hz, 1H), 6.95 (d, J = 8.4 Hz, 1H), 6.02 (s, 2H), 4.06 (t, J = 8.0 Hz, 2H), 3.73-3.67 (m, 8H), 3.31 (t, J = 8.0 Hz, 2H). LCMS (ESI) m/z: 404.2 [M + H]⁺. | 63 |
| 4-(7-(2,3-dihydrobenzo[b][1,4] dioxin-6-yl)-4- (pyridin-3-yl)-6,7- dihydro-5H-pyrrolo[2,3- d]pyrimidin-2-yl)morpholine | 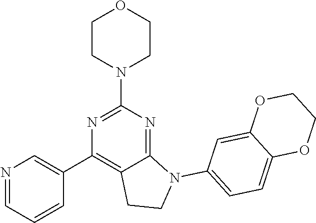 [SMILES: c1cncc(-c2nc(N3CCOCC3)nc3c2CCN3c2ccc3c(c2)OCCO3)c1] | ¹H NMR (400 MHz, CDCl₃) δ 9.10 (d, J = 1.8 Hz, 1H), 8.63 (dd, J = 4.8, 1.5 Hz, 1H), 8.25 (dt, J = 7.9, 1.8 Hz, 1H), 7.42 (d, J = 2.6 Hz, 1H), 7.39 (dd, J = 7.9, 4.8 Hz, 1H), 7.22 (dd, J = 8.9, 2.6 Hz, 1H), 6.88 (d, J = 8.9 Hz, 1H), 4.28 (dd, J = 10.3, 5.3 Hz, 4H), 4.05 (t, J = 8.2 Hz, 2H), 3.91-3.84 (m, 4H), 3.82- 3.74 (m, 4H), 3.31 (t, J = 8.2 Hz, 2H); LCMS (ESI) m/z 481.2 [M + H]⁺. | 64 |
| 4-(7-(3-fluorophenyl)-4- (pyridin-4-yl)-6,7-dihydro-5H- pyrrolo[2,3-d]pyrimidin-2-yl) morpholine | 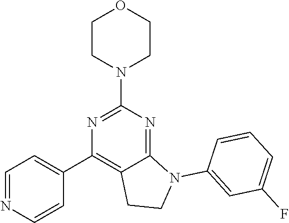 [SMILES: Fc1cccc(N2CCc3c(-c4ccncc4)nc(N4CCOCC4)nc32)c1] | ¹H NMR (400 MHz, CDCL3) δ 8.73 (dd, J = 4.4, 1.6 Hz, 2H), 7.83-7.77 (m, 3H), 7.43-7.37 (m, 2H), 6.81-6.76 (m, 1H), 4.11 (t, J = 8.2 Hz, 2H), 3.81-3.89 (m, 8H), 3.36 (t, J = 8.2 Hz, 2H). LCMS (ESI) m/z: 378.1 [M + H]⁺. | 72 |
| 4-(7-(pyridin-3-yl)-4- (pyridin-4-yl)-6,7-dihydro-5H- pyrrolo[2,3-d]pyrimidin- 2-yl)morpholine | 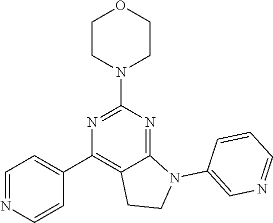 [SMILES: c1cncc(N2CCc3c(-c4ccncc4)nc(N4CCOCC4)nc32)c1] | ¹H NMR (400 MHz, CDCl₃) δ 9.13 (d, J = 2.8 Hz, 1H), 8.73 (dd, J = 4.8, 1.6 Hz, 2H), 8.33 (dd, J = 4.8, 1.6 Hz, 1H), 8.14 (dq, J = 8.4, 1.2 Hz, 1H), 7.79 (dd, J = 4.8, 2.0 Hz, 2H), 7.33 (dd, J = 8.0, 4.0 Hz, 1H), 4.14 (t, J = 8 Hz, 2H), 3.88-3.80 (m, 8H), 3.40 (t, J = 8.8 Hz, 2H); LCMS (ESI) m/z: 361.2 [M + H]⁺. | 73 |
| 4-(4,7-di(pyridin-3-yl)-6,7- dihydro-5H-pyrrolo[2,3- d]pyrimidin-2-yl)morpholine | 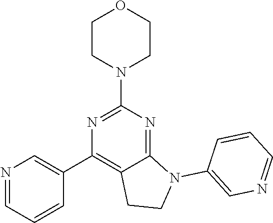 [SMILES: c1cncc(-c2nc(N3CCOCC3)nc3c2CCN3c2cccnc2)c1] | ¹H NMR (400 MHz, CDCl₃) δ 9.13-9.12 (m, 2H), 8.65 (dd, J = 7.6, 1.2 Hz, 1H), 8.32 (dd, J = 3.6, 0.8 Hz, 1H), 8.26 (dt, J = 6.4, 1.6 Hz, 1H), 8.14 (dq, J = 6.8, 1.2 Hz, 1H), 7.41 (dd, J = 6.4, 3.6 Hz, 1H), 7.33 (dd, J = 6.8, 4.0 Hz, 1H), 4.13 (t, J = 6.4 Hz, 2H), 3.88-3.80 (m, 8H), 3.39 (t, J = 6.8 Hz, 2H). LCMS (ESI) m/z: 361.2 [M + H]⁺. | 75 |

### Synthesis of tert-butyl 3-(((2-morpholino-7-(pyridin-3-yl)-6,7-dihydro-5H-pyrrolo[2,3-d]pyrimidin-4-yl)oxy)methyl)pyrrolidine-1-carboxylate (Compound 102), 4-(7-(pyridin-3-yl)-4-(pyrrolidin-3-ylmethoxy)-6,7-dihydro-5H-pyrrolo[2,3-d]pyrimidin-2-yl)morpholine (Compound 55) and 4-(4-((1-methylpyrrolidin-3-yl)methoxy)-7-(pyridin-3-yl)-6,7-dihydro-5H-pyrrolo[2,3-d]pyrimidin-2-yl)morpholine (Compound 104)

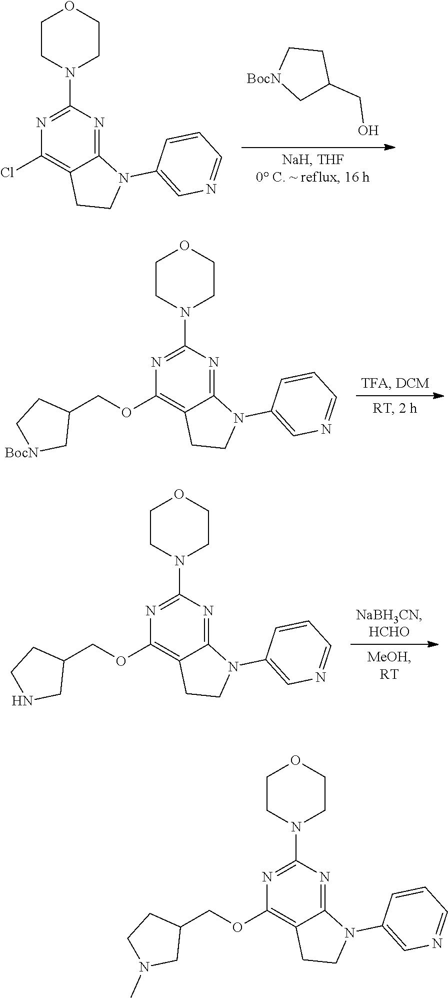

### Step 1: Synthesis of tert-butyl 3-(((2-morpholino-7-(pyridin-3-yl)-6,7-dihydro-5H-pyrrolo[2,3-d]pyrimidin-4-yl)oxy)methyl)pyrrolidine-1-carboxylate

To a solution of tert-butyl 3-(hydroxymethyl)pyrrolidine-1-carboxylate (84 mg, 0.42 mmol) in THE (15 mL) was added NaH (30 mg, 0.76 mmol) at 0° C. cautiously. The mixture was stirred at room temperature for 15 min and then 4-(4-chloro-7-(pyridin-3-yl)-6,7-dihydro-5H-pyrrolo[2,3-d]pyrimidin-2-yl)morpholine (120 mg, 0.38 mmol) was added. The resultant mixture was stirred further at 100° C. for 16 h. It was quenched with water (10 mL) and extracted with EA (30*3 mL). The organic layer was washed with brine (30 mL), dried over sodium sulfate, filtered and concentrated. The residue was purified by SGC (PE/EA=1:1 to 0:1) to obtain tert-butyl 3-(((2-morpholino-7-(pyridin-3-yl)-6,7-dihydro-5H-pyrrolo[2,3-d]pyrimidin-4-yl)oxy)methyl)pyrrolidine-1-carboxylate (135 mg, 74%) as white solid. ¹H NMR (400 MHz, CDCl3) δ 9.05 (d, J=2.4 Hz, 1H), 8.23 (d, J=4.0 Hz, 1H), 8.06 (d, J=9.2 Hz, 1H), 7.28 (s, 1H), 4.35-4.27 (m, 2H), 4.04 (t, J=8.4 Hz, 2H), 3.62 (s, 8H), 3.60-3.34 (m, 3H), 3.22-3.16 (m, 1H), 3.03-2.97 (m, 2H), 2.69-2.64 (m, 1H), 2.10-2.04 (s, 1H), 1.82-1.74 (m, 1H), 1.49 (s, 9H); LCMS (ESI) m/z: 483.3 [M+H]⁺.

### Step 2: Synthesis of 4-(7-(pyridin-3-yl)-4-(pyrrolidin-3-ylmethoxy)-6,7-dihydro-5H-pyrrolo[2,3-d]pyrimidin-2-yl)morpholine

To a solution of tert-butyl 3-(((2-morpholino-7-(pyridin-3-yl)-6,7-dihydro-5H-pyrrolo[2,3-d]pyrimidin-4-yl)oxy)methyl)pyrrolidine-1-carboxylate (120 mg, 0.25 mmol) in DCM (5 mL) was added TFA (1 mL) at 0° C. The mixture was stirred at room temperature for 2 h and concentrated. The resultant residue was purified by prep-HPLC (0.05% NH₄HCO₃/H₂O:CH3CN=5%˜95%) to offer 4-(7-(pyridin-3-yl)-4-(pyrrolidin-3-ylmethoxy)-6,7-dihydro-5H-pyrrolo[2,3-d]pyrimidin-2-yl)morpholine (16.8 mg, 63%,) as white solid. ¹H NMR (400 MHz, CDCl3) δ 9.06 (d, J=2.4 Hz, 1H), 8.24 (d, J=4.4 Hz, 1H), 8.10 (d, J=10.0 Hz, 1H), 7.28 (s, 1H), 4.31 (dd, J=10.8, 6.0 Hz, 1H), 4.23 (dd, J=10.8, 8.0 Hz, 1H), 4.04 (t, J=8.4 Hz, 2H), 3.78 (s, 8H), 3.13-3.10 (m, 1H), 3.08-2.93 (m, 4H), 2.82-2.77 (m, 1H), 2.60-2.53 (m, 1H), 2.01-1.95 (m, 1H), 1.60-1.53 (m, 1H); LCMS (ESI) m/z: 383.1 [M+H]⁺.

### Step 3: Synthesis of 4-(4-((l-methylpyrrolidin-3-yl)methoxy)-7-(pyridin-3-yl)-6,7-dihydro-5H-pyrrolo[2,3-d]pyrimidin-2-yl)morpholine

To a solution of 4-(7-(pyridin-3-yl)-4-(pyrrolidin-3-ylmethoxy)-6,7-dihydro-.H-pyrrolo[2,3-d]pyrimidin-2-yl)morpholine (30 mg, 0.076 mmol) in methanol (5 mL) was added formaldehyde (2.5 mg, 0.083 mmol). The mixture was stirred at room temperature for 2 h followed by the addition of sodium cyanoborohydride (24 mg, 0.38 mmol) to the mixture. It was then stirred at room temperature for 12 h. The reaction was then quenched with water (5 mL) and extracted with EA (20*3 mL). The organic layer was washed with brine (30 mL), dried over sodium sulfate, filtered and concentrated. The residue was purified by prep-HPLC (0.05% NH₄HCO₃/H₂O:CH₃CN=5%˜95%) to obtain 4-(4-((1-methylpyrrolidin-3-yl)methoxy)-7-(pyridin-3-yl)-6,7-dihydro-5H-pyrrolo[2,3-d]pyrimidin-2-yl)morpholine (8.7 mg, 28%) as white solid. ¹H NMR (400 MHz, CDCl3) δ 9.06 (d, J=2.4 Hz, 1H), 8.25 (d, J=3.6 Hz, 1H), 8.09 (d, J=9.6 Hz, 1H), 7.28 (s, 1H), 4.33-4.23 (min, 2H), 4.04 (t, J=8.4 hz, 2H), 3.78 (m, 8H), 3.00 (t, J=8.4 hz, 2H), 2.85-2.83 (n, 1H), 2.75-2.64 (m, 3H), 2.53-2.49 (m, 1H), 2.45 (1, 3H), 2.13-2.03 (m, 4H), 1.87-1.66 (n, 18H); LCMS (ESI) m/z: 397.2 [M+H]⁺.

The following compounds were synthesized according to the protocol described above:

| Name | Structure | NMR, MS | # |
|---|---|---|---|
| 1-(3-(((2-morpholino-7- (pyridin-3-yl)-6,7- dihydro-5H-pyrrolo[2,3- d]pyrimidin-4-yl)oxy) methyl)pyrrolidin-1-yl) ethan-1-one | 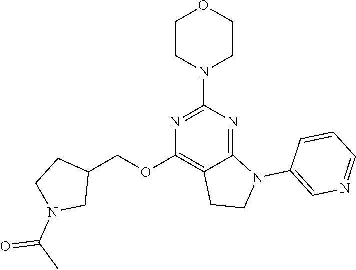 [SMILES: CC(=O)N1CCC(COc2nc(N3CCOCC3)nc3c2CCN3c2cccnc2)C1] | ¹H NMR (400 MHz, CDCl3) δ 9.08 (d, J = 2.4 Hz, 1H), 8.26-8.25 (m, 1H), 8.08 (d, J = 8.4 Hz, 1H), 7.31-7.28 (m, 1H), 4.40-4.26 (m, 2H), 4.08-4.02 (m, 2H), 3.78 (s, 8H), 3.73-3.47 (m, 3H), 3.37- 3.32 (m, 1H), 3.00 (dd, J = 16.8, 8.0 Hz, 2H), 2.80-2.67 (m, 1H), 2.20-2.06 (m, 4H), 1.94-1.89 (m, 1H); LCMS (ESI) m/z: 425.3 [M + H]+. | 107 |
| tert-butyl 3-(((2-morpholino- 7-(pyridin-3-yl)-6,7- dihydro-5H-pyrrolo[2,3-d] pyrimidin-4-yl)oxy)methyl) azetidine-1-carboxylate | 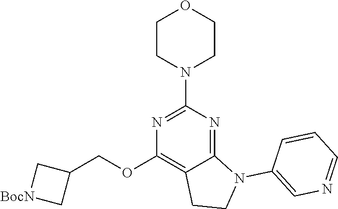 [SMILES: CC(C)(C)OC(=O)N1CC(COc2nc(N3CCOCC3)nc3c2CCN3c2cccnc2)C1] | ¹H NMR (400 MHz, CDCl3) δ 9.06 (d, J = 2.4 Hz, 1H), 8.25 (d, J = 4.0 Hz, 1H), 8.08 (dd, J = 8.4, 1.2 Hz, 1H), 7.30-7.28 (m, 1H), 4.47 (d, J = 6.8 Hz, 2H), 4.09-4.01 (m, 4H), 3.82-3.78 (m, 10 H), 2.99 (t, J = 8.4 Hz, 3H), 1.46 (s, 9H); LCMS (ESI) m/z: 469.2 [M + H]+. | 106 |
| 4-(4-(azetidin-3-ylmethoxy)- 7-(pyridin-3-yl)-6,7- dihydro-5H-pyrrolo[2,3-d] pyrimidin-2-yl)morpholine | 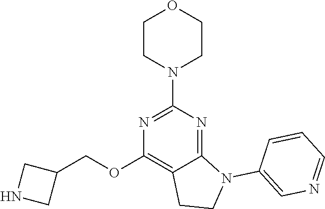 [SMILES: c1cncc(N2CCc3c(OCC4CNC4)nc(N4CCOCC4)nc32)c1] | ¹H NMR (400 MHz, CDCl3) δ 9.05 (d, J = 2.8 Hz, 1H), 8.24 (d, J = 4.0 Hz, 1H), 8.09 (d, J = 9.2 Hz, 1H), 7.30-7.28 (m, 1H), 4.50 (d, J = 6.4 Hz, 2H), 4.04 (t, J = 8.4 Hz, 2H), 3.86 (t, J = 8.4 Hz, 2H), 3.78 (s, 8H), 3.69 (t, J = 7.6 Hz, 2H), 3.26-3.20 (m, 1H), 3.01 (t, J = 8.4 Hz, 2H); LCMS (ESI) m/z: 369.1 [M + H]+. | 56 |
| 4-(4-((1- methylazetidin-3-yl) methoxy)-7-(pyridin-3-yl)- 6,7-dihydro-5H- pyrrolo[2,3-d]pyrimidin-2- yl)morpholine | 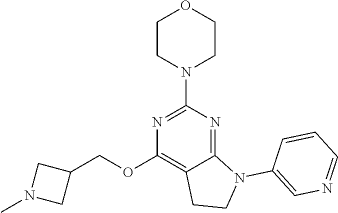 [SMILES: CN1CC(COc2nc(N3CCOCC3)nc3c2CCN3c2cccnc2)C1] | ¹H NMR (400 MHz, CDCl3) δ 9.06 (d, J = 1.2 Hz, 1H), 8.25 (d, J = 4.0 Hz, 1H), 8.11-8.08 (m, 1H), 7.30-7.29 (m, 1H), 4.46 (d, J = 6.8 Hz, 2H), 4.04 (t, J = 8.4 Hz, 2H), 3.75 (s, 8H), 3.46 (t, J = 7.6 Hz, 2H), 3.11 (t, J = 6.8 Hz, 2H), 3.01 (t, J = 8.4 Hz, 2H), 2.89 (hept, J = 6.8 Hz, 1H), 2.36 (s, 3H); LCMS (ESI) m/z: 383.3 [M + H]+. | 108 |

### Synthesis of tert-butyl 4-(2-morpholino-7-(pyridin-3-yl)-6,7-dihydro-5H-pyrrolo[2,3-d]pyrimidin-4-yl)piperidine-1-carboxylate (Compound 100), 4-(4-(piperidin-4-yl)-7-(pyridin-3-yl)-6,7-dihydro-5H-pyrrolo[2,3-d]pyrimidin-2-yl)morpholine (Compound 57) and 4-(4-(1-methylpiperidin-4-yl)-7-(pyridin-3-yl)-6,7-dihydro-5H-pyrrolo[2,3-d]pyrimidin-2-yl)morpholine (Compound 58)

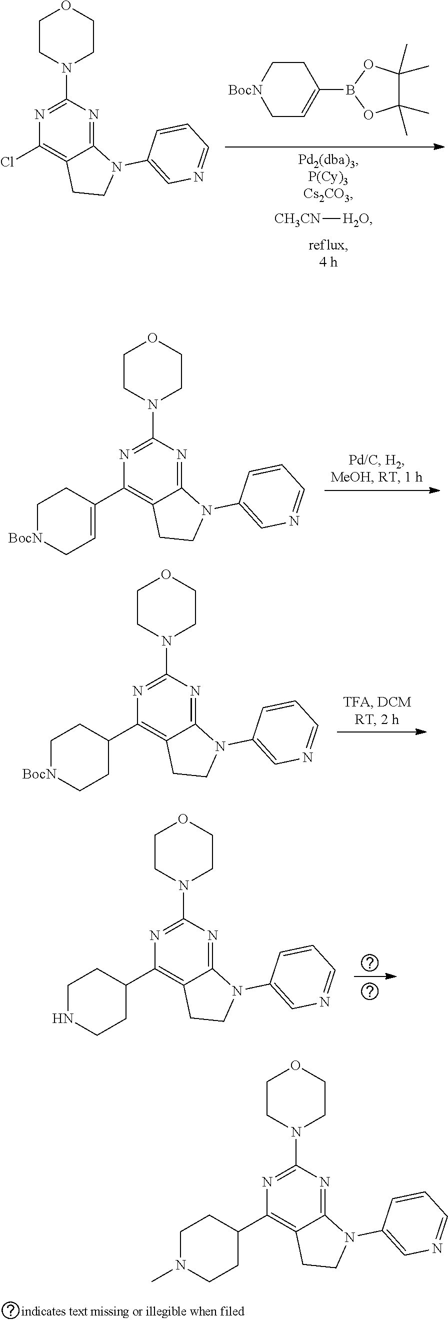

### Step 1: Synthesis of tert-butyl 4-(2-morpholino-7-(pyridin-3-yl)-6,7-dihydro-5H-pyrrolo[2,3-d]pyrimidin-4-yl)-5,6-dihydropyridine-1(2H)-carboxylate

A mixture of 4-(4-chloro-7-(pyridin-3-yl)-6,7-dihydro-5H-pyrrolo[2,3-d]pyrimidin-2-yl)morpholine (50 mg, 0.157 mmol), tert-butyl 4-(4,4,5,5-tetramethyl-1,3,2-dioxaborolan-2-yl)-5,6-dihydropyridine-1(2H)-carboxylate (97 mg, 0.314 mmol), tris(dibenzylideneacetone)dipalladium (15 mg, 0.016 mmol), tricyclohexylphosphine (9 mg, 0.032 mmol) and cesium carbonate (103 mg, 0.316 mmol) in acetonitrile (8 mL) and water (2 mL) was refluxed for 16 h under nitrogen atmosphere. After cooling to room temperature, the reaction mixture was diluted with ethyl acetate (80 mL), washed with water (30 mL) and brine (20 mL), dried over sodium sulfate, filtered and concentrated. The crude product obtained was purified by silica gel column chromatography, eluting with petroleum ether/ethyl acetate=1/1 then 0/100 to obtain tert-butyl 4-(2-morpholino-7-(pyridin-3-yl)-6,7-dihydro-5H-pyrrolo[2,3-d]pyrimidin-4-yl)-5,6-dihydropyridine-1(2H)-carboxylate (50 mg, 68%) as brown solid. LCMS (ESI) m/z: 465.3 [M+H]⁺.

### Step 2: Synthesis of tert-butyl 4-(2-morpholino-7-(pyridin-3-yl)-6,7-dihydro-5H-pyrrolo[2,3-d]pyrimidin-4-yl)piperidine-1-carboxylate

A suspension of tert-butyl 4-(2-morpholino-7-(pyridin-3-yl)-6,7-dihydro-5H-pyrrolo[2,3-d]pyrimidin-4-yl)-5,6-dihydropyridine-1(2H)-carboxylate (50 mg, 0.108 mmol) and palladium on activated charcoal (10%, 30 mg) in methanol (5 mL) and ethyl acetate (5 mL) was stirred at room temperature for 5 h under hydrogen atmosphere. The resultant mixture was filtered through celite and concentrated. The crude product obtained was purified by prep-HPLC (SunFire C18, 4.6*50 mm, 3.5 um column Xbridge C18 3.5 μm 4.6×50 mm column. The elution system used was a gradient of 5%˜95% over 1.5 min at 2 ml/min and the solvent was acetonitrile/0.01% aqueous ammonium bicarbonate.) to obtain tert-butyl 4-(2-morpholino-7-(pyridin-3-yl)-6,7-dihydro-5H-pyrrolo[2,3-d]pyrimidin-4-yl)piperidine-1-carboxylate (4.9 mg, 10%) as white solid. ¹H NMR (400 MHz, DMSO-d₆) δ 9.03 (d, J=2.4 Hz, 1H), 8.22-8.19 (m, 2H), 7.40-7.37 (m, 1H), 4.09-4.03 (m, 4H), 3.65 (s, 8H), 3.05 (t, J=8.4 Hz, 2H), 2.89-2.67 (m, 3H), 1.67-1.61 (m, 4H), 1.41 (s, 9H). LCMS (ESI) m/z: 467.2 [M+H]⁺.

### Step 3: Synthesis of 4-(4-(piperidin-4-yl)-7-(pyridin-3-yl)-6,7-dihydro-5H-pyrrolo[2,3-d]pyrimidin-2-yl)morpholine

Trifluoroacetic acid (1 mL) was added to a solution of tert-butyl 4-(2-morpholino-7-(pyridin-3-yl)-6,7-dihydro-5H-pyrrolo[2,3-d]pyrimidin-4-yl)piperidine-1-carboxylate (50 mg, 0.107 mmol) in dichloromethane (2 mL) at room temperature. After stirring the mixture at room temperature for 2 h, it was concentrated and the resultant residue was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous ammonium bicarbonate.) to obtain 4-(4-(piperidin-4-yl)-7-(pyridin-3-yl)-6,7-dihydro-5H-pyrrolo[2,3-d]pyrimidin-2-yl)morpholine (12.3 mg, 31%) as white solid. ¹H NMR (400 MHz, DMSO-d₆) δ 9.04 (d, J=2.4 Hz, 1H), 8.22-8.19 (m, 2H), 7.40-7.37 (m, 1H), 4.01 (t, J=8.4 Hz, 2H), 3.67 (s, 8H), 3.14-3.11 (m, 2H), 3.04 (t, J=8.4 Hz, 2H), 2.74-2.66 (m, 3H), 1.83-1.64 (m, 4H). LCMS (ESI) m/z: 367.3 [M+H]⁺.

### Step 4: Synthesis of 4-(4-(1-methylpiperidin-4-yl)-7-(pyridin-3-yl)-6,7-dihydro-5H-pyrrolo[2,3-d]pyrimidin-2-yl)morpholine

Acetic acid (16 mg, 0.266 mmol) was added to a mixture of 4-(4-(piperidin-4-yl)-7-(pyridin-3-yl)-6,7-dihydro-5H-pyrrolo[2,3-d]pyrimidin-2-yl)morpholine (50 mg, 0.136 mmol) and formaldehyde solution (37 wt. % in water) (1 mL) in methanol (10 mL) at room temperature. After stirring at room temperature for 2 h, sodium cyanoborohydride (17 mg, 0.271 mmol) was added to the mixture and stirred further for another 2 h. It was then concentrated and the residue obtained was subjected to prep-HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous ammonium bicarbonate.) to obtain 4-(4-(1-methylpiperidin-4-yl)-7-(pyridin-3-yl)-6,7-dihydro-5H-pyrrolo[2,3-d]pyrimidin-2-yl)morpholine (34.7 mg, 67%) as white solid. ¹H NMR (400 MHz, DMSO-d₆) δ 9.04 (s, 1H), 8.21-8.20 (m, 2H), 7.40-7.37 (m, 1H), 4.06 (t, J=8.2 Hz, 2H), 3.66 (s, 9H), 3.03 (t, J=8.2 Hz, 2H), 2.85-2.82 (m, 2H), 2.45-2.41 (m, 1H), 2.17 (s, 3H), 1.94-1.84 (m, 4H), 1.63-1.60 (m, 2H). LCMS (ESI) m/z: 381.3 [M+H]⁺.

### Synthesis of tert-butyl 3-(2-morpholino-7-phenyl-6,7-dihydro-5H-pyrrolo[2,3-d]pyrimidin-4-yl)azetidine-1-carboxylate (Compound 101)

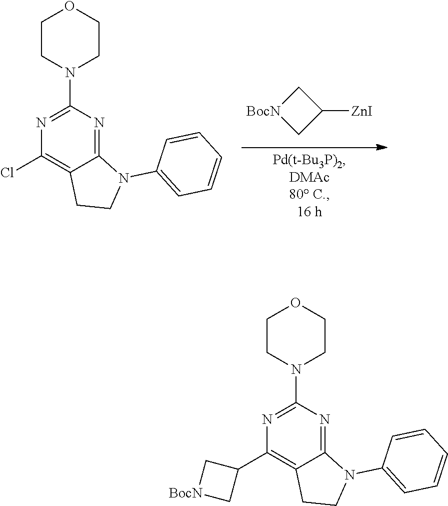

A solution of (1-(tert-butoxycarbonyl)azetidin-3-yl)zinc(II) iodide (0.5M in N,N-dimethylacetamide) (1.264 mL, 0.632 mmol) was added to a solution of 4-(4-chloro-7-phenyl-6,7-dihydro-5H-pyrrolo[2,3-d]pyrimidin-2-yl)morpholine (50 mg, 0.158 mmol) and bis(tri-tert-butylphosphine)palladium(0) (16 mg, 0.031 mmol) in N,N-dimethylacetamide (2 mL) at room temperature. The resultant mixture was stirred at 80° C. for 16 h and then quenched with saturated ammonium chloride solution (10 mL). The mixture was then extracted with ethyl acetate (20 mL×3), the combined organic layers were washed with water (20 mL×2), brine (20 mL), dried over sodium sulfate, filtered and concentrated. The crude product obtained was purified by prep-HPLC (SunFire C18, 4.6*50 mm, 3.5 um column Xbridge C18 3.5 μm 4.6×50 mm column.

The elution system used was a gradient of 5%-95% over 1.5 min at 2 ml/min and the solvent was acetonitrile/0.01% aqueous ammonium bicarbonate.) to obtain tert-butyl 3-(2-morpholino-7-phenyl-6,7-dihydro-5H-pyrrolo[2,3-d]pyrimidin-4-yl)azetidine-1-carboxylate (4.7 mg, 7%) as white solid. ¹H NMR (400 MHz, DMSO-d₆) δ 7.82 (d, J=8.0 Hz, 2H), 7.36 (t, J=7.6 Hz, 2H), 7.03 (t, J=7.2 Hz, 1H), 4.21-4.17 (m, 4H), 4.08 (t, J=8.4 Hz, 2H), 3.80-3.73 (2, 9H), 3.00 (t, J=8.4 Hz, 2H), 1.48 (s, 9H). LCMS (ESI) m/z: 438.1 [M+H]e.

The following compounds were synthesized according to the protocol described above:

| Name | Structure | NMR, MS | # |
|---|---|---|---|
| 4-(4- (piperidin-3- (pyridin- 3-yl)-7- (pyridin-3- yl)-6,7-dihydro- 5H-pyrrolo[2,3- d]pyrimidin- 2-yl)morpholine | 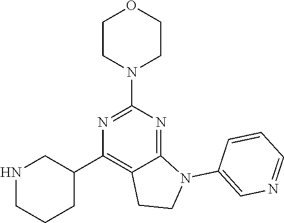 [SMILES: c1cncc(N2CCc3c(C4CCCNC4)nc(N4CCOCC4)nc32)c1] | ¹H NMR (400 MHz, CDCl3) δ 9.08 (d, J = 2.8 Hz, 1H), 8.28 (d, J = 3.6 Hz, 1H), 8.15 (dd, J = 8.4 Hz, 1.2 Hz, 1H), 7.32-7.28 (m, 1H), 4.06 (t, J = 8.4 Hz, 2H), 3.29 (s, 8H), 3.11-2.98 (m, 5H), 2.77-2.67 (m, 2H), 1.90-1.85 (m, 2H), 1.64-1.59 (m, 2H). LCMS (ESI) m/z: 367.0 [M + H]+. | 59 |
| 4-(4- (azetidin-3- yl)-7-phenyl- 6,7-dihydro- 5H-pyrrolo [2,3-d] pyrimidin- 2-yl) morpholine | 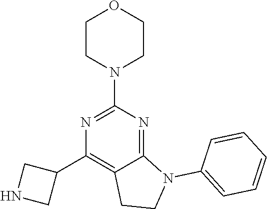 [SMILES: c1ccc(N2CCc3c(C4CNC4)nc(N4CCOCC4)nc32)cc1] | ¹H NMR (400 MHz, DMSO-d₆) δ 7.80 (d, J = 8.0 Hz, 2H), 7.37 (t, J = 7.6 Hz, 2H), 7.02 (t, J = 7.2 Hz, 1H), 4.05-4.00 (m, 4H), 3.89-3.84 (m, 1H), 3.80-3.39 (m, 10H), 2.92 (t, J = 8.0 Hz, 2H). LCMS (ESI) m/z: 338.3 [M + H]⁺. | 61 |
| 4-(7-phenyl- 4-(pyrrolidin-3- yl)-6,7- dihydro-5H- pyrrolo[2,3-d] pyrimidin- 2-yl) morpholine | 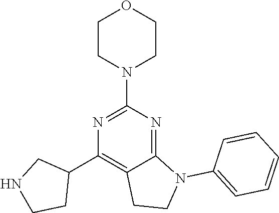 [SMILES: c1ccc(N2CCc3c(C4CCNC4)nc(N4CCOCC4)nc32)cc1] | ¹H NMR (400 MHz, DMSO-d₆) δ 7.80 (d, J = 8.0 Hz, 2H), 7.37 (t, J = 7.6 Hz, 2H), 7.01 (t, J = 6.8 Hz, 1H), 4.05 (t, J = 8.8 Hz, 2H), 3.65 (s, 8H), 3.49-3.43 (m, 2H), 3.23-3.16 (m, 2H), 3.06-2.96 (m, 4H), 2.07-1.93 (m, 2H); LC-MS: m/z = 352 (M + H)⁺. | 62 |
| 4-(4-(1- methylazetidin- 3- yl)-7-phenyl- 6,7-dihydro-5H- pyrrolo[2,3-d] pyrimidin-2- yl)morpholine | 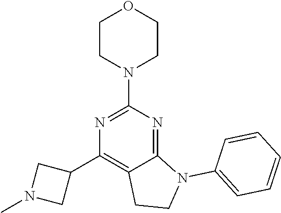 [SMILES: CN1CC(c2nc(N3CCOCC3)nc3c2CCN3c2ccccc2)C1] | ¹H NMR (400 MHz, DMSO-d₆) δ 7.80 (d, J = 8.0 Hz, 2H), 7.37 (t, J = 8.8 Hz, 2H), 7.01 (t, J = 5.6 Hz, 1H), 4.02 (t, J = 8.4 Hz, 2H), 3.68-3.57 (m, 11H), 3.46-3.30 (m, 2H), 2.92 (t, J = 8.4 Hz, 2H), 2.29 (s, 3H). LCMS (ESI) m/z: 352.1 [M + H]⁺. | 103 |

### Synthesis of 8-(2-morpholino-7-(pyridin-3-yl)-6,7-dihydro-5H-pyrrolo[2,3-d]pyrimidin-4-yl)octahydropyrazino[2,1-c][1,4]oxazine (Compound 78)

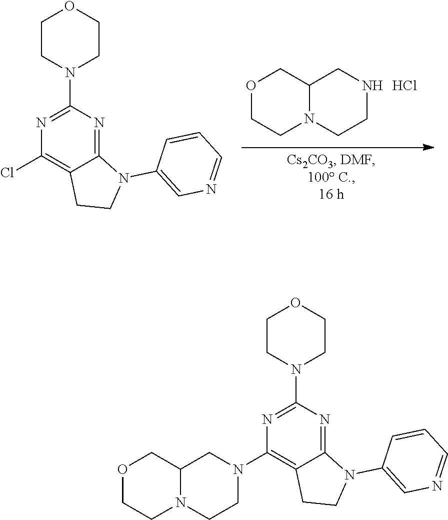

A mixture of octahydropyrazino[2,1-c][1,4]oxazine hydrochloride (60 mg, 0.337 mmol), 4-(4-chloro-7-(pyridin-3-yl)-6,7-dihydro-5H-pyrrolo[2,3-d]pyrimidin-2-yl)morpholine (107 mg, 0.337 mmol) and cesium carbonate (328 mg, 1.011 mmol) in N,N-dimethylformamide (5 mL) was stirred at 85° C. for 4 h. Water (10 mL) was then added and the mixture was extracted ethyl acetate (20 mL×3). The organic layer was dried and concentrated. The crude product obtained was purified by SGC (dichloromethane:methanol from 50:1 to 10:1) to obtain 8-(2-morpholino-7-(pyridin-3-yl)-6,7-dihydro-5H-pyrrolo[2,3-d]pyrimidin-4-yl)octahydropyrazino[2,1-c][1,4]oxazine (23.2 mg, 16%) as yellow solid. ¹H NMR (400 MHz, DMSO-d₆) δ 8.97 (d, J=2.8 Hz, 1H), 8.15-8.10 (m, 2H), 7.34 (dd, J=7.6, 4.4 Hz, 1H), 4.26 (d, J=14 Hz, 1H), 4.09 (d, J=12.4 Hz, 1H), 3.94 (t, J=8.4 Hz, 2H), 3.75-3.71 (m, 2H), 3.64-3.52 (m, 9H), 3.31 (s, 1H), 3.17-3.13 (m, 3H), 2.98-2.97 (m, 1H), 2.76-2.73 (m, 1H), 2.65-2.62 (m, 1H), 2.20-2.11 (m, 3H); LC-MS: m/z=424 (M+H)⁺.

The following compounds were synthesized according to the protocol described above:

| Name | Structure | NMR, MS | # |
|---|---|---|---|
| 4-(4-(isoindolin-2-yl)-7- (pyridin-3-yl)-6,7-dihydro- 5H-pyrrolo[2,3-d]pyrimidin-2- yl)morpholine | 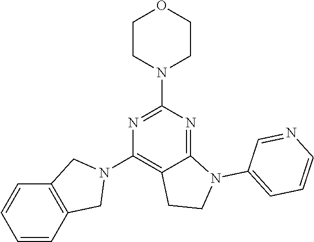 [SMILES: c1cncc(N2CCc3c(N4Cc5ccccc5C4)nc(N4CCOCC4)nc32)c1] | ¹H NMR (400 MHz, Chloroform-d) δ 9.03 (d, J = 2.7 Hz, 1H), 8.21 (dd, J = 4.6, 1.4 Hz, 1H), 8.14- 8.09 (m, 1H), 7.31 (s, 4H), 7.26-7.22 (m, 1H), 5.02 (s, 4H), 3.98 (t, J = 8.5 Hz, 2H), 3.79 (s, 8H), 3.47 (t, J = 8.4 Hz, 2H). LCMS (ESI) m/z: 401.1 [M + H]⁺. | 85 |
| 4-(4-(4-methylpiperazin-1-yl)-7- (pyridin-3-yl)-6,7-dihydro- 5H-pyrrolo[2,3-d]pyrimidin-2- yl)morpholine | 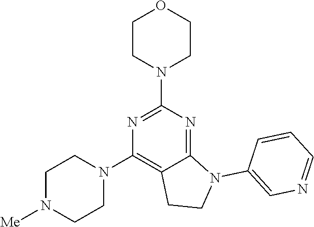 [SMILES: CN1CCN(c2nc(N3CCOCC3)nc3c2CCN3c2cccnc2)CC1] | ¹H NMR (400 MHz, CD3OD) δ 9.14 (d, J = 2.4 Hz, 1H), 8.14-8.11 (m, 2H), 7.39 (dd, J = 8.4, 4.8 Hz, 1H), 3.98 (t, J = 8.4 Hz, 2H), 3.76-3.70 (m, 12H), 3.22 (t, J = 8.4 Hz, 2H), 2.53 (t, J = 5.0 Hz, 4H), 2.35 (s, 3H). LCMS (ESI) m/z: 382.3 [M + H]⁺. | 60 |

### Synthesis of tert-butyl 4-(2-morpholino-7-phenyl-6,7-dihydro-5H-pyrrolo[2,3-d]pyrimidin-4-yl)piperidine-1-carboxylate (Compound 105), 4-(7-phenyl-4-(piperidin-4-yl)-6,7-dihydro-5H-pyrrolo[2,3-d]pyrimidin-2-yl)morpholine (Compound 88) and 4-(4-(1-methylpiperidin-4-yl)-7-phenyl-6,7-dihydro-5H-pyrrolo[2,3-d]pyrimidin-2-yl)morpholine (Compound 65)

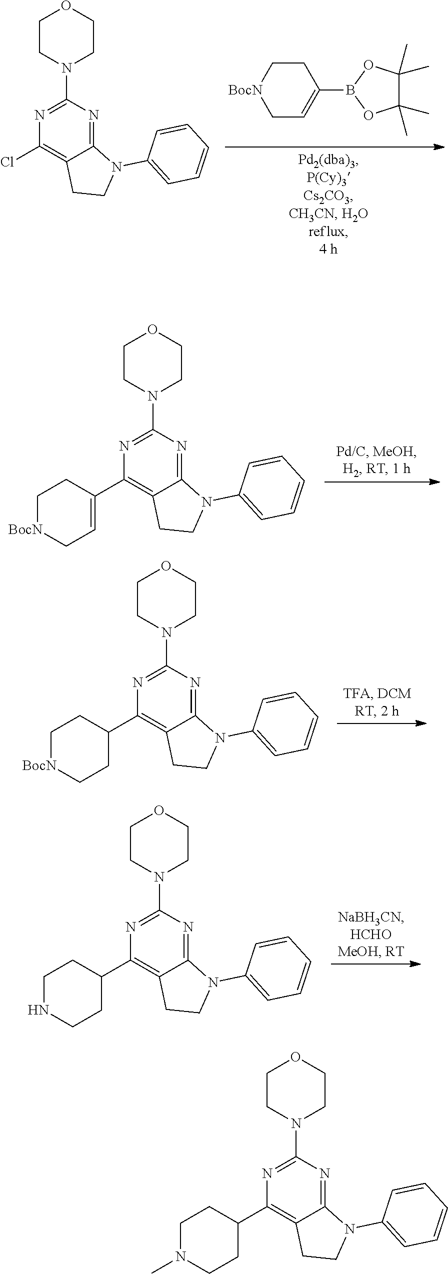

### Step 1: Synthesis of tert-butyl 4-(2-morpholino-7-phenyl-6,7-dihydro-5H-pyrrolo[2,3-d]pyrimidin-4-yl)-3,6-dihydropyridine-1(2H)-carboxylate

A mixture of 4-(4-chloro-7-phenyl-6,7-dihydro-5H-pyrrolo[2,3-d]pyrimidin-2-yl)morpholine (200 mg, 0.63 mmol), tert-butyl 4-(4,4,5,5-tetramethyl-1,3,2-dioxaborolan-2-yl)-3,6-dihydropyridine-1(2H)-carboxylate (391 mg, 1.26 mmol), Cs₂CO₃ (619 mg, 1.90 mmol), Pd₂(dba)₃ (58 mg, 0.063 mmol) and P(Cy)₃ (35 mg, 0.126 mmol) in CH₃CN (20 mL)/H₂O (5 mL) was stirred at 100° C. for 4 h under nitrogen atmosphere. The mixture was concentrated and the residue was extracted with EtOAc (20*3 mL)/H2O (10 mL). The organic layer was washed with brine (30 mL), dried over sodium sulfate, filtered and concentrated. The residue was purified by SGC (PE/EA=4:1) to obtain tert-butyl 4-(2-morpholino-7-phenyl-6,7-dihydro-5H-pyrrolo[2,3-d]pyrimidin-4-yl)-3,6-dihydropyridine-1(2H)-carboxylate (160 mg, 55%) as yellow solid. LCMS (ESI) m/z: 464.3 [M+H]⁺.

### Step 2: Synthesis of tert-butyl 4-(2-morpholino-7-phenyl-6,7-dihydro-5H-pyrrolo[2,3-d]pyrimidin-4-yl)piperidine-1-carboxylate

To a solution of tert-butyl 4-(2-morpholino-7-phenyl-6,7-dihydro-5H-pyrrolo[2,3-d]pyrimidin-4-yl)-3,6-dihydropyridine-1(2H)-carboxylate (160 mg, 0.35 mmol) in MeOH (20 mL) was added 10% Pd/C (16 mg) and the resultant mixture was stirred at room temperature for 1 h under hydrogen atmosphere. The mixture was filtered and concentrated and crude product obtained was purified by prep-HPLC (0.05% NH4HCO3/H2O:CH3CN=5%˜95%) to obtain tert-butyl 4-(2-morpholino-7-phenyl-6,7-dihydro-5H-pyrrolo[2,3-d]pyrimidin-4-yl)piperidine-1-carboxylate (130 mg, 81%) as yellow solid. ¹H NMR (400 MHz, CDCl3) δ 7.78 (d, J=8.0 Hz, 2H), 7.39 (t, J=8.0 Hz, 2H), 7.05 (t, J=7.2 Hz, 1H), 4.22 (bs, 2H), 4.06 (t, J=8.4 Hz, 2H), 3.79 (s, 8H), 3.06-3.02 (m, 2H), 2.86-2.79 (m, 2H), 2.61-2.60 (m, 1H), 1.89-1.82 (m, 2H), 1.73-1.69 (m, 2H), 1.51 (s, 9H); LCMS (ESI) m/z: 466.2 [M+H]⁺.

### Step 3: Synthesis of 4-(7-phenyl-4-(piperidin-4-yl)-6,7-dihydro-5H-pyrrolo[2,3-d]pyrimidin-2-yl)morpholine

To a solution of tert-butyl 4-(2-morpholino-7-phenyl-6,7-dihydro-5H-pyrrolo[2,3-d]pyrimidin-4-yl)piperidine-1-carboxylate (100 mg, 0.2 mmol) in dichloromethane (2 mL) was added TFA (0.5 mL) at 0° C. The mixture was then stirred at room temperature for 2 h and concentrated. The residue was purified by prep-HPLC (0.05% NH4HCO3/H2O:CH3CN=5%˜95%) to obtain 4-(7-phenyl-4-(piperidin-4-yl)-6,7-dihydro-5H-pyrrolo[2,3-d]pyrimidin-2-yl)morpholine (55 mg, 70%,) as white solid. ¹H NMR (400 MHz, CDCl3) δ 7.79 (d, J=8.0 Hz, 2H), 7.38 (t, J=8.0 Hz, 2H), 7.05 (t, J=7.2 Hz, 1H), 4.06 (t, J=8.0 Hz, 2H), 3.80 (s, 8H), 3.27 (d, J=12.0 Hz, 2H), 3.04 (t, J=8.4 Hz, 2H), 2.82-2.76 (m, 2H), 2.64-2.60 (m, 1H), 1.97-1.89 (m, 2H), 1.69-1.66 (m, 2H); LCMS (ESI) m/z: 366.1 [M+H]⁺.

### Step 4: Synthesis of 4-(4-(1-methylpiperidin-4-yl)-7-phenyl-6,7-dihydro-5H-pyrrolo[2,3-d]pyrimidin-2-yl)morpholine

To a solution of 4-(7-phenyl-4-(piperidin-4-yl)-6,7-dihydro-5H-pyrrolo[2,3-d]pyrimidin-2-yl)morpholine (40 mg, 0.11 mmol) in methanol (5 mL) was added formaldehyde (4 mg, 0.12 mmol). The mixture was stirred at room temperature for 2 h followed by the addition of sodium cyanoborohydride (35 mg, 0.55 mmol) to the mixture. The mixture was stirred further for 12 h at room temperature and concentrated. The resultant crude product was purified by prep-HPLC (0.05% NH4HCO3/H2O:CH3CN=5%˜95%) to obtain 4-(4-(1-methylpiperidin-4-yl)-7-phenyl-6,7-dihydro-5H-pyrrolo[2,3-d]pyrimidin-2-yl)morpholine (35.9 mg, 85%) as white solid. ¹H NMR (400 MHz, CDCl3) δ 7.78 (d, J=8.0 Hz, 2H), 7.38 (t, J=8.0 Hz, 2H), 7.04 (t, J=7.2 Hz, 1H), 4.05 (t, J=8.4 Hz, 2H), 3.79 (s, 8H), 3.06-2.99 (m, 4H), 2.43 (bs, 1H), 2.34 (s, 3H), 2.09-2.00 (m, 4H), 1.74-1.63 (m, 2H); LCMS (ESI) m/z: 380.3 [M+H]⁺.

### Synthesis of 4,4′-(7-phenyl-6,7-dihydro-5H-pyrrolo[2,3-d]pyrimidine-2,4-diyl)dimorpholine (Compound 66)

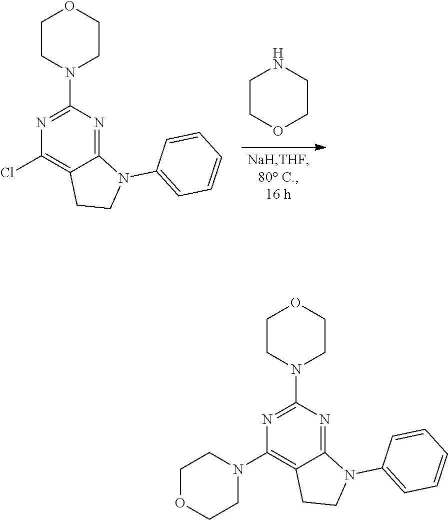

To a solution of morpholine (45 mg, 0.52 mmol) in THE (10 mL) was added NaH (38 mg, 0.95 mmol) at 0° C. The suspension was stirred at room temperature for 15 min followed by the addition 4-(4-chloro-7-phenyl-6,7-dihydro-5H-pyrrolo[2,3-d]pyrimidin-2-yl)morpholine (150 mg, 0.47 mmol) to the mixture. Then mixture was then stirred at 80° C. for 16 h and quenched with water (10 mL), extracted with ethyl acetate (30*3 mL), washed with brine (30 mL), dried over sodium sulfate, filtered and concentrated. The crude product obtained was purified by prep-HPLC (0.05% NH4HCO3/H2O:CH3CN=5%˜95%) to obtain 4,4′-(7-phenyl-6,7-dihydro-5H-pyrrolo[2,3-d]pyrimidine-2,4-diyl)dimorpholine (18.6 mg, 11%) as yellow solid. ¹H NMR (400 MHz, CDCl3) δ 7.72 (d, J=8.0 Hz, 2H), 7.36 (t, J=8.0 Hz, 2H), 7.01 (t, J=7.6 Hz, 1H), 3.98 (t, J=8.4 Hz, 1H), 3.87-3.74 (m, 12H), 3.64-3.62 (m, 4H), 3.16 (t, J=8.4 Hz, 2H); LCMS (ESI) m/z: 368.1 [M+H]⁺.

### Synthesis of 4-(6-(1-methylpyrrolidin-3-yl)-9-phenyl-9H-purin-2-yl)morpholine (Compound 67)

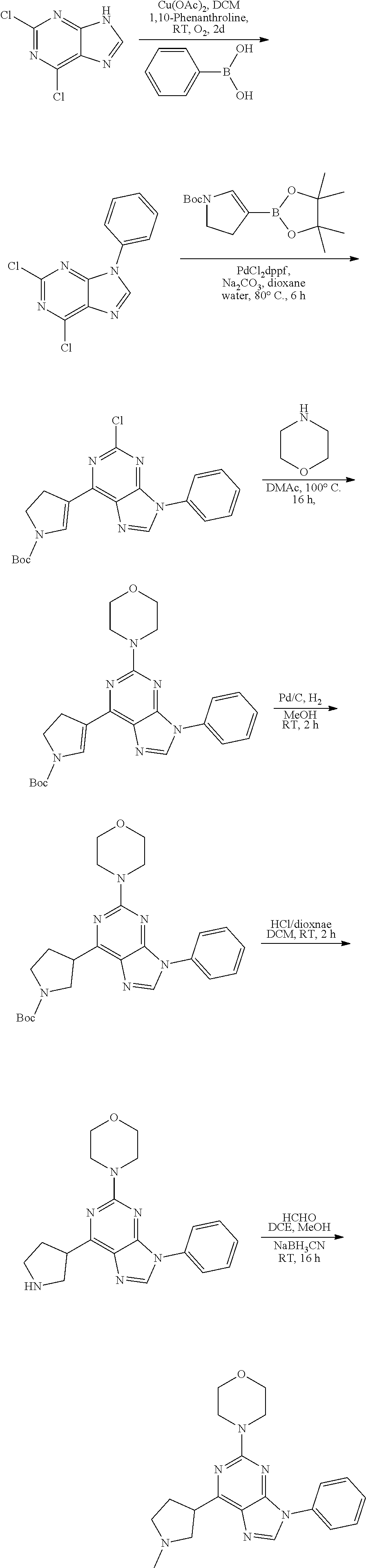

### Step 1: Synthesis of 2,6-dichloro-9-phenyl-9H-purine

To a solution of 2,6-dichloro-9H-purine (1.88 g, 10 mmol), phenylboronic acid (1.83 g, 15 mmol) in dichloromethane (50 mL) were added cupric acetate (900 mg, 5 mmol) and 1,10-Phenanthroline (900 mg, 5 mmol) and the resultant mixture was stirred at room temperature for 2 d under oxygen. The mixture was filtered and the filtrate was concentrated. The residue was subjected to flash chromatography eluting with 0-5% methanol in dichloromethane to obtain 2,6-dichloro-9-phenyl-9H-purine as white solid (1.1 g, 42%). ¹H NMR (400 MHz, CDCl₃) δ 8.40 (s, 1H), 7.60-7.60 (m, 2H), 7.56-7.46 (m, 2H), 7.48-7.44 (m, 1H); LCMS (ESI) m/z: 265.0 [M+H]⁺.

### Step 2: Synthesis of tert-butyl 4-(2-chloro-9-phenyl-9H-purin-6-yl)-2,3-dihydro-1H-pyrrole-1-carboxylate

To a solution of 2,6-dichloro-9-phenyl-9H-purine (132 mg, 0.5 mmol) in dioxane (5 mL) and water (1 mL) were added tert-butyl 4-(4,4,5,5-tetramethyl-1,3,2-dioxaborolan-2-yl)-2,3-dihydro-1H-pyrrole-1-carboxylate (148 mg, 0.5 mmol), [1,1′-bis(diphenylphosphino)ferrocene]dichloropalladium(II) (40 mg, 0.05 mmol) and sodium carbonate (159 mg, 1.5 mmol) at 25° C. and the resultant mixture was stirred at 80° C. for 6 h under argon protection. It was cooled and the mixture was diluted with water (20 mL). The resultant precipitate was collected by filtration, washed with water (20 mL) and dried to give tert-butyl 4-(2-chloro-9-phenyl-9H-purin-6-yl)-2,3-dihydro-1H-pyrrole-1-carboxylate as yellow solid. (180 mg, 90%). LCMS (ESI) m/z: 342.1 [M−56+H]⁺.

### Step 3: Synthesis of tert-butyl 4-(2-morpholino-9-phenyl-9H-purin-6-yl)-2,3-dihydro-1H-pyrrole-1-carboxylate

To a mixture of tert-butyl 4-(2-chloro-9-phenyl-9H-purin-6-yl)-2,3-dihydro-1H-pyrrole-1-carboxylate (40 mg, 0.1 mmol) in N,N-dimethylacetamide (2 mL) was added morpholine (44 mg, 0.5 mmol) and the mixture was stirred at 100° C. for 16 h. It was then extracted with ethyl acetate (10 mL*3) and washed with water (10 mL*3). The combined organic layer was dried and concentrated. The residue was subjected to prep-TLC (UV254, Silica, petroleum ether/ethyl acetate=1/1) to give tert-butyl 4-(2-morpholino-9-phenyl-9H-purin-6-yl)-2,3-dihydro-1H-pyrrole-1-carboxylate as yellow solid. (20 mg, 44%). LCMS (ESI) m/z: 449.1 [M+H]⁺.

### Step 4: Synthesis of tert-butyl 3-(2-morpholino-9-phenyl-9H-purin-6-yl)pyrrolidine-1-carboxylate

To a mixture of tert-butyl 4-(2-morpholino-9-phenyl-9H-purin-6-yl)-2,3-dihydro-1H-pyrrole-1-carboxylate (45 mg, 0.1 mmol) in methanol (5 mL) was added palladium/carbon (10%, 20 mg) and the suspension was stirred at room temperature for 2 h under hydrogen. The mixture was filtered and the filtrate was concentrated to obtain tert-butyl 3-(2-morpholino-9-phenyl-9H-purin-6-yl)pyrrolidine-1-carboxylate as yellow solid. (45 mg, 99%). LCMS (ESI) m/z: 451.2 [M+H]⁺.

### Step 5: Synthesis of 4-(9-phenyl-6-(pyrrolidin-3-yl)-9H-purin-2-yl)morpholine

A mixture of tert-butyl 3-(2-morpholino-9-phenyl-9H-purin-6-yl)pyrrolidine-1-carboxylate (45 mg, 0.1 mmol) and hydrochloric acid/dioxane (4M, 2 mL) in dichloromethane (5 mL) was stirred at room temperature for 2 h. It was then diluted with 10 mL of dichloromethane and the mixture was washed with aqueous sodium bicarbonate solution (10 mL). The organic layer was concentrated to obtain 4-(9-phenyl-6-(pyrrolidin-3-yl)-9H-purin-2-yl)morpholine as yellow solid. (35 mg, 99%). LCMS (ESI) m/z: 351.2 [M+H]⁺.

### Step 6: Synthesis of 4-(6-(1-methylpyrrolidin-3-yl)-9-phenyl-9H-purin-2-yl)morpholine

To a solution of 4-(9-phenyl-6-(pyrrolidin-3-yl)-9H-purin-2-yl)morpholine (35 mg, 0.1 mmol) and formaldehyde (35%, 5 drops) in methanol (1 mL) and dichloroethane (2 mL) was added a drop of acetic acid and the mixture was stirred for 1 h. Then sodium cyanoborohydride (31 mg, 0.5 mmol) was added and the resultant mixture was stirred for 16 h at room temperature. The reaction was quenched with water (10 mL) and the mixture was extracted with dichloromethane (10 mL*2). The organic phase was concentrated and the crude product was purified by prep-HPLC (BOSTON pHlex ODS 10 um 21.2×250 mm120 A. The mobile phase was acetonitrile/0.1% Ammonium bicarbonate) to afford 4-(6-(1-methylpyrrolidin-3-yl)-9-phenyl-9H-purin-2-yl)morpholine (6.5 mg, 18%) as white solid. ¹H NMR (400 MHz, CD₃OD) δ 8.40 (s, 1H), 7.86 (d, J=8.0 Hz, 2H), 7.60 (t, J=8.0 Hz, 2H), 7.47 (t, J=7.6 Hz, 1H), 4.19 (pent, J=8.4 Hz, 1H), 3.88-3.77 (m, 8H), 3.26 (t. J=9.2 Hz, 1H), 3.08-2.83 (m, 3H), 2.52 (s, 3H), 2.44-2.37 (m, 2H); LCMS (ESI) m/z: 365.3 [M+H]⁺.

The following compound was synthesized using similar protocols described above:

| Name | Structure | NMR, MS | # |
|---|---|---|---|
| 4-(6-(1- methylpiperidin- 3-yl)-9- (pyridin-3-yl)-9H- purin-2-yl) morpholine | 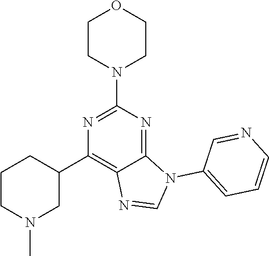 [SMILES: CN1CCCC(c2nc(N3CCOCC3)nc3c2ncn3-c2cccnc2)C1] | ¹H NMR (400 MHz, CD₃OD) δ 9.15 (d, J = 2.0 Hz, 1H), 8.65 (d, J = 4.8 Hz, 1H), 8.43 (s, 1H), 8.38 (d, J = 8.8 Hz, 1H), 7.69 (dd, J = 8.4, 5.2 Hz, 1H), 4.37 (bs, 4H), 3.85-3.83 (m, 4H), 3.23-2.94 (m, 3H), 2.43-2.38 (m, 4H), 2.16-1.63 (m, 5H); LCMS (ESI) m/z: 380.3 [M + H]+. | 68 |

### Synthesis of 4-(9-phenyl-6-(pyridin-4-yl)-9H-purin-2-yl)morpholine (Compound 69)

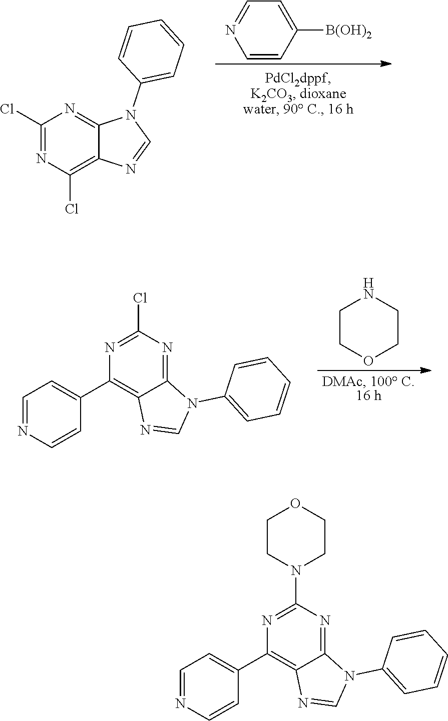

### Step 1: Synthesis of 2-chloro-9-phenyl-6-(pyridin-4-yl)-9H-purine

To a solution of 2,6-dichloro-9-phenyl-9H-purine (264 mg, 1 mmol) in dioxane (10 mL) and water (2 mL) were added pyridin-4-ylboronic acid (123 mg, 1 mmol), [1,1′-bis(diphenylphosphino)ferrocene]dichloropalladium(II) (81 mg, 0.1 mmol) and potassium carbonate (414 mg, 3 mmol) at 25° C. and the resultant mixture was stirred at 90° C. for 16 h under argon protection. The mixture was extracted with ethyl acetate (20 mL*3) and washed with water (20 mL). The organic layer was concentrated and the crude product was purified by prep-TLC (Silica, UV254, ethyl acetate/petroleum ether=311) to afford 2-chloro-9-phenyl-6-(pyridin-4-yl)-9H-purine as yellow solid. (50 mg, 16%). LCMS (ESI) m/z: 308.1 [M+H]⁺. (This step also produced 9-phenyl-2,6-di(pyridin-4-yl)-9H-purine (13 mg, 4%) as the biproduct).

### Step 2: Synthesis of 4-(9-phenyl-6-(pyridin-4-yl)-9H-purin-2-yl)morpholine

To a mixture of 2-chloro-9-phenyl-6-(pyridin-4-yl)-9H-purine (31 mg, 0.1 mmol) in N,N-dimethylacetamide (2 mL) was added morpholine (44 mg, 0.5 mmol) and stirred at 100° C. for 16 h. The mixture was purified with Prep-HPLC (BOSTON pHlex ODS 10 um 21.2×250 mm120 A. The mobile phase was acetonitrile/0.1% Ammonium bicarbonate) to give 4-(9-phenyl-6-(pyridin-4-yl)-9H-purin-2-yl)morpholine as a yellow solid. (26 mg, 72% yield). ¹H NMR (400 MHz, DMSO-d6) δ 8.83 (d, J=6.0 Hz, 2H), 8.79 (s, 1H), 8.67 (d, J=6.0 Hz, 2H), 7.94 (d, J=7.6 Hz, 2H), 7.63 (t, J=8.0 Hz, 2H), 7.49 (t, J=7.6 Hz, 1H), 3.83-3.72 (m, 8H); LCMS (ESI) m/z: 359.2 [M+H]⁺.

### Synthesis of 4-(7-phenyl-4-(pyridin-4-yl)-5H-pyrrolo[3,2-d]pyrimidin-2-yl)morpholine (Compound 70)

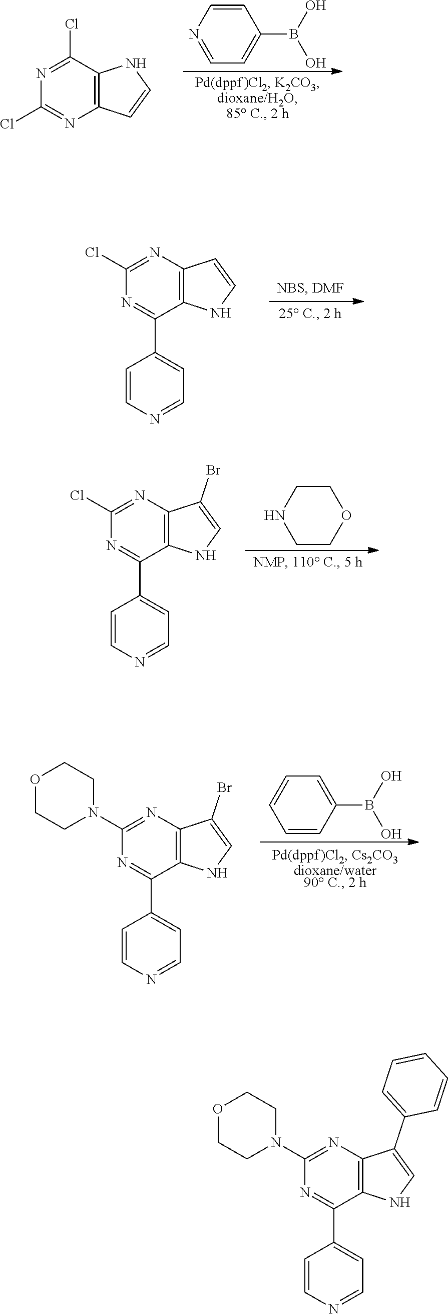

### Step 1: Synthesis of 2-chloro-4-(pyridin-4-yl)-5H-pyrrolo[3,2-d]pyrimidine

To a solution of 2,4-dichloro-5H-pyrrolo[3,2-d]pyrimidine (15 g, 77 mmol) in dioxane/water (200 mL/40 mL) were added pyridin-4-ylboronic acid (5.8 g, 77 mmol), potassium carbonate (21.3 g, 154 mmol) and [1,1′bis(diphenylphosphino)ferrocene]dichloro-palladium(II) (5.6 g, 7.7 mmol) at 25° C. and the resultant mixture was stirred at 85° C. for 2 h under argon protection. The mixture was then filtered and the filtrate was concentrated to obtain the target product as dark solid (15 g, 84%). LCMS (ESI) m/z: 231.1 [M+H]⁺.

### Step 2: Synthesis of 7-bromo-2-chloro-4-(pyridin-4-yl)-5H-pyrrolo[3,2-d]pyrimidine

A mixture of 2-chloro-4-(pyridin-4-yl)-5H-pyrrolo[3,2-d]pyrimidine (3.0 g, 13 mmol) and N-bromosuccinimide (2.3 g, 13 mmol) in N,N-dimethylformamide (30 mL) was stirred at 25° C. for 2 h. To the mixture was added methanol (100 mL) and it was filtered and the filtrate concentrated. The resultant residue was subjected to silica gel column chromatography (petroleum ether:ethyl acetate=1:2) to obtain the target product as yellow solid (2 g, 50%). LCMS (ESI) m/z: 309.2 [M+H]⁺.

### Step 3: Synthesis of 4-(7-bromo-4-(pyridin-4-yl)-5H-pyrrolo[3,2-d]pyrimidin-2-yl)morpholine

A mixture of 7-bromo-2-chloro-4-(pyridin-4-yl)-5H-pyrrolo[3,2-d]pyrimidine (0.3 g, 0.97 mmol) and morpholine (0.5 g, 5.8 mmol) in NMP (3 mL) was stirred at 110° C. for 5 h. It was cooled to room temperature and quenched with water (15 mL). The mixture was extracted with ethyl acetate (15 mL*3) and the organic layer was concentrated and subjected to prep-TLC (dichloromethane:acetic ester=1:1) to obtain the target product as yellow solid (0.08 g, 23%). LCMS (ESI) m/z: 360.1 [M+H]⁺.

### Step 4: Synthesis of 4-(7-phenyl-4-(pyridin-4-yl)-5H-pyrrolo[3,2-d]pyrimidin-2-yl)morpholine

A mixture of 4-(7-bromo-4-(pyridin-4-yl)-5H-pyrrolo[3,2-d]pyrimidin-2-yl)morpholine (0.07 g, 0.19 mmol), [1,1′bis(diphenylph-osphino)ferrocene]dichloropalladium(II) (0.015 g, 0.02 mmol), cesium carbonate (0.19 g, 0.58 mmol) and phenylboronic acid (0.05 g, 0.39 mmol) in dioxane/water (3 mL/0.5 mL) was stirred at 90° C. for 2 h. The mixture was concentrated and the obtained residue was subjected to prep-HPLC (SunFire C18, 4.6*50 mm, 3.5 um column Xbridge C18 3.5 μm 4.6×50 mm column. The mobile phase was acetonitrile/10 mM formic acid aqueous solution) to obtain the target product as yellow solid (0.0198 g, 29%). ¹H NMR (400 MHz, DMSO-d₆) δ 11.96 (s, 1H), 8.81 (d, J=5.3 Hz, 2H), 8.38 (bs, 1H), 8.32 (s, 1H), 8.26 (d, J=7.8 Hz, 2H), 8.05 (d, J=5.1 Hz, 2H), 7.42 (t, J=7.6 Hz, 2H), 7.20 (t, J=7.3 Hz, 1H), 3.83-3.74 (m, 8H); LCMS (ESI) m/z: 358.2 [M+H]⁺.

### Synthesis of 2-methyl-1-(4-((1-methylpiperidin-3-yl)methoxy)-2-morpholino-5H-pyrrolo[2,3-d]pyrimidin-7(6H)-yl)propan-2-ol (Compound 79)

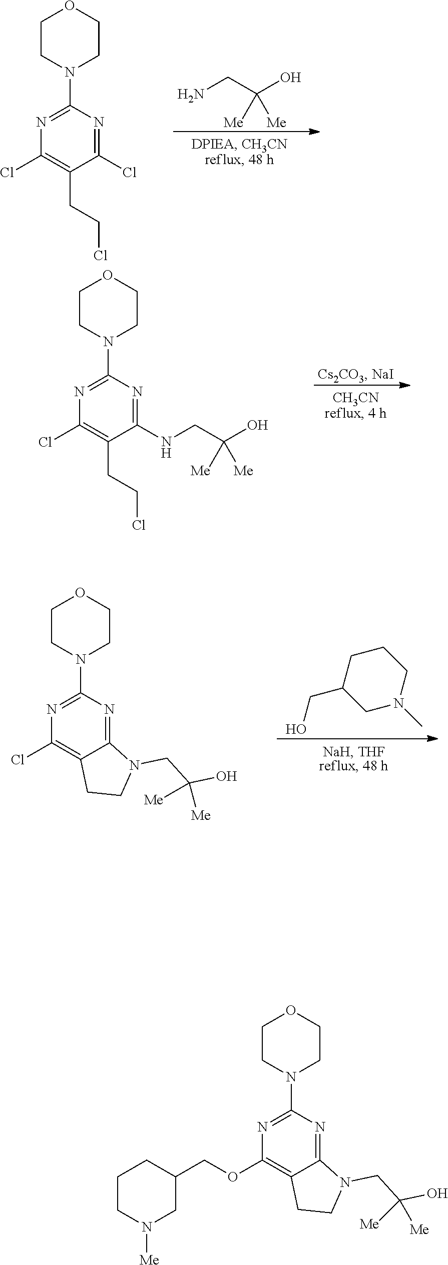

### Step 1: Synthesis of 1-(6-chloro-5-(2-chloroethyl)-2-morpholinopyrimidin-4-ylamino)-2-methylpropan-2-ol

To a stirred solution of 4-(4,6-dichloro-5-(2-chloroethyl)pyrimidin-2-yl)morpholine (200 mg, 0.674 mmol) and 1-amino-2-methylpropan-2-ol (60 mg, 0.674 mmol) in acetonitrile (20 mL) was added N-ethyl-N-isopropylpropan-2-amine (218 mg, 1.687 mmol) at room temperature. The reaction mixture was then refluxed for 48 h. After cooling to room temperature, the mixture was diluted with ethyl acetate (100 mL), washed with water (30 mL) and brine (30 mL). The organics were dried over sodium sulfate, filtered and concentrated to give 1-(6-chloro-5-(2-chloroethyl)-2-morpholinopyrimidin-4-ylamino)-2-methylpropan-2-ol (200 mg, 85%) as brown solid. LCMS (ESI) m/z: 348.9 [M+H]⁺.

### Step 2: Synthesis of 1-(4-chloro-2-morpholino-5H-pyrrolo[2,3-d]pyrimidin-7(6H)-yl)-2-methylpropan-2-ol

Cesium carbonate (466 mg, 1.43 mmol) was added to a solution of 1-(6-chloro-5-(2-chloroethyl)-2-morpholinopyrimidin-4-ylamino)-2-methylpropan-2-ol (200 mg, 0.573 mmol) and sodium iodide (17 mg, 0.113 mmol) in acetonitrile (20 mL) at room temperature. The reaction mixture was refluxed for 4 h under nitrogen atmosphere, cooled and then diluted with ethyl acetate (150 mL). The mixture was washed with water (50 mL), brine (30 mL), dried over sodium sulfate, filtered and concentrated. The crude product obtained was purified by silica gel column chromatography, eluting with dichloromethane/methanol=9/1 to give 1-(4-chloro-2-morpholino-5H-pyrrolo[2,3-d]pyrimidin-7(6H)-yl)-2-methylpropan-2-ol (100 mg, 55%) as white solid. LCMS (ESI) m/z: 313.1 [M+H]⁺.

### Step 3: Synthesis of 2-methyl-1-(4-((1-methylpiperidin-3-yl)methoxy)-2-morpholino-5H-pyrrolo[2,3-d]pyrimidin-7(6H)-yl)propan-2-ol

A suspension of (1-methylpiperidin-3-yl)methanol (83 mg, 0.64 mmol) and sodium hydride (32 mg, 0.8 mmol) in tetrahydrofuran (10 mL) was stirred at room temperature for 10 min and then 1-(4-chloro-2-morpholino-5H-pyrrolo[2,3-d]pyrimidin-7(6H)-yl)-2-methylpropan-2-ol (100 mg, 0.32 mmol) was added. The resultant mixture was refluxed for 48 h and cooled. It was then diluted with ethyl acetate (80 mL), washed with water (30 mL) and brine (30 mL), dried over sodium sulfate, filtered and concentrated. The crude product was purified by prep-HPLC (SunFire C18, 4.6*50 mm, 3.5 um column Xbridge C18 3.5 μm 4.6×50 mm column. The elution system used was a gradient of 5%˜95% over 1.5 min at 2 ml/min and the solvent was acetonitrile/0.01% aqueous ammonium bicarbonate.) to obtain 2-methyl-1-(4-((1-methylpiperidin-3-yl)methoxy)-2-morpholino-5H-pyrrolo[2,3-d]pyrimidin-7(6H)-yl)propan-2-ol (14 mg, 11%) as pale yellow solid. ¹H NMR (500 MHz, MeOD) δ 6.03 (s, 1H), 4.17-4.07 (m, 2H), 3.73-3.61 (m, 10), 3.22 (s, 2H), 2.93-2.77 (m, 4H), 2.27 (s, 3H), 2.07 (bs, 1H), 1.95-1.89 (m, 1H), 1.76-1.57 (m, 4H), 1.23 (s, 6H), 1.00 (m, 1H). LCMS (ESI) m/z: 406.2 [M+H]⁺.

### Synthesis of cyclopropyl(3-(2-morpholino-7-phenyl-6,7-dihydro-5H-pyrrolo[2,3-d]pyrimidin-4-yl)pyrrolidin-1-yl)methanone (Compound 86)

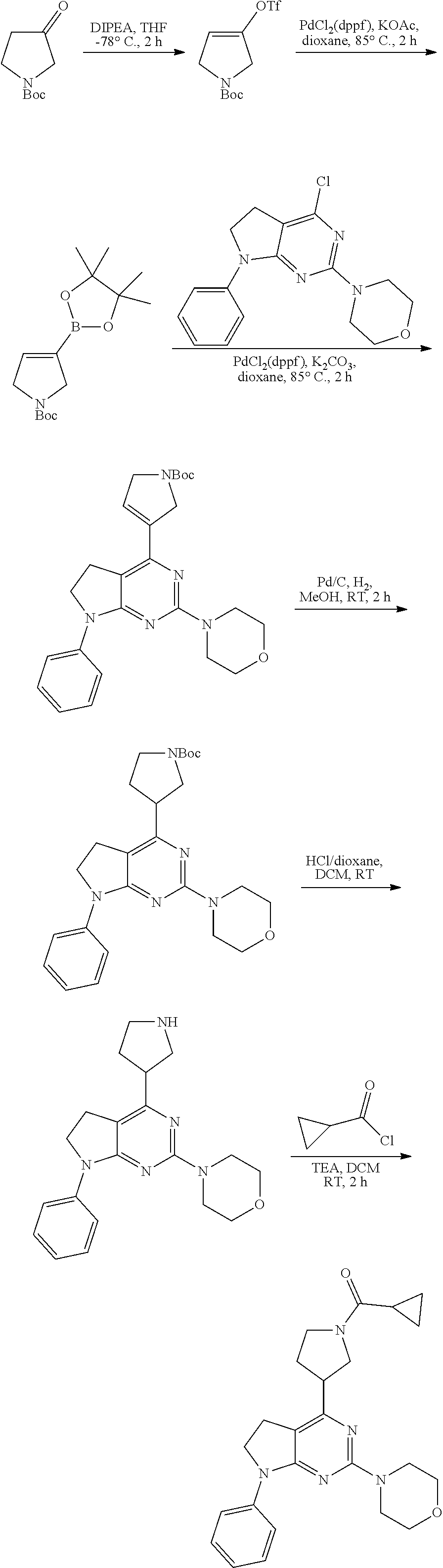

### Step 1: Synthesis of tert-butyl 3-(((trifluoromethyl)sulfonyl)oxy)-2,5-dihydro-1H-pyrrole-1-carboxylate

A solution of tert-butyl 3-oxopyrrolidine-1-carboxylate (1400 mg, 7.567 mmol) and N,N-diisopropylethylamine (2928 mg, 22.701 mmol) in dichloromethane (50 mL) was cooled to −78° C. and stirred for 10 mins. Then trifluoromethanesulfonic anhydride (2560 mg, 9.081 mmol) was added and the mixture was warmed up and stirred at 25° C. for 16 h. The reaction was quenched with aqueous ammonium chloride solution and extracted with dichloromethane (50 mL×3). The organic layer was dried and concentrated to give tert-butyl 3-(((trifluoromethyl) sulfonyl)oxy)-2,5-dihydro-1H-pyrrole-1-carboxylate (800 mg, 33%) as yellow oil. LC-MS: m/z=262 (M−56+H)⁺.

### Step 2: Synthesis of tert-Butyl 3-(4,4,5,5-tetramethyl-1,3,2-dioxaborolan-2-yl)-2,5-dihydro-1H-pyrrole-1-carboxylate

A solution of tert-butyl 3-(((trifluoromethyl)sulfonyl)oxy)-2,5-dihydro-1H-pyrrole-1-carboxylate (2800 mg, 8.832 mmol), 4,4,4′,4′, 5,5,5′,5′-octamethyl-2,2′-bi(1,3,2-dioxaborolane) (4487 mg, 17.665 mmol), [1,1′-bis(diphenylphosphino) ferrocene] dichloro palladium(II) (325 mg, 0.441 mmol) and potassium acetate (2600 mg, 26.532 mmol) in dioxane (80 mL) was stirred at 75° C. for 4 h. Then water was added and the resultant mixture was extracted with ethyl acetate (50 mL×3). The organic layer was dried and concentrated. The crude product obtained was purified by silica gel column (petroleum ether:ethyl acetate from 50:1 to 10:1) to give tert-butyl 3-(4,4,5,5-tetramethyl-1,3,2-dioxaborolan-2-yl)-2,5-dihydro-1H-pyrrole-1-carboxylate (2050 mg, 78%) as yellow solid. LC-MS: m/z=240 (M−56+H)⁺.

### Step 3: Synthesis of tert-Butyl 3-(2-morpholino-7-phenyl-6,7-dihydro-5H-pyrrolo[2,3-d]pyrimidin-4-yl)-2,5-dihydro-1H-pyrrole-1-carboxylate

A solution of tert-butyl 3-(4,4,5,5-tetramethyl-1,3,2-dioxaborolan-2-yl)-2,5-dihydro-1H-pyrrole-1-carboxylate (280 mg, 0.95 mmol), 4-(4-chloro-7-phenyl-6,7-dihydro-5H-pyrrolo[2,3-d]pyrimidin-2-yl)morpholine (200 mg, 0.63 mmol), bis(diphenylphosphino) ferrocene]dichloropalladium(II) (20 mg, 0.03 mmol) and potassium carbonate (260 mg, 1.89 mmol) in dioxane/water (30 mL) was stirred at 85° C. for 4 h. Then water was added and the mixture was extracted with ethyl acetate (50 mL×3). The organic layer was dried, concentrated and the crude product obtained was purified by silica gel column chromatography (petroleum ether:ethyl acetate from 50:1 to 10:1) to obtain tert-butyl 3-(2-morpholino-7-phenyl-6,7-dihydro-5H-pyrrolo[2,3-d]pyrimidin-4-yl)-2,5-dihydro-1H-pyrrole-1-carboxylate (200 mg, 71%) as yellow solid. LC-MS: m/z=450 (M+H)⁺.

### Step 4: Synthesis of tert-Butyl 3-(2-morpholino-7-phenyl-6,7-dihydro-5H-pyrrolo[2,3-d]pyrimidin-4-yl)pyrrolidine-1-carboxylate

A suspension of tert-butyl 3-(2-morpholino-7-phenyl-6,7-dihydro-5H-pyrrolo[2,3-d]pyrimidin-4-yl)-2,5-dihydro-1H-pyrrole-1-carboxylate (200 mg, 0.445 mmol) and palladium/carbon (100 mg) in methanol (5 mL) was stirred at 25° C. for 16 h. The mixture was filtered and the filtrate was concentrated and dried to obtain tert-butyl 3-(2-morpholino-7-phenyl-6,7-dihydro-5H-pyrrolo[2,3-d]pyrimidin-4-yl)pyrrolidine-1-carboxylate (180 mg, 90%) as yellow solid. LC-MS: m/z=452 (M+H)⁺.

### Step 5: Synthesis of 4-(7-Phenyl-4-(pyrrolidin-3-yl)-6,7-dihydro-5H-pyrrolo[2,3-d]pyrimidin-2-yl)morpholine

A solution of tert-butyl 3-(2-morpholino-7-phenyl-6,7-dihydro-5H-pyrrolo[2,3-d]pyrimidin-4-yl)pyrrolidine-1-carboxylate (420 mg, 0.931 mmol) and HCl in dioxane (4 mL) in dichloromethane (6 mL) was stirred at 25° C. for 2 h. The mixture was concentrated to obtain 4-(7-phenyl-4-(pyrrolidin-3-yl)-6,7-dihydro-5H-pyrrolo[2,3-d]pyrimidin-2-yl) morpholine (280 mg, 85%) as yellow solid. LC-MS: m/z=352 (M+H)⁺.

### Step 6: Synthesis of cyclopropyl(3-(2-morpholino-7-phenyl-6,7-dihydro-5H-pyrrolo[2,3-d]pyrimidin-4-yl)pyrrolidin-1-yl)methanone

A solution of 4-(7-phenyl-4-(pyrrolidin-3-yl)-6,7-dihydro-5H-pyrrolo[2,3-d]pyrimidin-2-yl)morpholine (80 mg, 0.228 mmol) and triethylamine (69 mg, 0.684 mmol) in dichloromethane (5 mL) was stirred at 25° C. for 10 min. Then cyclopropane carbonyl chloride (28 mg, 0.274 mmol) was added and the resultant mixture was stirred at room temperature for 2 h. It was filtered and the filtrate was concentrated. The crude product obtained was purified by Pre-HPLC(Column Xbridge 21.2*250 mm C18, 10 um, mobile phase A: water (10 mmol/L ammonium bicarbonate) B: acetonitrile) to obtain cyclopropyl(3-(2-morpholino-7-phenyl-6,7-dihydro-5H-pyrrolo[2,3-d]pyrimidin-4-yl)pyrrolidin-1-yl)methanone (43.1 mg, 45%) as yellow solid. ¹H NMR (400 MHz, DMSO-d₆) δ 7.80 (d, J=8.0 hz, 2H), 7.37 (t, J=7.2 Hz, 2H), 7.01 (t, J=7.2 Hz, 1H), 4.08-4.05 (m, 2H), 3.93-3.84 (m, 2H), 3.76-3.64 (m, 9H), 3.56-3.49 (m, 2H), 3.07-3.00 (m, 2H), 2.22-2.04 (m, 2H), 1.79-1.76 (m, 1H), 0.75-0.73 (m, 4H); LC-MS: m/z=420 (M+H)⁺.

The following compounds were synthesized according to the protocol described above:

| Name | Structure | NMR, MS | # |
|---|---|---|---|
| 1-(3-(2- morpholino- 7-phenyl- 6,7-dihydro- 5H- pyrrolo[2,3- d]pyrimidin- 4- yl)pyrrolidin- 1- yl)ethanone | 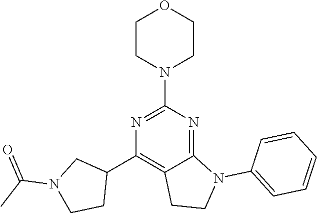 [SMILES: CC(=O)N1CCC(c2nc(N3CCOCC3)nc3c2CCN3c2ccccc2)C1] | ¹H NMR (400 MHz, DMSO-d₆) δ 7.80 (d, J = 8.0 Hz, 2H), 7.37 (t, J = 8 Hz, 2H), 7.00 (t, J = 7.2 Hz, 1H), 4.07-4.03 (m, 2H), 3.75-3.73 (m, 1H), 3.58-3.49 (m, 9H), 3.43-3.39 (m, 2H), 3.32-3.29 (m, 1H), 3.05-3.00 (m, 2H), 2.17-2.11 (m, 2H), 2.082 (s, 3H); LC-MS: m/z = 394.2 (M + H)⁺. | 87 |
| 1-(4-(2- morpholino- 7-phenyl- 6,7-dihydro- 5H- pyrrolo[2,3- d]pyrimidin- 4- yl)piperidin- 1-yl)ethan-1- one | 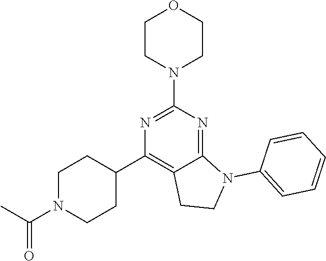 [SMILES: CC(=O)N1CCC(c2nc(N3CCOCC3)nc3c2CCN3c2ccccc2)CC1] | ¹H NMR (400 MHz, CDCl3) δ 7.76 (d, J = 6.0 Hz, 2H), 7.39 (t, J = 6.4 Hz, 2H), 7.05 (t, J = 6.0 Hz, 1H), 4.74 (d, J = 10.8 Hz, 1H), 4.05 (t, J = 6.8 Hz, 2H), 3.93 (d, J = 10.4 Hz, 1H), 3.76 (s, 8H), 3.19-3.13 (m, 1H), 3.02 (t, J = 6.8 Hz, 2H), 2.70-2.65 (m, 2H), 2.14 (s, 3H), 1.92-1.73 (m, 4H); LCMS (ESI) m/z: 408.1 [M + H]+. | 89 |

### Synthesis of 4-(4-(furan-3-yl)-7-(pyridin-3-yl)-6,7-dihydro-5H-pyrrolo[2,3-d]pyrimidin-2-yl)morpholine (Compound 109) and 4-(7-(pyridin-3-yl)-4-(tetrahydrofuran-3-yl)-6,7-dihydro-5H-pyrrolo[2,3-d]pyrimidin-2-yl)morpholine (Compound 90)

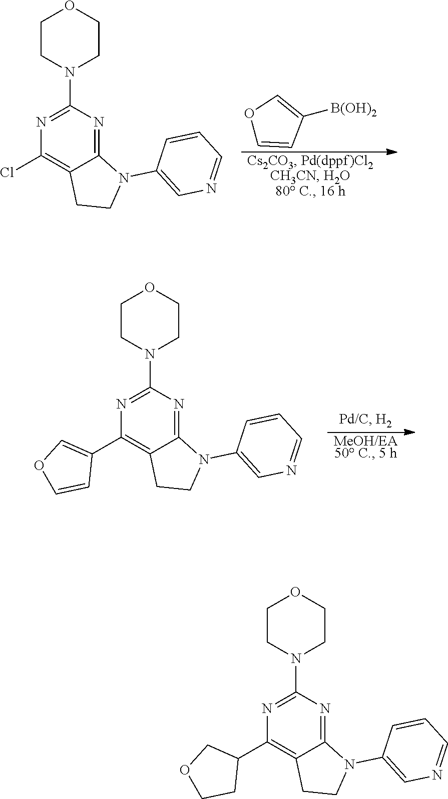

### Step 1: Synthesis of 4-(4-(furan-3-yl)-7-(pyridin-3-yl)-6,7-dihydro-5H-pyrrolo[2,3-d]pyrimidin-2-yl)morpholine

To a stirred mixture of 4-(4-chloro-7-(pyridin-3-yl)-6,7-dihydro-5H-pyrrolo[2,3-d]pyrimidin-2-yl)morpholine (200 mg, 0.62 mmol), furan-3-ylboronic acid (211 mg, 1.88 mmol), [1,1′-bis(diphenylphosphino) ferrocene]dichloropalladium(II) (69 mg, 0.094 mmol) in acetonitrile (6 mL) and water (1.5 mL) at 20° C. was added cesium carbonate (615 mg, 1.88 mmol). The resultant mixture was stirred at 80° C. for 18 h under nitrogen and cooled. The reaction mixture was then quenched with water (50 mL) and extracted with dichloromethane (50 mL×2). The combined organic fractions were washed with brine (50 mL), dried over sodium sulfate, filtered and concentrated. The residue was subjected to prep-HPLC to obtain the title compound (28.1 mg, 13%) as white solid. 1H NMR (400 MHz, CDCl3) δ 9.13 (s, 1H), 8.29 (d, J=3.5 Hz, 1H), 8.21 (d, J=8.7 Hz, 1H), 7.95 (s, 1H), 7.51 (t, J=1.7 Hz, 1H), 7.34 (dd, J=8.5, 4.7 Hz, 1H), 6.95 (s, 1H), 4.14 (t, J=8 Hz, 2H), 3.84-3.80 (m, 98), 3.24 (t, J=8 Hz, 2H); LCMS (ESI) m/z: 350.1 [M+H]⁺.

### Step 2: Preparation of 4-(7-(pyridin-3-yl)-4-(tetrahydrofuran-3-yl)-6,7-dihydro-5H-pyrrolo[2,3-d]pyrimidin-2-yl)morpholine

To a solution of 4-(4-(furan-3-yl)-7-(pyridin-3-yl)-6,7-dihydro-5H-pyrrolo[2,3-d]pyrimidin-2-yl)morpholine (74 mg, 0.21 mmol) in methanol (25 mL) and acetic ester (25 mL) was added palladium on activated carbon 10% Pd (74 mg). The resultant suspension was stirred at 50° C. for 5 h under hydrogen atmosphere. The reaction mixture was filtered and the filtrate was concentrated. The residue was subjected to prep-HPLC (BOSTON pHlex ODS 10 um 21.2×250 mm 120 A. The mobile phase was acetonitrile/0.1% Ammonium bicarbonate) to obtain 4-(7-(pyridin-3-yl)-4-(tetrahydrofuran-3-yl)-6,7-dihydro-5H-pyrrolo[2,3-d]pyrimidin-2-yl)morpholine (16.1 mg, 21%) as white solid. ¹H NMR (500 MHz, Chloroform-d) δ 9.08 (d, J=2.8 Hz, 1H), 8.29 (dd, J=4.0, 0.4 Hz, 1H), 8.15 (dt, J=9.2, 0.8 Hz, 1H), 7.30 (dd, J=8.5, 4.7 Hz, 1H), 4.11 (t, J=8.0 Hz, 1H), 4.08-4.00 (m, 3H), 3.96-3.88 (m, 2H), 3.80-3.75 (m, 8H), 3.36 (pent, J=6.4 Hz, 1H), 3.14-3.02 (m, 2H), 2.34-2.26 (m, 1H), 2.22-2.14 (m, 1H). LCMS (ESI) m/z: 354.2 [M+H]⁺.

### Synthesis of 4-(6-methyl-7-(pyridin-4-yl)-4-(tetrahydrofuran-3-yl)-6,7-dihydro-5H-pyrrolo[2,3-d]pyrimidin-2-yl)morpholine (Compound 91)

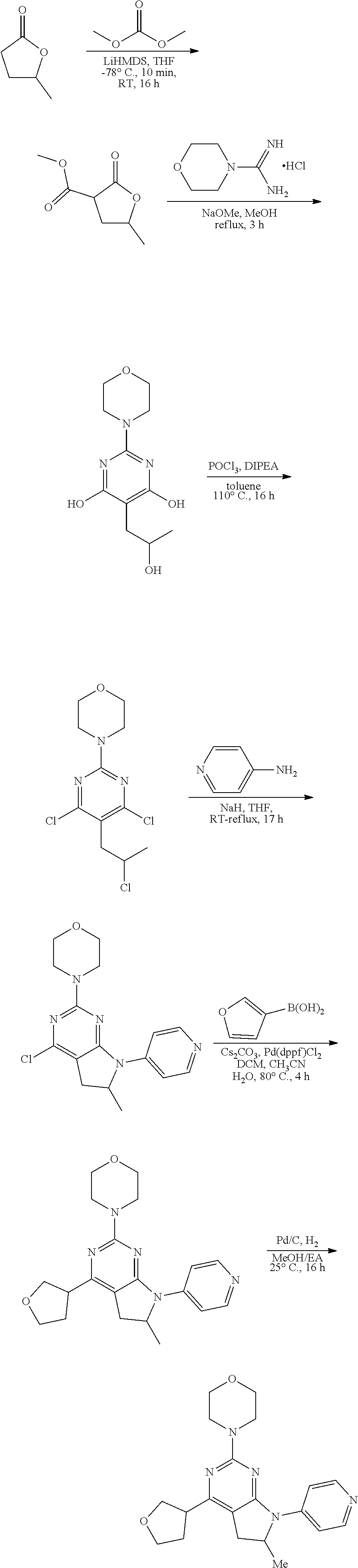

### Step 1: Synthesis of methyl 5-methyl-2-oxotetrahydrofuran-3-carboxylate. PGP-10

A solution of methyldihydrofuran-2(3H)-one (25 g, 250 mmol) in tetrahydrofuran (100 mL) was added dropwise to lithium bis(trimethylsilyl)amide (1.6 M in tetrahydrofuran, 330 mL, 528 mmol) at −78° C. After stirring at −78° C. for 10 min, dimethyl carbonate (23.6 g, 263 mmol) was added to the mixture at −78° C. and the reaction mixture was warmed up and stirred at room temperature for 16 h. It was then poured into a mixture of concentrated hydrochloric acid (80 mL) and ice (800 mL), followed by extraction with ethyl acetate (800 mL×2). The organic layer was washed by brine, dried over sodium sulfate, filtered and concentrated to obtain the title compound (40 g). This product was used in the next step without further purification. LCMS (ESI) m/z: 159.1 [M+H]⁺.

### Step 2: Synthesis of 5-(2-hydroxypropyl)-2-morpholinopyrimidine-4,6-diol

Methyl 5-methyl-2-oxotetrahydrofuran-3-carboxylate (40 g, 250 mmol) was added to a solution of morpholine-4-carboximidamide hydrochloride (31 g, 192 mmol) and sodium methanolate (104 g, 576 mmol) in methanol (150 mL) at room temperature. The reaction mixture was then refluxed for 16 h and cooled. Water (200 mL) was added to the mixture and stirred for 0.5 h, followed by the addition of acetic acid (30 mL) and the mixture was stirred further for 2 h at room temperature. The precipitated solid was filtered and dried to give the title compound (41 g, 83%) as white solid. LCMS (ESI) m/z: 256.2 [M+H]⁺.

### Step 3: Synthesis of 4-(4,6-dichloro-5-(2-chloropropyl)pyrimidin-2-yl)morpholine

To a solution of 5-(2-hydroxypropyl)-2-morpholinopyrimidine-4,6-diol (41 g, 81 mmol) and N-ethyl-N-isopropylpropan-2-amine (44 mL) in toluene (400 mL) was added phosphorus oxychloride (64 mL) at room temperature. The resultant mixture was stirred at 110° C. for 16 h and concentrated. The residue was then dissolved in ethyl acetate (1600 mL) and washed with water (300 mL×2), brine (300 mL), and dried over sodium sulfate. Concentration and purification of the resultant residue on silica gel column chromatography (petroleum ether/ethyl acetate=10/1) afforded the title compound (38 g, 76%) as off-white solid. LCMS (ESI) m/z: 312.0 [M+H]⁺.

### Step 4: Synthesis of 4-(4-chloro-6-methyl-7-(pyridin-4-yl)-6,7-dihydro-5H-pyrrolo[2,3-d]pyrimidin-2-yl)morpholine

A mixture of pyridin-4-amine (151 mg, 1.60 mmol) and sodium hydride (161 mg, 4.025 mmol) in tetrahydrofuran (40 mL) was refluxed for 1 h. After cooling the mixture to room temperature, 4-(4,6-dichloro-5-(2-chloropropyl)pyrimidin-2-yl)morpholine (500 mg, 1.61 mmol) was added. The resultant mixture was refluxed for 16 h and then poured onto ice water (80 mL) and extracted with ethyl acetate (120 mL×2). The organic layer was washed with brine (50 mL) and dried over sodium sulfate. It was filtered, concentrated and the residue was subjected to silica gel column chromatography (petroleum ether/ethyl acetate=1/1 to 0/100) to obtain 4-(4-chloro-6-methyl-7-(pyridin-4-yl)-6,7-dihydro-5H-pyrrolo[2,3-d]pyrimidin-2-yl)morpholine (200 mg, 37%) as brown solid. LCMS (ESI) m/z: 332.2 [M+H]⁺.

### Step 5: Synthesis of 4-(4-(furan-3-yl)-6-methyl-7-(pyridin-4-yl)-6,7-dihydro-5H-pyrrolo[2,3-d]pyrimidin-2-yl)morpholine

To a stirred mixture of 4-(4-chloro-6-methyl-7-(pyridin-4-yl)-6,7-dihydro-5H-pyrrolo[2,3-d]pyrimidin-2-yl)morpholine (200 mg, 0.603 mmol), furan-3-ylboronic acid (135 mg, 1.207 mmol), [1,1′-bis(diphenylphosphino)ferrocene]dichloropalladium(II):CH₂Cl₂ (49 mg, 0.06 mmol) in acetonitrile (8 mL) and water (2 mL) was added cesium carbonate (393 mg, 1.206 mmol) at room temperature. The resultant reaction mixture was stirred at 80° C. for 4 h under nitrogen and cooled. The reaction was quenched with water (50 mL) and the mixture was extracted with dichloromethane (100 mL×2). The combined extractions were washed with brine (30 mL), dried over sodium sulfate, filtered and concentrated. The resultant residue was subjected to silica gel column chromatography, eluting with petroleum ether/ethyl acetate=1/1 to obtain 4-(4-(furan-3-yl)-6-methyl-7-(pyridin-4-yl)-6,7-dihydro-5H-pyrrolo[2,3-d]pyrimidin-2-yl)morpholine (200 mg, 91%) as yellow solid. LCMS (ESI) m/z: 364.0 [M+H]⁺.

### Step 6: Synthesis of 4-(6-methyl-7-(pyridin-4-yl)-4-(tetrahydrofuran-3-yl)-6,7-dihydro-5H-pyrrolo[2,3-d]pyrimidin-2-yl)morpholine

A suspension of 4-(4-(Furan-3-yl)-6-methyl-7-(pyridin-4-yl)-6,7-dihydro-5H-pyrrolo[2,3-d]pyrimidin-2-yl)morpholine (200 mg, 0.55 mmol) and palladium on activated charcoal (10%, 100 mg) in methanol (20 mL) and ethyl acetate (10 mL) was stirred at 30° C. for 16 h under hydrogen atmosphere. The mixture was filtered through celite and concentrated. The resultant residue was purified by prep-HPLC (SunFire C18, 4.6*50 mm, 3.5 um column Xbridge C18 3.5 μm 4.6×50 mm column. The elution system used was a gradient of 5%˜95% over 1.5 min at 2 ml/min and the solvent was acetonitrile/0.01% aqueous ammonium bicarbonate.) to obtain 4-(6-methyl-7-(pyridin-4-yl)-4-(tetrahydrofuran-3-yl)-6,7-dihydro-5H-pyrrolo[2,3-d]pyrimidin-2-yl)morpholine (17.1 mg, 8%) as white solid. ¹H NMR (500 MHz, MeOD) δ 8.42 (d, J=6.0 Hz, 2H), 7.83 (d, J=6.0 Hz, 2H), 4.80 (bs, 1H), 4.01-3.97 (m, 1H), 3.89-3.87 (m, 1H), 3.82-3.62 (m, 10H), 3.32-3.30 (m, 1H), 3.27-3.22 (m, 1H), 2.69-2.63 (m, 1H), 2.19-2.10 (m, 2H), 1.25-1.22 (m, 3H). LCMS (ESI) m/z: 368.1 [M+H]⁺.

### Synthesis of 4-(4-(1-methylpyrrolidin-3-yl)-7-phenyl-6,7-dihydro-5H-pyrrolo[2,3-d]pyrimidin-2-yl)morpholine (Compound 98)

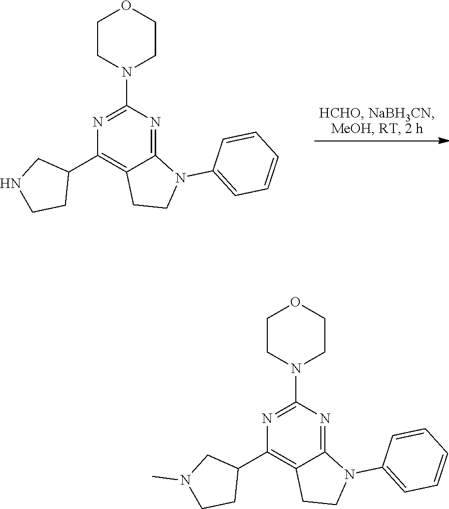 [SMILES: c1ccc(N2CCc3c(C4CCNC4)nc(N4CCOCC4)nc32)cc1 ; "HCHO, NaBH3CN,MeOH, RT, 2 h" ; CN1CCC(c2nc(N3CCOCC3)nc3c2CCN3c2ccccc2)C1]

A mixture of 4-(7-phenyl-4-(pyrrolidin-3-yl)-6,7-dihydro-5H-pyrrolo[2,3-d]pyrimidin-2-yl)morpholine (70 mg, 0.112 mmol), formaldehyde (13 mg, 0.398 mmol) and sodium cyanoborohydride (25 mg, 0.398 mmol) in methanol (4 mL) was stirred at 25° C. for 2 h. Then water (10 mL) was added and the mixture was extracted with ethyl acetate (30 mL×3). The organic layer was dried over sodium sulfate, filtered and concentrated. The crude product obtained was purified by silica gel column (dichloromethane:methanol from 50:1 to 10:1) to obtain 4-(4-(1-methylpyrrolidin-3-yl)-7-phenyl-6,7-dihydro-5H-pyrrolo[2,3-d]pyrimidin-2-yl)morpholine (22.4 mg, 28%). ¹H NMR (400 MHz, DMSO-d₆) δ 7.80 (d, J=8.0 Hz, 2H), 7.36 (t, J=7.6 Hz, 2H), 7.00 (t, J=7.2 Hz, 1H), 4.03 (t, J=8.4 Hz, 2H), 3.66 (s, 8H), 3.31-3.27 (m, 2H), 3.01-2.92 (m, 3H), 2.79-2.78 (m, 1H), 2.48-2.44 (m, 1H), 2.31 (s, 3H), 2.09-2.04 (m, 2H); LC-MS: m/z=366.3 (M+H)+.

### Example 2. PIKfyve Inhibitory Activity

PIKfyve Biochemical Assay. The biochemical PIKFyve inhibition assays were run by Carna Biosciences according to proprietary methodology based on the Promega ADP-Glo™ Kinase assay. A full-length human PIKFYVE [1-2098 (end) amino acids and S696N, L932S, Q995L, T998S, S1033A and Q1183K of the protein having the sequence set forth in NCBI Reference Sequence No. NP_055855.2] was expressed as N-terminal GST-fusion protein (265 kDa) using baculovirus expression system. GST-PIKFYVE was purified by using glutathione sepharose chromatography and used in an ADP-Glo™ Kinase assay (Promega). Reactions were set up by adding the test compound solution, substrate solution, ATP solution and kinase solution, each at 4×final concentrations. Reactions were prepared with assay buffer (50 mM MOPS, 1 mM DTT, pH7.2), mixed, and incubated in black 384 well polystyrene plates for 1 hour at room temperature. ADP-Glo™ reagent was then added for 40 minutes, followed by kinase detection reagent for an additional 40 minutes. The kinase activity was evaluated by detecting relative light units on a luminescence plate reader. Samples were run in duplicate from 10 μM to 3 nM. Data was analyzed by setting the control wells (+PIKfyve, no compound) to 0% inhibition and the readout value of background (no PIKfyve) set to 100% inhibition, then the % inhibition of each test solution calculated. IC50 values were calculated from concentration vs % inhibition curves by fitting to a four-parameter logistic curve.

NanoBRET™ TE Intracellular Kinase Assay, K-8 (Promega) Cell-Based Assay. Intracellular inhibition of PIKfyve was assayed using Promega's NanoBRET™ TE Intracellular Kinase Assay, K-8 according to manufacturer's instructions. A dilution series of test compounds was added for 2 hours to HEK293 cells transfected for a minimum of 20 hours with PIKFYVE-NanoLuc® Fusion Vector (Promega) containing a full-length PIKfyve according to manufacturer's specifications in a 96-well plate. Kinase activity was detected by addition of a NanoBRET™ tracer reagent, which was a proprietary PIKfyve inhibitor appended to a fluorescent probe (BRET, bioluminescence resonance energy transfer). Test compounds were tested at concentrations of 10, 3, 1, 0.3, 0.1, 0.03, 0.01, 0.003 μM. BRET signals were measured by a GloMax® Discover Multimode Microplate Reader (Promega) using 0.3 sec/well integration time, 450BP donor filter and 600LP acceptor filters. Active test compounds that bound PIKfyve and displaced the tracer reduced BRET signal. IC50 values were then calculated by fitting the data to the normalized BRET ratio.

The results of the PIKfyve inhibition assays are summarized in the table below.

| | | hPIKfyve |
|---|---|---|
| | hPIKfyve | BRET |
| Compound | IC₅₀ (μM)^{a} | IC₅₀ (μM)^{a} |
| 1 | +++ | +++ |
| 2 | +++ | |
| 3 | +++ | +++ |
| 4 | +++ | |
| 5 | +++ | ++ |
| 6 | ++ | |
| 7 | + | |
| 8 | +++ | |
| 9 | ++ | |
| 10 | ++++ | ++ |
| 11 | ++ | |
| 12 | ++ | |
| 13 | ++ | |
| 14 | ++ | |
| 15 | + | |
| 16 | +++ | ++ |
| 17 | +++ | |
| 18 | +++ | |
| 19 | ++ | |
| 20 | ++ | |
| 21 | +++ | ++ |
| 22 | ++++ | ++ |
| 23 | +++ | |
| 24 | +++ | + |
| 25 | ++ | ++ |
| 26 | ++ | |
| 27 | +++ | ++ |
| 29 | + | |
| 30 | ++ | |
| 31 | | |
| 32 | ++ | ++ |
| 34 | ++ | |
| 35 | ++ | |
| 37 | +++ | |
| 38 | ++ | |
| 39 | ++ | |
| 40 | +++ | |
| 41 | ++++ | +++ |
| 48 | + | |
| 49 | ++ | |
| 51 | +++ | |
| 52 | +++ | |
| 54 | +++ | |
| 60 | + | |
| 64 | +++ | |
| 73 | +++ | |
| 75 | +++ | |
| 76 | ++ | |
| 110 | ++ | |
| 111 | ++ | + |

^{a}++++ stands for < 10 nM, +++ stands for 10-100 nM, ++ stands for 100-1000 nM, + stands for 1-10 μM, and — stands for > 10 μM.

### Example 3. Viability Assay to Assess TDP-43 Toxicity in FAB1 TDP-43 and PIKfyve TDP-43 Yeast Cells

Generation of TDP-43 yeast model expressing human PIKfyve. Human PIKFYVE (“entry clone”) was cloned into pAG416GPDccdB (“destination vector”) according to standard Gateway cloning protocols (Invitrogen, Life Technologies). The resulting pAG416GPD-PIKFYVE plasmids were amplified in E. coli and plasmid identity confirmed by restriction digest and Sanger sequencing. Lithium acetate/polyethylene glycol-based transformation was used to introduce the above PIKFYVE plasmid into a BY4741 yeast strain auxotrophic for the ura3 gene and deleted for two transcription factors that regulate the xenobiotic efflux pumps, a major efflux pump, and FAB1, the yeast ortholog of PIKFYVE (MATa, snq2::KILeu2; pdr3::KIura3; pdr1::NATMX; fab1::G418^{R}, his3; leu2; ura3; met15; LYS2+) (FIG. 2).

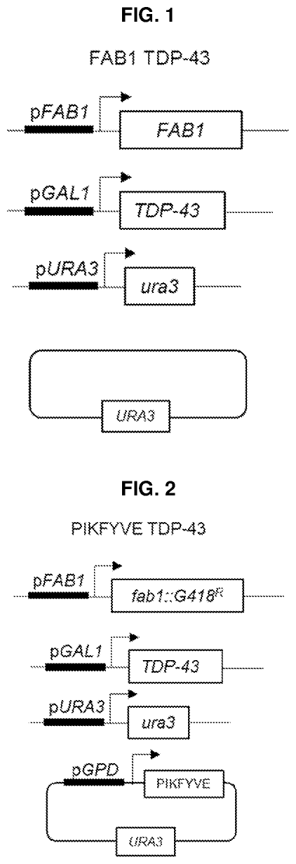

Transformed yeast were plated on solid agar plates with complete synthetic media lacking uracil (CSM-ura) and containing 2% glucose. Individual colonies harboring the control or PIKFYVE TDP-43 plasmids were recovered. A plasmid containing wild-type TDP-43 under the transcriptional control of the GAL1 promoter and containing the hygromycin-resistance gene as a selectable marker was transformed into the fab1::G418^{R} pAG416GPD-PIKFYVE yeast strain (FIG. 1). Transformed yeast were plated on CSM-ura containing 2% glucose and 200 μg/mL G418 after overnight recovery in media lacking antibiotic. Multiple independent isolates were further evaluated for cytotoxicity and TDP-43 expression levels.

Viability Assay. A control yeast strain with the wild-type yeast FAB1 gene and TDP-43 (“FAB1 TDP-43”, carries empty pAG416 plasmid), and the “PIKFYVE TDP-43” yeast strain, were assessed for toxicity using a propidium iodide viability assay. Both yeast strains were transferred from solid CSM-ura/2% glucose agar plates into 3 mL of liquid CSM-ura/2% glucose media for 6-8 hours at 30° C. with aeration. Yeast cultures were then diluted to an optical density at 600 nm wavelength (OD₆₀₀) of 0.005 in 3 mL of CSM-ura/2% raffinose and grown overnight at 30° C. with aeration to an OD₆₀₀ of 0.3-0.8. Log-phase overnight cultures were diluted to OD₆₀₀ of 0.005 in CSM-ura containing either 2% raffinose or galactose and 150 μL dispensed into each well of a flat bottom 96-well plates. Compounds formulated in 100% dimethyl sulfoxide (DMSO) were serially diluted in DMSO and 1.5 μL diluted compound transferred to the 96-well plates using a multichannel pipet. Wells containing DMSO alone were also evaluated as controls for compound effects. Tested concentrations ranged from 15 μM to 0.11 M. Cultures were immediately mixed to ensure compound distribution and covered plates incubated at 30° C. for 24 hours in a stationary, humified incubator.

Upon the completion of incubation, cultures were assayed for viability using propidium iodide (PI) to stain for dead/dying cells. A working solution of PI was made where, for each plate, 1 μL of 10 mM PI was added to 10 mL of CSM-ura (raffinose or galactose). The final PI solution (50 μL/well) was dispensed into each well of a new round bottom 96-well plate. The overnight 96-well assay plate was then mixed with a multichannel pipet and 50 μL transferred to the PI-containing plate. This plate was then incubated for 30 minutes at 30° C. in the dark. A benchtop flow cytometer (Miltenyi MACSquant) was then used to assess red fluorescence (82 channel), forward scatter, and side scatter (with following settings: gentle mix, high flow rate, fast measurement, 10,000 events). Intensity histograms were then gated for “PI-positive” or “PI-negative” using the raffinose and galactose cultures treated with DMSO as controls. The DMSO controls for raffinose or galactose-containing cultures were used to determine the window of increased cell death and this difference set to 100. All compounds were similarly gated and then compared to this maximal window to establish the percent reduction in PI-positive cells. IC50 values were then calculated for compounds that demonstrated a concentration-dependent enhancement of viability by fitting a logistic regression curve.

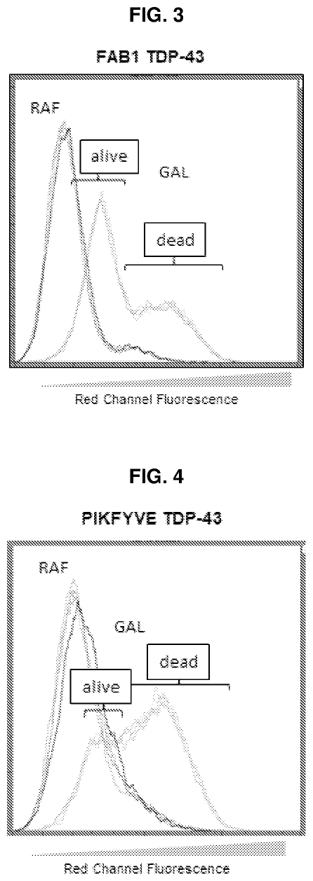

Upon induction of TDP-43 in both strains, there was a marked increase in inviable cells (rightmost population) with both FAB1 TDP-43 and PIKFYVE TDP-43, with a more pronounced effect in PIKFYVE TDP-43 (FIGS. 3 and 4).

PIKfyve Inhibition Suppresses Toxicity in PIKfyve TDP-43 Model. The biochemical PIKFyve inhibition assays were run by Carna Biosciences according to proprietary methodology based on the Promega ADP-Glo™ Kinase assay. A full-length human PIKFYVE [1-2098 (end) amino acids and S696N, L932S, Q995L,T998S, S1033A and Q1183K of accession number NP_055855.2] was expressed as N-terminal GST-fusion protein (265 kDa) using baculovirus expression system. GST-PIKFYVE was purified by using glutathione sepharose chromatography and used in an ADP-Glo™ Kinase assay (Promega).

Reactions were set up by adding the test compound solution, substrate solution, ATP solution and kinase solution, each at 4×final concentrations. Reactions were prepared with assay buffer (50 mM MOPS, 1 mM DTT, pH7.2), mixed, and incubated in black 384 well polystyrene plates for 1 hour at room temperature. ADP-Glo™ reagent was then added for 40 minutes, followed by kinase detection reagent for an additional 40 minutes. The kinase activity was evaluated by detecting relative light units on a luminescence plate reader. Samples were run in duplicate from 10 uM to 3 nM. Data was analyzed by setting the control wells (+ PIKfyve, no compound) to 0% inhibition and the readout value of background (no PIKfyve) set to 100% inhibition, then the % inhibition of each test solution calculated. IC50 values were calculated from concentration vs % inhibition curves by fitting to a four-parameter logistic curve.

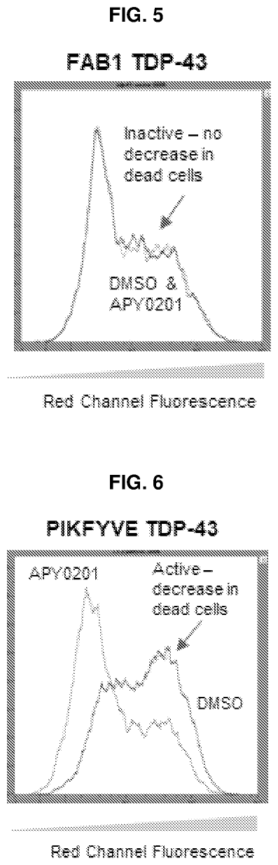

Activity of APY0201, a known PIKFYVE inhibitor, in FAB1 TDP-43 (FIG. 5) and PIKFYVE TDP-43 (FIG. 6). There was no increase in viable cells in FAB1 TDP-43 across a range of compound concentrations as evidenced by a lack in reduction of the right most population of propidium iodide-positive cells (only 0.23 μM is shown). In the PIKFYVE TDP-43 model, 0.23 μM reduced the population of propidium iodide-positive dead cells, indicating PIKFYVE inhibition ameliorated TDP-43 toxicity. Concentrations ranging from 0.5 mM to less than 100 nM afforded increased viability.

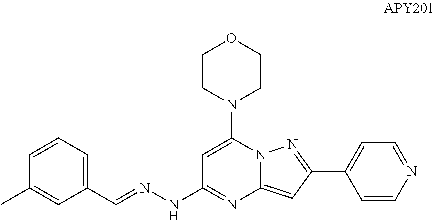 [SMILES: Cc1cccc(/C=N/Nc2cc(N3CCOCC3)n3nc(-c4ccncc4)cc3n2)c1]

A panel of compounds was tested in a biochemical PIKFYVE assay (ADP-Glo™ with full-length PIKfyve) and IC50's determined (nM) (see the Table below). The same compounds were also tested in both FAB1 and PIKFYVE TDP-43 yeast models. Their activity is reported here as “active” or “inactive.” Compounds with low nanomolar potency in the biochemical assay were active in the PIKFYVE TDP-43 yeast model. Compounds that were less potent or inactive in the biochemical assay were inactive in the PIKFYVE TDP-43 model. Compounds that were inactive in the biochemical or PIKFYVE TDP-43 assays were plotted with the highest concentrations tested in that assay.

| | | FAB1 TDP-43 | PIKfyve TDP-43 |
|---|---|---|---|
| Structure | PIKfyve IC₅₀ (nM) | (active/inactive) | (active/inactive) |
| 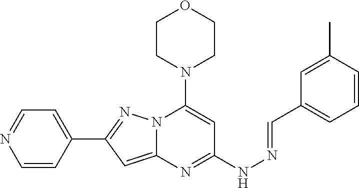 [SMILES: Cc1cccc(/C=N/Nc2cc(N3CCOCC3)n3nc(-c4ccncc4)cc3n2)c1] | 7.5 | Inactive | Active |
| 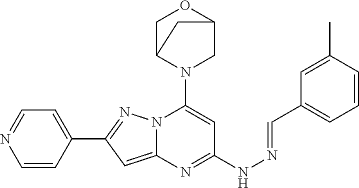 [SMILES: Cc1cccc(/C=N/Nc2cc(N3CC4CC3CO4)n3nc(-c4ccncc4)cc3n2)c1] | 12 | Inactive | Active |
| 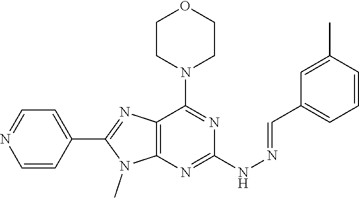 [SMILES: Cc1cccc(/C=N/Nc2nc(N3CCOCC3)c3nc(-c4ccncc4)n(C)c3n2)c1] | 4.9 | Inactive | Active |
| 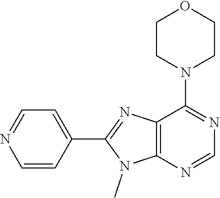 [SMILES: Cn1c(-c2ccncc2)nc2c(N3CCOCC3)ncnc21] | 640 | Inactive | Inactive |
| 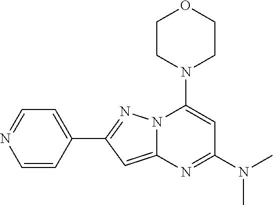 [SMILES: CN(C)c1cc(N2CCOCC2)n2nc(-c3ccncc3)cc2n1] | 2007 | Inactive | Inactive |
| 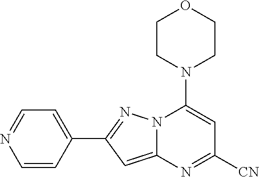 [SMILES: N#Cc1cc(N2CCOCC2)n2nc(-c3ccncc3)cc2n1] | >10000 | Inactive | Inactive |

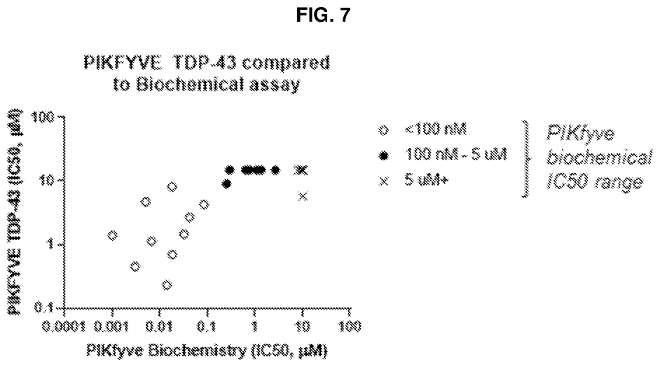

Biochemical and Efficacy Assays. A larger set of PIKfyve inhibitors were evaluated in both a PIKfyve kinase domain binding assay (nanobret) and in the PIKFYVE TDP-43 yeast strain. IC50 values (μM) were plotted. Data points are formatted based on binned potency from the nanobret assay as indicated in the legend (FIG. 7). Below is a table of compounds and their biochemical and PIKFYVE TDP-43 IC50 values plotted in FIG. 7.

| | PIKFYVE Biochemistry | PIKFYVE TDP-43 |
|---|---|---|
| Structure | (IC50, μM) | (IC50, μM) |
| 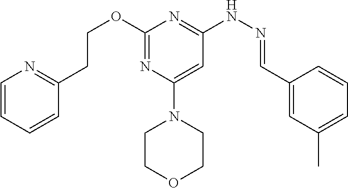 [SMILES: Cc1cccc(/C=N/Nc2cc(N3CCOCC3)nc(OCCc3ccccn3)n2)c1] | 0.003 | 0.450 |
| 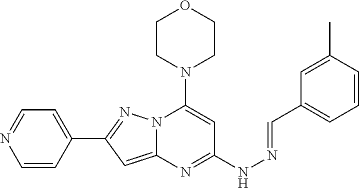 [SMILES: Cc1cccc(/C=N/Nc2cc(N3CCOCC3)n3nc(-c4ccncc4)cc3n2)c1] | 0.001 | 1.390 |
| 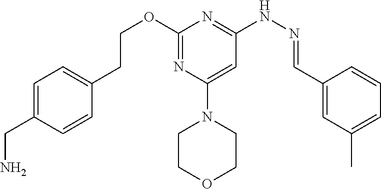 [SMILES: Cc1cccc(/C=N/Nc2cc(N3CCOCC3)nc(OCCc3ccc(CN)cc3)n2)c1] | 0.007 | 1.120 |
| 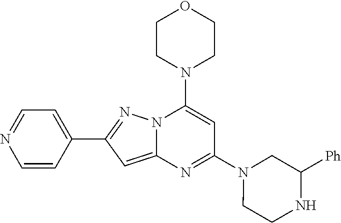 [SMILES: c1ccc(C2CN(c3cc(N4CCOCC4)n4nc(-c5ccncc5)cc4n3)CCN2)cc1] | 2.660 | >15 |
| 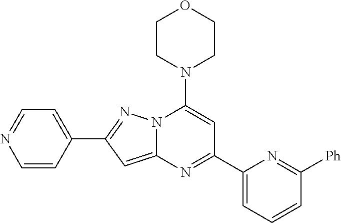 [SMILES: c1ccc(-c2cccc(-c3cc(N4CCOCC4)n4nc(-c5ccncc5)cc4n3)n2)cc1] | 0.014 | 0.230 |
| 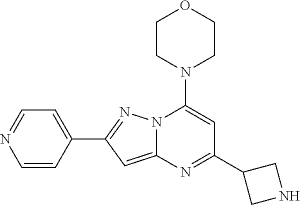 [SMILES: c1cc(-c2cc3nc(C4CNC4)cc(N4CCOCC4)n3n2)ccn1] | 8.020 | >15 |
| 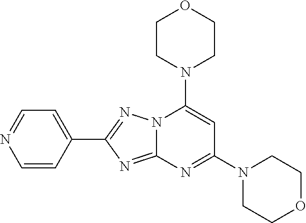 [SMILES: c1cc(-c2nc3nc(N4CCOCC4)cc(N4CCOCC4)n3n2)ccn1] | 9.200 | >15 |
| 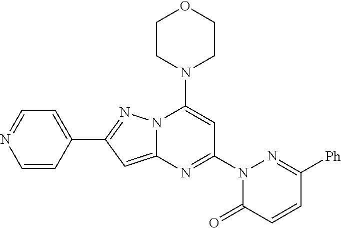 [SMILES: O=c1ccc(-c2ccccc2)nn1-c1cc(N2CCOCC2)n2nc(-c3ccncc3)cc2n1] | 0.295 | >15 |
| 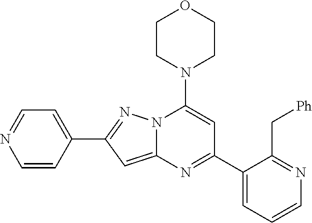 [SMILES: c1ccc(Cc2ncccc2-c2cc(N3CCOCC3)n3nc(-c4ccncc4)cc3n2)cc1] | 1.090 | >15 |
| 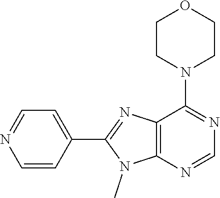 [SMILES: Cn1c(-c2ccncc2)nc2c(N3CCOCC3)ncnc21] | 0.640 | >15 |
| 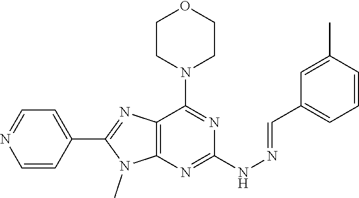 [SMILES: Cc1cccc(/C=N/Nc2nc(N3CCOCC3)c3nc(-c4ccncc4)n(C)c3n2)c1] | 0.005 | 4.720 |
| 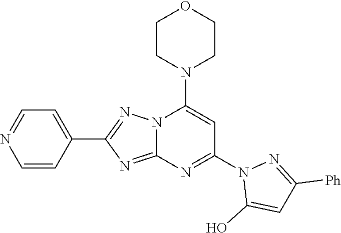 [SMILES: Oc1cc(-c2ccccc2)nn1-c1cc(N2CCOCC2)n2nc(-c3ccncc3)nc2n1] | 0.018 | 0.693 |
| 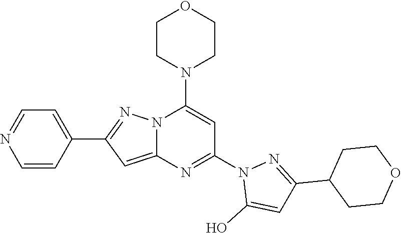 [SMILES: Oc1cc(C2CCOCC2)nn1-c1cc(N2CCOCC2)n2nc(-c3ccncc3)cc2n1] | 0.253 | 9.105 |
| 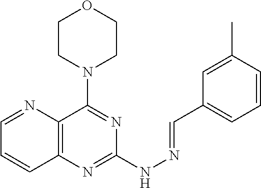 [SMILES: Cc1cccc(/C=N/Nc2nc(N3CCOCC3)c3ncccc3n2)c1] | 0.018 | 8.214 |
| 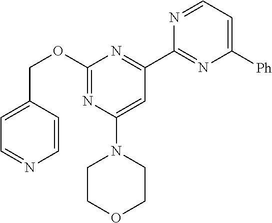 [SMILES: c1ccc(-c2ccnc(-c3cc(N4CCOCC4)nc(OCc4ccncc4)n3)n2)cc1] | 0.032 | 1.447 |
| 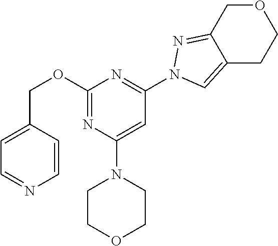 [SMILES: c1cc(COc2nc(N3CCOCC3)cc(-n3cc4c(n3)COCC4)n2)ccn1] | 1.343 | >15 |
| 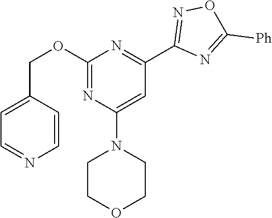 [SMILES: c1ccc(-c2nc(-c3cc(N4CCOCC4)nc(OCc4ccncc4)n3)no2)cc1] | >10 | >15 |
| 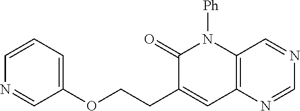 [SMILES: O=c1c(CCOc2cccnc2)cc2ncncc2n1-c1ccccc1] | >10 | >15 |
| 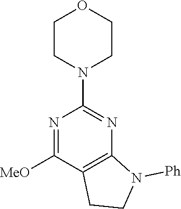 [SMILES: COc1nc(N2CCOCC2)nc2c1CCN2c1ccccc1] | 0.085 | 4.273 |
| 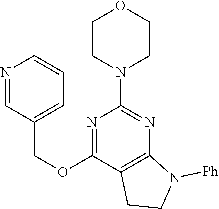 [SMILES: c1ccc(N2CCc3c(OCc4cccnc4)nc(N4CCOCC4)nc32)cc1] | 0.042 | 2.685 |
| 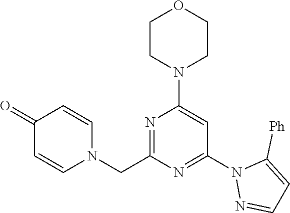 [SMILES: O=c1ccn(Cc2nc(N3CCOCC3)cc(-n3nccc3-c3ccccc3)n2)cc1] | >10 | >15 |
| 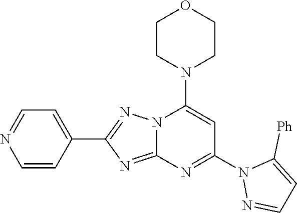 [SMILES: c1ccc(-c2ccnn2-c2cc(N3CCOCC3)n3nc(-c4ccncc4)nc3n2)cc1] | 0.767 | >15 |
| 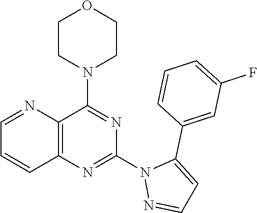 [SMILES: Fc1cccc(-c2ccnn2-c2nc(N3CCOCC3)c3ncccc3n2)c1] | >10 | 5.754 |

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A compound of formula (I): 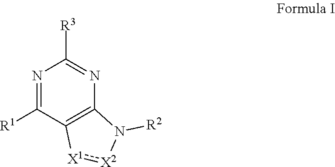
or a pharmaceutically acceptable salt thereof,
wherein [symbol]
is a single bond, X¹ is (C(R^{A})₂)ₘ or —OC(R^{A})₂—R^{X}, and X² is C(R^{A})₂ or CO; [symbol] is a double bond, and each of X¹ and X² is independently CR^{A} or N, wherein R^{X} is a bond to X²;
R¹ is -(L)ₙ-R¹; optionally substituted C₁₋₉ alkoxy; optionally substituted C₁₋₉ heterocyclyl comprising at least one endocyclic oxygen; unsubstituted pyrimidinyl; optionally substituted pyridazinyl; optionally substituted oxazolyl, or pyrid-2-on-1-yl;
R² is optionally substituted C₆₋₁₀ aryl, optionally substituted C₁₋₉ heterocyclyl, or optionally substituted C₁₋₉ heteroaryl;
R³ is a group of the following structure: 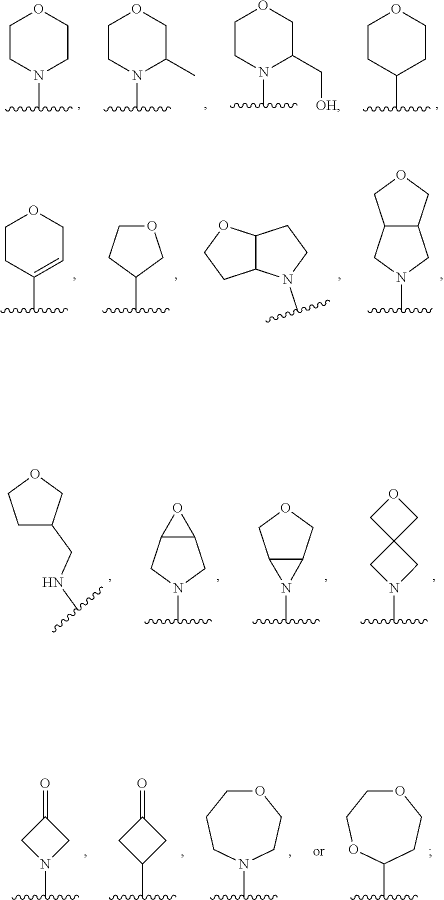
each R^{A} is independently H, optionally substituted C₁₋₆ alkyl, or optionally substituted C₆₋₁₀ aryl;
R^{B} is optionally substituted C₆₋₁₀ aryl, optionally substituted C₁₋₉ heteroaryl, optionally substituted C₃₋₈ cycloalkyl, or optionally substituted C₁₋₉ heterocyclyl;
R^{C} is H or optionally substituted C₁₋₆ alkyl;
each L is independently optionally substituted C₁₋₆ alkylene, O, or NR^{C};
n is 1, 2, or 3; and
m is 0, 1, or 2.

2. The compound of claim 1, wherein [symbol] is a single bond.

3. The compound of claim 1 or 2, wherein X¹ is (C(R^{A})₂)ₘ.

4. The compound of claim 3, wherein m is 1.

5. The compound of any one of claims 1 to 4, wherein X² is C(R^{A})₂.

6. The compound of any one of claims 1 to 5, wherein each R^{A} is hydrogen.

7. The compound of claim 1, wherein the compound is of formula (Ia): 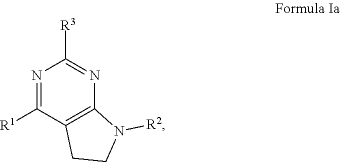
or a pharmaceutically acceptable salt thereof.

8. The compound of claim 1, wherein the compound is of formula (Ia′): 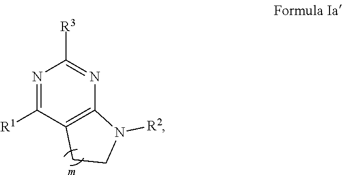
or a pharmaceutically acceptable salt thereof.

9. The compound of claim 1, wherein the compound is of formula (Ib): 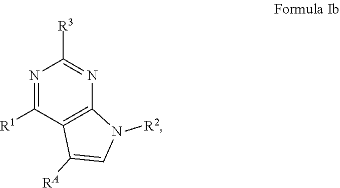
or a pharmaceutically acceptable salt thereof.

10. The compound of claim 1, wherein the compound is of formula (Ic): 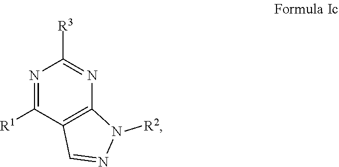
or a pharmaceutically acceptable salt thereof.

11. The compound of claim 1, wherein the compound is of formula (Id): 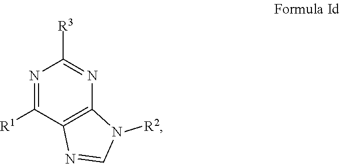
or a pharmaceutically acceptable salt thereof.

12. The compound of claim 1, wherein the compound is of formula (Ie): 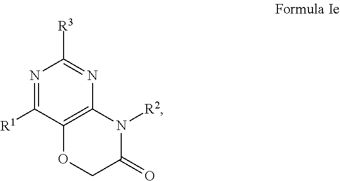
or a pharmaceutically acceptable salt thereof.

13. The compound of any one of claims 1 to 12, wherein R¹ is —O-(L)₍ₙ₋₁₎-R^{B}.

14. The compound of any one of claims 1 to 13, wherein n is 2.

15. The compound of any one of claims 1 to 13, wherein n is 1.

16. The compound of any one of claims 1 to 15, wherein at least one L is optionally substituted C₁₋₆ alkylene.

17. The compound of claim 16, wherein the optionally substituted C₁₋₆ alkylene is methylene.

18. The compound of claim 16, wherein the optionally substituted C₁₋₆ alkylene is ethylene.

19. The compound of any one of claims 1 to 18, wherein R^{B} is optionally substituted non-aromatic C₁₋₉ heterocyclyl.

20. The compound of any one of claims 1 to 18, wherein R^{B} is optionally substituted C₁₋₉ heteroaryl.

21. The compound of any one of claims 1 to 18, wherein R^{B} is optionally substituted C₁₋₆ alkyl.

22. The compound of any one of claims 1 to 12, wherein R¹ is: 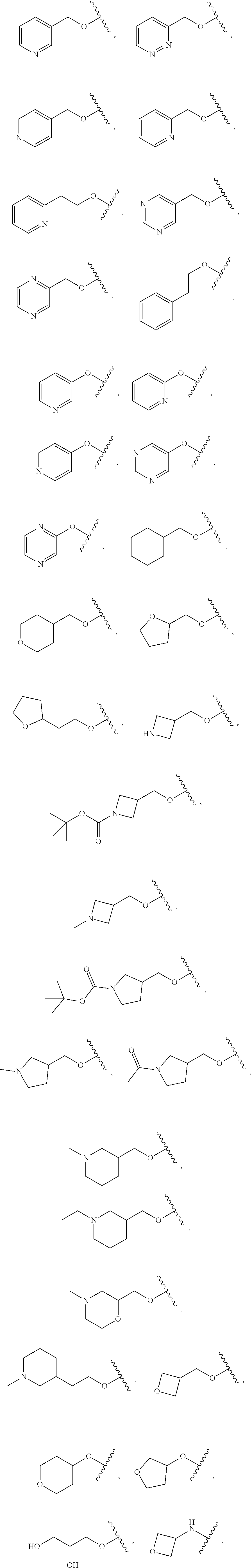 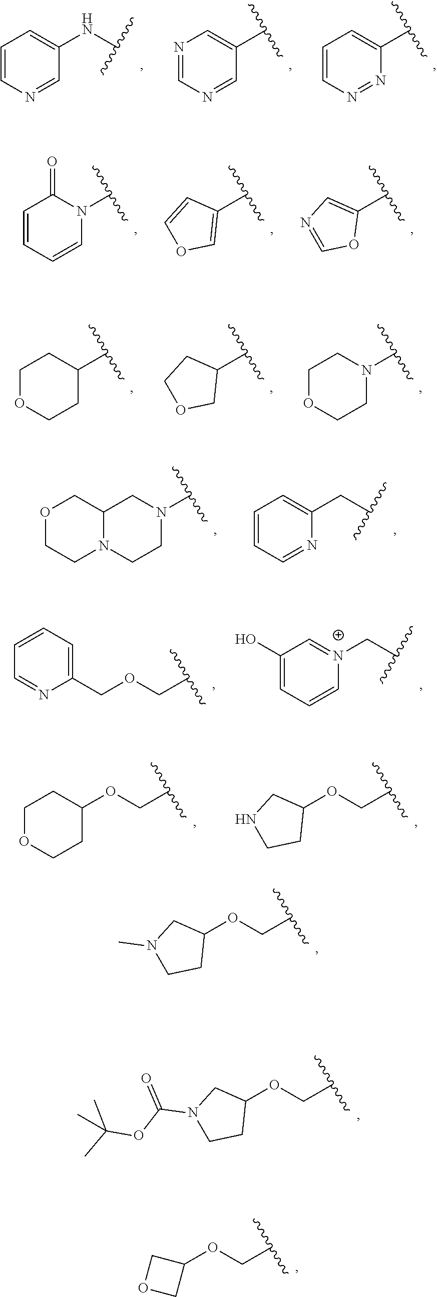
or methoxy.

23. The compound of claim 22, wherein R¹ is:

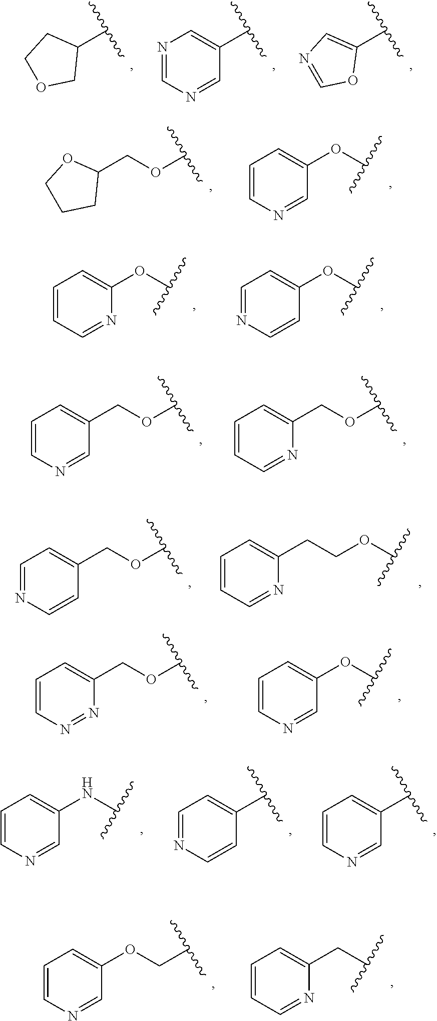

24. The compound of any one of claims 1 to 23, wherein R² is:

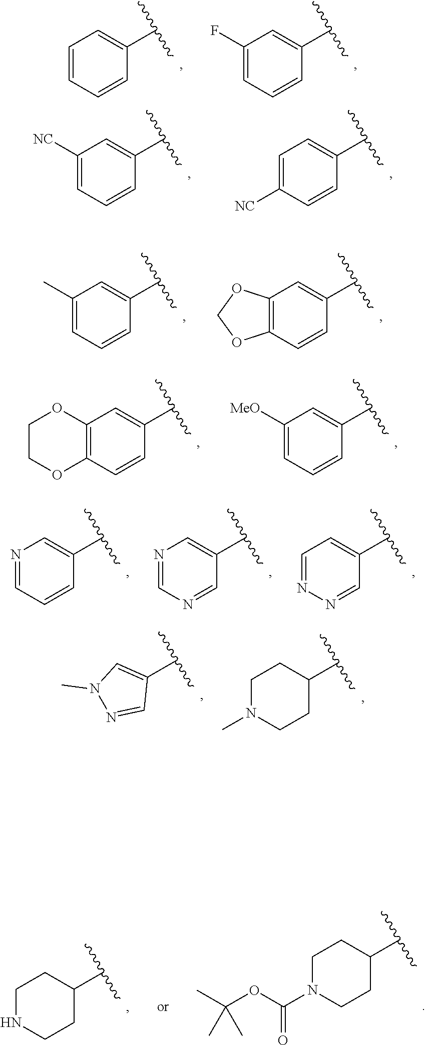

25. The compound of claim 24, wherein R² is:

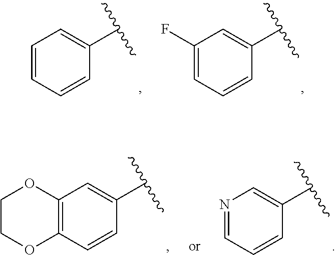

26. The compound of any one of claims 1 to 25, wherein R³ is:

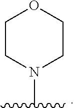 [SMILES: *N1CCOCC1]

27. A compound of the following structure: 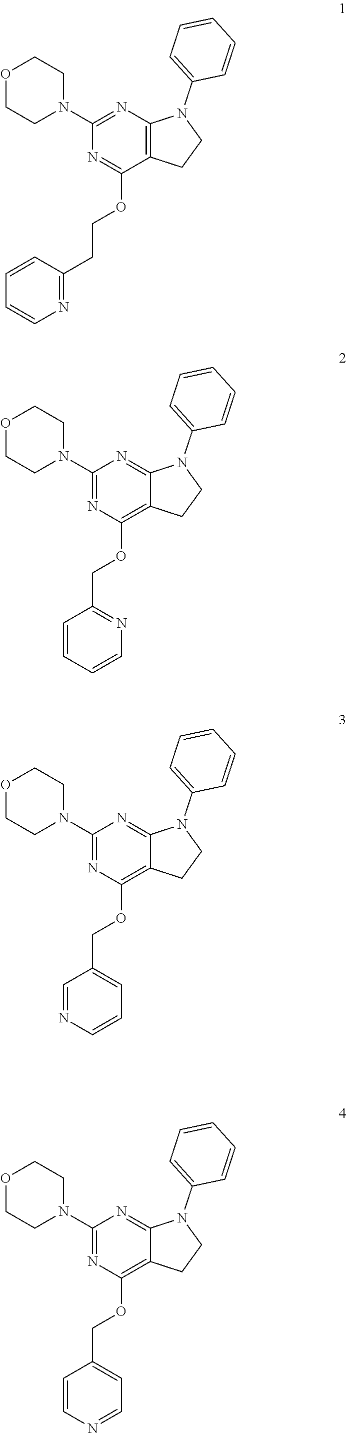 [SMILES: c1ccc(N2CCc3c(OCCc4ccccn4)nc(N4CCOCC4)nc32)cc1 ; c1ccc(N2CCc3c(OCc4ccccn4)nc(N4CCOCC4)nc32)cc1 ; c1ccc(N2CCc3c(OCc4cccnc4)nc(N4CCOCC4)nc32)cc1 ; c1ccc(N2CCc3c(OCc4ccncc4)nc(N4CCOCC4)nc32)cc1] 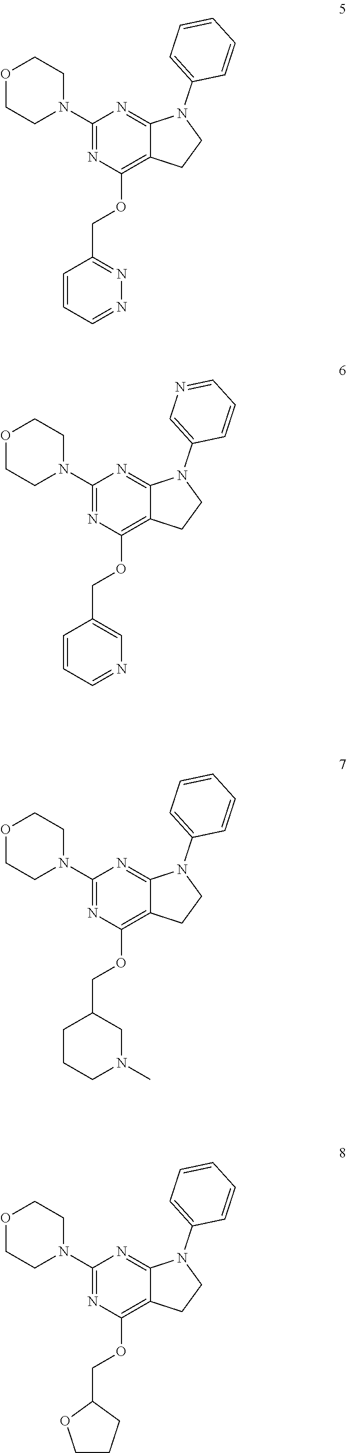 [SMILES: c1ccc(N2CCc3c(OCc4cccnn4)nc(N4CCOCC4)nc32)cc1 ; c1cncc(COc2nc(N3CCOCC3)nc3c2CCN3c2cccnc2)c1 ; CN1CCCC(COc2nc(N3CCOCC3)nc3c2CCN3c2ccccc2)C1 ; c1ccc(N2CCc3c(OCC4CCCO4)nc(N4CCOCC4)nc32)cc1] 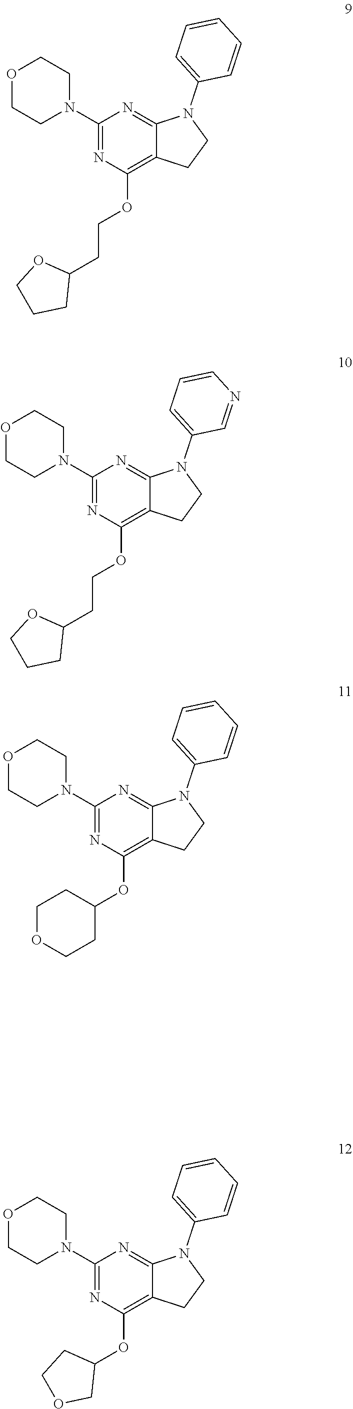 [SMILES: c1ccc(N2CCc3c(OCCC4CCCO4)nc(N4CCOCC4)nc32)cc1 ; c1cncc(N2CCc3c(OCCC4CCCO4)nc(N4CCOCC4)nc32)c1 ; c1ccc(N2CCc3c(OC4CCOCC4)nc(N4CCOCC4)nc32)cc1 ; c1ccc(N2CCc3c(OC4CCOC4)nc(N4CCOCC4)nc32)cc1] 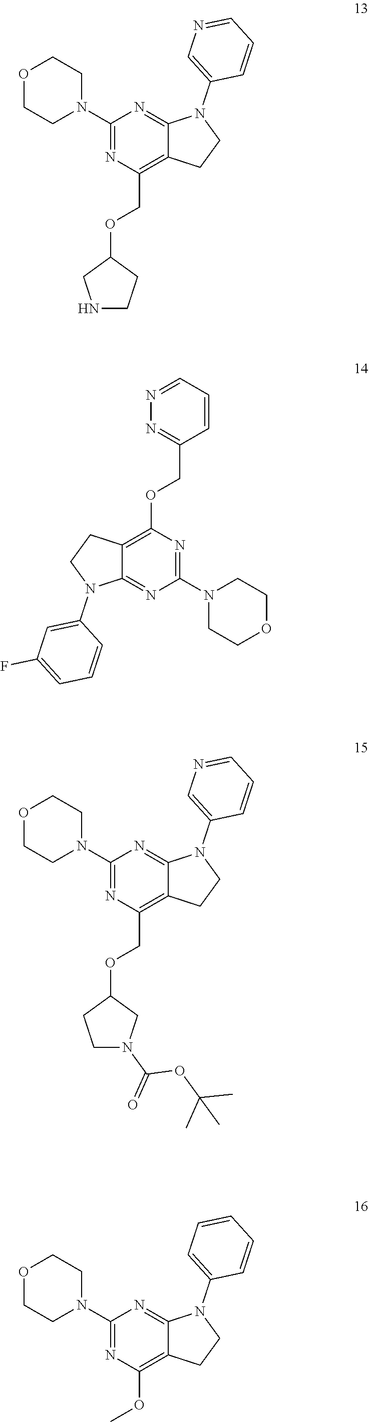 [SMILES: c1cncc(N2CCc3c(COC4CCNC4)nc(N4CCOCC4)nc32)c1 ; Fc1cccc(N2CCc3c(OCc4cccnn4)nc(N4CCOCC4)nc32)c1 ; CC(C)(C)OC(=O)N1CCC(OCc2nc(N3CCOCC3)nc3c2CCN3c2cccnc2)C1 ; COc1nc(N2CCOCC2)nc2c1CCN2c1ccccc1] 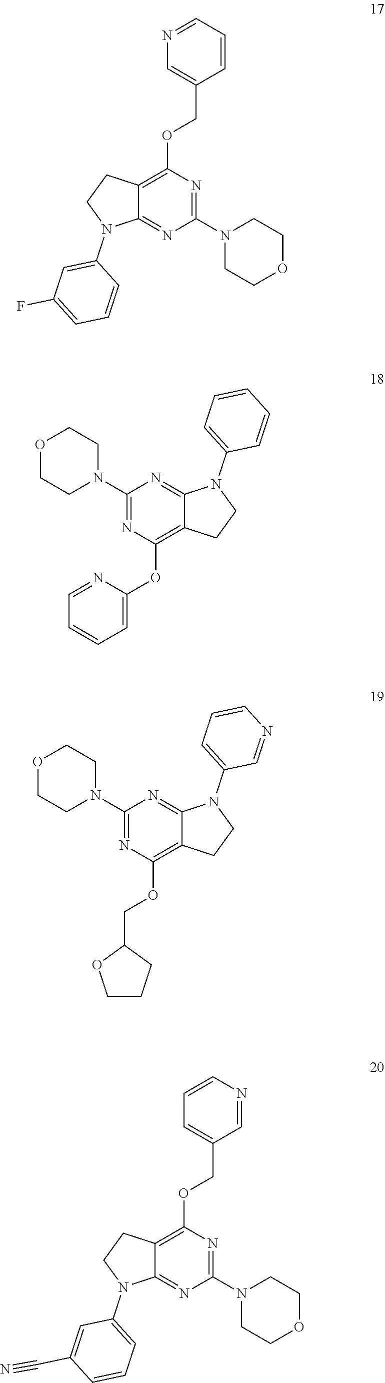 [SMILES: Fc1cccc(N2CCc3c(OCc4cccnc4)nc(N4CCOCC4)nc32)c1 ; c1ccc(N2CCc3c(Oc4ccccn4)nc(N4CCOCC4)nc32)cc1 ; c1cncc(N2CCc3c(OCC4CCCO4)nc(N4CCOCC4)nc32)c1 ; N#Cc1cccc(N2CCc3c(OCc4cccnc4)nc(N4CCOCC4)nc32)c1] 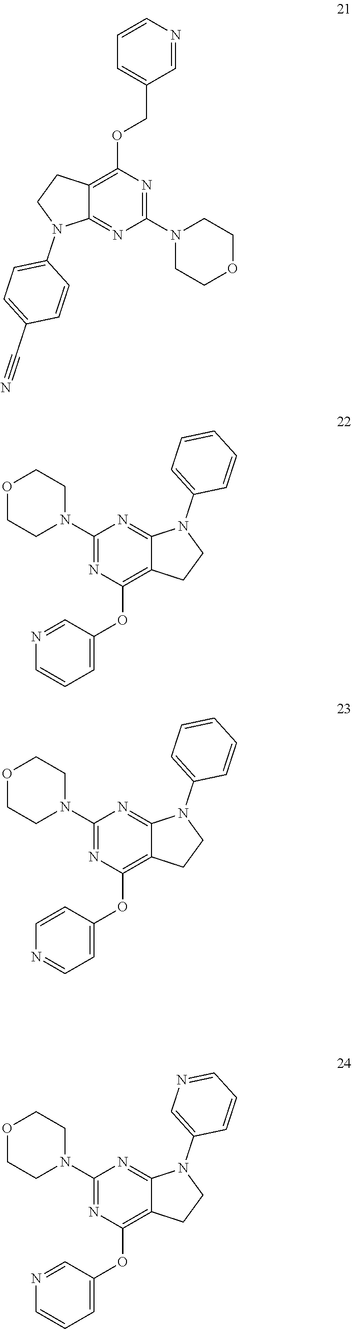 [SMILES: N#Cc1ccc(N2CCc3c(OCc4cccnc4)nc(N4CCOCC4)nc32)cc1 ; c1ccc(N2CCc3c(Oc4cccnc4)nc(N4CCOCC4)nc32)cc1 ; c1ccc(N2CCc3c(Oc4ccncc4)nc(N4CCOCC4)nc32)cc1 ; c1cncc(Oc2nc(N3CCOCC3)nc3c2CCN3c2cccnc2)c1] 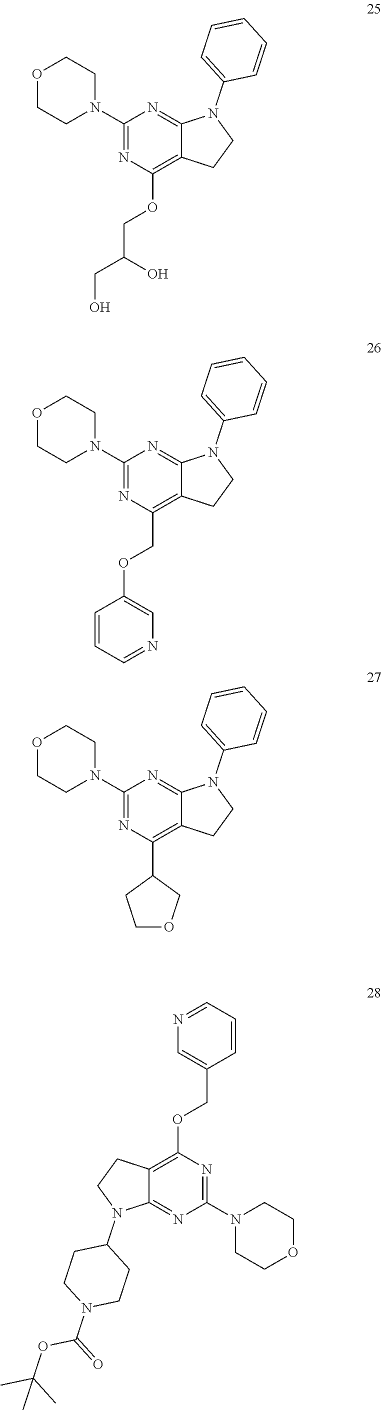 [SMILES: OCC(O)COc1nc(N2CCOCC2)nc2c1CCN2c1ccccc1 ; c1ccc(N2CCc3c(COc4cccnc4)nc(N4CCOCC4)nc32)cc1 ; c1ccc(N2CCc3c(C4CCOC4)nc(N4CCOCC4)nc32)cc1 ; CC(C)(C)OC(=O)N1CCC(N2CCc3c(OCc4cccnc4)nc(N4CCOCC4)nc32)CC1] 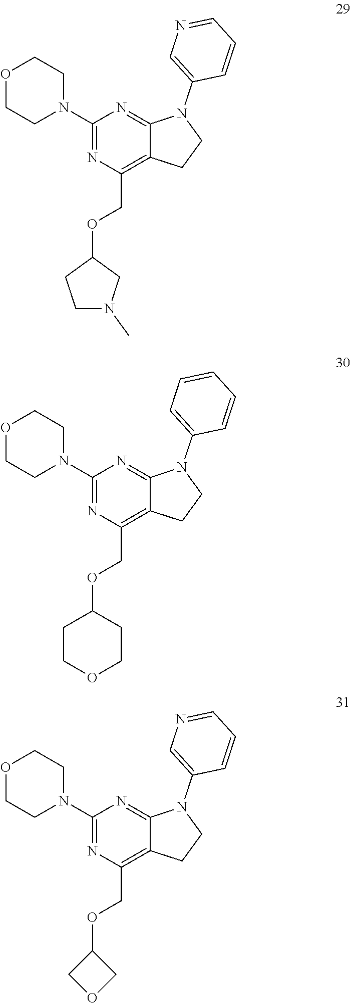 [SMILES: CN1CCC(OCc2nc(N3CCOCC3)nc3c2CCN3c2cccnc2)C1 ; c1ccc(N2CCc3c(COC4CCOCC4)nc(N4CCOCC4)nc32)cc1 ; c1cncc(N2CCc3c(COC4COC4)nc(N4CCOCC4)nc32)c1]
or a pharmaceutically acceptable salt thereof.

28. A compound of the following structure: 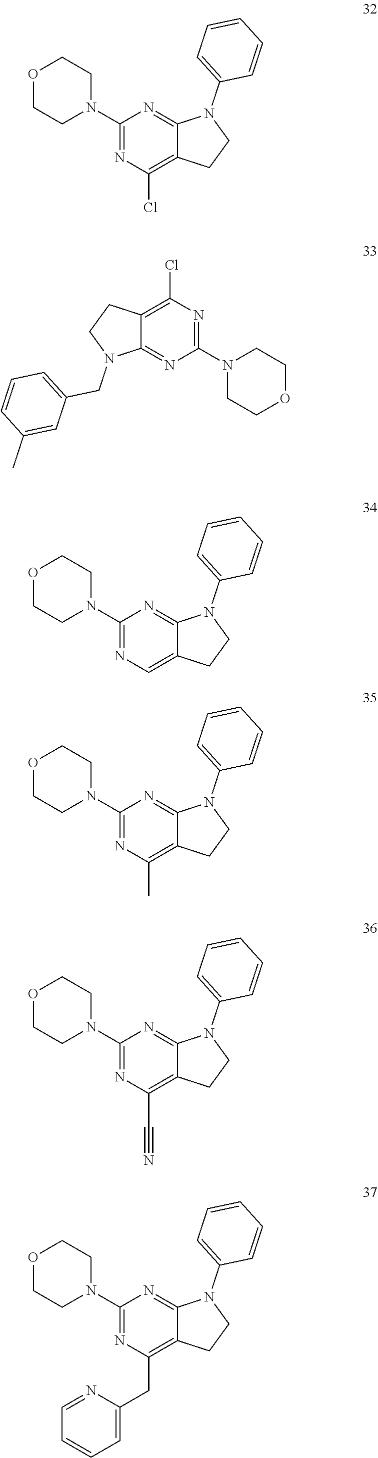 [SMILES: Clc1nc(N2CCOCC2)nc2c1CCN2c1ccccc1 ; Cc1cccc(CN2CCc3c(Cl)nc(N4CCOCC4)nc32)c1 ; c1ccc(N2CCc3cnc(N4CCOCC4)nc32)cc1 ; Cc1nc(N2CCOCC2)nc2c1CCN2c1ccccc1 ; N#Cc1nc(N2CCOCC2)nc2c1CCN2c1ccccc1 ; c1ccc(N2CCc3c(Cc4ccccn4)nc(N4CCOCC4)nc32)cc1] 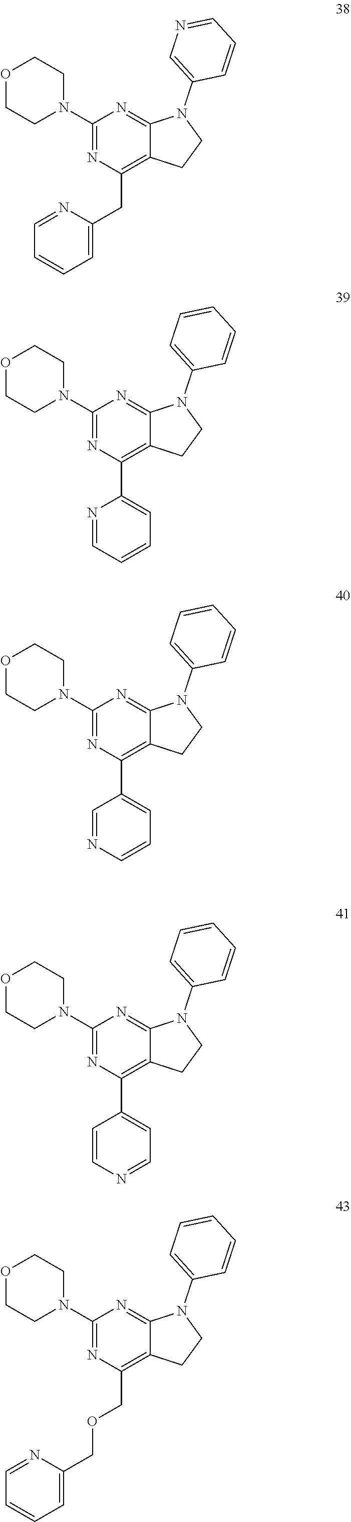 [SMILES: c1ccc(Cc2nc(N3CCOCC3)nc3c2CCN3c2cccnc2)nc1 ; c1ccc(N2CCc3c(-c4ccccn4)nc(N4CCOCC4)nc32)cc1 ; c1ccc(N2CCc3c(-c4cccnc4)nc(N4CCOCC4)nc32)cc1 ; c1ccc(N2CCc3c(-c4ccncc4)nc(N4CCOCC4)nc32)cc1 ; c1ccc(N2CCc3c(COCc4ccccn4)nc(N4CCOCC4)nc32)cc1] 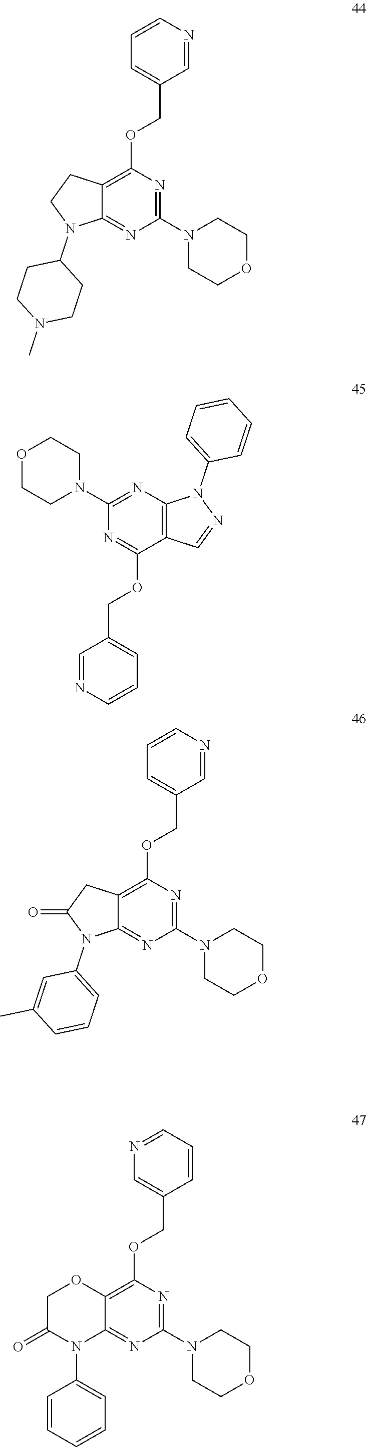 [SMILES: CN1CCC(N2CCc3c(OCc4cccnc4)nc(N4CCOCC4)nc32)CC1 ; c1ccc(-n2ncc3c(OCc4cccnc4)nc(N4CCOCC4)nc32)cc1 ; Cc1cccc(N2C(=O)Cc3c(OCc4cccnc4)nc(N4CCOCC4)nc32)c1 ; O=C1COc2c(OCc3cccnc3)nc(N3CCOCC3)nc2N1c1ccccc1] 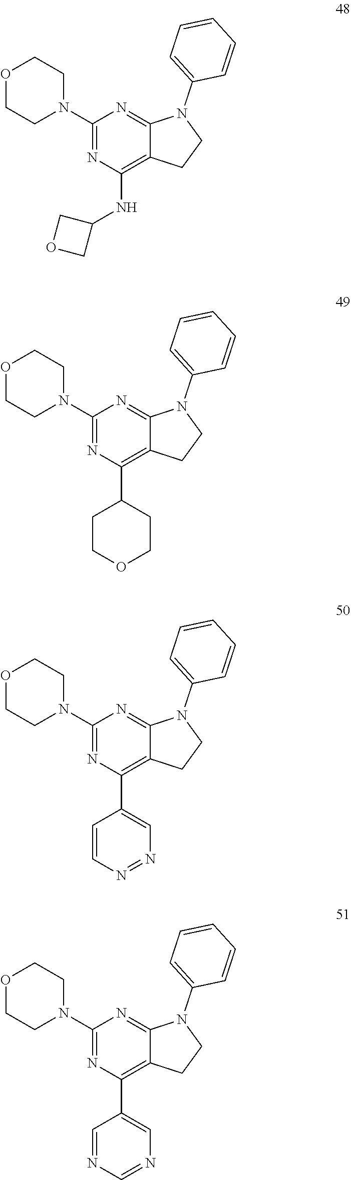 [SMILES: c1ccc(N2CCc3c(NC4COC4)nc(N4CCOCC4)nc32)cc1 ; c1ccc(N2CCc3c(C4CCOCC4)nc(N4CCOCC4)nc32)cc1 ; c1ccc(N2CCc3c(-c4ccnnc4)nc(N4CCOCC4)nc32)cc1 ; c1ccc(N2CCc3c(-c4cncnc4)nc(N4CCOCC4)nc32)cc1] 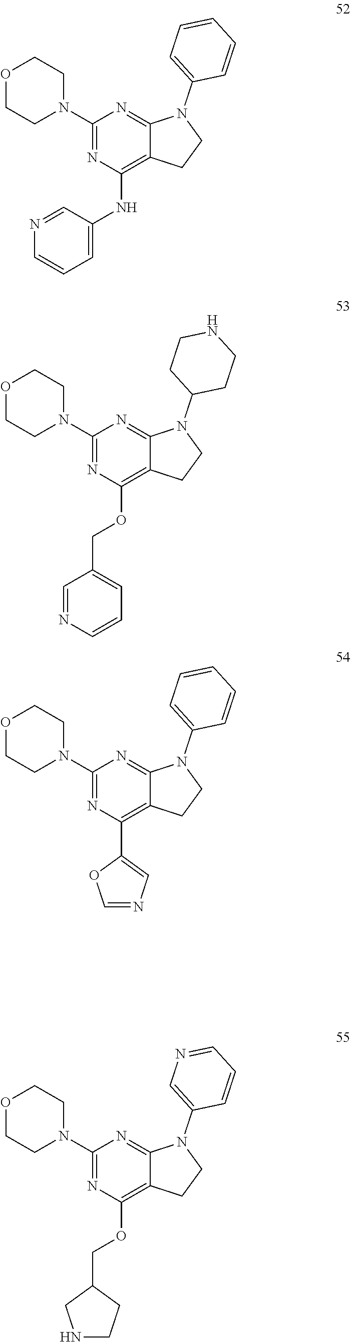 [SMILES: c1ccc(N2CCc3c(Nc4cccnc4)nc(N4CCOCC4)nc32)cc1 ; c1cncc(COc2nc(N3CCOCC3)nc3c2CCN3C2CCNCC2)c1 ; c1ccc(N2CCc3c(-c4cnco4)nc(N4CCOCC4)nc32)cc1 ; c1cncc(N2CCc3c(OCC4CCNC4)nc(N4CCOCC4)nc32)c1] 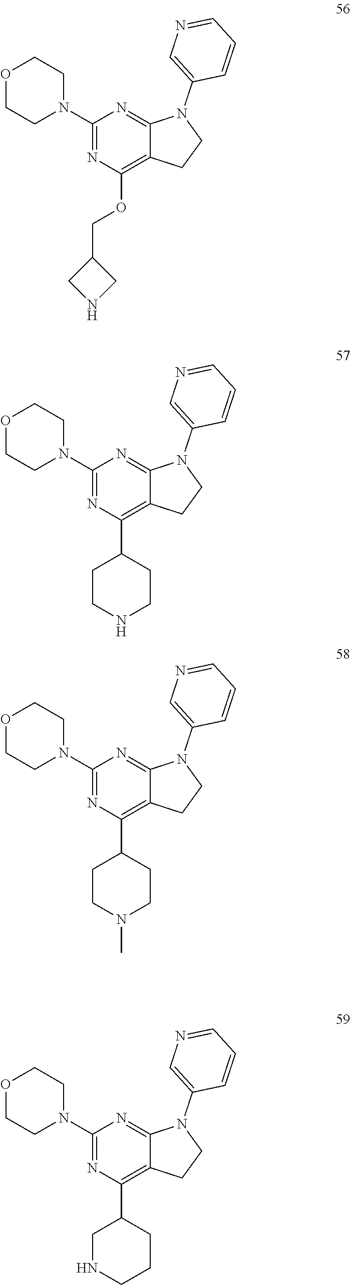 [SMILES: c1cncc(N2CCc3c(OCC4CNC4)nc(N4CCOCC4)nc32)c1 ; c1cncc(N2CCc3c(C4CCNCC4)nc(N4CCOCC4)nc32)c1 ; CN1CCC(c2nc(N3CCOCC3)nc3c2CCN3c2cccnc2)CC1 ; c1cncc(N2CCc3c(C4CCCNC4)nc(N4CCOCC4)nc32)c1] 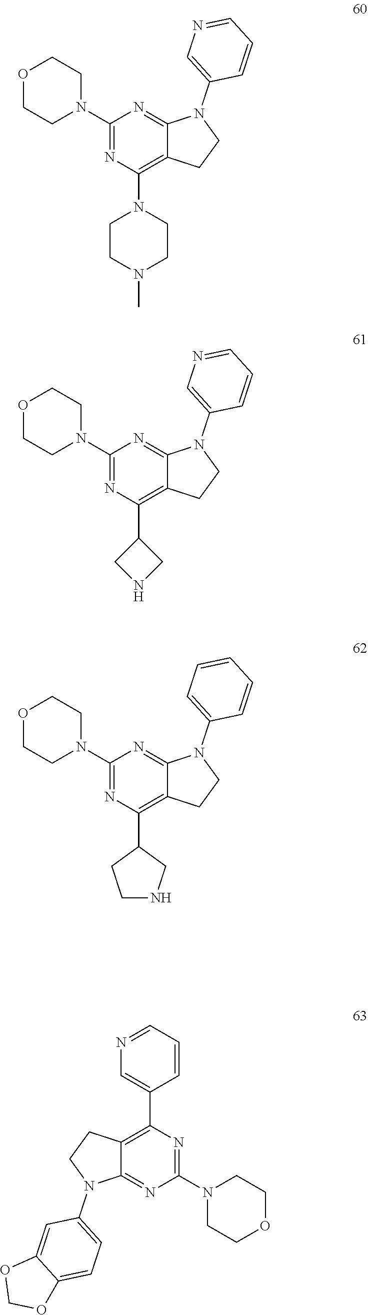 [SMILES: CN1CCN(c2nc(N3CCOCC3)nc3c2CCN3c2cccnc2)CC1 ; c1cncc(N2CCc3c(C4CNC4)nc(N4CCOCC4)nc32)c1 ; c1ccc(N2CCc3c(C4CCNC4)nc(N4CCOCC4)nc32)cc1 ; c1cncc(-c2nc(N3CCOCC3)nc3c2CCN3c2ccc3c(c2)OCO3)c1] 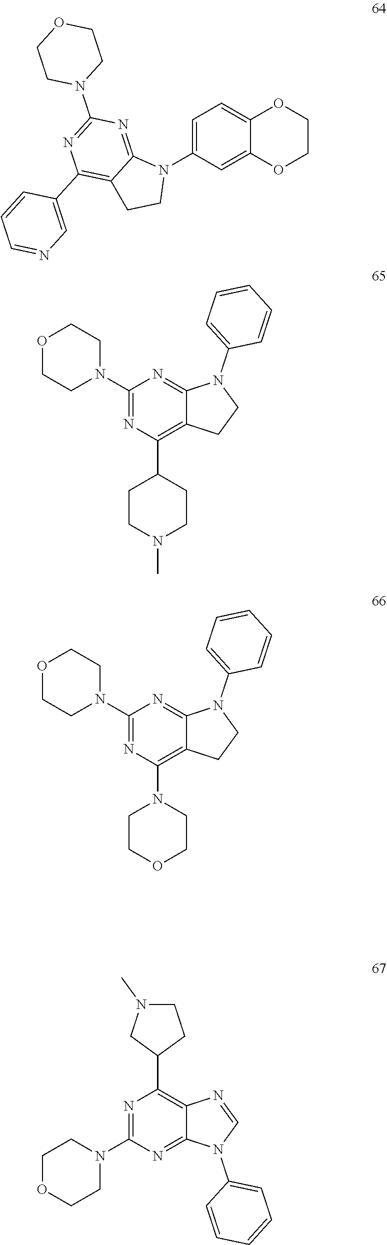 [SMILES: c1cncc(-c2nc(N3CCOCC3)nc3c2CCN3c2ccc3c(c2)OCCO3)c1 ; CN1CCC(c2nc(N3CCOCC3)nc3c2CCN3c2ccccc2)CC1 ; c1ccc(N2CCc3c(N4CCOCC4)nc(N4CCOCC4)nc32)cc1 ; CN1CCC(c2nc(N3CCOCC3)nc3c2ncn3-c2ccccc2)C1] 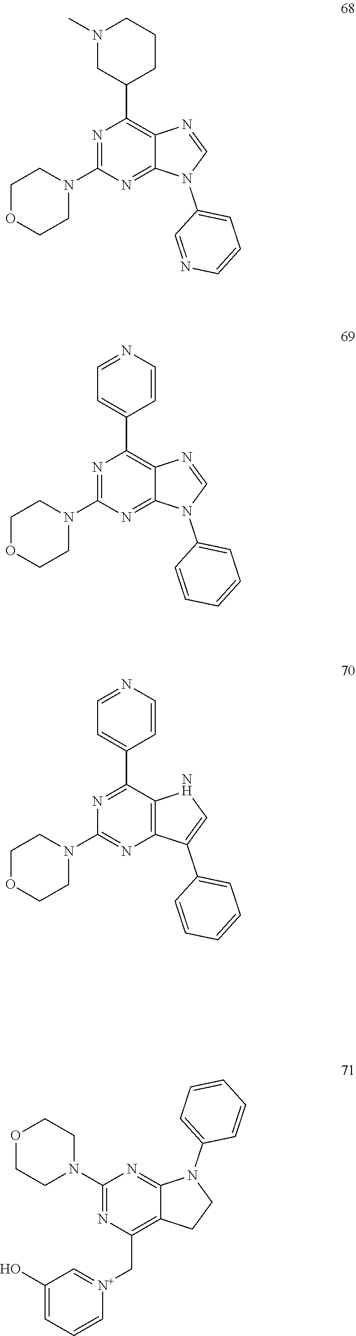 [SMILES: CN1CCCC(c2nc(N3CCOCC3)nc3c2ncn3-c2cccnc2)C1 ; c1ccc(-n2cnc3c(-c4ccncc4)nc(N4CCOCC4)nc32)cc1 ; c1ccc(-c2c[nH]c3c(-c4ccncc4)nc(N4CCOCC4)nc23)cc1 ; Oc1ccc[n+](Cc2nc(N3CCOCC3)nc3c2CCN3c2ccccc2)c1] 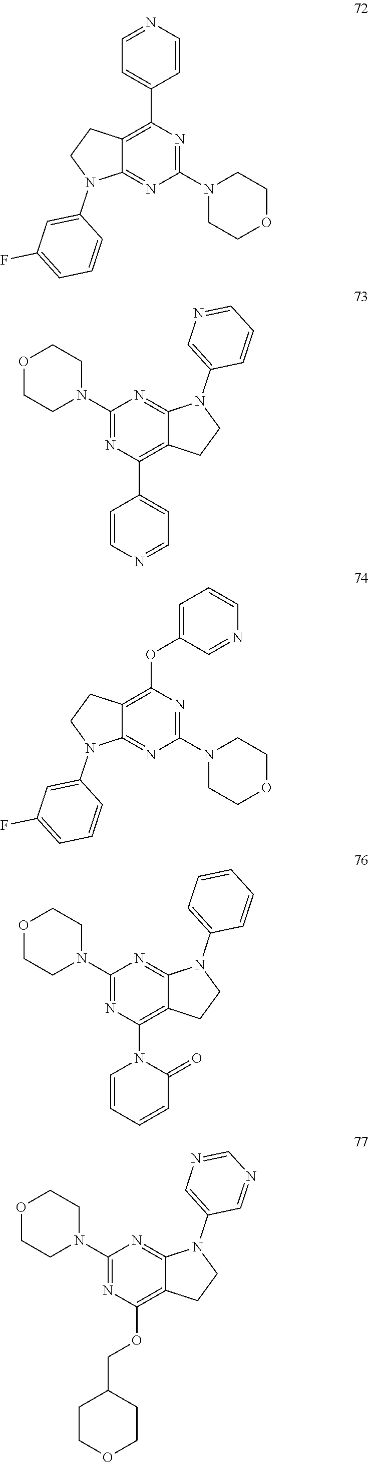 [SMILES: Fc1cccc(N2CCc3c(-c4ccncc4)nc(N4CCOCC4)nc32)c1 ; c1cncc(N2CCc3c(-c4ccncc4)nc(N4CCOCC4)nc32)c1 ; Fc1cccc(N2CCc3c(Oc4cccnc4)nc(N4CCOCC4)nc32)c1 ; O=c1ccccn1-c1nc(N2CCOCC2)nc2c1CCN2c1ccccc1 ; c1ncc(N2CCc3c(OCC4CCOCC4)nc(N4CCOCC4)nc32)cn1] 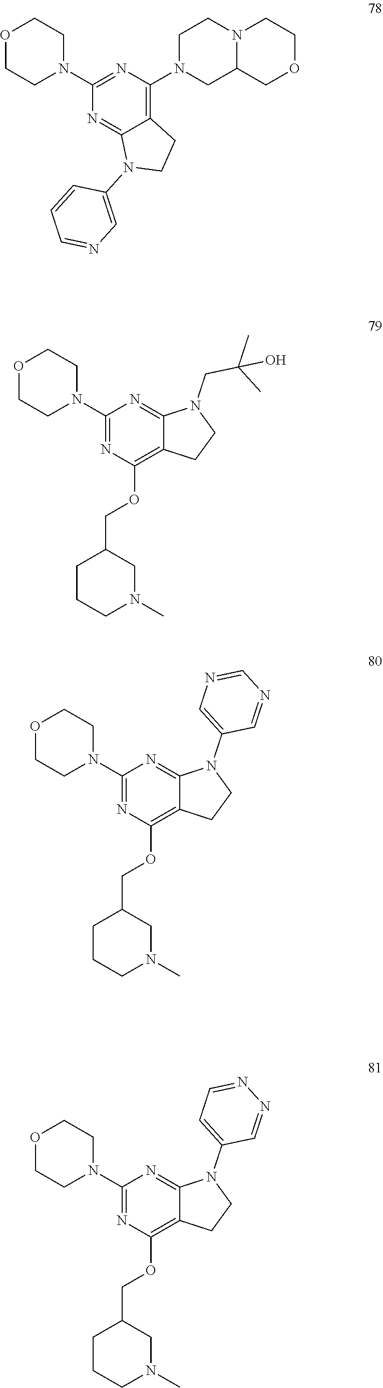 [SMILES: c1cncc(N2CCc3c(N4CCN5CCOCC5C4)nc(N4CCOCC4)nc32)c1 ; CN1CCCC(COc2nc(N3CCOCC3)nc3c2CCN3CC(C)(C)O)C1 ; CN1CCCC(COc2nc(N3CCOCC3)nc3c2CCN3c2cncnc2)C1 ; CN1CCCC(COc2nc(N3CCOCC3)nc3c2CCN3c2ccnnc2)C1] 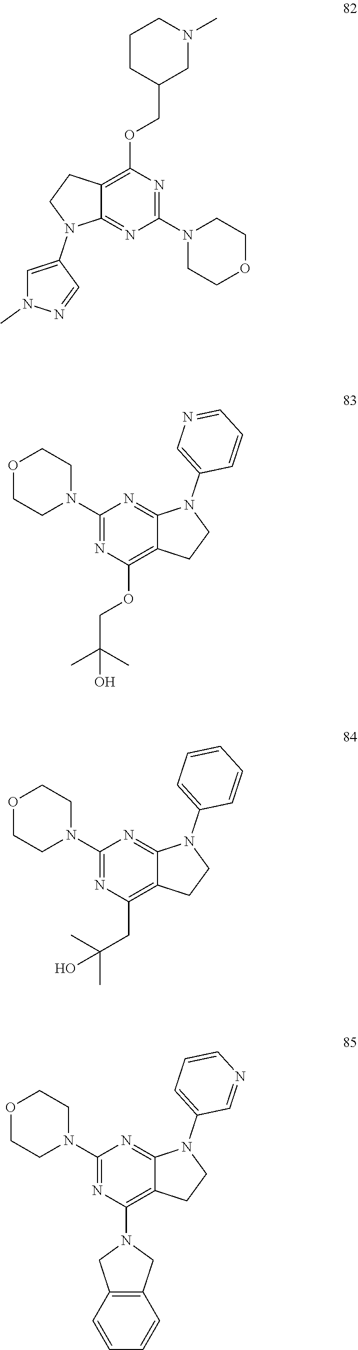 [SMILES: CN1CCCC(COc2nc(N3CCOCC3)nc3c2CCN3c2cnn(C)c2)C1 ; CC(C)(O)COc1nc(N2CCOCC2)nc2c1CCN2c1cccnc1 ; CC(C)(O)Cc1nc(N2CCOCC2)nc2c1CCN2c1ccccc1 ; c1cncc(N2CCc3c(N4Cc5ccccc5C4)nc(N4CCOCC4)nc32)c1] 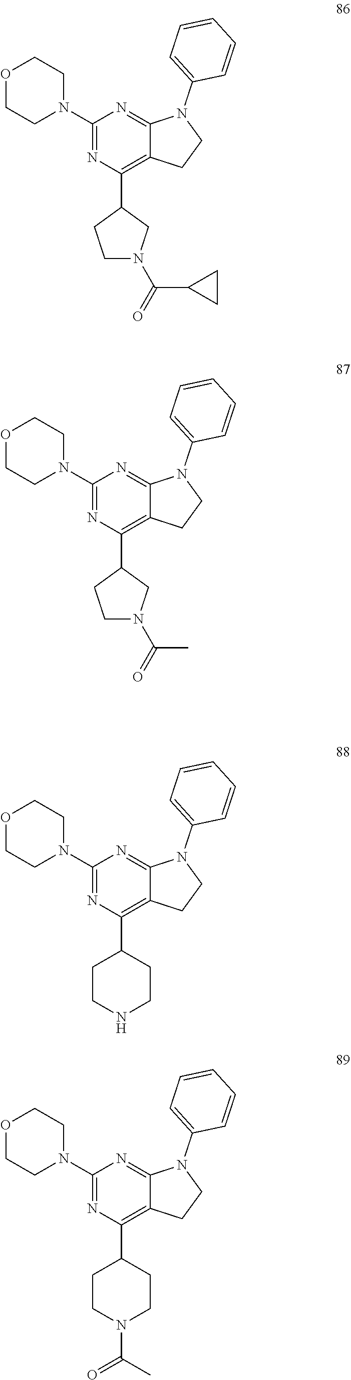 [SMILES: O=C(C1CC1)N1CCC(c2nc(N3CCOCC3)nc3c2CCN3c2ccccc2)C1 ; CC(=O)N1CCC(c2nc(N3CCOCC3)nc3c2CCN3c2ccccc2)C1 ; c1ccc(N2CCc3c(C4CCNCC4)nc(N4CCOCC4)nc32)cc1 ; CC(=O)N1CCC(c2nc(N3CCOCC3)nc3c2CCN3c2ccccc2)CC1] 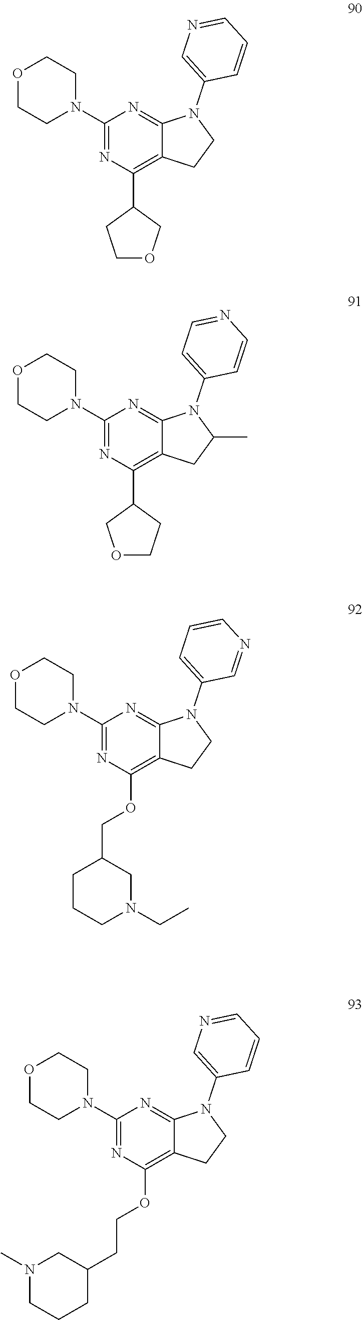 [SMILES: c1cncc(N2CCc3c(C4CCOC4)nc(N4CCOCC4)nc32)c1 ; CC1Cc2c(C3CCOC3)nc(N3CCOCC3)nc2N1c1ccncc1 ; CCN1CCCC(COc2nc(N3CCOCC3)nc3c2CCN3c2cccnc2)C1 ; CN1CCCC(CCOc2nc(N3CCOCC3)nc3c2CCN3c2cccnc2)C1] 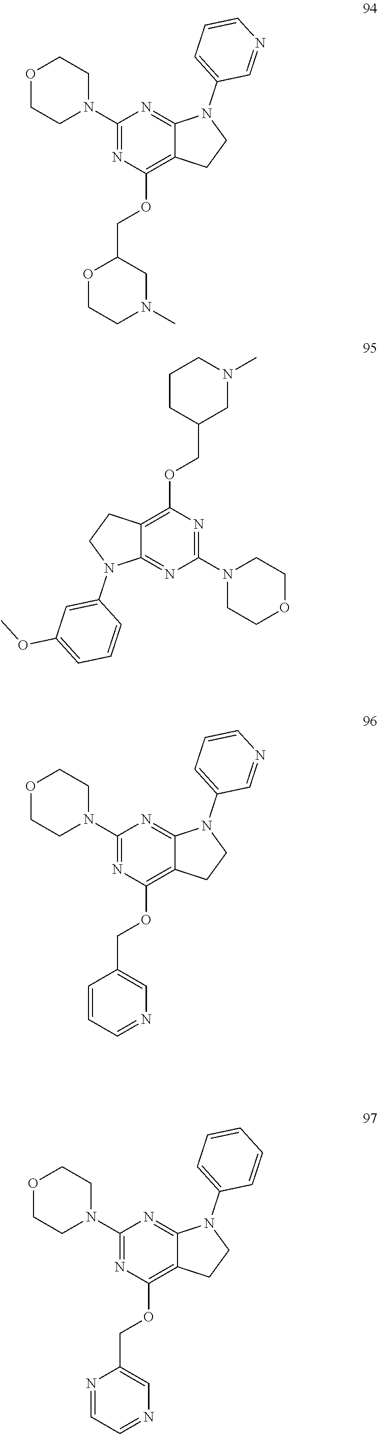 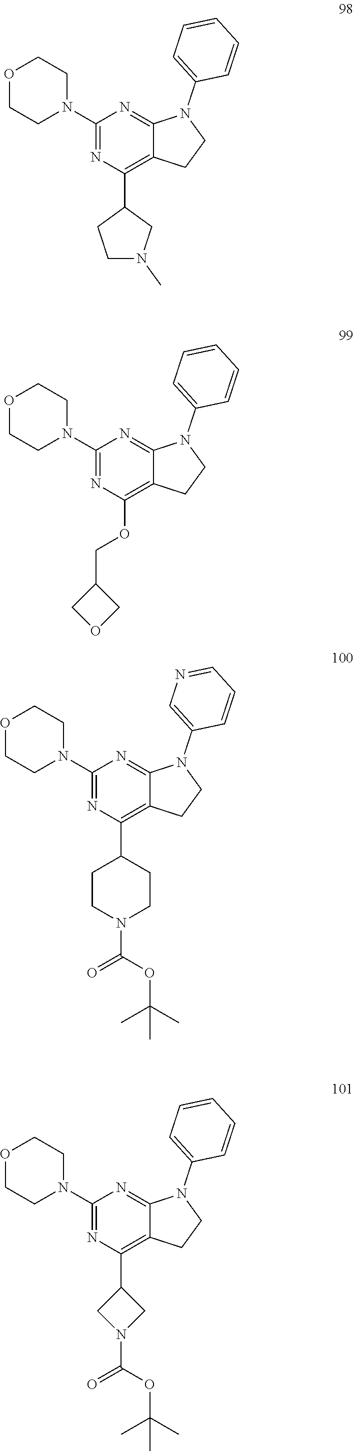 [SMILES: CN1CCC(c2nc(N3CCOCC3)nc3c2CCN3c2ccccc2)C1 ; c1ccc(N2CCc3c(OCC4COC4)nc(N4CCOCC4)nc32)cc1 ; CC(C)(C)OC(=O)N1CCC(c2nc(N3CCOCC3)nc3c2CCN3c2cccnc2)CC1 ; CC(C)(C)OC(=O)N1CC(c2nc(N3CCOCC3)nc3c2CCN3c2ccccc2)C1] 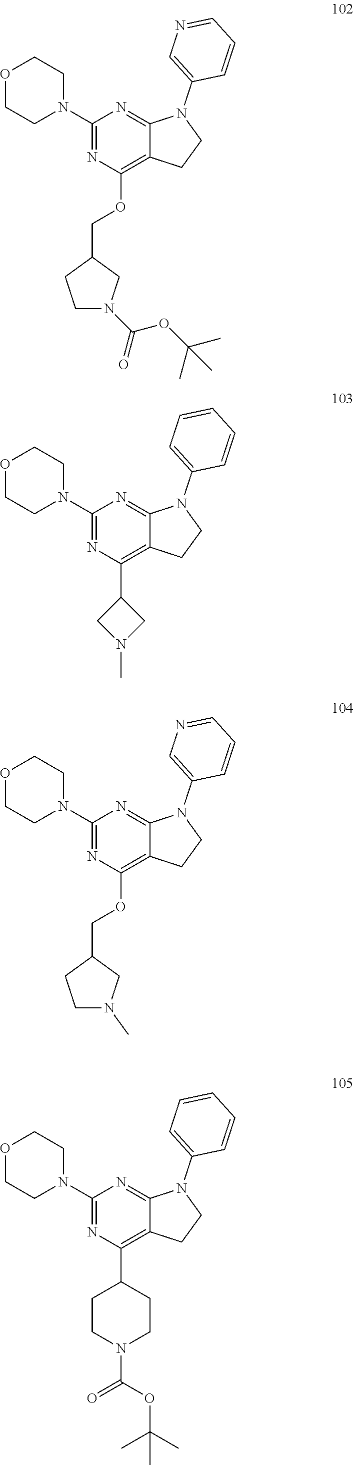 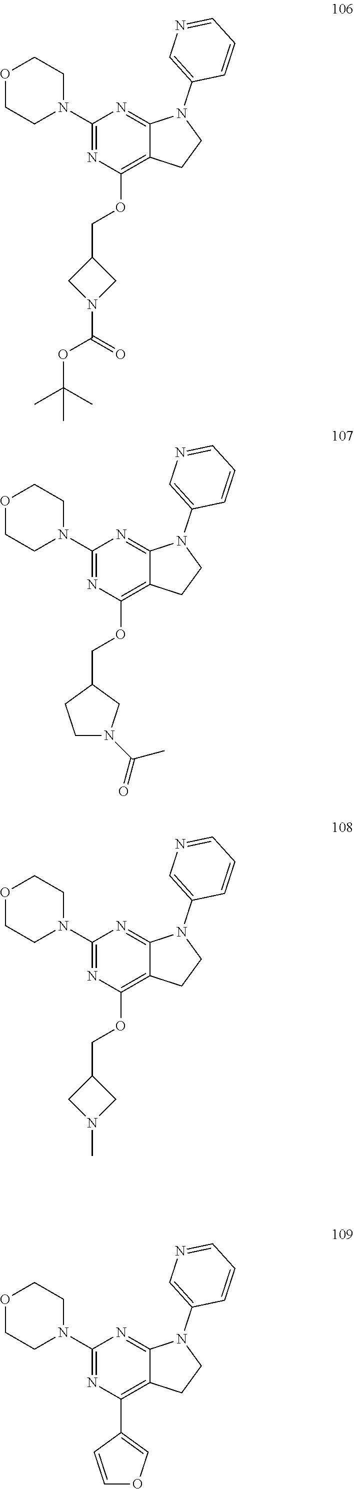 [SMILES: CC(C)(C)OC(=O)N1CC(COc2nc(N3CCOCC3)nc3c2CCN3c2cccnc2)C1 ; CC(=O)N1CCC(COc2nc(N3CCOCC3)nc3c2CCN3c2cccnc2)C1 ; CN1CC(COc2nc(N3CCOCC3)nc3c2CCN3c2cccnc2)C1 ; c1cncc(N2CCc3c(-c4ccoc4)nc(N4CCOCC4)nc32)c1] 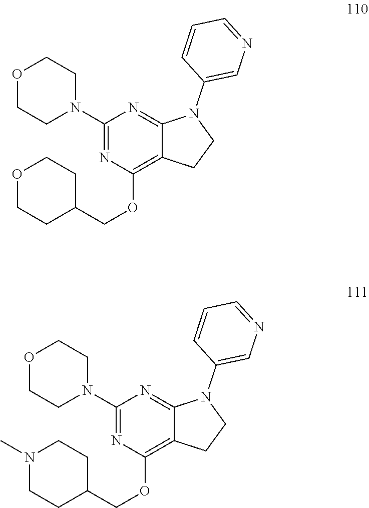 [SMILES: c1cncc(N2CCc3c(OCC4CCOCC4)nc(N4CCOCC4)nc32)c1 ; CN1CCC(COc2nc(N3CCOCC3)nc3c2CCN3c2cccnc2)CC1]
or a pharmaceutically acceptable salt thereof.

29. A pharmaceutical composition comprising the compound of any one of claims 1 to 28, or a pharmaceutically acceptable salt thereof, and a pharmaceutically acceptable excipient.

30. A method of treating a neurological disorder in a subject in need thereof, the method comprising administering to the subject an effective amount of the compound of any one of claims 1 to 28, or a pharmaceutically acceptable salt thereof or the pharmaceutical composition of claim 29.

31. The method of claim 30, wherein the neurological disorder is FTLD-TDP, chronic traumatic encephalopathy, ALS, Alzheimer's disease, LATE, or frontotemporal lobar degeneration.

32. The method of claim 31, wherein the neurological disorder is ALS.

33. A method of inhibiting toxicity in a cell related to a protein, the method comprising contacting the cell with the compound of any one of claims 1 to 28 or a pharmaceutically acceptable salt thereof.

34. The method of claim 33, wherein the toxicity is TDP-43-related toxicity.

35. The method of claim 33, wherein the toxicity is C9orf72-related toxicity.

36. A method of inhibiting PIKfyve in a cell expressing PIKfyve protein, the method comprising contacting the cell with the compound of any one of claims 1 to 28 or a pharmaceutically acceptable salt thereof.

37. The method of any one of claims 33 to 36, wherein the cell is a mammalian neural cell.

38. The method of any one of claims 33 to 37, wherein the cell is in a subject.

39. The method of claim 38, wherein the subject suffers from a neurological disorder.

40. A method of treating a TDP-43-associated disorder in a subject, the method comprising administering to the subject in need thereof an effective amount of the compound of formula (I): 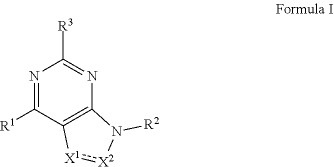
or a pharmaceutically acceptable salt thereof,
wherein [symbol]
is a single bond, X¹ is (C(R^{A})₂)ₘ or —OC(R^{A})₂—R^{X}, and X² is C(R^{A})₂ or CO; or [symbol] is a double bond, and each of X¹ and X² is independently CR^{A} or N, wherein R^{X} is a bond to X²;
R¹ is -(L)ₙ-R^{B}; hydrogen; halogen; cyano; optionally substituted C₁₋₆ alkyl; optionally substituted C₁₋₆ heteroalkyl; optionally substituted C₁₋₆ alkoxy; optionally substituted C₆₋₁₀ aryl, optionally substituted C₁₋₉ heterocyclyl, or optionally substituted C₁₋₉ heteroaryl;
R² is hydrogen, optionally substituted C₁₋₆ alkyl, optionally substituted C₆₋₁₀ aryl, optionally substituted C₁₋₉ heterocyclyl, or optionally substituted C₁₋₉ heteroaryl;
R³ is a group of the following structure: 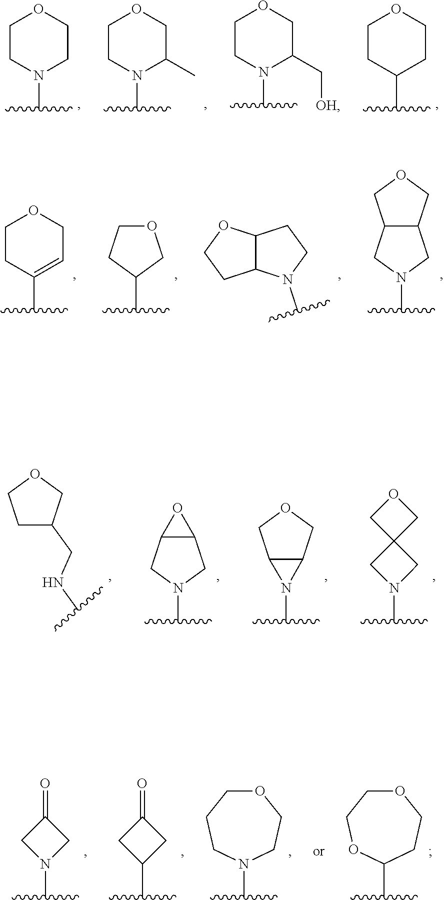
each R^{A} is independently H, optionally substituted C₁₋₆ alkyl, or optionally substituted C₆₋₁₀ aryl;
R^{B} is optionally substituted C₆₋₁₀ aryl, optionally substituted C₁₋₉ heteroaryl, optionally substituted C₃₋₈ cycloalkyl, or optionally substituted C₁₋₉ heterocyclyl;
R^{C} is H or optionally substituted C₁₋₆ alkyl;
each L is independently optionally substituted alkylene, O, or NR^{C}; and
n is 1, 2, or 3; and
m is 0, 1, or 2.

41. The method of claim 40, wherein [symbol] is a single bond.

42. The method of claim 40 or 41, wherein X¹ is (C(R^{A})₂)ₘ.

43. The method of claim 42, wherein m is 1.

44. The method of any one of claims 40 to 43, wherein X² is C(R^{A})₂.

45. The method of any one of claims 40 to 44, wherein each R^{A} is hydrogen.

46. The method of claim 40, wherein the compound is of formula (Ia): 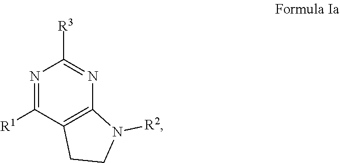
or a pharmaceutically acceptable salt thereof.

47. The method of claim 40, wherein the compound is of formula (Ia′): 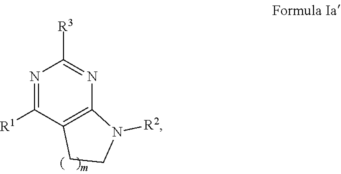
or a pharmaceutically acceptable salt thereof.

48. The method of claim 40, wherein the compound is of formula (Ib): 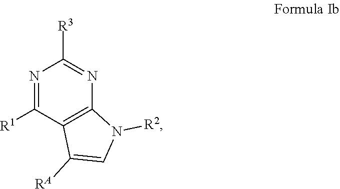
or a pharmaceutically acceptable salt thereof.

49. The method of claim 40, wherein the compound is of formula (Ic): 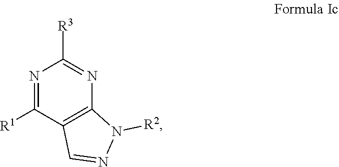
or a pharmaceutically acceptable salt thereof.

50. The method of claim 40, wherein the compound is of formula (Id): 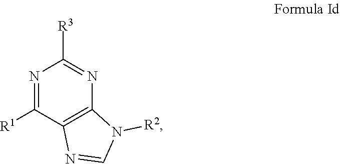
or a pharmaceutically acceptable salt thereof.

51. The method of claim 40, wherein the compound is of formula (Ie): 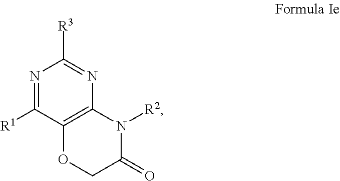
or a pharmaceutically acceptable salt thereof.

52. The method of any one of claims 40 to 51, wherein R¹ is —O-(L)₍ₙ₋₁₎-R^{B}.

53. The method of any one of claims 40 to 52, wherein n is 2.

54. The method of any one of claims 40 to 52, wherein n is 1.

55. The method of any one of claims 40 to 53, wherein at least one L is optionally substituted C₁₋₆ alkylene.

56. The method of claim 55, wherein the optionally substituted C₁₋₆ alkylene is methylene.

57. The method of claim 55, wherein the optionally substituted C₁₋₆ alkylene is ethylene.

58. The method of any one of claims 40 to 57, wherein R^{B} is optionally substituted non-aromatic C₁₋₉ heterocyclyl.

59. The method of any one of claims 40 to 57, wherein R^{B} is optionally substituted C₁₋₉ heteroaryl.

60. The method of any one of claims 40 to 57, wherein R^{B} is optionally substituted C₁₋₆ alkyl.

61. The method of any one of claims 40 to 60, wherein R¹ is: 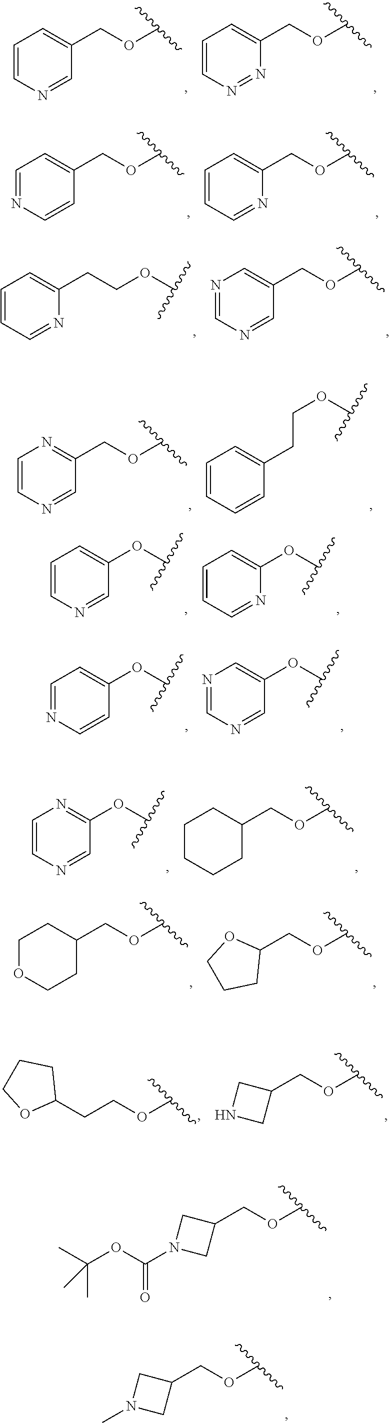 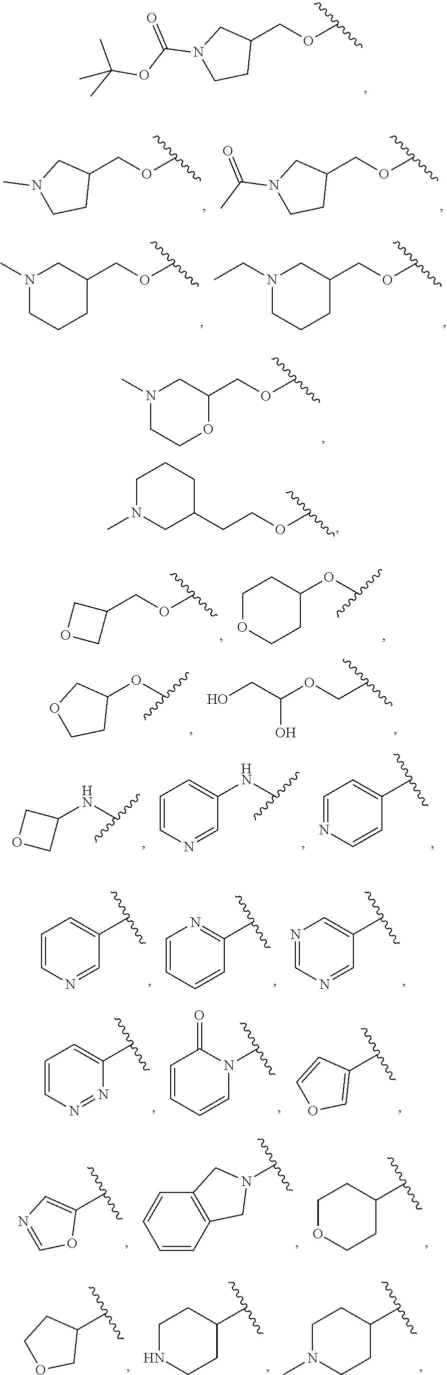 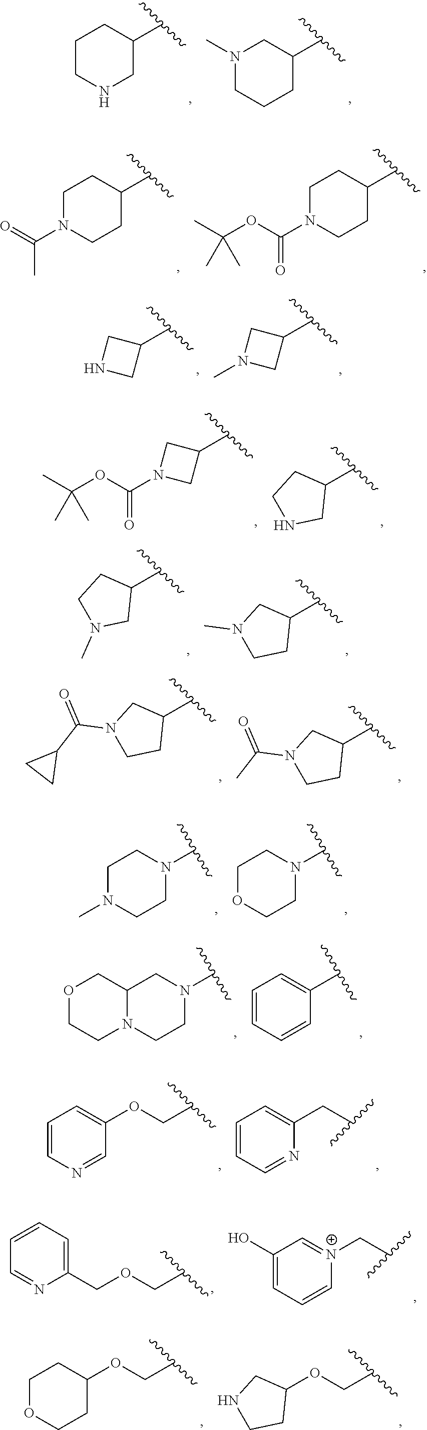 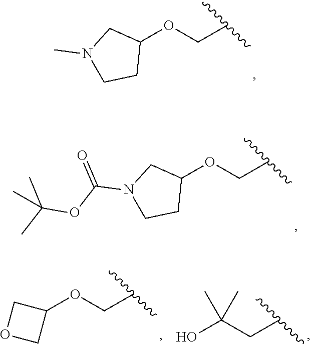
hydrogen, chloro, methyl, cyano, or methoxy.

62. The method of claim 61, wherein R¹ is: 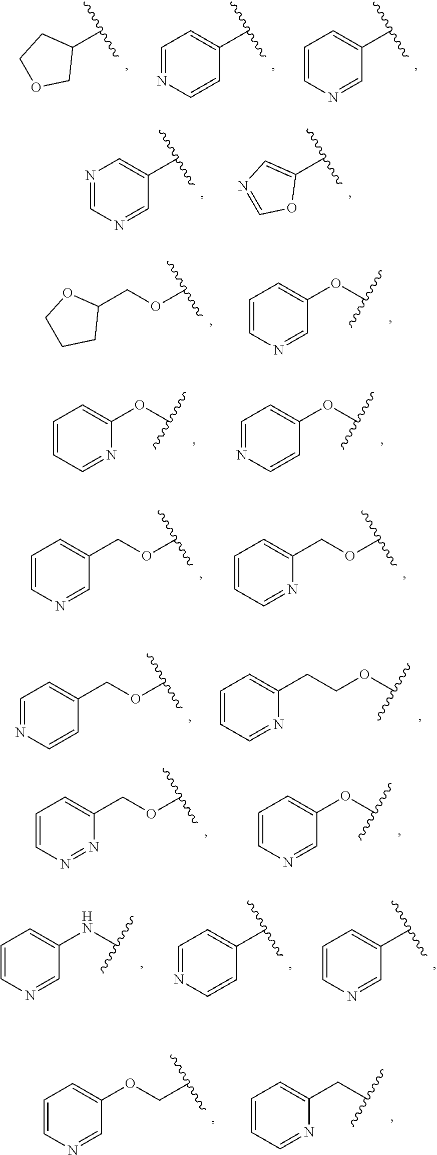
or methoxy.

63. The method of any one of claims 40 to 62, wherein R² is:

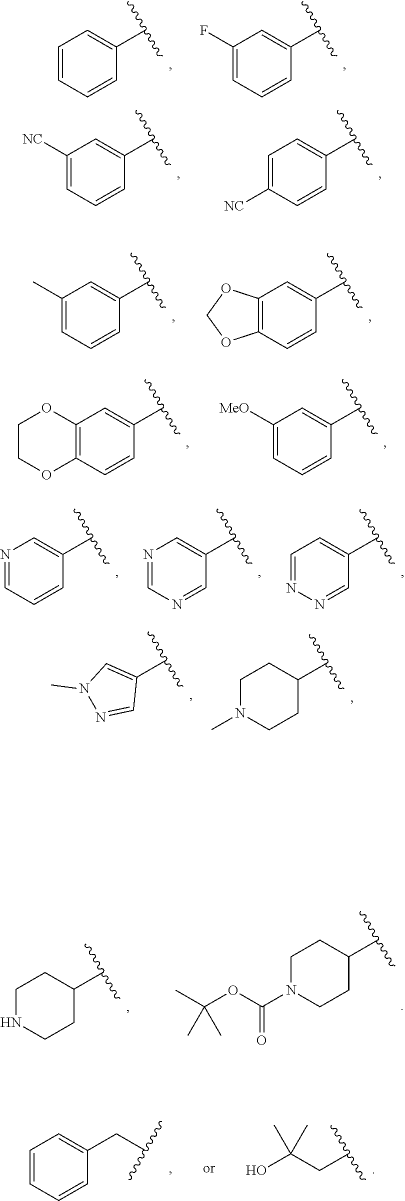

64. The method of claim 51, wherein R² is:

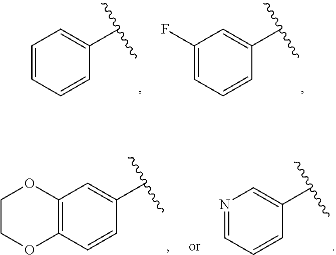

65. The method of any one of claims 40 to 64, wherein R³ is:

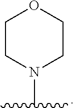 [SMILES: *N1CCOCC1]

66. The method of claim 65, wherein the compound is a compound of claim 27 or 28 or a pharmaceutically acceptable salt thereof.
